(19) United States
(12) Patent Application Publication
(10) Pub. No.: US 2024/0065998 A1
(43) Pub. Date: Feb. 29, 2024

(54) MONOACYLGLYCEROL AND FREE FATTY ACID-BASED COMPOSITIONS, METHODS OF MANUFACTURING AND USE OF THE SAME

(71) Applicant: GLYCOSBIO, INC., Houston, TX (US)

(72) Inventors: Daniel J. MONTICELLO, The Woodlands, TX (US); Nathaniel Scott SCHOCKER, Pearland, TX (US); David Michael STEVENS, Houston, TX (US)

(21) Appl. No.: 18/379,470

(22) Filed: Oct. 12, 2023

Related U.S. Application Data
(63) Continuation of application No. PCT/US2022/024711, filed on Apr. 13, 2022.
(60) Provisional application No. 63/174,487, filed on Apr. 13, 2021.
(60) Provisional application No. 63/174,489, filed on Apr. 13, 2021.
(60) Provisional application No. 63/197,896, filed on Jun. 7, 2021.
(60) Provisional application No. 63/197,905, filed on Jun. 7, 2021.
(60) Provisional application No. 63/292,862, filed on Dec. 22, 2021.

(51) Int. Cl. A61K 31/20; A61K 31/23; A61P 31/04; A61K 9/00; A61K 9/06; A61K 47/18; A61K 47/36; A61K 47/06; A61K 47/10; A61K 47/46; A61K 47/34; A61K 47/12

(52) U.S. Cl. CPC A61K 31/20; A61K 31/23; A61P 31/04; A61K 9/0014; A61K 9/06; A61K 47/18; A61K 47/36; A61K 47/06; A61K 47/10; A61K 47/46; A61K 47/34; A61K 47/12

(57) ABSTRACT

Oil compositions and compositions thereof that can form nanoemulsions or microemulsions are disclosed. Such compositions and compositions thereof can be useful for antimicrobial properties and be used to treat or prevent microbial infections.

### CROSS-REFERENCE TO RELATED APPLICATIONS

This application is a continuation of International Application No. PCT/US2022/024711, filed Apr. 13, 2022, which claims priority to U.S. Provisional Patent Application No. 63/174,487, filed Apr. 13, 2021, U.S. Provisional Patent Application No. 63/174,489, filed Apr. 13, 2021, U.S. Provisional Patent Application No. 63/197,896, filed Jun. 7, 2021, U.S. Provisional Patent Application No. 63/197,905, filed Jun. 7, 2021, and U.S. Provisional Patent Application No. 63/292,862, filed Dec. 22, 2021, each of which is incorporated herein by reference in its entirety.

### BACKGROUND

There are several methods that can create emulsions outside the body. However, these emulsion formulations generally require addition of other agents (e.g., surfactants such as Tween-80 (polysorbate 80)) to stabilize the nanoemulsions. There is an unmet need for emulsion formulations that do not require additional stabilizers, emulsifiers or surfactants and which are stable.

Nanoemulsions (and microemulsions) can be useful as antimicrobial compositions, including for use in preventing biofilm formation. Thus, such nanoemulsions can be useful for human and animal health applications such as treating skin diseases, stomach ulcers, food poisoning, oral and pulmonary diseases, wound care, hospital-acquired infections, catheter contamination, neonatal infection, among others.

The enzymatic hydrolysis of commercial source oils, mainly composed of triglycerides, provides access to MAGs and FFAs and can be achieved by selective use of lipases to render the desired overall ratios of materials, while molecular distillation can be used to refine the individual components. Key ingredients present in source oils such as terpenes, terpenoids, sterols, polyphenols, phytosterols, and the like, may contribute properties to the hydrolyzed triglycerides that can enhance the biological activity relative to normally studied individual MAG and FFA components, such as those from distillation.

There is a need for oil formulations, including from specific oil combinations, that can exhibit antimicrobial activity against microbial species.

### SUMMARY

The present disclosure is directed to oil compositions that can include an enzyme-modified oil (EMO) and/or a free fatty acid (FFA) oil and which can have antimicrobial properties and be used to formulate antimicrobial compositions. The oil compositions of the present disclosure can be used in any formulation, and can also be used to formulate nanoemulsions and microemulsions that contain free fatty acids and, optionally, monoacylglycerols (MAGs) and/or diacylglycerols (DAGs), and that do not require additional agents to stabilize the emulsions. Methods for making the same are also provided in addition to methods of of using the antimicrobial compositions for specific antimicrobial applications and treatments.

In some embodiments, an antimicrobial oil composition is provided that can include a first component present in a first amount, where the first component is an enzyme-modified oil (EMO) derived from a first oil source, and where the oil composition comprises 80% or less triacylglycerols (TAGs).

In any of the foregoing embodiments, the oil composition can further include a second component present in a second amount, where the second component is a free fatty acid (FFA) oil. It should be understood that the first oil source and the second oil source can be the same type of oil or different types of oil and that each of the first oil source and the second oil source can be a single type of oil or a combination of oils as described further herein. Alternatively, the antimicrobial oil composition can include a FFA oil as described herein but not an EMO.

In some embodiments, an antimicrobial oil composition can include at least 10% free fatty acids (FFAs) by weight out of the total weight of the antimicrobial oil composition and at least 5% monoacylglycerols (MAGs) by weight out of the total weight of the antimicrobial oil composition, where the antimicrobial oil composition comprises less than 80% triacylglycerols (TAGs) by weight out of the total weight of the antimicrobial oil composition.

In some embodiments, an method for treating or preventing a microbial infection in a subject in need thereof can include administering to the subject a therapeutically effective amount of an antimicrobial oil composition, nanoemulsion, microemulsion or antimicrobial composition comprising any of an antimicrobial oil composition, nanoemulsion or microemulsion.

In some embodiments, a composition is provided that includes an oil dispersed in a liquid, where the oil is in the form of droplets or particles and includes at least 2% free fatty acids (FFAs) by weight of the oil, where the FFAs include two or more different fatty acids, where the oil includes 80% or less triacylglycerols (TAGs) by weight out of the total weight of the oil, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% FFAs by weight of the oil and 80% or less TAGs by weight of the oil, where the oil includes non-oil ingredients that are naturally present in the oil, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) and diacylglycerols (DAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs and DAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In some embodiments, a method for preparing a nanoemulsion is provided that includes providing an oil of the present disclosure, providing a polar, liquid component, combining the oil and polar, liquid component to form a nanoemulsion pre-mix, and forming a nanoemulsion from the nanoemulsion pre-mix, where the nanoemulsion comprises an oil phase and a polar, liquid phase, where the oil phase is dispersed as droplets or particles within the polar, liquid phase, and where the droplets or particles have a Z average size of about 300 nm or less as measured by dynamic light scattering.

In some embodiments, a composition is provided that includes an oil dispersed in a liquid, where the oil is in the form of droplets or particles and includes at least 2% free fatty acids (FFAs) by weight of the oil, where the FFAs include two or more different fatty acids, where the oil includes 80% or less triacylglycerols (TAGs) by weight out of the total weight of the oil, and where the droplets or particles have a Z average size of greater than about 300 nm as measured by dynamic light scattering.

In some embodiments, a composition is provided that includes an oil dispersed in a liquid, where the oil is in the form of droplets or particles and includes at least 2% FFAs by weight of the oil and 80% or less TAGs by weight of the oil, where the oil includes non-oil ingredients that are naturally present in the oil, and where the droplets or particles have a Z average particle size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) in an amount of at least 5% by weight out of the total weight of the oil, where the MAGs and FFAs are derived from a common, first oil source, and where the droplets or particles have a Z average particle size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs in an amount of at least 5% by weight of the total weight of the oil, where the MAGs and FFAs are derived from a first oil source and a second oil source, respectively, and where the droplets or particles have a Z average size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) and diacylglycerols (DAGs) in an amount of at least 5% by weight out of the total weight of the oil, where the MAGs and FFAs are derived from a common, first oil source, and where the droplets or particles have a Z average particle size of greater than about 300 nm as measured by dynamic light scattering.

In some embodiments, a composition is provided that includes an oil dispersed in a liquid, where the oil is in the form of droplets or particles and includes at least 2% FFAs by weight of the oil, where the FFAs include two or more different fatty acids, where the oil also includes MAGs and DAGs in an amount of at least 5% by weight of the total weight of the oil, where the MAGs and FFAs are derived from a first oil source and a second oil source, respectively, and where the droplets or particles have a Z average size of greater than about 300 nm as measured by dynamic light scattering.

In some embodiments, a method for preparing a nanoemulsion or microemulsion is provided that includes providing an oil of the present disclosure, providing a polar, liquid component, combining the oil and polar, liquid component to form a nanoemulsion pre-mix, and forming a nanoemulsion from the nanoemulsion pre-mix, where the nanoemulsion comprises an oil phase and a polar, liquid phase, where the oil phase is dispersed as droplets or particles within the polar, liquid phase, and where the droplets or particles have a Z average size of greater than about 300 nm as measured by dynamic light scattering.

In some embodiments, an antimicrobial composition comprising a composition of the present disclosure is provided.

In some embodiments, a method for treating or preventing a microbial infection or disease or condition caused by a microbial infection in a patient in need thereof can include administering an effective amount of a composition of the present disclosure or an antimicrobial composition comprising an effective amount of a composition of the present disclosure.

In some embodiments, a method for sanitizing or sterilizing an object or surface can include the step of applying an effective amount composition of the present disclosure to the object or surface. In such embodiments, the method can include applying an effective amount of the composition to the object or surface. It should be understood that the effective amount can be the MIC, MBC or any amount suitable to yield a reduction in growth, kill a sufficient amount or inhibit growth of a microbial target. It should be understood that the application can be for a period of time sufficient to exert the desired effect, such as a desired LVR, inhibition or bacterial killing.

In some embodiments, a method for biofilm eradication in a gas or oil pipeline can include the step of flowing a composition according to the present disclosure through the gas or oil pipeline.

In some embodiments a nebulizer, vaping device, bandage, gauze, suture, plastic device such as a catheter or IV port, floss, or a condom can include an antimicrobial composition, including a nanoemulsion or microemulsion, of the present disclosure.

### BRIEF DESCRIPTION OF THE DRAWINGS

FIG. 1 depicts the particle size distribution as measured by dynamic light scattering for Example 2.

FIG. 2A depicts the Z average particle size as measured by dynamic light scattering for flaxseed EMO nanoemulsion samples over time, stored at room temperature (data are represented as average±SD from at least three measurements).

FIG. 2B depicts the Z average particle size as measured by dynamic light scattering for sesame EMO nanoemulsion samples over time, stored at room temperature (data are represented as average±SD from at least three measurements; formulations that precipitated were assigned a Z average size of 1000 nm).

FIG. 2C depicts the Z average particle size as measured by dynamic light scattering for coconut EMO/medium chain triglyceride (MCT) FFA oil nanoemulsion samples over time, stored at room temperature (data are represented as average±SD from at least three measurements; formulations that precipitated were assigned a Z average size of 1000 nm).

FIG. 2D depicts the Z average particle size as measured by dynamic light scattering for MCT EMO/coconut FFA oil nanoemulsion samples over time, stored at room temperature (data are represented as average±SD from at least three measurements; formulations that precipitated were assigned a Z average size of 1000 nm).

FIG. 2E depicts the Z average particle size as measured by dynamic light scattering for fish EMO/flaxseed FFA oil nanoemulsion samples over time, stored at room temperature (data are represented as average±SD from at least three measurements; formulations that precipitated were assigned a Z average size of 1000 nm).

FIG. 2F depicts the Z average particle size as measured by dynamic light scattering for flaxseed EMO/fish FFA oil nanoemulsion samples over time, stored at room temperature (data are represented as average±SD from at least three measurements; formulations that precipitated were assigned a Z average size of 1000 nm).

FIG. 2G depicts the Z average particle size as measured by dynamic light scattering for fish EMO/coconut FFA oil nanoemulsion samples over time, stored at room temperature (data are represented as average±SD from at least three measurements; formulations that precipitated were assigned a Z average size of 1000 nm).

FIG. 2H depicts the Z average particle size as measured by dynamic light scattering for flaxseed EMO/coconut FFA oil nanoemulsion samples over time, stored at room temperature (data are represented as average±SD from at least three measurements; formulations that precipitated were assigned a Z average size of 1000 nm).

FIG. 2I depicts the Z average particle size as measured by dynamic light scattering for coconut EMO/flaxseed FFA oil nanoemulsion samples over time, stored at room temperature (data are represented as average±SD from at least three measurements; formulations that precipitated were assigned a Z average size of 1000 nm).

FIG. 2J depicts the Z average particle size as measured by dynamic light scattering for MCT EMO oil nanoemulsion samples over time, stored at room temperature (data are represented as average±SD from at least three measurements; formulations that precipitated were assigned a Z average size of 1000 nm).

FIG. 2K depicts the Z average particle size as measured by dynamic light scattering for olive oil EMO nanoemulsion samples over time, stored at room temperature (data are represented as average±SD from at least three measurements; formulations that precipitated were assigned a Z average size of 1000 nm).

FIG. 2L depicts the Z average particle size as measured by dynamic light scattering for rosehip EMO/coconut FFA oil nanoemulsion samples over time, stored at room temperature (data are represented as average±SD from at least three measurements; formulations that precipitated were assigned a Z average size of 1000 nm).

FIG. 2M depicts the Z average particle size as measured by dynamic light scattering for coconut EMO/rosehip FFA oil nanoemulsion samples over time, stored at room temperature (data are represented as average±SD from at least three measurements; formulations that precipitated were assigned a Z average size of 1000 nm).

FIG. 2N depicts the Z average particle size as measured by dynamic light scattering for rosehip EMO/flaxseed FFA oil nanoemulsion samples over time, stored at room temperature (data are represented as average±SD from at least three measurements; formulations that precipitated were assigned a Z average size of 1000 nm).

FIG. 2O depicts the Z average particle size as measured by dynamic light scattering for flaxseed EMO/rosehip FFA oil nanoemulsion samples over time, stored at room temperature (data are represented as average±SD from at least three measurements; formulations that precipitated were assigned a Z average size of 1000 nm).

FIG. 2P depicts the Z average particle size as measured by dynamic light scattering for rosehip EMO nanoemulsion samples over time, stored at room temperature (data are represented as average±SD from at least three measurements; formulations that precipitated were assigned a Z average size of 1000 nm).

FIG. 2Q depicts the Z average particle size as measured by dynamic light scattering for hemp seed EMO nanoemulsion samples over time, stored at room temperature (data are represented as average±SD from at least three measurements; formulations that precipitated were assigned a Z average size of 1000 nm).

FIG. 2R depicts the Z average particle size as measured by dynamic light scattering for algae EMO/flaxseed FFA oil nanoemulsion samples over time, stored at room temperature (data are represented as average±SD from at least three measurements; formulations that precipitated were assigned a Z average size of 1000 nm).

FIG. 2S depicts the Z average particle size as measured by dynamic light scattering for algae EMO/MCT FFA oil nanoemulsion samples over time, stored at room temperature (data are represented as average±SD from at least three measurements; formulations that precipitated were assigned a Z average size of 1000 nm).

FIG. 3A depicts growth curves for B. cereus culture without or with nCo-50 at various concentrations.

FIG. 3B depicts growth curves for S. mutans culture without or with nF/Co-50 at various concentrations.

FIG. 3C depicts growth curves for S. epidermidis culture without or with nCo/Fx-75 at various concentrations.

FIG. 3D depicts growth curves for E. faecalis culture without or with nFx-25 at various concentrations.

FIG. 3E depicts growth curves for L. monocytogenes culture without or with nF/Co-50 at various concentrations.

FIG. 3F depicts growth curves for S. pyogenes culture without or with nFx/R-50 at various concentrations.

FIG. 3G depicts growth curves for S. aureus culture without or with nOa/Fx-25 at various concentrations.

FIG. 3H depicts growth curves for S. agalactiae culture without or with nF/Fx-25 at various concentrations.

FIG. 4 depicts the bactericidal effects of nFx/R-50 on S. pyogenes and nCo/Fx-25 on S. aureus and C. acnes.

FIG. 5 depicts the bactericidal effects of nCo/Fx-25 and nFx/R-50 on S. aureus and S. pyogenes, respectively.

FIG. 6A depicts the bactericidal effects of nCo/Fx-25 on S. aureus, S. capitis and S. epidermidis at pH 6.0.

FIG. 6B depicts the bactericidal effects of nCo/Fx-25 on S. aureus, S. capitis and S. epidermidis at pH 5.0.

FIG. 7 depicts the effects of nF/Co-50, nFx-Co-25 and nCo/Fx-25 on S. aureus biofilm formation.

FIG. 8A depicts the effect of nCo/Fx-25 on membrane permeability in S. aureus.

FIG. 8B depicts the effect of nCo/Fx-25 on membrane depolarization in S. aureus.

FIG. 9A depicts the effect of a triglyceride nanoemulsion on bacterial growth of S. aureus.

FIG. 9B depicts the effect of an oat EMO/FFA oil microemulsion on bacterial growth of S. aureus.

FIG. 9C depicts the effect of nOa-50 on bacterial growth of S. aureus.

FIG. 9D depicts the effect of nOA/Fx-25 on bacterial growth of S. aureus.

FIG. 10A depicts the effect of a triglyceride nanoemulsion on growth of S. pyogenes.

FIG. 10B depicts the effect of an oat EMO/FFA oil microemulsion on bacterial growth of S. pyogenes.

FIG. 10C depicts the effect of an nOa-50 on bacterial growth of S. pyogenes.

FIG. 10D depicts the effect of nOA/Fx-25 on bacterial growth of S. pyogenes.

FIG. 11A depicts the bactericidal effect of triglyceride nanoemulsions and the corresponding micro- or nano-emulsions of the present disclosure on C. acnes.

FIG. 11B depicts the bactericidal effect of triglyceride nanoemulsions and the corresponding micro- or nano-emulsions of the present disclosure on C. acnes.

FIG. 12A depicts a nanoemulsion library screen against C. acnes.

FIG. 12B depicts a nanoemulsion library screen against H. pylori.

FIG. 13 depicts the effect of nAg/Fx-25 on growth of P. aeruginosa.

FIG. 14 depicts the effect of several nanoemulsions against M. luteus and C. xerosis.

FIG. 15 depicts the bactericidal effect of nanoemulsions and ACv14 on S. aureus strains and S. epidermidis.

FIG. 16A depicts the size of nanoemulsions supplemented with S2, S20 or glyceryl stearate.

FIG. 16B depicts the PDI of nanoemulsions supplemented with S2, S20 or glyceryl stearate.

FIG. 17A depicts the size of nanoemulsions supplemented with S2.

FIG. 17B depicts the PDI of nanoemulsions supplemented with S2.

FIG. 18 depicts the bactericidal effect of ACv14 on S. aureus.

FIG. 19A depicts the bactericidal effect of macadamia nut versus oat nanoemulsions on S. aureus.

FIG. 19B depicts the bactericidal effect of macadamia nut versus oat nanoemulsions on S. epidermidis.

FIG. 20 depicts the bactericidal effect of OL1-OL4 with pre-formed nanoemulsions or with EMO/FFA oil addition on S. aureus.

FIG. 21A depicts the bactericidal effect of OL5 and OLv5 with nanoemulsions on S. aureus.

FIG. 21B depicts the bactericidal effect of OL5 and OLv5 with nanoemulsions on other bacterial strains.

FIG. 22A depicts the bactericidal effect of cream supplemented with EMO/FFA oil on S. aureus.

FIG. 22B depicts the bactericidal effect of cream supplemented with EMO/FFA oil on S. aureus and commensal strains.

FIG. 23 depicts the virucidal effect of nanoemulsions on respiratory syncytial virus.

FIG. 24 depicts the virucidal effect of nanoemulsions on H1N1 influenza virus.

FIG. 25 depicts the virucidal effect of nanoemulsions on herpes simplex virus.

FIG. 26 depicts the virucidal effect of ACv14 on Planktonic, sedimentary or plate grown S. aureus.

FIG. 27 depicts the results of a standard bactericidal assay using OL5 with or without additional nEMO supplementation (nOA/Fx-25) against a MRSA isolate from PathEx and a S. aureus isolate from a volunteer participant.

FIG. 28 depicts the results of a standard bactericidal assay on different bacterial species.

FIG. 29 depicts the results of a standard bactericidal assay on E. coli and P. aeruginosa.

FIG. 30 depicts the results of a standard bactericidal assay on S. aureus ATCC 29213.

FIG. 31A depicts the results of a standard bactericidal assay as measured by qPCR on S. aureus.

FIG. 31B depicts the results of a standard bactericidal assay as measured by qPCR on S. epidermidis.

FIG. 32A depicts the results of a standard bactericidal assay on S. mutans.

FIG. 32B depicts the results of a standard bactericidal assay on Streptococcus strains.

FIG. 32C depicts the results of a standard bactericidal assay on Lactobacillus acidophilus.

FIG. 32D depicts the results of a standard bactericidal assay on S. mutans.

FIG. 33 depicts the results of a standard bactericidal assay on S. mutans.

FIG. 34A depicts the results of a standard bactericidal assay on S. pyogenes.

FIG. 34B depicts plate growth of S. pyogenes.

FIG. 34C depicts amplification of S. pyogenes from samples.

FIG. 35 depicts the results of a bactericidal assay on E. coli and P. aeruginosa.

FIG. 36 depicts the results of a bactericidal assay on Staphylococcus strains and C. acnes.

FIG. 37 depicts measurement of non-oil ingredients in unmodified canola oil and canola EMO.

### DETAILED DESCRIPTION

The present disclosure is directed to oil compositions that can include an enzyme-modified oil (EMO) and/or a free fatty acid (FFA) oil and which can have antimicrobial properties and be used as or to formulate antimicrobial compositions. The oil compositions of the present disclosure can be used in any formulation, but can also be used to formulate nanoemulsions and microemulsions that contain free fatty acids and, optionally, monoacylglycerols (MAGs) and/or diacylglycerols (DAGs), and that do not require additional agents to stabilize the nanoemulsions. Methods for making the same are also provided in addition to methods of of using the antimicrobial compositions for specific antimicrobial applications and treatments.

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### Definitions

As used herein, “nanoemulsion” refers to a colloidal particulate system consisting of two immiscible liquids, or a liquid and a solid, in which one liquid or the solid (dispersed phase) is dispersed as a droplet or particle, respectively, into the other liquid (continuous phase). A “nanoemulsion” can include droplets or particles having a size from 10 to 1000 nm, but generally nanoemulsions must have small droplet sizes (<300 nm) in order to be kinetically stable. The dispersed phase is also known as the discontinuous phase while the outer phase can be called the continuous phase.

As used herein, “microemulsion” refers to a colloidal particulate system consisting of two immiscible liquids, or a liquid and a solid, in which one liquid or the solid (dispersed phase) is dispersed as a droplet or particle, respective into the other liquid (continuous phase). A “microemulsion” can include droplets or particles have a size greater than 1000 nm to 1000000 nm.

As used herein, “free fatty acid (FFA)” refers to a non-esterified fatty acid in its carboxylic acid form or carboxylate salt form.

As used herein, a “monoacylglycerol (MAG),” also known as a monoglyceride, is a glyceride consisting of one fatty acid chain covalently bonded to a glycerol molecule through an ester linkage.

As used herein, a “diacylglycerol (DAG),” also known as a diglyceride, is a glyceride consisting of two fatty acid chains covalently bonded to a glycerol molecule through ester linkages.

As used herein, a “triacylglycerol (TAG),” also known as a triglyceride, is a glyceride consisting of three fatty acid chains covalently bonded to a glycerol molecule through ester linkages. TAGs may also be classified as having a long or medium chain length. Long chain TAGs contain fatty acids with 14 or more carbons, while medium chain TAGs contain fatty acids with 6 to 12 carbons. Long chain TAGs can include omega-3 and omega-6 fatty acids. Medium chain TAGs have saturated fatty acids and thus do not contain omega-3 or omega-6 fatty acids. Long chain TAGs (LCT) and medium chain triglycerides (MCT) can serve as energy sources.

As used herein, “exogenous additive with surfactant and/or emulsifier properties” refers to a compound acting as a surfactant or emulsifier that is added to an oil but not naturally present in the oil in an amount sufficient to enhance droplet or particle formation and stability in a nanoemulsion. Non-limiting examples of surfactants 4-(5-dodecyl)benzenesulfonate, docusate (dioctyl sodium sulfosuccinate), alkyl ether phosphates, benzalkaonium chloride (BAC), and perfluorooctanesulfonate (PFOS), Non-limiting examples of emulsifiers include ethoxylated alcohols, carboxylates, sodium isothionate, cetyl alcohol, stearyl alcohol, and silicone emulsifiers such as dimethicones

As used herein, “exogenous additive with thickening or crystallization inhibiting properties” refers to a thickener or crystallization inhibitor that is added to an oil but not naturally present in the oil in an amount sufficient to thicken a nanoemulsion or inhibit crystallization, respectively. By way of example, but not limitation, such exogenous additives with thickening properties can include methyl cellulose, cornstarch, sodium alginate and gelatin. By way of example, but not limitation, such exogenous additives with crystallization inhibiting properties can include polyglycerol fatty acid esters, sucrose fatty acid esters, and sorbitan fatty acid esters.

As used herein, “surfactant” or “emulsifier” refers to a substance that reduces the surface tension between two liquids or, in the case of a solid lipid nanoemulsion, between the liquid phase and the solid lipid phase. Surfactants, or surface-active agents, are compounds that lower the surface tension between two liquids or between a liquid and a solid. An emulsifier is a surfactant that stabilizes emulsions. Emulsifiers coat droplets within an emulsion and prevent them from coming together, or coalescing.

Surfactants are commonly used as emulsifiers as they are known to stabilize emulsions. By way of example, but not limitation, surfactants (and emulsifiers) can include polysorbates, phospholipids, sterols, cationic lipids, poloxamers, sorbitan esters, sugar esters, polyoxyethylene conjugates, ammonium phosphatidyl lapsidate, polyphenols, polyvinylalcohol, lecithin, ethanol, lysophospholipids, ceramides, Tweens, Spans, Kolliphor, propylene glycol, vitamin D, polyethylene glycol, polyethylene glycol derivatives, pectin, gum acacia, modified gum acacia, agar, ghatti gum, modified ghatti gum, pectin, carrageenan, xanthan gum, modified starches, modified alginate, fatty alcohols, and ethoxylated polyols.

As used herein, “enzyme-modified oil (EMO)” refers to an oil that includes MAGs and, optionally DAGs and/or FFA, that results from enzymatic hydrolysis of a starting oil source(s) to yield FFA and glycerol followed by separation of the glycerol, and at least partial enzymatic re-esterification of glycerol with the FFA to yield MAGs and, optionally some DAGs, and, in some instances, some residual FFAs. Unless otherwise stated herein, an EMO should be understood to be substantially free of TAGs (below a detectable level). In some embodiments, an EMO can have less than 5% TAGs.

As used herein, “free fatty acid oil (FFA oil)” refers to an oil that includes free fatty acids that result from enzymatic hydrolysis of TAGs in a starting oil source(s) to FFA and glycerol, with subsequent removal of the glycerol. It should be understood that the FFA oil can include some glycerol which may be dissolved in the FFA oil.

Methods for producing EMO and FFA oil, and properties thereof, are disclosed in International Patent Application Nos. PCT/US2018/055583, filed Oct. 12, 2018, published as WO 2019/075307 and PCT/US2020/014182, filed Jan. 17, 2020, published as WO 2020/150661, and U.S. Patent Application Publication No. 2020/0146307 (U.S. Ser. No. 16/746,700), each of which is incorporated herein by reference in its entirety.

As used herein, “non-oil ingredient” is an ingredient that is naturally present in an oil source that is not a MAG, DAG, TAG, or FFA.

As used herein, “effective amount” refers to an amount of a composition that is sufficient to bring about an intended result. By way of example, but not limitation, an effective amount can be an amount effective to delay the growth of, reduce, inhibit or kill a microbe. The specific effective amount will vary with such factors as the microbe being treated, the duration of the treatment, and the structure of the composition.

As used herein, a “therapeutically effective amount” refers to an amount of a composition of the present disclosure effective yield a desired therapeutic response. By way of example, but not limitation, an amount effective to delay the growth of, reduce growth, inhibit or kill a microbe or reduce severity of an skin condition or infection can be a “therapeutically effective amount.” The specific therapeutically effective amount will vary with such factors as the particular condition being treated, the physical condition of the subject, the type of subject being treated, the duration of the treatment, the nature of concurrent therapy (if any), and the specific formulations employed and the structure of the composition.

As used herein, a “cream” can be understood to be an emulsion of an oil phase and an aqueous phase.

Two critical factors can determine the potential success of emulsion production and stability—interfacial tension and repulsion by electric charge.

The surface tension of the fluids determines their propensity to form emulsions. The surface tension can be lowered by adding surfactants. Most surfactants are amphiphilic compounds. The emulsifying agents can concentrate at the oil-water interface, producing a significant reduction of the interfacial tension and consequently significantly reduce the energy to form emulsions. Despite a lowering of interfacial tension when surface-active agents are added, the free energy of the interface remains positive, leaving a persisting state of thermodynamic instability.

The stability of an emulsion can be improved by adding emulsifiers. Emulsifiers frequently have a hydrophobic end (e.g. a long chain hydrocarbon) and a charged end (for example a carboxylic acid). Emulsifiers coat the surface of the droplets or particles. Emulsion stability is often explained by the presence of repulsive electrical charges on the surfaces of emulsion droplets. If the magnitude of this energy barrier exceeds the kinetic energy of the particles, the suspension is stable. ζ-potential (“zeta potential”) is a typical measure of repulsion. Generally, a zeta potential of +/−30 mV or more ensures good stability.

The size of the nanoemulsion discontinuous phase particles/droplets can typically be measured using Dynamic Light Scattering (DLS). DLS is a non-invasive, well-established technique for measuring the size and size distribution of molecules and particles typically in the submicron region. Brownian motion of particles or molecules in suspension causes laser light to be scattered at different intensities, varying over time because of constructive and destructive interference. Analysis of these intensity fluctuations yields the underlying diffusion coefficient and, hence, the particle size using the Stokes-Einstein relationship. A typical results can include: (1) a mean or “Z average size”, i.e. an overall average size defined via a simple cumulant fit; (2) a polydispersity index (PDI), i.e. an indication of how broad a (forced) single Gaussian peak would have to be to fit the result (0.0 is perfectly monodisperse, 1.0 is extremely polydisperse); or (3) a size distribution obtained from a multipeak analysis with the mean peak size and standard deviation for each peak indicated, as well as the relative signal intensity (% intensity) originating from the species. Sizes can generally be expressed as “diameter in nanometers” (d, nm).

Zeta potential can be defined as the potential difference between the dispersion medium and the stationary layer of fluid attached to the particle and can be measured by Electrophoretic Light Scattering (ELS). ELS is a technique that can be used to measure the electrophoretic mobility of particles in dispersion, or molecules in solution. This mobility is often converted to zeta potential to enable comparison of materials under different experimental condition. The concept is similar to DLS, yet the movement and intensity fluctuations are studied under the influence of an applied electric field. The fundamental physical principle is that of electrophoresis. A dispersion is introduced into a cell containing two electrodes. An electrical field is then applied to the electrodes, and particles or molecules that have a net charge will migrate towards the oppositely charged electrode with a velocity, known as the mobility, that is related to their zeta potential. A typical result can include: (1) zeta potential, i.e. the overall average zeta potential of the dispersed, e.g. oil, particles in the sample; or (2) conductivity, the electrophoretic mobility is often also reported in mS/cm.

Antimicrobial Oil Compositions

In some embodiments, an antimicrobial oil composition can include at least 10% free fatty acids (FFAs) by weight out of the total weight of the antimicrobial oil composition and at least 5% monoacylglycerols (MAGs) by weight out of the total weight of the antimicrobial oil composition, where the antimicrobial oil composition comprises less than 80% triacylglycerols (TAGs) by weight out of the total weight of the antimicrobial oil composition. In certain aspects, the antimicrobial oil composition can can include a first amount of a first component and a second amount of a second component, where the first component is an enzyme-modified oil (EMO) derived from a first oil source and the second component is a FFA oil derived from a second oil source. Thus, in certain aspects, the MAGs can be derived from the EMO and the FFA can be derived from the EMO and the FFA oil. In other aspects, the antimicrobial composition can include an EMO but no FFA oil. Alternatively, the antimicrobial oil composition can include a FFA oil but no EMO. In such alternative embodiments, the antimicrobial composition does not include MAGs from the EMO and can, in certain aspects, not include MAGs.

In some embodiments, an antimicrobial oil composition can include a first amount of a first component and a second amount of a second component, where the first component is an EMO derived from a first oil source and the second component is a FFA oil derived from a second oil source, where the antimicrobial composition comprises less than 80% TAGs by weight out of the total weight of the antimicrobial composition. It should be understood that, in some embodiments, the antimicrobial composition can include either an EMO, a FFA oil or both.

In any of the foregoing embodiments, the antimicrobial oil composition can further include a TAG oil such as an unmodified oil.

In any of the foregoing embodiments, the antimicrobial oil composition can include 80% or less, 70% or less, 60% or less, 50% or less, 40% or less, 30% or less, 25% or less, 20% or less, 15% or less, 10% or less, 5% or less, 4% or less, 3% or less, 2% or less, 1% or less, or is substantially free of triacylglycerols (TAGs). By way of further example, the antimicrobial oil composition can have no measurable TAGs as assessed qualitatively by Thin Layer Chromatography (TLC). In this method, components of the oil are separated using TLC plates (Analtech Uniplate Silica Gel GHL with inorganic binder, 20×20 cm, 250 μm), using a solvent (Hexane:Diethyl Ether:Acetic Acid (70:30:1) solution. Typical sample sizes are 0.2 μL. After the solvent front runs to near the top of the plate (˜1 cm), plates are removed from the TLC tank and the solvent evaporated in a fume hood. The components are visualized with iodine vapors (at room temperature) in a TLC tank and relative intensities estimated by colorimetric imaging. Components can also be measure quantitatively by standard HLPC and GC/MS methods. By way of still further example, the oil can have no measurable TAGs as assessed by gas chromatography (GC) or high performance liquid chromatography (HPLC). By way of example, but not limitation, HPLC can be performed under the following HPLC conditions: C18 column, 4.6 mm I.D.×250 mm, 5 um; column temp at 30° C.; charged aerosol detector, mobile phase A consisting of 75% acetonitrile, 12% methanol, 8% water, 4% tetrahydrofuran, and 0.3% acetic acid; mobile phase B consisting of 90% acetone and 10% acetonitrile; a gradient profile of: 100% A at 1.5 mL/min from 0-5 min, 100% A to 96% A at 1.5 mL/min from 5 to 11 min, 96% A to 37% A at 1.5 mL/min from 11 to 24 min, 37% A to 32% A at 1.5 mL/min from 24 to 35 min, 32% A to 18% A at 1.5 mL/min from 35 to 45 min, 18% A to 15% A at 1.9 mL/min from 45 to 58 min, 15% A to 5% A at 1.9 mL/min from 58 to 60 min, 5% A to 100% A at 1.5 mL/min from 60 to 63 min, 100% A at 1.5 mL/min from 63 to 65 min, using MAG, DAG, TAG and FFA calibration standards. Methods of quantitative measurements of glycerides and free fatty acids are described, by way of example but not limitation, Simultaneous Analysis of Glycerides (Mono-, Di-, and Triglycerides) and Free Fatty Acids in Palm Oil, Application Note 1039, Thermo Fisher Scientific, Inc., 2012, Annex 5.

In any of the foregoing embodiments, the antimicrobial oil composition can further include non-oil ingredients, derived from and naturally present in an oil source. By way of example, but not limitation, the non-oil ingredients derived from and naturally present in the oil source can include glycosphingolipids such as ceramide phosphates, glycoglycerolipids such as monoglactosyl diacylglycerols, phosphatidyl alcohols such as phosphatidyl methanol, steroids such as sitosteryl esters, natural lipids such as campesterol esters, sphingolipids, phosphatidyl glycerol, wax esters, sphingomyelin, phosphatides, phytosterols (campesterol, stigmasterol, sitosterol), cholesterol, tocopherols, tocotrienols, carotenes, xanthophylls, betaxanthins, chlorophyll, long chain alcohols, polyphenols, terpenes (cycloartanol, cycloarterenol, 24-methylene cycloarterenol), terpenoids (squalene), tocopherol, vitamins, paraffins, isothiocyanates, oxazolidinthiones, glucosinolates, sanguinarine, gossypols, glycosylated alkaloids, phospholipids, sphingosine, diacylglyceride ethers, glucolipids, phytic acid, quinic acid, oxalic acid, tartaric acid, anacardic acid, malic acid, caftaric acid, coutaric acid, fertaric acid, phenylethanoids, hydroxycinnamic acids, phenolic acids, phytoestrogens, lignans, isoflavonoids, flavonoids, monoterpenes, diterpenes, triterpenoids, coenzyme, or combinations thereof. By way of further example, but not limitation, the non-oil ingredients derived from and naturally present in the first oil source or the second oil source can include ceramide phosphate, monoglactodiacylglycerol, phosphatidylmethanol, sitosteryl ester, campesterol ester, sphingolipids, phosphatidyl glycerol, wax esters, sphingomyelin, or combinations thereof. By way of still further example, but not limitation, the non-oil ingredients derived from and naturally present in the oil source can include α-tocopherol, β-tocopherol, δ-tocopherol, γ-tocopherol, α-tocotrienol, β-tocotrienol, δ-tocotrienol, γ-tocotrienol, or combinations thereof. In certain aspects, the non-oil ingredients derived from and naturally present in the oil source are present in antimicrobial oil composition in an amount or relative amount characteristic of or enhanced relative to the amounts or relative amounts in the source oil. It should be understood that the level of the non-oil ingredients can generally be the same as or greater than that in the oil source. By way of example, but not limitation, the non-oil components can be determined by LC/MS/MS analysis. It should be understood that source oils can include a “tocopherol fingerprint” that is characteristic of the oil source and can be used to distinguish from added tocopherols.

In any of the foregoing embodiments, the MAGs in the EMO (or antimicrobial oil composition) can have a sn-1 substitution to sn-2 substitution ratio of of greater than 1. By way of example but not limitation, the MAGs in the EMO (or antimicrobial oil composition) can have a sn-1 substitution to sn-2 substitution ratio of greater than 1, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 6, 7, 8, 9, 10, 15, 20, 25, 30, 40, 50 or 100. By way of example, but not limitation, sn-1/sn-2 ratio can be determined by TLC or HPLC.

In any of the foregoing embodiments, the antimicrobial oil composition can include at least 10% FFA by weight out of the total weight of the antimicrobial oil composition. By way of example, but not limitation, the antimicrobial oil composition can include from about 10% to about 90%, about 15% to about 90%, about 20% to about 90%, about 25% to about 90%, about 30% to about 90%, about 40% to about 90%, about 50% to about 90%, about 60% to about 90%, about 70% to about 90%, about 75% to about 90%, about 80% to about 90%, about 10% to about 80%, about 15% to about 80%, about 20% to about 80%, about 25% to about 80%, about 30% to about 80%, about 40% to about 80%, about 50% to about 80%, about 60% to about 80%, about 70% to about 80%, about 75% to about 80%, about 10% to about 75%, about 15% to about 75%, about 20% to about 75%, about 25% to about 75%, about 30% to about 75%, about 40% to about 75%, about 50% to about 75%, about 60% to about 75%, about 70% to about 75%, about 10% to about 70%, about 15% to about 70%, about 20% to about 70%, about 30% to about 70%, about 40% to about 70%, about 50% to about 70%, about 60% to about 70%, about 10% to about 60%, about 20% to about 60%, about 25% to about 60%, about 30% to about 60%, about 40% to about 60%, about 50% to about 60%, about 10% to about 50%, about 15% to about 50%, about 20% to about 50%, about 25% to about 50%, about 30% to about 50%, about 40% to about 50%, about 10% to about 40%, about 15% to about 40%, about 20% to about 40%, about 25% to about 40%, about 30% to about 40%, about 10% to about 30%, about 15% to about 30%, about 20% to about 30%, about 25% to about 30%, about 10% to about 25%, about 15% to about 25%, about 20% to about 25%, about 10% to about 20%, about 15% to about 20%, about 10% to about 15%, or about 10%, 15%, 20%, 25%, 30%, 40%, 50%, 60%, 70%, 75%, 80%, or 90% FFA by weight out of the total weight of the antimicrobial oil composition.

In any of the foregoing embodiments, the antimicrobial oil composition can include at least 5% MAGs by weight out of the total weight of the antimicrobial oil composition. By way of example, but not limitation, the antimicrobial oil composition can include a MAG content from about 5% to about 80%, about 10% to about 80%, about 15% to about 80%, about 20% about 80%, about 25% to about 80%, about 30% to about 80%, about 40% to about 80%, about 50% to about 80%, about 60% to about 80%, about 70% to about 80%, about 75% to about 80%, about 5% to about 75%, about 10% to about 75%, about 15% to about 75%, about 20% to about 75%, about 25% to about 75%, about 30% to about 75%, about 40% to about 75%, about 50% to about 75%, about 60% to about 75%, about 70% to about 75%, about 5% to about 70%, about 10% to about 70%, about 15% to about 70%, about 20% to about 70%, about 25% to about 70%, about 30% to about 70%, about 40% to about 70%, about 50% to about 70%, about 60% to about 70%, about 5% to about 60%, about 10% to about 60%, about 15% to about 60%, about 20% to about 60%, about 25% to about 60%, about 30% to about 60%, about 40% to about 60%, about 50% to about 60%, about 5% to about 50%, about 10% to about 50%, about 15% to about 50%, about 20% to about 50%, about 25% to about 50%, about 30% to about 50%, about 40% to about 50%, about 5% to about 40%, about 10% to about 40%, about 15% to about 40%, about 20% to about 40%, about 25% to about 40%, about 30% to about 40%, about 5% to about 30%, about 10% to about 30%, about 20% to about 30%, about 25% to about 30%, about 5% to about 25%, about 10% to about 25%, about 15% to about 25%, about 20% to about 25%, about 5% to about 20%, about 10% to about 20%, about 15% to about 20%, about 5% to about 15%, about 10% to about 15%, about 5% to about 10%, about 5%, 6%, 7%, 8%, 9%, 10%, 15%, 20%, 25%, 30%, 35%, 40%, 45%, or 50%, or at least about 5%, 6%, 7%, 8%, 9%, 10%, 15%, 20%, 25%, 30%, 35%, 40%, 45%, or 50% MAGs by weight out of the total weight of the antimicrobial oil composition.

In any of the foregoing embodiments, the antimicrobial oil composition can include a DAG content. By way of example, but not limitation, the antimicrobial oil composition can have a DAG content of about 5% to about 66%, about 10% to about 66%, about 20% to about 66%, about 30% to about 66%, about 40% to about 66%, about 50% to about 66%, about 5% to about 60%, about 10% to about 60%, about 20% to about 60%, about 30% to about 60%, about 40% to about 60%, about 50% to about 60%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight of the total weight of the antimicrobial oil composition.

In any of the foregoing embodiments, the antimicrobial oil composition can have a ratio of MAGs to FFAs from about 0.01 to about 2.0. By way of example, but not limitation, the ratio of MAGs to FFAs can be from about 0.01 to 2.0, 0.05 to about 2.0, about 0.1 to about 2.0, about 0.2 to about 2.0, about 0.3 to about 2.0, about 0.4 to about 2.0, about 0.5 to about 2.0, about 0.6 to about 2.0, about 0.7 to about 2.0, about 0.8 to about 2.0, about 0.9 to about 2.0, about 1.0 to about 2.0, about 1.1 to about 2.0, about 1.2 to about 2.0, about 1.3 to about 2.0, about 1.4 to about 2.0, about 1.5 to about 2.0, about 1.6 to about 2.0 about 1.7 to about 2.0, about 1.8 to about 2.0, about 1.9 to about 2.0, about 0.01 to about 1.8, about 0.05 to about 1.8, about 0.1 to about 1.8, about 0.2 to about 1.8, about 0.3 to about 1.8, about 0.4 to about 1.8, about 0.5 to about 1.8, about 0.6 to about 1.8, about 0.7 to about 1.8, about 0.8 to about 1.8, about 0.9 to about 1.8, about 1.0 to about 1.8, about 1.1 to about 1.8, about 1.2 to about 1.8, about 1.3 to about 1.8, about 1.4 to about 1.8, about 1.5 to about 1.8, about 1.6 to about 1.8, about 1.7 to about 1.8, about 0.01 to about 1.6, about 0.05 to about 1.6, about 0.1 to about 1.6, about 0.2 to about 1.6, about 0.3 to about 1.6, about 0.4 to about 1.6, about 0.5 to about 1.6, about 0.6 to about 1.6, about 0.7 to about 1.6, about 0.8 to about 1.6, about 0.9 to about 1.6, about 1.0 to about 1.6, about 1.1 to about 1.6, about 1.2 to about 1.6, about 1.3 to about 1.6, about 1.4 to about 1.6, about 1.5 to about 1.6, about 0.01 to about 1.5, about 0.05 to about 1.5, about 0.1 to about 1.5, about 0.2 to about 1.5, about 0.3 to about 1.5, about 0.4 to about 1.5, about 0.5 to about 1.5, about 0.6 to about 1.5, about 0.7 to about 1.5, about 0.8 to about 1.5, about 0.9 to about 1.5, about 1.0 to about 1.5, about 1.1 to about 1.5, about 1.2 to about 1.5, about 1.3 to about 1.5, about 1.4 to about 1.5, about 0.01 to about 1.3, about 0.05 to about 1.3, about 0.1 to about 1.3, about 0.2 to about 1.3, about 0.3 to about 1.3, about 0.4 to about 1.3, about 0.5 to about 1.3, about 0.6 to about 1.3, about 0.7 to about 1.3, about 0.8 to about 1.3, about 0.9 to about 1.3, about 1.0 to about 1.3, about 1.1 to about 1.3, about 1.2 to about 1.3, about 0.01 to about 1.0, about 0.05 to about 1.0, about 0.1 to about 1.0, about 0.2 to about 1.0, about 0.3 to about 0.9, about 0.4 to about 0.9, about 0.5 to about 0.9, about 0.6 to about 0.9, about 0.7 to about 0.9, about 0.8 to about 0.9, about 0.01 to about 0.8, about 0.05 to about 0.8, about 0.1 to about 0.8, about 0.2 to about 0.8, about 0.3 to about 0.8, about 0.4 to about 0.8, about 0.5 to about 0.8, about 0.6 to about 0.8, about 0.7 to about 0.8, about 0.01 to about 0.7, about 0.05 to about 0.7, about 0.1 to about 0.7, about 0.2 to about 0.7, about 0.3 to about 0.7, about 0.4 to about 0.7, about 0.5 to about 0.7, about 0.6 to about 0.7, about 0.01 to about 0.6, about 0.05 to about 0.6, about 0.1 to about 0.6, about 0.2 to about 0.6, about 0.3 to about 0.6, about 0.4 to about 0.6, about 0.5 to about 0.6, about 0.01 to about 0.5, about 0.05 to about 0.5, about 0.1 to about 0.5, about 0.2 to about 0.5, about 0.3 to about 0.5, about 0.4 to about 0.5, about 0.01 to about 0.4, about 0.05 to about 0.4, about 0.1 to about 0.4, about 0.2 to about 0.4, about 0.3 to about 0.4, about 0.01 to about 0.3, about 0.05 to about 0.3, about 0.1 to about 0.3, about 0.2 to about 0.3, about 0.01 to about 0.2, about 0.05 to about 0.2, about 0.1 to about 0.2, about 0.01 to about 0.1, about 0.05 to about 0.1, or about 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0.

In any of the foregoing embodiments, the MAGs can be derived from a first oil source such as, by way of example but not limitation, from the first oil source for an EMO.

In any of the foregoing embodiments, the FFAs can be derived from a first oil source and a second oil source. By way of example, but not limitation, a first portion of the FFAs can be derived from the first oil source and a second portion of the FFAs can be derived from the second oil source such as, by way of example but not limitation, where the first oil source is used to generate an EMO and the second oil source is used to generate a FFA oil and the antimicrobial oil composition is a combination of EMO and FFA oil. It should be understood that where the antimicrobial oil composition only includes a FFA oil, and no EMO, then the FFA can have only a single oil source.

It should be understood that the first oil source and the second oil source can be the same oil or different oils and that the oil sources can be single oils or combinations of oils.

In any of the foregoing embodiments, the antimicrobial oil composition can be in the form of or part of a cream, a lotion, a balm, an eye drop, an ear drop, a sinus rinse, a spray, such as nasal, oral, mucosal or for skin or feet, an ointment, a deodorant formulation, a body wash, a shampoo, a scalp treatment, a mouthwash, a toothpaste, a lozenge, a beverage, a paste or a lubricant, such as vaginal lubricant. By way of example, but not limitation, the antimicrobial composition can be a cream such as for skin application, a lotion such as for skin application, a balm, an eye drop, an ear drop, a sinus rinse, a spray such as for nasal, oral mucosal, skin or foot treatment, an ointment, a deodorant, a body wash, a shampoo, a scalp treatment, a mouthwash, a toothpaste, a paste for tooth application, a lozenge, a beverage, a lubricant, or a powder, such as a spray-dried nanoemulsion (or microemulsion). Methods for formulating such compositions are well-known in the art. It should be understood that for compositions in a cream, such compositions can also be formulated in a lotion.

It should be understood that an antimicrobial composition of the present disclosure can include an antimicrobial oil composition, nanoemulsion or microemulsion of the present disclosure and can also exclude any compounds which those compositions can likewise exclude.

In any of the foregoing embodiments, the oil source of antimicrobial oil composition or the first oil source can be a plant, an animal, a fish, an algal oil, or combinations thereof. By way of example but not limitation, the oil source of antimicrobial oil composition or the first oil source can be olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

In any of the foregoing embodiments, the second oil source can be a plant, an animal, a fish, an algal oil, or combinations thereof. By way of example but not limitation, the second oil source can be olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, or combinations thereof.

It should be understood that the antimicrobial oil compositions, EMO and/or FFA oil of the present disclosure can be derived from two, three, four or more oils which can be the source oil for the component (EMO or FFA oil) or oil composition. It should be further understood that the EMO and/or FFA oil can be prepared from multiple oils either as a single process or the EMO and/or FFA oil resulting from separate processes can be later combined to form the EMO and/or FFA oil. It should also be understood that the first oil source and the second oil source can be the same or different.

In any of the foregoing embodiments, the first amount as a percentage of the total of the first amount and the second amount (i.e. the amount of EMO relative to the amount of EMO and FFA oil) can be from about 0.1% to about 99.9%, about 0.1% to about 99.8%, about 0.1% to about 99.5%, about 0.1% to about 99%, about 0.1% to about 98%, about 0.1% to about 97%, about 0.1% to about 96%, about 0.1% to about 95%, about 0.1% to about 94%, about 0.1% to about 93%, about 0.1% to about 92%, about 0.1% to about 91%, about 0.1% to about 90%, about 0.1% to about 80%, about 0.1% to about 75%, about 0.1% to about 70%, about 0.1% to about 60%, about 0.1% to about 50%, about 0.1% to about 40%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, about 0.1% to about 1%, about 0.1% to about 0.5%, about 0.1% to about 0.2%, 0.2% to about 99.9%, about 0.2% to about 99.8%, about 0.2% to about 99.5%, about 0.2% to about 99%, about 0.2% to about 98%, about 0.2% to about 97%, about 0.2% to about 96%, about 0.2% to about 95%, about 0.2% to about 94%, about 0.2% to about 93%, about 0.2% to about 92%, about 0.2% to about 91%, about 0.2% to about 90%, about 0.2% to about 80%, about 0.2% to about 75%, about 0.2% to about 70%, about 0.2% to about 60%, about 0.2% to about 50%, about 0.2% to about 40%, about 0.2% to about 30%, about 0.2% to about 25%, about 0.2% to about 20%, about 0.2% to about 15%, about 0.2% to about 10%, about 0.2% to about 5%, about 0.2% to about 4%, about 0.2% to about 3%, about 0.2% to about 2%, about 0.2% to about 1%, about 0.2% to about 0.5%, 0.5% to about 99.9%, about 0.5% to about 99.8%, about 0.5% to about 99.5%, about 0.5% to about 99%, about 0.5% to about 98%, about 0.5% to about 97%, about 0.5% to about 96%, about 0.5% to about 95%, about 0.5% to about 94%, about 0.5% to about 93%, about 0.5% to about 92%, about 0.5% to about 91%, about 0.5% to about 90%, about 0.5% to about 80%, about 0.5% to about 75%, about 0.5% to about 70%, about 0.5% to about 60%, about 0.5% to about 50%, about 0.5% to about 40%, about 0.5% to about 30%, about 0.5% to about 25%, about 0.5% to about 20%, about 0.5% to about 15%, about 0.5% to about 10%, about 0.5% to about 5%, about 0.5% to about 4%, about 0.5% to about 3%, about 0.5% to about 2%, about 0.5% to about 1%, about 1% to about 99.9%, about 1% to about 99.8%, about 1% to about 99.5%, about 1% to about 99%, about 1% to about 98%, about 1% to about 97%, about 1% to about 96%, about 1% to about 95%, about 1% to about 94%, about 1% to about 93%, about 1% to about 92%, about 1% to about 91%, about 1% to about 90%, about 1% to about 80%, about 1% to about 75%, about 1% to about 70%, about 1% to about 60%, about 1% to about 50%, about 1% to about 40%, about 1% to about 30%, about 1% to about 25%, about 1% to about 20%, about 1% to about 15%, about 1% to about 10%, about 1% to about 5%, about 1% to about 4%, about 1% to about 3%, about 1% to about 2%, about 2% to about 99.9%, about 2% to about 99.8%, about 2% to about 99.5%, about 2% to about 99%, about 2% to about 98%, about 2% to about 97%, about 2% to about 96%, about 2% to about 95%, about 2% to about 94%, about 2% to about 93%, about 2% to about 92%, about 2% to about 91%, about 2% to about 90%, about 2% to about 80%, about 2% to about 75%, about 2% to about 70%, about 2% to about 60%, about 2% to about 50%, about 2% to about 40%, about 2% to about 30%, about 2% to about 25%, about 2% to about 20%, about 2% to about 10%, about 2% to about 5%, about 5% to about 99.9%, about 5% to about 99.8%, about 5% to about 99.5%, about 5% to about 99%, about 5% to about 98%, about 5% to about 97%, about 5% to about 96%, about 5% to about 95%, about 5% to about 94%, about 5% to about 93%, about 5% to about 92%, about 5% to about 91%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 99.9%, about 10% to about 99.8%, about 10% to about 99.5%, about 10% to about 99%, about 10% to about 98%, about 10% to about 97%, about 10% to about 96%, about 10% to about 95%, about 10% to about 94%, about 10% to about 93%, about 10% to about 92%, about 10% to about 91%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 99.9%, about 20% to about 99.8%, about 20% to about 99.5%, about 20% to about 99%, about 20% to about 98%, about 20% to about 97%, about 20% to about 96%, about 20% to about 95%, about 20% to about 94%, about 20% to about 93%, about 20% to about 92%, about 20% to about 91%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 99.9%, about 25% to about 99.8%, about 25% to about 99.5%, about 25% to about 99%, about 25% to about 98%, about 25% to about 97%, about 25% to about 96%, about 25% to about 95%, about 25% to about 94%, about 25% to about 93%, about 25% to about 92%, about 25% to about 91%, about 25% to about 90%, about 25% to about 80%, about 25% to about 75%, about 25% to about 70%, about 25% to about 60%, about 25% to about 50%, about 25% to about 40%, about 25% to about 30%, about 30% to about 99.9%, about 30% to about 99.8%, about 30% to about 99.5%, about 30% to about 99%, about 30% to about 98%, about 30% to about 97%, about 30% to about 96%, about 30% to about 95%, about 30% to about 94%, about 30% to about 93%, about 30% to about 92%, about 30% to about 91%, about 30% to about 90%, about 30% to about 80%, about 30% to about 75%, about 30% to about 70%, about 30% to about 60%, about 30% to about 50%, about 30% to about 40%, about 40% to about 99.9%, about 40% to about 99.8%, about 40% to about 99.5%, about 40% to about 99%, about 40% to about 98%, about 40% to about 97%, about 40% to about 96%, about 40% to about 95%, about 40% to about 94%, about 40% to about 93%, about 40% to about 92%, about 40% to about 91%, about 40% to about 90%, about 40% to about 80%, about 40% to about 75%, about 40% to about 70%, about 40% to about 60%, about 40% to about 50%, about 50% to about 99.9%, about 50% to about 99.8%, about 50% to about 99.5%, about 50% to about 99%, about 50% to about 98%, about 50% to about 97%, about 50% to about 96%, about 50% to about 95%, about 50% to about 94%, about 50% to about 93%, about 50% to about 92%, about 50% to about 91%, about 50% to about 90%, about 50% to about 80%, about 50% to about 75%, about 50% to about 70%, about 50% to about 60%, about 60% to about 99.9%, about 60% to about 99.8%, about 60% to about 99.5%, about 60% to about 99%, about 60% to about 98%, about 60% to about 97%, about 60% to about 96%, about 60% to about 95%, about 60% to about 94%, about 60% to about 93%, about 60% to about 92%, about 60% to about 91%, about 60% to about 90%, about 60% to about 80%, about 60% to about 75%, about 60% to about 70%, about 70% to about 99.9%, about 70% to about 99.8%, about 70% to about 99.5%, about 70% to about 99%, about 70% to about 98%, about 70% to about 97%, about 70% to about 96%, about 70% to about 95%, about 70% to about 94%, about 70% to about 93%, about 70% to about 92%, about 70% to about 91%, about 70% to about 90%, about 70% to about 80%, about 80% to about 99.9%, about 80% to about 99.8%, about 80% to about 99.5%, about 80% to about 99%, about 80% to about 98%, about 80% to about 97%, about 80% to about 96%, about 80% to about 95%, about 80% to about 94%, about 80% to about 93%, about 80% to about 92%, about 80% to about 91%, about 80% to about 90%, about 90% to about 99.9%, about 90% to about 99.8%, about 90% to about 99.5%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 99.9%, about 91% to about 99.8%, about 91% to about 99.5%, about 91% to about 99%, about 91% to about 98%, about 91% to about 97%, about 91% to about 96%, about 91% to about 95%, about 91% to about 94%, about 91% to about 93%, about 91% to about 92%, about 92% to about 99.9%, about 92% to about 99.8%, about 92% to about 99.5%, about 92% to about 99%, about 92% to about 98%, about 92% to about 97%, about 92% to about 96%, about 92% to about 95%, about 92% to about 94%, about 92% to about 93%, about 93% to about 99.9%, about 93% to about 99.8%, about 93% to about 99.5%, about 93% to about 99%, about 93% to about 98%, about 93% to about 97%, about 93% to about 96%, about 93% to about 95%, about 93% to about 94%, about 94% to about 99.9%, about 94% to about 99.8%, about 94% to about 99.5%, about 94% to about 99%, about 94% to about 98%, about 94% to about 97%, about 94% to about 96%, about 94% to about 95%, about 95% to about 99.9%, about 95% to about 99.8%, about 95% to about 99.5%, about 95% to about 99%, about 95% to about 98%, about 95% to about 97%, about 95% to about 96%, about 96% to about 99.9%, about 96% to about 99.8%, about 96% to about 99.5%, about 96% to about 99%, about 96% to about 98%, about 96% to about 97%, about 97% to about 99.9%, about 97% to about 99.8%, about 97% to about 99.5%, about 97% to about 99%, about 97% to about 98%, about 98% to about 99.9%, about 98% to about 99.8%, about 98% to about 99.5%, about 98% to about 99%, about 99% to about 99.9%, about 99% to about 99.8%, about 99% to about 99.5%, about 99.5% to about 99.9%, about 99.5% to about 99.8%, about 99.8% to about 99.9%, or about 0.1%, 0.2%, 0.5%, 1%, 2%, 3%, 4%, 5%, 6%, 7%, 8%, 9%, 10%, 15%, 20%, 25%, 30%, 40%, 50%, 60%, 70%, 75%, 80%, 90%, 91%, 92%, 93%, 94%, 95%, 96%, 97%, 98%, 99%, 99.5%, 99.8%, or 99.9%. By way of still further example, the first amount as a percentage of the total of the first amount and the second amount can be from about 25% to about 75%. Alternatively, the first amount as a percentage of the total of the first amount the second amount can be 0% or 100%, i.e. either pure EMO or pure FFA oil.

In any of the foregoing embodiments, the antimicrobial oil composition can have a fatty acid profile, comprising two or more different fatty acids selected from the group consisting of caproic acid, enanthic acid, caprylic acid, pelargonic acid, capric acid, undecylic acid, lauric acid, tridecylic acid, myristic acid, pentadecylic acid, palmitic acid, acid, oleic acid, linoleic acid, linolenic acid, stearidonic acid, octadecatetraenoic acid, arachidic acid, heneicosylic acid, gondoic acid, eicosadienoic acid, paullinic acid, behenic acid, tricosylic acid, erucic acid, lignoceric acid, docosahexaenoic acid, eicosapentaenoic acid, nervonic acid, and isomers thereof. Alternatively, the fatty acids, including the two or more different fatty acids can each independently be selected from the group consisting of a C6 fatty acid such as, by way of example but not limitation, C6:0, a C7 fatty acid, a C8 fatty acid such as, by way of example but not limitation, C8:0 fatty acid, a C9 fatty acid, a C10 fatty acid, a C11 fatty acid, a C12 fatty acid, a C13 fatty acid, a C14 fatty acid, a C15 fatty acid, a C16 fatty acid, a C17 fatty acid, a C18 fatty acid such as, by way of example, but not limitation, a C18:0 fatty acid, a C18:0 fatty acid, a C18:1 fatty acid, a C18:2 fatty acid, or a C18:3 fatty acid, a C19 fatty acid, a C20 fatty acid such as, by way of example, but not limitation, C20:5 fatty acid, a C21 fatty acid, a C22 fatty acid such as, by way of example, but not limitation, C22:6 fatty acid, a C23 fatty acid, and a C24 fatty acid. It should be understood that the two or more different fatty acids can be the same between the EMO and FFA oil or can be different. As used herein, C18 should be understood to refer to stearic acid (C18:0).

In any of the foregoing embodiments, the two or more different fatty acids can be present in the EMO or FFA oil in an amount that is within 10%, 9%, 8%, 7%, 6%, 5%, 4%, 3%, 2% or 1% of the amount of that fatty acid in the a fatty acid profile of the first oil source or the second oil source, respectively. In any of the foregoing embodiments, the two or more different fatty acids, can be two fatty acids, three fatty acids, four fatty acids, five fatty acids, six fatty acids, seven fatty acids, eight fatty acids, nine fatty acids, ten fatty acids or more. For example, if a first oil source has 20% C18:1 fatty acid and 20% C12 fatty acid, then the EMO would have 10-30% C18:1 and 10-30% C12 if it is within 10% of the amount of the fatty acids in the fatty acid profile of the first source oil. Thus, where the oil composition is a blend of EMO and FFA oil from different oil sources, the fatty acid profile of the oil composition will vary according to the ratio of EMO:FFA oil and the expected ranges can be calculated by one of skill in the art. However, where the EMO and FFA are derived from the same source oil, the fatty acid profile of the oil composition would be expected to be similar to that of the source oil

In any of the foregoing embodiments, the antimicrobial oil composition can have a fatty acid profile comprising two, three, four, five, six, seven, eight, nine, ten or 11 of the following:

| Fatty Acid | Preferred Range |
|---|---|
| C8:0 | 20%-35% |
| C10 | 0.1%-30% |
| C12 | 5%-55% |
| C14 | 0.1%-25% |
| C16 | 0.1%-30% |
| C18 | 0.1%-10% |
| C18:1 | 0.1%-30% |
| C18:2 | 0.1%-60% |
| C18:3 | 0.1%-65% |
| C20 | 0.1%-15% |
| C22 | 5%-25% |

By way of example, but not limitation, the antimicrobial oil composition can have a fatty acid profile comprising two, three, four, five, six, seven, eight, nine, ten or 11 of the following:

In any of the foregoing embodiments, the antimicrobial oil composition can have a fatty acid profile comprising two, three, four, five, six, seven, eight, nine, ten, eleven, or twelve of C6:0, C8:0, C10, C12, C14, C16, C18, C18:1, C18:2, C18:3, C20 and C22, wherein a range of each fatty is acid is between about 0.1% and 85% out of the total fatty acid content. By way of example, but not limitation, for each fatty acid, the amount can be from about 0.1% to about 85%, about 0.5% to about 85%, about 1% to about 85%, about 2% to about 85%, about 3% to about 85%, about 4% to about 85%, about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 0.1% to about 80%, about 0.5% to about 80%, about 1% to about 80%, about 2% to about 80%, about 3% to about 80%, about 4% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 0.1% to about 75%, about 0.5% to about 75%, about 1% to about 75%, about 2% to about 75%, about 3% to about 75%, about 4% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.1% to about 70%, about 0.5% to about 70%, about 1% to about 70%, about 2% to about 70%, about 3% to about 70%, about 4% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 0.1% to about 65%, about 0.5% to about 65%, about 1% to about 65%, about 2% to about 65%, about 3% to about 65%, about 4% to about 65%, about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 0.1% to about 60%, about 0.5% to about 60%, about 1% to about 60%, about 2% to about 60%, about 3% to about 60%, about 4% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 0.1% to about 55%, about 0.5% to about 55%, about 1% to about 55%, about 2% to about 55%, about 3% to about 55%, about 4% to about 55%, about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 0.1% to about 50%, about 0.5% to about 50%, about 1% to about 50%, about 2% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 45%, about 0.5% to about 45%, about 1% to about 45%, about 2% to about 45%, about 3% to about 45%, about 4% to about 45%, about 5% to about 45%, about 10% to about 45%, about 15% to about 45%, about 20% to about 45%, about 25% to about 45%, about 30% to about 45%, about 35% to about 45%, about 40% to about 45%, about 0.1% to about 40%, about 0.5% to about 40%, about 1% to about 40%, about 2% to about 40%, about 3% to about 40%, about 4% to about 40%, about 5% to about 40%, about 10% to about 40%, about 15% to about 40%, about 20% to about 40%, about 25% to about 40%, about 30% to about 40%, about 35% to about 40%, about 0.1% to about 35%, about 0.5% to about 35%, about 1% to about 35%, about 2% to about 35%, about 3% to about 35%, about 4% to about 35%, about 5% to about 35%, about 10% to about 35%, about 15% to about 35%, about 20% to about 35%, about 25% to about 35%, about 30% to about 35%, about 0.1% to about 30%, about 0.5% to about 30%, about 1% to about 30%, about 2% to about 30%, about 3% to about 30%, about 4% to about 30%, about 5% to about 30%, about 10% to about 30%, about 15% to about 30%, about 20% to about 30%, about 25% to about 30%, about 0.1% to about 25%, about 0.5% to about 25%, about 1% to about 25%, about 2% to about 25%, about 3% to about 25%, about 4% to about 25%, about 5% to about 25%, about 10% to about 25%, about 15% to about 25%, about 20% to about 25%, about 0.1% to about 20%, about 0.5% to about 20%, about 1% to about 20%, about 2% to about 20%, about 3% to about 20%, about 4% to about 20%, about 5% to about 20%, about 10% to about 20%, about 15% to about 20%, about 0.1% to about 15%, about 0.5% to about 15%, about 1% to about 15%, about 2% to about 15%, about 3% to about 15%, about 4% to about 15%, about 5% to about 15%, about 10% to about 15%, about 0.1% to about 10%, about 0.5% to about 10%, about 1% to about 10%, about 2% to about 10%, about 3% to about 10%, about 4% to about 10%, about 5% to about 10%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 1% to about 4%, about 2% to about 4%, about 3% to about 4%, about 0.1% to about 3%, about 0.5% to about 3%, about 1% to about 3%, about 2% to about 3%, about 0.1% to about 2%, about 0.5% to about 2%, about 1% to about 2%, about 0.1% to about 1%, about 0.5% to about 1%, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80% or 85% out of the total fatty acid content.

In any of the foregoing embodiments, the MAGs (and the EMO) can have a fatty acid profile comprising two, three, four, five, six, seven, eight, nine, ten, eleven, or twelve of C6:0, C8:0, C10, C12, C14, C16, C18, C18:1, C18:2, C18:3, C20 and C22, wherein a range of each fatty is acid is between about 0.1% and 85% out of the total fatty acid content. By way of example, but not limitation, for each fatty acid, the amount can be from about 0.1% to about 85%, about 0.5% to about 85%, about 1% to about 85%, about 2% to about 85%, about 3% to about 85%, about 4% to about 85%, about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 0.1% to about 80%, about 0.5% to about 80%, about 1% to about 80%, about 2% to about 80%, about 3% to about 80%, about 4% to about 80%, about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 0.1% to about 75%, about 0.5% to about 75%, about 1% to about 75%, about 2% to about 75%, about 3% to about 75%, about 4% to about 75%, about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.1% to about 70%, about 0.5% to about 70%, about 1% to about 70%, about 2% to about 70%, about 3% to about 70%, about 4% to about 70%, about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 0.1% to about 65%, about 0.5% to about 65%, about 1% to about 65%, about 2% to about 65%, about 3% to about 65%, about 4% to about 65%, about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 0.1% to about 60%, about 0.5% to about 60%, about 1% to about 60%, about 2% to about 60%, about 3% to about 60%, about 4% to about 60%, about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%, about 0.1% to about 55%, about 0.5% to about 55%, about 1% to about 55%, about 2% to about 55%, about 3% to about 55%, about 4% to about 55%, about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%, about 0.1% to about 50%, about 0.5% to about 50%, about 1% to about 50%, about 2% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 45%, about 0.5% to about 45%, about 1% to about 45%, about 2% to about 45%, about 3% to about 45%, about 4% to about 45%, about 5% to about 45%, about 10% to about 45%, about 15% to about 45%, about 20% to about 45%, about 25% to about 45%, about 30% to about 45%, about 35% to about 45%, about 40% to about 45%, about 0.1% to about 40%, about 0.5% to about 40%, about 1% to about 40%, about 2% to about 40%, about 3% to about 40%, about 4% to about 40%, about 5% to about 40%, about 10% to about 40%, about 15% to about 40%, about 20% to about 40%, about 25% to about 40%, about 30% to about 40%, about 35% to about 40%, about 0.1% to about 35%, about 0.5% to about 35%, about 1% to about 35%, about 2% to about 35%, about 3% to about 35%, about 4% to about 35%, about 5% to about 35%, about 10% to about 35%, about 15% to about 35%, about 20% to about 35%, about 25% to about 35%, about 30% to about 35%, about 0.1% to about 30%, about 0.5% to about 30%, about 1% to about 30%, about 2% to about 30%, about 3% to about 30%, about 4% to about 30%, about 5% to about 30%, about 10% to about 30%, about 15% to about 30%, about 20% to about 30%, about 25% to about 30%, about 0.1% to about 25%, about 0.5% to about 25%, about 1% to about 25%, about 2% to about 25%, about 3% to about 25%, about 4% to about 25%, about 5% to about 25%, about 10% to about 25%, about 15% to about 25%, about 20% to about 25%, about 0.1% to about 20%, about 0.5% to about 20%, about 1% to about 20%, about 2% to about 20%, about 3% to about 20%, about 4% to about 20%, about 5% to about 20%, about 10% to about 20%, about 15% to about 20%, about 0.1% to about 15%, about 0.5% to about 15%, about 1% to about 15%, about 2% to about 15%, about 3% to about 15%, about 4% to about 15%, about 5% to about 15%, about 10% to about 15%, about 0.1% to about 10%, about 0.5% to about 10%, about 1% to about 10%, about 2% to about 10%, about 3% to about 10%, about 4% to about 10%, about 5% to about 10%, about 0.1% to about 5%, about 0.5% to about 5%, about 1% to about 5%, about 2% to about 5%, about 3% to about 5%, about 4% to about 5%, about 0.1% to about 4%, about 0.5% to about 4%, about 1% to about 4%, about 2% to about 4%, about 3% to about 4%, about 0.1% to about 3%, about 0.5% to about 3%, about 1% to about 3%, about 2% to about 3%, about 0.1% to about 2%, about 0.5% to about 2%, about 1% to about 2%, about 0.1% to about 1%, about 0.5% to about 1%, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80% or 85% out of the total fatty acid content.

It should be understood that the fatty acid profile that is expected can be estimated based on known fatty acid profiles and the amount of EMO and/or FFA oil in the antimicrobial oil composition because the EMO and/or FFA oil can generally preserve the fatty acid profile within a range.

In any of the foregoing embodiments, the antimicrobial oil composition can have fatty acid profile comprising an amount of two or more fatty acids, wherein the amount of each of the two or more fatty acids within 10% of the amount of each of the two or more fatty acids in the first oil source. By way of example, but not limitation, the antimicrobial oil composition can have a fatty acid profile comprising an amount of three or more, four or more, five or more, six or more, seven or more, eight or more, nine, ten or more, eleven, twelve or more fatty acids, wherein the amount of each of the fatty acids is within 10% of the amount of each of the fatty acids, 9%, 8%, 7%, 6%, 5%, 4%, 3%, 2%, or 1% or be about the same amount. By way of example, but not limitation, such fatty acids can be C6:0, C8:0, C10, C12, C14, C16, C18, C18:1, C18:2, C18:3, C20 or C22. For example, if the first oil source has 15% C18 fatty acid, the EMO can have between about 5-25%, 6-24%, 7-23%, 8-22%, 9-21%, 10-20%, 11-19%, 12-18%, 13-17%, 14-16% or about 15% C18 fatty acid. It should be understood that where the antimicrobial oil composition is a blend of EMO and FFA oil from different sources, the amounts of fatty acid will vary according to the ratio of the EMO and FFA oil and the starting fatty acid profile. Exemplary fatty acid profiles are provided in the Tables below which also include exemplary EMO compositions based on experimental data. It should be understood that for fatty acid profiles, amounts of fatty acid can naturally vary by about 3-5% depending on species, growing conditions and other variables.

| Fatty | Flaxseed | MCT | Rosehip | Sesame | Hempseed | Coconut |
|---|---|---|---|---|---|---|
| Acid | Oil | Oil | Oil | Oil | Oil | Oil* |
| C6:0 | 0.0 | 0.06 | 0.0 | 0.0 | 0.0 | 0.0 |
| C8:0 | 0.0 | 57.42 | 0.0 | 0.0 | 0.0 | 0.0 |
| C10 | 0.0 | 41.96 | 0.0 | 0.0 | 0.0 | 6.0 |
| C12 | 0.0 | 0.32 | 0.0 | 0.0 | 0.0 | 47.0 |
| C14 | 0.0 | 0.0 | 0.0 | 0.0 | 0.0 | 18.0 |
| C16 | 5.2 | 0.0 | 4.5 | 7.7 | 6.6 | 9.0 |
| C18 | 3.3 | 0.0 | 2.2 | 14.3 | 2.6 | 3.0 |
| C18:1 | 15.4 | 0.0 | 19.9 | 40.1 | 11.5 | 2.0 |
| C18:2 | 16.0 | 0.0 | 50.8 | 37.7 | 58.6 | 2.0 |
| C18:3 | 59.7 | 0.0 | 20.7 | 0.2 | 19.2 | 0.0 |
| C20 | 0.4 | 0.0 | 1.9 | 0.0 | 1.5 | 0.0 |
| C22 | 0.0 | 0.0 | 0.0 | 0.0 | 0.0 | 0.0 |

EMO

| | | | | | | |
|---|---|---|---|---|---|---|
| MAG | 57.9 | est. 64 | 54.6 | 62.5 | 69.9 | est. 72 |
| DAG | 16.5 | est. 15 | 16.5 | 17.9 | 11 | est. 16 |
| TAG | BLD | BLD | BLD | BLD | BLD | BLD |
| FFA | 18 | 21 | 16 | 12 | 9 | 12 |

| Fatty | Fish | Algae | Oat | Canola | Olive | Almond |
|---|---|---|---|---|---|---|
| Acid | Oil* | Oil* | Oil | Oil | Oil* | Oil |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 0.0 | 0.0 | 0.0 | 0.0 |
| C12 | 0.0 | 0.0 | 0.0 | 0.0 | 0.0 | 0.0 |
| C14 | 9.4 | 1.0 | 0.0 | 0.0 | 0.0 | 0.0 |
| C16 | 26.9 | 18.5 | 11.4 | 4.6 | 13.0 | 7.0 |
| C18 | 0.0 | 1.9 | 4.3 | 1.8 | 3.0 | 2.0 |
| C18:1 | 21.8 | 30.6 | 22.5 | 62.8 | 71.0 | 69.0 |
| C18:2 | 2.5 | 22.8 | 53.8 | 21.8 | 10.0 | 17.0 |
| C18:3 | 1.7 | 16.5 | 7.2 | 6.5 | 1.0 | 0.0 |
| C20 | 14.5 | 1.5 | 0.8 | 2.6 | 0.0 | 0.0 |
| C22 | 23.2 | 0.5 | 0.0 | 0.0 | 0.0 | 0.0 |

EMO

| | | | | | |
|---|---|---|---|---|---|
| MAG | est. 56 | 65.9 | 60.9 | 55.9 | 74.9 |
| DAG | est. 14 | 9.4 | 16.3 | 14.4 | 12.5 |
| TAG | 16 | BLD | BLD | BLD | BLD |
| FFA | 12 | 13 | 13.9 | 20 | 10 |

| Fatty | Cottonseed | Peanut | Sunflower | Corn | Safflower | Soybean |
|---|---|---|---|---|---|---|
| Acid | Oil* | Oil* | Oil* | Oil* | Oil* | Oil* |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 0.0 | 0.0 | 0.0 | 0.0 |
| C12 | 0.0 | 0.0 | 0.0 | 0.0 | 0.0 | 0.0 |
| C14 | 1.0 | 0.0 | 0.0 | 0.0 | 0.0 | 0.0 |
| C16 | 22.0 | 11.0 | 7.0 | 11.0 | 7.0 | 11.0 |
| C18 | 3.0 | 2.0 | 5.0 | 2.0 | 2.0 | 4.0 |
| C18:1 | 19.0 | 48.0 | 19.0 | 28.0 | 13.0 | 24.0 |
| C18:2 | 54.0 | 32.0 | 68.0 | 58.0 | 78.0 | 54.0 |
| C18:3 | 1.0 | 0.0 | 1.0 | 1.0 | 0.0 | 7.0 |
| C20 | 0.0 | 0.0 | 0.0 | 0.0 | 0.0 | 0.0 |
| C22 | 0.0 | 0.0 | 0.0 | 0.0 | 0.0 | 0.0 |

EMO

| | |
|---|---|
| MAG | 58.3 |
| DAG | 25.5 |
| TAG | BLD |
| FFA | 9 |

| | | | | | Palm | | | |
|---|---|---|---|---|---|---|---|---|
| Fatty | Cacao | Grapeseed | Palm | Palm | Kernel | Shea | Walnut | Macadamia |
| Acid | Oil* | Oil* | Oil* | Olein* | Oil* | Oil* | Oil* | Nut Oil |
| C6:0 | 0.0 | 0.0 | 0.0 | 0.0 | 0.0 | 0.0 | 0.0 | 0.0 |
| C8:0 | 0.0 | 0.0 | 0.0 | 0.0 | 0.0 | 0.0 | 0.0 | 0.0 |
| C10 | 0.0 | 0.0 | 0.0 | 0.0 | 4.0 | 0.0 | 0.0 | 0.0 |
| C12 | 0.0 | 0.0 | 0.0 | 0.0 | 48.0 | 1.0 | 0.0 | 0.0 |
| C14 | 0.0 | 0.0 | 1.0 | 1.0 | 16.0 | 0.0 | 0.0 | 1.2 |
| C16 | 25.0 | 8.0 | 45.0 | 37.0 | 8.0 | 4.0 | 11.0 | 41 |
| C18 | 38.0 | 4.0 | 4.0 | 4.0 | 3.0 | 39.0 | 5.0 | 5.4 |
| C18:1 | 32.0 | 15.0 | 40.0 | 46.0 | 15.0 | 44.0 | 28.0 | 41 |
| C18:2 | 3.0 | 73.0 | 10.0 | 11.0 | 2.0 | 5.0 | 51.0 | 2.7 |
| C18:3 | 0.0 | 0.0 | 0.0 | 0.0 | 0.0 | 0.0 | 5.0 | 1 |
| C20 | 0.0 | 0.0 | 0.0 | 0.0 | 0.0 | 0.0 | 0.0 | 6.5 |
| C22 | 0.0 | 0.0 | 0.0 | 0.0 | 0.0 | 0.0 | 0.0 | 1.1 |

EMO

MAG
DAG
TAG
FFA

*Fatty acid profile values derived from literature

EMO and FFA Oils

In any of the foregoing embodiments, the EMO and/or FFA oil can have a triacylglycerol (TAG) content of 5% or less TAGs by weight based on the total weight of the EMO and/or FFA oil, respectively. By way of example, but not limitation, the TAG content can be equal to or less than 4%, equal to or less than 3%, equal to or less than 2%, equal to or less than 1% by weight based on the total weight of the EMO, FFA oil, or the oil composition, respectively, or the EMO and/or the FFA oil can be substantially free of TAGs.

It should be understood that the EMO and/or FFA oil can be derived from the oil sources described herein, including the first oil source and the second oil source, respectively, as provided in the foregoing embodiments.

In any of the foregoing embodiments, the EMO and/or FFA oil can further include non-oil ingredients derived from and naturally present in the first oil source or the second oil source, respectively. By way of example, but not limitation, the non-oil ingredients derived from and naturally present in the oil source, the first oil source or the second oil source can include glycosphingolipids such as ceramide phosphates, glycoglycerolipids such as monoglactosyl diacylglycerols, phosphatidyl alcohols such as phosphatidyl methanol, steroids such as sitosteryl esters, natural lipids such as campesterol esters, sphingolipids, phosphatidyl glycerol, wax esters, sphingomyelin, phosphatides, phytosterols (campesterol, stigmasterol, sitosterol), cholesterol, tocopherols, tocotrienols, carotenes, xanthophylls, betaxanthins, chlorophyll, long chain alcohols, polyphenols, terpenes (cycloartanol, cycloarterenol, 24-methylene cycloarterenol), terpenoids (squalene), tocopherol, vitamins, paraffins, isothiocyanates, oxazolidinthiones, glucosinolates, sanguinarine, gossypols, glycosylated alkaloids, phospholipids, sphingosine, diacylglyceride ethers, glucolipids, phytic acid, quinic acid, oxalic acid, tartaric acid, anacardic acid, malic acid, caftaric acid, coutaric acid, fertaric acid, phenylethanoids, hydroxycinnamic acids, phenolic acids, phytoestrogens, lignans, isoflavonoids, flavonoids, monoterpenes, diterpenes, triterpenoids, coenzyme, or combinations thereof. By way of further example, but not limitation, the non-oil ingredients derived from and naturally present in the first oil source or the second oil source can include ceramide phosphate, monoglactodiacylglycerol, phosphatidylmethanol, sitosteryl ester, campesterol ester, sphingolipids, phosphatidyl glycerol, wax esters, sphingomyelin, or combinations thereof. By way of still further example, but not limitation, the non-oil ingredients derived from and naturally present in the first oil source or the second oil source can include α-tocopherol, β-tocopherol, δ-tocopherol, γ-tocopherol, α-tocotrienol, β-tocotrienol, δ-tocotrienol, γ-tocotrienol, or combinations thereof. In certain aspects, the non-oil ingredients derived from and naturally present in the first oil source or the second oil source are present in the EMO or FFA oil in an amount or relative amount characteristic of or increased relative to the amounts or relative amounts, respectively, in the first oil source or the second oil source, respectively. It should be understood that the level of the non-oil ingredients can generally be the same as or greater than that in the oil source. By way of example, but not limitation, the non-oil components can be determined by LC/MS/MS analysis.

In any of the foregoing embodiments, the MAGs in the EMO (or antimicrobial oil composition) can have a sn-1 substitution to sn-2 substitution ratio of of greater than 1. By way of example but not limitation, the MAGs in the EMO (or antimicrobial oil composition) can have a sn-1 substitution to sn-2 substitution ratio of greater than 1, 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 6, 7, 8, 9, 10, 15, 20, 25, 30, 40, 50 or 100. By way of example, but not limitation, sn-1/sn-2 ratio can be determined by TLC or HPLC.

In any of the foregoing embodiments, the EMO can include free fatty acids (FFA). In any of the foregoing embodiments, the EMO can include a FFA content equal to or greater than 5% by weight of the total weight of the EMO. By way of example, but not limitation, the EMO can include a FFA content of about 5% to about 66%, about 10% to about 66%, about 20% to about 66%, about 30% to about 66%, about 40% to about 66%, about 50% to about 66%, about 5% to about 60%, about 10% to about 60%, about 20% to about 60%, about 30% to about 60%, about 40% to about 60%, about 50% to about 60%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight of the total weight of the EMO.

In any of the foregoing embodiments, the FFA oil can include free fatty acids (FFA). In any of the foregoing embodiments, the FFA oil can include a FFA content equal to or greater than 1% by weight of the total weight of the FFA oil. By way of example, but not limitation, the FFA oil can include a FFA content equal of about 1% to about 95%, about 5% to about 95%, about 10% to about 95%, about 15% to about 95%, about 20% to about 95%, about 25% to about 95%, about 30% to about 95%, about 40% to about 95%, about 50% to about 95%, about 60% to about 95%, about 70% to about 95%, about 75% to about 95%, about 80% to about 95%, about 90% to about 95%, about 1% to about 90%, about 5% to about 90%, about 10% to about 90%, about 15% to about 90%, about 20% to about 90%, about 25% to about 90%, about 30% to about 90%, about 40% to about 90%, about 50% to about 90%, about 60% to about 90%, about 70% to about 90%, about 75% to about 90%, about 80% to about 90%, about 1% to about 80%, about 5% to about 80%, about 10% to about 80%, about 15% to about 80%, about 20% to about 80%, about 25% to about 80%, about 30% to about 80%, about 40% to about 80%, about 50% to about 80%, about 60% to about 80%, about 70% to about 80%, about 75% to about 80%, about 1% to about 75%, about 5% to about 75%, about 10% to about 75%, about 15% to about 75%, about 20% to about 75%, about 25% to about 75%, about 30% to about 75%, about 40% to about 75%, about 50% to about 75%, about 60% to about 75%, about 70% to about 75%, about 1% to about 70%, about 5% to about 70%, about 10% to about 70%, about 15% to about 70%, about 20% to about 70%, about 25% to about 70%, about 30% to about 70%, about 40% to about 70%, about 50% to about 70%, about 60% to about 70%, about 1% to about 60%, about 5% to about 60%, about 10% to about 60%, about 15% to about 60%, about 20% to about 60%, about 25% to about 60%, about 30% to about 60%, about 40% to about 60%, about 50% to about 60%, about 1% to about 50%, about 5% to about 50%, about 10% to about 50%, about 15% to about 50%, about 20% to about 50%, about 25% to about 50%, about 30% to about 50%, about 40% to about 50%, about 1% to about 40%, about 5% to about 40%, about 10% to about 40%, about 15% to about 40%, about 20% to about 40%, about 25% to about 40%, about 30% to about 40%, about 1% to about 30%, about 5% to about 30%, about 10% to about 30%, about 15% to about 30%, about 20% to about 30%, about 25% to about 30%, about 1% to about 25%, about 5% to about 25%, about 10% to about 25%, about 15% to about 25%, about 20% to about 25%, about 1% to about 20%, about 5% to about 20%, about 10% to about 20%, about 15% to about 20%, about 1% to about 15%, about 5% to about 15%, about 10% to about 15%, about 1% to about 10%, about 5% to about 10%, about 1% to about 5%, about 1%, 5%, 10%, 15%, 20%, 25%, 30%, 40%, 50%, 60%, 70%, 80%, 90%, 91%, 92%, 93%, 94%, or 95%, or greater than 1%, 5%, 10%, 15%, 20%, 25%, 30%, 40%, 50%, 60%, 70%, 80%, 90%, 91%, 92%, 93%, 94%, or 95%.

In any of the foregoing embodiments, the EMO (and thus the oil composition, if EMO is present) can include monoacylglycerols (MAGs). In any of the foregoing embodiments, the EMO can include a MAG content equal to or greater than 30% by weight of the total weight of the EMO. By way of example, but not limitation, the EMO can include a MAG content of about 30% to about 95%, about 40% to about 95%, about 50% to about 95%, about 60% to about 95%, about 70% to about 95%, about 80% to about 95%, about 90% to about 95%, about 30% to about 90%, about 40% to about 90%, about 50% to about 90%, about 60% to about 90%, about 80% to about 90%, about 30% to about 80%, about 40% to about 80%, about 50% to about 80%, about 60% to about 80%, about 70% to about 80%, about 30% to about 70%, about 40% to about 70%, about 50% to about 70%, about 50% to about 60%, about 30% to about 50%, about 40% to about 50%, or about 30%, 35%, 40%, 45%, 50%, 55%, 60%, 65%, 70%, 75%, 80%, 85%, 90%, or 95% by weight of the EMO.

In any of the foregoing embodiments, the EMO (and thus the oil composition, if EMO is present) can include diacylglycerols (DAGs). In any of the foregoing embodiments, the EMO can include a DAG content equal to or greater than 5% by weight of the total weight of the EMO. By way of example, but not limitation, the EMO can include a DAG content of about 5% to about 66%, about 10% to about 66%, about 20% to about 66%, about 30% to about 66%, about 40% to about 66%, about 50% to about 66%, about 5% to about 60%, about 10% to about 60%, about 20% to about 60%, about 30% to about 60%, about 40% to about 60%, about 50% to about 60%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight of the total weight of the EMO.

In any of the foregoing embodiments, the EMO can have fatty acid profile comprising an amount of two or more fatty acids, wherein the amount of each of the two or more fatty acids within 10% of the amount of each of the two or more fatty acids in the first oil source. By way of example, but not limitation, the EMO can have a fatty acid profile comprising an amount of three or more, four or more, five or more, six or more, seven or more, eight or more, nine or more, ten or more, eleven, or twelve or more fatty acids, wherein the amount of each of the fatty acids is within 10% of the amount of each of the fatty acids, 9%, 8%, 7%, 6%, 5%, 4%, 3%, 2%, or 1% or be about the same amount. For example, if the first oil source has 15% C18 fatty acid, the EMO can have between about 5-25%, 6-24%, 7-23%, 8-22%, 9-21%, 10-20%, 11-19%, 12-18%, 13-17%, 14-16% or about 15% C18 fatty acid.

It should be understood, in any of the foregoing embodiments, that the first oil source and the second oil source can be the same type of oil or different types of oil and that each of the first oil source and the second oil source can be a single type of oil (e.g. oat oil or flaxseed oil) or a combination of oils as described further herein. It should likewise be further understood that the EMO and/or FFA oil can be the result of a blend of EMO and/or FFA oils, respectively, which have been processed independently and later combined such that the first oil source or the second oil source are each the combination of oil sources used to produce the different EMO and/or FFA oils that have been combined.

In any of the foregoing embodiments, the oil composition can include at least 2% FFAs by weight of the oil composition. By way of example, but not limitation, the oil composition can include at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% FFAs by weight of the oil. By way of further example, but not limitation, the oil can include about 2% to about 99%, about 5% to about 99%, about 10% to about 99%, about 15% to about 99%, about 20% to about 99%, about 25% to about 99%, about 30% to about 99%, about 35% to about 99%, about 40% to about 99%, about 50% to about 99%, about 60% to about 99%, about 70% to about 99%, about 80% to about 99%, about 90% to about 99%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 90%, about 25% to about 80%, about 25% to about 75%, about 25% to about 70%, about 25% to about 60%, about 25% to about 50%, about 25% to about 40%, about 25% to about 30%, about 30% to about 90%, about 30% to about 80%, about 30% to about 75%, about 30% to about 70%, about 30% to about 60%, about 30% to about 50%, about 30% to about 40%, about 40% to about 90%, about 40% to about 80%, about 40% to about 75%, about 40% to about 70%, about 40% to about 60%, about 40% to about 50%, about 50% to about 90%, about 50% to about 80%, about 50% to about 75%, about 50% to about 70%, about 50% to about 60%, about 60% to about 90%, about 60% to about 80%, about 60% to about 75%, about 60% to about 70%, about 70% to about 90%, about 70% to about 80%, about 80% to about 90%, about 9% to about 37%, or about 2%, 3%, 4%, 5%, 6%, 7%, 8%, 9%, 10%, 15%, 20%, 25%, 30%, 40%, 50%, 60%, 70%, 75%, 80%, 90%, 95%, or 99% FFAs by weight of the oil. It should be understood that, where the oil composition is 100% FFA oil, the oil composition can include up to 100% FFA by weight of the oil composition.

In any of the foregoing embodiments, the EMO and the FFA oil can each comprise two or more different fatty acids. In any of the foregoing embodiments, the two or more different fatty acids can be, by way of example but not limitation, selected from the group consisting of caproic acid, enanthic acid, caprylic acid, pelargonic acid, capric acid, undecylic acid, lauric acid, tridecylic acid, myristic acid, pentadecylic acid, palmitic acid, acid, oleic acid, linoleic acid, linolenic acid, stearidonic acid, octadecatetraenoic acid, arachidic acid, heneicosylic acid, gondoic acid, eicosadienoic acid, paullinic acid, behenic acid, tricosylic acid, erucic acid, lignoceric acid, docosahexaenoic acid, eicosapentaenoic acid, nervonic acid, and isomers thereof. Alternatively, the fatty acids, including the two or more different fatty acids can each independently be selected from the group consisting of a C6 fatty acid such as, by way of example but not limitation, C6:0 fatty acid, a C7 fatty acid, a C8 fatty acid such as, by way of example, but not limitation a C8:0 fatty acid, a C9 fatty acid, a C10 fatty acid, a C11 fatty acid, a C12 fatty acid, a C13 fatty acid, a C14 fatty acid, a C15 fatty acid, a C16 fatty acid, a C17 fatty acid, a C18 fatty acid such as, by way of example, but not limitation, a C18:0 fatty acid, a C18:0 fatty acid, a C18:1 fatty acid, a C18:2 fatty acid, or a C18:3 fatty acid, a C19 fatty acid, a C20 fatty acid such as, by way of example, but not limitation, C20:5 fatty acid, a C21 fatty acid, a C22 fatty acid such as, by way of example, but not limitation, C22:6 fatty acid, a C23 fatty acid, and a C24 fatty acid. It should be understood that the two or more different fatty acids can be the same between the EMO and FFA oil or can be different.

In any of the foregoing embodiments, the two or more different fatty acids can be present in the EMO or FFA oil in an amount that is within 10%, 9%, 8%, 7%, 6%, 5%, 4%, 3%, 2% or 1% of the amount of that fatty acid in the fatty acid profile of the first oil source or the second oil source, respectively. In any of the foregoing embodiments, the two or more different fatty acids, can be two fatty acids, three fatty acids, four fatty acids, five fatty acids, six fatty acids, seven fatty acids, eight fatty acids, nine fatty acids, ten fatty acids or more. For example, if a first oil source has 20% C18:1 fatty acid and 20% C12 fatty acid, then the EMO would have 10-30% C18:1 and 10-30% C12 if it is within 10% of the amount of the fatty acids in the fatty acid profile of the first source oil. Thus, where the oil composition is a blend of EMO and FFA oil from different oil sources, the fatty acid profile of the oil composition will vary according to the ratio of EMO:FFA oil and the expected ranges can be calculated by one of skill in the art. However, where the EMO and FFA are derived from the same source oil, the fatty acid profile of the oil composition would be expected to be similar to that of the source oil.

In any of the foregoing embodiments, the EMO and/or the FFA oil (and thus the oil composition) can be substantially free of 3-monochloropropanediol and glycidol equivalents.

Nanoemulsion Compositions

It should be understood in any of the following embodiments, that the nanoemulsions or microemulsions of the present disclosure can include the antimicrobial oil compositions of any of the foregoing embodiments. Alternatively, the nanoemulsions or microemulsions can include an EMO, FFA oil or both as described in any of the embodiments herein. It should be further understood that that nanoemulsion, or oil component thereof need not have antimicrobial activity but can have antimicrobial activity as described in embodiments herein.

In some embodiments, a nanoemulsion composition is provided that includes an oil dispersed in a liquid, where the oil is in the form of droplets or particles and includes at least 2% free fatty acids (FFAs) by weight of the oil, where the FFAs include two or more different fatty acids, where the oil includes 80% or less triacylglycerols (TAGs) by weight out of the total weight of the oil, and where the droplets or particles have a Z average size of about 300 nm or less as measured by dynamic light scattering.

In some embodiments, a nanoemulsion composition is provided that includes an oil dispersed in a liquid, where the oil is in the form of droplets or particles and includes at least 2% FFAs by weight of the oil and 80% or less TAGs by weight of the oil, where the oil includes non-oil ingredients that are naturally present in the oil, and where the droplets or particles have a Z average particle size of about 300 nm or less as measured by dynamic light scattering.

In some embodiments, a nanoemulsion composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a nanoemulsion composition is provided that includes an oil dispersed in a liquid, where the oil is in the form of droplets or particles and includes at least 2% FFAs by weight of the oil, where the FFAs include two or more different fatty acids, where the oil also includes MAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In some embodiments, a nanoemulsion composition is provided that includes an oil dispersed in a liquid, where the oil is in the form of droplets or particles and includes at least 2% FFAs by weight of the oil, where the FFAs include two or more different fatty acids, where the oil also includes monoacylglycerols (MAGs) and diacylglycerols (DAGs) in an amount of at least 5% by weight out of the total weight of the oil, where the MAGs and FFAs are derived from a common, first oil source, and where the droplets or particles have a Z average particle size of about 300 nm or less as measured by dynamic light scattering.

In some embodiments, a nanoemulsion composition is provided that includes an oil dispersed in a liquid, where the oil is in the form of droplets or particles and includes at least 2% FFAs by weight of the oil, where the FFAs include two or more different fatty acids, where the oil also includes MAGs and DAGs in an amount of at least 5% by weight of the total weight of the oil, where the MAGs and FFAs are derived from a first oil source and a second oil source, respectively, and where the droplets or particles have a Z average size of about 300 nm or less as measured by dynamic light scattering.

In any of the foregoing embodiments, the oil can include at least 2% FFAs by weight of the oil. By way of example, but not limitation, the oil can include at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% FFAs by weight of the oil. By way of further example, but not limitation, the oil can include about 2% to about 99%, about 5% to about 99%, about 10% to about 99%, about 15% to about 99%, about 20% to about 99%, about 25% to about 99%, about 30% to about 99%, about 35% to about 99%, about 40% to about 99%, about 50% to about 99%, about 60% to about 99%, about 70% to about 99%, about 80% to about 99%, about 90% to about 99%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 90%, about 25% to about 80%, about 25% to about 75%, about 25% to about 70%, about 25% to about 60%, about 25% to about 50%, about 25% to about 40%, about 25% to about 30%, about 30% to about 90%, about 30% to about 80%, about 30% to about 75%, about 30% to about 70%, about 30% to about 60%, about 30% to about 50%, about 30% to about 40%, about 40% to about 90%, about 40% to about 80%, about 40% to about 75%, about 40% to about 70%, about 40% to about 60%, about 40% to about 50%, about 50% to about 90%, about 50% to about 80%, about 50% to about 75%, about 50% to about 70%, about 50% to about 60%, about 60% to about 90%, about 60% to about 80%, about 60% to about 75%, about 60% to about 70%, about 70% to about 90%, about 70% to about 80%, about 80% to about 90%, about 2%, 3%, 4%, 5%, 6%, 7%, 8%, 9%, 10%, 15%, 20%, 25%, 30%, 40%, 50%, 60%, 70%, 75%, 80%, 90%, 95%, or 99% FFAs by weight of the oil. By way of still further example, but not limitation, in any of the foregoing embodiments where a minimum amount of MAGs and/or DAGs is not recited, the FFA can be present at up to about 100% by weight of the oil.

In any of the foregoing embodiments, the oil can include non-oil ingredients that are naturally present in the oil. By way of example, but not limitation, the non-oil ingredients that are naturally present in the oil can include one or more of glycosphingolipids such as ceramide phosphates, glycoglyceroplipids such as monogalactosyl diacylglycerols, phophatidyl alcohols such as phosphatidyl methanol, steroids such as sitosteryl esters, natural lipids such as campesterol esters, sphingolipids, phosphatidyl glycerol, wax esters, sphingomyelin, phosphatides, phytosterols (campesterol, stigmasterol, sitosterol), cholesterol, tocopherols, tocotrienols, carotenes, xanthophylls, betaxanthins, chlorophyll, long chain alcohols, polyphenols, terpenes (cycloartanol, cycloarterenol, 24-methylene cycloarterenol), terpenoids (squalene), tocopherol, vitamins, paraffins, isothiocyanates, oxazolidinthiones, glucosinolates, sanguinarine, gossypols, glycosylated alkaloids, phospholipids, sphingosine, diacylglyceride ethers, glucolipids, phytic acid, quinic acid, oxalic acid, tartaric acid, anacardic acid, malic acid, caftaric acid, coutaric acid, fertaric acid, phenylethanoids, hydroxycinnamic acids, phenolic acids, phytoestrogens, lignans, isoflavonoids, flavonoids, monoterpenes, diterpenes, triterpenoids, coenzyme, or combinations thereof. In any of the foregoing embodiments, the oil can include non-oil ingredients derived from and naturally present in the oil source and selected from the group consisting of α-tocopherol, β-tocopherol, δ-tocopherol, γ-tocopherol, α-tocotrienol, β-tocotrienol, δ-tocotrienol, and γ-tocotrienol. In any of the foregoing embodiments, the oil can include non-oil ingredients derived from and naturally present in the oil source(s) and selected from the group consisting of ceramide phosphate, monoglactodiacylglycerol, phosphatidylmethanol, sitosteryl ester, campesterol ester, sphingolipids, phosphatidyl glycerol, wax esters, sphingomyelin, and combinations thereof. It should be understood that the level of the non-oil ingredients can be the same as or greater than that in the oil source. By way of example, but not limitation, the non-oil components can be determined by LC/MS/MS analysis.

In any of the foregoing embodiments, the oil composition can include monoacylglycerols (MAGs), including those contributed by the EMO, if present. By way of example, but not limitation, the MAGs can be present in the oil in an amount of at least 5%, at least 10%, at least 15%, at least 20%, at least 25%, at least 30%, at least 40%, at least 50%, at least 60%, at least 70%, at least 80%, at least 90%, about 5%, about 10%, about 15%, about 20%, about 25%, about 30%, about 40%, about 50%, about 60%, about 70%, about 75%, about 80%, about 90%, about 95%, about 98%, about 5% to about 98%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 98%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 98%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 98%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 98%, about 30% to about 95%, about 30% to about 90%, about 30% to about 80%, about 30% to about 75%, about 30% to about 70%, about 30% to about 60%, about 30% to about 50%, about 30% to about 40%, about 40% to about 98%, about 40% to about 95%, about 40% to about 90%, about 40% to about 80%, about 40% to about 75%, about 40% to about 70%, about 40% to about 60%, about 40% to about 50%, about 50% to about 98%, about 50% to about 95%, about 50% to about 90%, about 50% to about 80%, about 50% to about 75%, about 50% to about 70%, about 50% to about 60%, about 60% to about 98%, about 60% to about 95%, about 60% to about 90%, about 60% to about 80%, about 60% to about 75%, about 60% to about 70%, about 70% to about 98%, about 70% to about 95%, about 70% to about 90%, about 70% to about 80%, about 70% to about 75%, about 75% to about 98%, about 75% to about 95%, about 75% to about 90%, about 75% to about 80%, about 80% to about 98%, about 80% to about 95%, about 80% to about 90%, about 90% to about 98%, about 90% to about 95%, or about 95% to about 98% by weight out of the total weight of the oil. In any of the foregoing embodiments, the oil can further include diacylglycerols (DAGs). By way of example, but not limitation, the DAGs can be present in the oil in an amount of greater than about 5%, from about 5% to about 66%, about 10% to about 66%, about 20% to about 66%, about 30% to about 66%, about 30% to about 66%, about 40% to about 66%, about 50% to about 66%, about 5% to about 50%, about 10% to about 50%, about 20% to about 50%, about 30% to about 50%, about 40% to about 50%, about 5% to about 40%, about 10% to about 40%, about 20% to about 40%, about 30% to about 40%, about 5% to about 30%, about 10% to about 30%, about 20% to about 30%, about 5% to about 20%, about 10% to about 20%, about 5% to about 10%, or about 5%, 10%, 15%, 20%, 25%, 30%, 35%, 40%, 45%, 50%, 55%, 60%, or 66% by weight out of the total weight of the oil. Alternatively, in any of the foregoing embodiments, the oil can further include MAGs and DAGs. By way of example, but not limitation, the MAGs and DAGs can be present in the oil in an amount of at least 5%, at least 10%, at least 15%, at least 20%, at least 25%, at least 30%, at least 40%, at least 50%, at least 60%, at least 70%, at least 80%, at least 90%, about 5%, about 10%, about 15%, about 20%, about 25%, about 30%, about 40%, about 50%, about 60%, about 70%, about 75%, about 80%, about 90%, about 95%, about 98%, about 5% to about 98%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 98%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 98%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 98%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 98%, about 30% to about 95%, about 30% to about 90%, about 30% to about 80%, about 30% to about 75%, about 30% to about 70%, about 30% to about 60%, about 30% to about 50%, about 30% to about 40%, about 40% to about 98%, about 40% to about 95%, about 40% to about 90%, about 40% to about 80%, about 40% to about 75%, about 40% to about 70%, about 40% to about 60%, about 40% to about 50%, about 50% to about 98%, about 50% to about 95%, about 50% to about 90%, about 50% to about 80%, about 50% to about 75%, about 50% to about 70%, about 50% to about 60%, about 60% to about 98%, about 60% to about 95%, about 60% to about 90%, about 60% to about 80%, about 60% to about 75%, about 60% to about 70%, about 70% to about 98%, about 70% to about 95%, about 70% to about 90%, about 70% to about 80%, about 70% to about 75%, about 75% to about 98%, about 75% to about 95%, about 75% to about 90%, about 75% to about 80%, about 80% to about 98%, about 80% to about 95%, about 80% to about 90%, about 90% to about 98%, about 90% to about 95%, or about 95% to about 98% by weight out of the total weight of the oil.

In any of the foregoing embodiments, the droplets or particles can have an average zeta potential of less than about −30 mV. By way of example, but not limitation, the droplets or particles can have an average zeta potential of less than about −35 mV, less than about −40 mV, less than about −45 mV, less than about −50 mV, less than about −60 mV, less than about −70 mV, about −30 mV to about −80 mV, about −30 mV to about −75 mV, about −30 mV to about −70 my, about −30 mV to about −60 mV, about −30 mV to about −50 mV, about −30 mV to about −40 mV, about −40 mV to about −80 mV, about −40 mV to about −75 mV, about −40 mV to about −70 mV, about −40 mV to about −60 mV, about −40 mV to about −50 mV, about −50 mV to about −80 mV, about −50 mV to about −75 mV, about −50 mV to about −70 mV, about −50 mV to about −60 mV, about −60 mV to about −80 mV, about −60 mV to about −75 mV, about −60 mV to about −70 mV, about −70 mV to about −80 mV, about −70 mV to about −75 mV, about −30 mV, −35 mV, −40 mV, −45 mV, −50 mV, −60 mV, −70 mV, −75 mV, or −90 mV. In certain aspects, the zeta potential can be as measured by electrophoretic light scattering.

In any of the foregoing embodiments, the droplets or particles can have a polydispersity index (PDI) of less than about 0.3. By way of example, but not limitation, the droplets or particles can have a PDI of less than about 0.3, less than about 0.25, less than about 0.2, less than about 0.175, about 0.15 to about 0.3, about 0.2 to about 0.3, about 0.25 to about 0.3, about 0.15, 0.16, 0.17, 0.18, 0.19, 0.2, 0.21, 0.22, 0.23, 0.24, 0.25, 0.26, 0.27, 0.28, 0.29 or 0.3. In certain aspects, the PDI can be as measured by dynamic light scattering.

In any of the foregoing embodiments, the droplets or particles can have a Z average size of about 300 nm or less. By way of example, but not limitation, the droplets or particles can have a Z average size of about 300 nm or less, about 275 nm or less, about 250 nm or less, about 225 nm or less, about 200 nm or less, about 175 nm or less, about 150 nm or less, about 100 nm to about 300 nm, about 100 nm to about 250 nm, about 100 nm to about 200 nm, about 150 nm to about 300 nm, about 150 nm to about 250 nm, about 150 nm to about 200 nm, about 200 nm to about 300 nm, about 200 nm to about 250 nm, about 250 nm to about 300 nm, about 150 nm, about 160 nm, about 170 nm, about 175 nm, about 180 nm, about 190 nm, about 200 nm, about 210 nm, about 220 nm, about 225 nm, about 230 nm, about 240 nm, about 250 nm, about 260 nm, about 270 nm, about 275 nm, about 280 nm, about 290 nm, or about 300 nm. In certain aspects, the Z average size can be as measured by dynamic light scattering.

In any of the foregoing embodiments, the oil can include a processed oil from an oil source that has been subjected to an enzymatic treatment selected from the group consisting of enzymatic hydrolysis to yield free fatty acids and glycerol with removal of glycerol to yield FFA oil, enzymatic re-esterification of FFA to yield monoacylglycerols (MAGs) and free fatty acids and, optionally, diacylglycerols (DAGs), and both enzymatic hydrolysis to yield free fatty acids and glycerol with removal of glycerol to yield FFA oil and enzymatic re-esterification of FFA to yield monoacylglycerols (MAGs) and free fatty acids and, optionally, diacylglycerols (DAGs) (EMO). By way of example, but not limitation, the oil or a portion thereof can be an enzyme-modified oil (EMO) that has been subjected to enzymatic hydrolysis to yield free fatty acids and glycerol, separation of the glycerol from the free fatty acid oil, and subsequent enzymatic re-esterification to yield monoacylglycerols, free fatty acids and, optionally, diacylglcyerols. It should be understood that the oil so processed can be from a single oil source or a combination of oil sources which are subjected to the enzymatic treatment(s). It should be further understood that the EMO and/or FFA oil from a single or multiple oil sources can be blended after the enzymatic treatment(s) to formulate the oil in the compositions of the present disclosure. Thus, in some embodiments, the oil can include a blend of EMO and FFA oil from one, two, three, four, five or more oil sources. It should likewise be understood that EMOs and/or FFA oils can also be blended with unmodified, TAG oils to yield the oil in the compositions of the present disclosure. Thus, in some embodiments, the oil can include a blend of two or more processed oils, from each of a first oil source and a second oil source. By way of example, but not limitation, the oil can include a blend of two, three, four or more processed oils.

It should be understood that the MAGs and, optionally, DAGs, or a portion thereof, in compositions of the present disclosure can be derived from a first oil source. In further embodiments, the FFA, or a portion thereof, can be derived from a second oil source. For example, an EMO may contribute a portion of the FFA content of an oil composition while the FFA oil will provide additional FFA content. In other embodiments, the FFA, or portion thereof, is derived from the same source as the first oil source. It should be understood that various combinations of MAGs, DAGs, TAGs and FFA from different oils can be used. In any of the foregoing embodiments, the oil can be derived from a single oil source, two oil sources, three oil sources, four oil sources, five oil sources or more.

In any of the foregoing embodiments, the MAGs can include two or more different MAGs. By way of example, but not limitation, the MAGs can include two, three, four, five or more different MAGs. By way of further example, but not limitation, the MAGs can be selected from the group consisting of monocaproin, monocaprylin, monocaprin, monolaurin, monomyristin, monopalmitin, monostearin, monoolein, monolinolein, monolinolenin, monoeicosapentaenoin, monodocosahexaenoin, and combinations thereof.

While the oil can be from a single oil source and type, such as, by way of example, but not limitation, EMO from a single oil source, as noted, the oil can include components from multiple oil sources. By way of example, but not limitation, the oil can include an EMO from a first oil source and a FFA oil from a second oil source. By way of further example, but not limitation, the oil can include a first EMO from a first oil source and a second EMO from a second oil source. By way of still further example, the oil can include a first EMO from a first oil source, a second EMO from a second oil source, and a FFA oil from a third oil source. Thus, it should be understood that any combination of oil components from oil sources, even unmodified TAG oils, that is used to form a compositions and nanoemulsions of the present disclosure is within the scope of the present disclosure. By way of even further example, where an EMO is used, the EMO can be derived from two or more oil sources.

In any of the foregoing embodiments, the oil, or a portion thereof, can be derived, from an oil source selected from a plant, an animal, a fish, an algal oil, and combinations thereof. In any of the foregoing embodiments, by way of example but not limitation, the oil can be derived from olive oil, almond oil, canola oil, coconut oil, cottonseed oil, palm kernel oil, palm olein oil, palm stearin oil, peanut oil, flaxseed oil, sunflower seed oil, corn oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, and combinations thereof. By way of example, but not limitation, where a first and second oil source are utilized, these can be derived from olive oil, almond oil, canola oil, coconut oil, cottonseed oil, palm kernel oil, palm olein oil, palm stearin oil, peanut oil, flaxseed oil, sunflower seed oil, com oil, grapeseed oil, pomegranate oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, edible oils, medicinal oils, and combinations thereof. It should be further understood that the oil of the compositions of the present disclosure can include two, three, four or more oils from various source oils.

In some embodiments, the oil composition can include an unmodified, TAG oil component. The TAG oil component can be an oil selected from a plant, an animal, a fish, an algal oil, and combinations thereof. By way of example, but not limitation, the unmodified, TAG oil component can be olive oil, almond oil, canola oil, coconut oil, cottonseed oil, palm kernel oil, palm olein oil, palm stearin oil, peanut oil, flaxseed oil, sunflower seed oil, corn oil, grapeseed oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, other edible oils, medicinal oils, and combinations thereof. By way of example, but not limitation, where a first and second oil source are utilized, these can be derived from olive oil, almond oil, canola oil, coconut oil, cottonseed oil, palm kernel oil, palm olein oil, palm stearin oil, peanut oil, flaxseed oil, sunflower seed oil, com oil, grapeseed oil, rose hip oil, hemp seed oil, prickly pear oil, medium chain triglyceride oil, safflower oil, sesame oil, walnut oil, palm oil, soybean oil, fish oil, sardine oil, anchovy oil, algal oil, chicken fat, lard, krill oil, avocado oil, mustard oil, rice bran oil, oat oil, nutmeg butter, macadamia nut oil, cacao butter, rapeseed oil, poppy seed oil, castor oil, other edible oils, medicinal oils, and combinations thereof. It should be further understood that the oil of the compositions of the present disclosure can include two, three, four or more oils from various oil sources. In any of the foregoing embodiments, the TAG oil component can be present at up to 80% by weight of the oil. By way of example, but not limitation, the TAG oil component can be present at about 0.1% to about 80%, about 1% to about 80%, about 5% to about 80%, about 10% to about 80%, about 20% to about 80%, about 30% to about 80%, about 40% to about 80%, about 50% to about 80%, about 60% to about 80%, about 70% to about 80%, about 0.1% to about 70%, about 1% to about 70%, about 5% to about 70%, about 10% to about 70%, about 20% to about 70%, about 30% to about 70%, about 40% to about 70%, about 50% to about 70%, about 60% to about 70%, about 0.1% to about 60%, about 1% to about 60%, about 5% to about 60%, about 10% to about 60%, about 20% to about 60%, about 30% to about 60%, about 40% to about 60%, about 50% to about 60%, about 0.1% to about 50%, about 1% to about 50%, about 5% to about 50%, about 10% to about 50%, about 20% to about 50%, about 30% to about 50%, about 40% to about 50%, about 0.1% to about 40%, about 1% to about 40%, about 5% to about 40%, about 10% to about 40%, about 20% to about 40%, about 30% to about 40%, about 0.1% to about 30%, about 1% to about 30%, about 5% to about 30%, about 10% to about 30%, about 20% to about 30%, about 0.1% to about 20%, about 1% to about 20%, about 5% to about 20%, about 10% to about 20%, about 0.1% to about 10%, about 1% to about 10%, about 5% to about 10%, about 0.1% to about 5%, about 1% to about 5%, about 0.1% to about 1%, or about 0.1%, 1%, 2%, 3%, 4%, 5%, 10%, 15%, 20%, 25%, 30%, 40%, 50%, 60%, 70%, or 80% by weight of the oil.

In any of the foregoing embodiments, the oil can include a fatty acid profile that is within about 10% of the fatty acid profile from the oil source used to yield the oil for at least one, two or more fatty acids. In aspects where more than one oil source is used to form the oil, the fatty acid profile can be within about 10% of the fatty acid profile from the oil sources according to the respective amounts of each oil source used to form the oil for at least one, two or more fatty acids. By way of example, but not limitation, the fatty acid profile of the oil for one, two, three, four, five, six, seven, eight, nine, ten or more fatty acids can be within 10% of the fatty acid profile for the same fatty acids in the oil source(s), according to the respective amounts of each oil source used to form the oil if two or more oil sources are used, or for the oil source where only one oil source is used to form the oil. By way of further example, but not limitation, the fatty acid profile can be within about 10%, 9%, 8%, 7%, 6%, 5%, 4%, 3%, 2%, or 1% for each fatty acid in the oil as compared to the oil source(s).

In any of the foregoing embodiments, the oil can be about 0.001% to about 90% by weight out of the total weight of the nanoemulsion composition or an antimicrobial composition comprising the antimicrobial oil composition or nanoemulsion such as, by way of example, but not limitation, a cream. By way of example, but not limitation, the oil can be about 0.001% to about 90%, about 0.01% to about 90%, about 0.1% to about 90%, about 1% to about 90%, about 2% to about 90%, about 3% to about 90%, about 4% to about 90%, about 5% to about 90%, about 10% to about 90%, about 20% to about 90%, about 30% to about 90%, about 40% to about 90%, about 50% to about 90%, about 60% to about 90%, about 70% to about 90%, about 80% to about 90%, about 0.001% to about 80%, about 0.01% to about 80%, about 0.1% to about 80%, about 1% to about 80%, about 2% to about 80%, about 3% to about 80%, about 4% to about 80%, about 5% to about 80%, about 10% to about 80%, about 20% to about 80%, about 30% to about 80%, about 40% to about 80%, about 50% to about 80%, about 60% to about 80%, about 70% to about 80%, about 0.001% to about 70%, about 0.01% to about 70%, about 1% to about 70%, about 2% to about 70%, about 3% to about 70%, about 4% to about 70%, about 5% to about 70%, about 10% to about 70%, about 20% to about 70%, about 30% to about 70%, about 40% to about 70%, about 50% to about 70%, about 60% to about 70%, about 0.001% to about 60%, about 0.01% to about 60%, about 0.1% to about 60%, about 1% to about 60%, about 2% to about 60%, about 3% to about 60%, about 4% to about 60%, about 5% to about 60%, about 10% to about 60%, about 20% to about 60%, about 30% to about 60%, about 40% to about 60%, about 50% to about 60%, about 0.001% to about 50%, about 0.01% to about 50%, about 0.1% to about 50%, about 1% to about 50%, about 2% to about 50%, about 3% to about 50%, about 4% to about 50%, about 5% to about 50%, about 10% to about 50%, about 20% to about 50%, about 30% to about 50%, about 30% to about 50%, about 0.001% to about 40%, about 0.01% to about 40%, about 0.1% to about 40%, about 1% to about 40%, about 2% to about 40%, about 3% to about 40%, about 4% to about 40%, about 5% to about 40%, about 10% to about 40%, about 20% to about 40%, about 30% to about 40%, about 0.001% to about 30%, about 0.01% to about 30%, about 0.1% to about 30%, about 1% to about 30%, about 2% to about 30%, about 3% to about 30%, about 4% to about 30%, about 5% to about 30%, about 10% to about 30%, about 20% to about 30%, about 0.001% to about 20%, about 0.01% to about 20%, about 0.1% to about 20%, about 1% to about 20%, about 2% to about 20%, about 3% to about 20%, about 4% to about 20%, about 5% to about 20%, about 10% to about 20%, about 0.001% to about 10%, about 0.01% to about 10%, about 0.1% to about 10%, about 1% to about 10%, about 2% to about 10%, about 3% to about 10%, about 4% to about 10%, about 5% to about 10%, about 0.001% to about 5%, about 0.01% to about 5%, about 0.1% to about 5%, about 1% to about 5%, about 2% to about 10%, about 2% to about 5%, about 3% to about 10%, about 3% to about 5%, about 4% to about 10%, about 4% to about 5%, about 1%, 2%, 3%, 4%, 5%, 6%, 7%, 8%, 9%, 10%, 15%, 20%, 25%, 30%, 40%, 50%, 60%, 70%, 75%, 80%, or 90% by weight out of the total weight of the composition.

In any of the foregoing embodiments, the liquid can be water or any suitable polar medium. By way of example, but not limitation, the polar medium can be water, deionized water, saline, buffered water (e.g., phosphate buffered saline (PBS), citric acid buffer, sodium acetate buffer), salt solutions (e.g., sodium chloride or potassium chloride) and other non-aqueous solutions.

In any of the foregoing embodiments, the Z average size of the droplets or particles does not change by more than 15% over at least 14 days when stored at room temperature (22° C.). By way of example, but not limitation, the Z average size of the droplets or particles does not change by more than 15% over at least 14 days, 21 days, or 28 days when stored at room temperature (22° C.). By way of further example, but not limitation, the Z average size of the droplets or particles does not change by more than 15%, by more than 10%, or by more than 5% over 14 days, 21 days or 28 days when stored at room temperature (22° C.).

In any of the foregoing embodiments, the Z average size of the droplets or particles does not change by more than 15% over at least 14 days when stored at 4° C. By way of example, but not limitation, the Z average size of the droplets or particles does not change by more than 15% over at least 14 days, 21 days, or 28 days when stored at 4° C. By way of further example, but not limitation, the Z average size of the droplets or particles does not change by more than 15%, by more than 10%, or by more than 5% over 14 days, 21 days or 28 days when stored at 4° C.

In any of the foregoing embodiments, the oil can be in the form of droplets. Alternatively, in any of the foregoing embodiments, the oil can be in the form of particles. For example, where the nanoemulsion is a solid lipid nanoemulsion (SLN), the oil would be in the form of particles. It should be understood that droplets refers to liquid droplets, while particles refers to solid particles.

In any of the foregoing embodiments, the antimicrobial oil composition, nanoemulsion composition, or a composition comprising either can not include any exogenous additive with surfactant and/or emulsifier properties that is not naturally present in the oil in an amount sufficient to enhance droplet or particle stability. By way of example, but not limitation, the antimicrobial oil composition, nanoemulsion composition, or a composition comprising either can not include polysorbates, phospholipids, sterols, cationic lipids, poloxamers, sorbitan esters, sugar esters, polyoxyethylene conjugates, ammonium phosphatidyl lapsidate, polyphenols, polyvinylalcohol, lecithin, ethanol, lysophospholipids, ceramides, Tweens, Spans, Kolliphor, propylene glycol, vitamin D, polyethylene glycol, polyethylene glycol derivatives, gum acacia, modified gum acacia, agar, ghatti gum, modified ghatti gum, pectin, carrageenan, xanthan gum, modified starches, modified alginate, fatty alcohols, ethoxylated polyols, and combinations thereof.

In any of the foregoing embodiments, the antimicrobial oil composition, nanoemulsion composition, or a composition comprising either can not include any exogenous additive with thickening or crystallization inhibiting properties that is not naturally present in the oil in an amount sufficient to thicken the composition or inhibit crystal formation, respectively. By way of example, but not limitation, the antimicrobial oil composition, nanoemulsion composition, or a composition comprising either can not include methyl cellulose, cornstarch, sodium alginate, or gelatin. By way of further example, but not limitation, the antimicrobial oil composition, nanoemulsion composition, or a composition comprising either can not include polyglycerol fatty acid esters, sucrose fatty acid esters, sorbitan fatty acid esters, and combinations thereof. In certain aspects, the composition can not include any exogenous additive with thickening properties that is not naturally present in the oil in an amount sufficient to thicken the composition. In certain aspects, the antimicrobial oil composition, nanoemulsion composition, or a composition comprising either can not include any exogenous additive with crystallization inhibiting properties that is not naturally present in the oil in an amount sufficient to inhibit crystal formation. In other aspects, the antimicrobial oil composition, nanoemulsion composition, or a composition comprising either can not include any exogenous additive with thickening or crystallization inhibiting properties that is not naturally present in the oil in an amount sufficient to thicken the composition or inhibit crystal formation, respectively.

In any of the foregoing embodiments, the antimicrobial oil composition, nanoemulsion composition, or a composition comprising either, can not include a quaternary amine. It has surprisingly been found that the antimicrobial oil compositions of the present disclosure can avoid the need for harsh preservatives such as quaternary amines and can enable to the use of milder preservatives.

In any of the foregoing embodiments, the antimicrobial oil composition, nanoemulsion composition, or a composition comprising either can not include a polyhydric alcohol.

In any of the foregoing embodiments, the antimicrobial oil composition, nanoemulsion composition, or a composition comprising either can not include bile salts.

In any of the foregoing embodiments, the droplets can be mixed micelles. Alternatively, in any of the foregoing embodiments, the particles can be solid lipid particles.

In some embodiments, a method for preparing a nanoemulsion is provided that includes providing an oil of the present disclosure, providing a polar liquid component, combining the oil and polar liquid component to form a nanoemulsion pre-mix, and forming a nanoemulsion from the nanoemulsion pre-mix, where the nanoemulsion comprises an oil phase and an polar, liquid phase, where the oil phase is dispersed as droplets or particles within the polar, liquid phase, and where the droplets or particles have a Z average size of about 300 nm or less as measured by dynamic light scattering. In certain aspects, the oil can be heated, for example to 60° C., prior to combining with the polar, liquid component which itself can be pre-heated, for example to 60° C. By way of further example, but not limitation, the oil can be heated to about 40° C. to about 80° C., to about 40° C. to about 70° C., to about about 50° C. to about 80° C., to about 50° C. to about 70° C., to about 55° C. to about 65° C., to about 40° C., 45° C., 50° C., 55° C., 60° C., 65° C., 70° C., 75° C. or 80° C. By way of still further example, but not limitation, the polar liquid can be pre-heated to about 40° C. to about 80° C., to about 40° C. to about 70° C., to about about 50° C. to about 80° C., to about 50° C. to about 70° C., to about 55° C. to about 65° C., to about 40° C., 45° C., 50° C., 55° C., 60° C., 65° C., 70° C., 75° C. or 80° C.

In any of the foregoing method embodiments, the nanoemulsion can be formed under conditions sufficient to have the properties of any of the compositions of the foregoing embodiments.

In any of the foregoing method embodiments, the method can further include a step of forming the oil by blending one or more oil components. In some embodiments, the one or more oil components are derived from different oil sources or combinations thereof as described in the present disclosure. In some embodiments, the one or more oil components are derived from the same oil source. By way of example, but not limitation, the one or more oil components can be selected from an EMO, a FFA oil, an unmodified, TAG oil, and combinations thereof.

It should be understood that the polar, liquid medium can be any suitable polar, liquid medium, including those disclosed in the present disclosure with respect to compositions of the present disclosure.

By way of example, but not limitation, the nanoemulsion can be formed by adding the polar, liquid phase rapidly to the oil phase or by homogenization after combining to form a coarse emulsion, and sonicating the coarse emulsion to form the nanoemulsion. By way of further example, but not limitation, the nanoemulsion can be formed by adding the oil dropwise to the polar, liquid phase to form a coarse emulsion and sonicating the coarse emulsion to form the nanoemulsion.

It should be understood that other methods for manufacturing nanoemulsions are well-known and can be used by one of skill in the art to prepare nanoemulsions of the present disclosure. Such methods include, but are not limited to, high shear homogenization, microfluidization, bath sonication, solvent evaporation, supercritical fluid methods, spray drying methods and double emulsion methods.

Microemulsions and Larger Nanoemulsions

It should also be understood that the compositions of the present disclosure, although disclosed as being nanoemulsions, can also be formulated as larger nanoemulsions or microemulsions depending on the application. In any of the foregoing embodiments, rather than the Z average size of the droplets or particles being about 300 nm or less, the droplets or particles can have a Z average size of greater than about 300 nm or greater than about 1000 nm. By way of example, but not limitation, in addition to the size of the nanoemulsion compositions already disclosed, the droplets or particles can have a Z average size of greater than about 300 nm, greater than about 400 nm, greater than about 500 nm, greater than about 600 nm, greater than about 700 nm, greater than about 800 nm, greater than about 900 nm, greater than about 1000 nm, greater than about 10000 nm, greater than about 100000 nm, from about 300 nm to about 1000 nm, from about 500 nm to about 1000 nm, from about 750 to about 1000 nm, from about 1000 nm to about 999999 nm, from about 1000 nm to about 100000 nm, from about 1000 nm to about 10000 nm, from about 10000 to about 5000 nm, from about 1000 to about 3000 nm, about 300 nm, about 400 nm, about 500 nm, about 600 nm, about 700 nm, about 800 nm, about 900 nm, about 1000 nm, about 2000 nm, about 3000 nm, about 4000 nm, about 5000 nm, about 10000 nm, about 20000 nm, about 30000 nm, about 40000 nm, about 50000 nm, about 100000 nm, or about 1000000 nm. By way of further example, but not limitation, for certain antimicrobial applications, a microemulsion may be used instead of a nanoemulsion. By way of example, but not limitation, the size of microemulsions and larger nanoemulsions can be measured by microscopy or dynamic light scattering.

Methods of making microemulsions and larger nanoemulsions are well-known in the art and include, by way of example, but not limitation, homogenization, sonication, solvent evaporation, supercritical fluid methods, spray drying methods and double emulsion methods. By way of further example, but not limitation, the step of forming coarse nanoemulsions by homogenization disclosed in the present application can be used to form microemulsions and larger nanoemulsions. In methods of the present disclosure for forming nanoemulsions, the methods can be modified to form microemulsions by using conventional methods for forming microemulsions instead of forming a nanoemulsion. It should be understood that similar methods can be used to obtain larger nanoemulsions if desired. It should also be understood that methods for preparing nanoemulsions of the present disclosure can be used to the extent that they produce larger nanoemulsions or microemulsions. By way of example, but not limitation, to form a larger nanoemulsion or microemulsion, the oil can optionally be heated first and then combined with a polar liquid and subjected to homogenization. By way of further example, but not limitation, the polar liquid can be pre-heated prior to combining with the oil.

Antimicrobial Applications

In some embodiments, a method of treating or preventing a microbial infection in a subject in need thereof can include administering to the subject an antimicrobial oil composition of the present disclosure of any of the foregoing embodiments.

In some embodiments, a method of sanitizing or sterilizing an object or surface, can include the step of applying to the object or surface an effective amount of an antimicrobial oil composition of the present disclosure of any of the foregoing embodiments. It should be understood that such embodiments can have a specific microbial target or be a broad-spectrum target for sanitizing or sterilizing.

In the foregoing methods of treating or preventing, by way of example, but not limitation, the composition can be administered orally, by injection, such as intravenously, intramuscularly, intrathecally or subcutaneously, sublingually, buccally, rectally, vaginally, by the ocular route, by the otic route, nasally, such as by spray, inhalation, such as by nebulization, cutaneously, such as by topical application, via implantation, transdermally, or systemically. In the foregoing methods, by way of further example, but not limitation, the antimicrobial composition can be administered via a nebulizer, a vaping device, a bandage, a gauze, a suture, floss, or a condom. In any of the foregoing methods, the microbial infection or disease or condition caused by a microbial infection affects the skin, such as the hands, feet, face, genitals, torso or scalp, the mouth, teeth, ear, nose, throat, gastrointestinal system, such as the stomach, small/large bowel, rectum or anus, gallbladder, appendix, surgical incision site, internal tissue exposed during surgery or trauma, a cut or wound, blood, or brain. In some embodiments, the composition can be applied to the affected tissue. In some embodiments, the microbial infection or target microbe for the sanitizing or sterilizing is bacterial, fungal or viral. In some embodiments, the microbial infection is a bacterial infection caused by or the microbial target is Bacillus cereus, Streptococcus mutans, Lactobacillus acidophilus, Staphylococcus epidermidis, Enterococcus faecalis, Listeria monocytogenes, Streptococcus pyogenes, Streptococcus pneumoniae, Streptococcus agalactiae, Cutibacterium acnes, Staphylococcus aureus, Staphylococcus pseudintermedius, Helicobacter pylori, Clostridium difficile, Pseudomonas aeruginosa, or a Gram-positive bacteria. In some embodiments, the microbial target is a bacteria of the genus Staphylococcus, Streptococcus, Enterococcus, Lactobacillus, Cutibacterium, Helicobacter, Clostridium, or Pseudomonas. In some embodiments, the microbial infection is caused by or the microbial target is an enveloped virus. In some embodiments, the microbial infection is caused by or the microbial target can be a virus such as, by way of example, but not limitation, respiratory syncytial virus (RSV), an influenza virus, a coronavirus, such as by way of example but not limitation SARS-CoV-2, and a herpes virus, such as by way of example but not limitation HSV-1. By way of further example, the virus can be Tacaribe virus, Dengue virus, Influenze A H5N1, West Nile virus Zika virus, Measles virus, SARS-CoV-1, Epstein-Barr virus, Ebola virus, Lassa virus and Vesicular Stomatitis Virus (VSV). In some embodiments, the microbial infection is caused by or the microbial target is a fungus such as, by way of example, but not limitation, Candida albicans. In any of the foregoing embodiments, the subject can be a human or animal subject. By way of example, but not limitation, for animal applications, the compositions can be applied to prevent or treat infections of hooves, udders, such as mastitis. Compositions of the present disclosure can also be used as a mild non-toxic disinfectant for animal stalls and cages.

In some embodiments, a composition of the present disclosure or antimicrobial composition thereof can have a minimum inhibitory concentration (MIC) of 100 μg/mL or less against a bacterium selected from the group consisting of Bacillus cereus, Streptococcus mutans, Lactobacillus acidophilus, Staphylococcus epidermidis, Enterococcus faecalis, Listeria monocytogenes, Streptococcus pyogenes, Streptococcus pneumoniae, Streptococcus agalactiae, Cutibacterium acnes, Staphylococcus aureus, Staphylococcus pseudintermedius, Helicobacter pylori, Clostridium difficile, Pseudomonas aeruginosa, a Gram-positive bacteria, or bacteria of the genus Staphylococcus, Streptococcus, Enterococcus, Lactobacillus, Cutibacterium, Helicobacter, Clostridium, or Pseudomonas. By way of example, but not limitation, the composition of the present disclosure or antimicrobial composition thereof can have a MIC of about 100 μg/mL or less, about 75 μg/mL or less, about 50 μg/mL or less, about 25 μg/mL, about 12.5 μg/mL or less, about 1 μg/mL to about 100 μg/mL, about 5 μg/mL to about 100 μg/mL, about 10 μg/mL to about 100 μg/mL, about 25 μg/mL to about 100 μg/mL, about 50 μg/mL to about 100 μg/mL, about 1 μg/mL to about 50 μg/mL, about 1 μg/mL to about 25 μg/mL, about 10 μg/mL to about 50 μg/mL, about 10 μg/mL to about 25 μg/mL, about 1 μg/mL, 2 μg/mL, 5 μg/mL, 10 μg/mL, 15 μg/mL, 20 μg/mL, 25 μg/mL, 30 μg/mL, 40 μg/mL, 50 μg/mL, 60 μg/mL, 70 μg/mL, 75 μg/mL, 80 μg/mL, 90 μg/mL, or 100 μg/mL against a bacterium selected from the group consisting of Bacillus cereus, Streptococcus mutans, Lactobacillus acidophilus, Staphylococcus epidermidis, Enterococcus faecalis, Listeria monocytogenes, Streptococcus pyogenes, Streptococcus pneumoniae, Streptococcus agalactiae, Cutibacterium acnes, Staphylococcus aureus, Staphylococcus pseudintermedius, Helicobacter pylori, Clostridium difficile, Pseudomonas aeruginosa, a Gram-positive bacteria or bacteria of the genus Staphylococcus, Streptococcus, Enterococcus, Lactobacillus, Cutibacterium, Helicobacter, Clostridium, or Pseudomonas. It should be understood that, in any of the foregoing embodiments, MIC can be the lowest concentration that does not allow an exponential growth phase during the time course of 16 hours as measured by OD600.

In some embodiments, a composition of the present disclosure or antimicrobial composition thereof can have a minimum bactericidal concentration (MBC) of 100 μg/mL or less against a bacterium selected from the group consisting of S. pyogenes, S. aureus, and C. acnes By way of example, but not limitation, the composition of the present disclosure or antimicrobial composition thereof can have a MBC of about 100 μg/mL or less, about 75 μg/mL or less, about 50 μg/mL or less, about 25 μg/mL, about 12.5 μg/mL or less, about 1 μg/mL to about 100 μg/mL, about 5 μg/mL to about 100 μg/mL, about 10 μg/mL to about 100 μg/mL, about 25 μg/mL to about 100 μg/mL, about 50 μg/mL to about 100 μg/mL, about 1 μg/mL to about 50 μg/mL, about 1 μg/mL to about 25 μg/mL, about 10 μg/mL to about 50 μg/mL, about 10 μg/mL to about 25 μg/mL, about 1 μg/mL, 2 μg/mL, 5 μg/mL, 10 μg/mL, 15 μg/mL, 20 μg/mL, 25 μg/mL, 30 μg/mL, 40 μg/mL, 50 μg/mL, 60 μg/mL, 70 μg/mL, 75 μg/mL, 80 μg/mL, 90 μg/mL, or 100 μg/mL against a bacterium selected from the group consisting of Bacillus cereus, Streptococcus mutans, Lactobacillus acidophilus, Staphylococcus epidermidis, Enterococcus faecalis, Listeria monocytogenes, Streptococcus pyogenes, Streptococcus pneumoniae, Streptococcus agalactiae, Cutibacterium acnes, Staphylococcus aureus, Staphylococcus pseudintermedius, Helicobacter pylori, Clostridium difficile, Pseudomonas aeruginosa, a Gram-positive bacteria or bacteria of the genus Staphylococcus, Streptococcus, Enterococcus, Lactobacillus, Cutibacterium, Helicobacter, Clostridium, or Pseudomonas. As used herein, the MBC is the lowest concentration of antibacterial agent that reduces the viability of the initial bacterial inoculum by 3 logs, or 99%. In some embodiments, a composition of the present disclosure or an antimicrobial composition thereof can have bacteriostatic or bactericidal activity against bacteria.

In any of the foregoing embodiments, a composition of the present disclosure or antimicrobial composition thereof can have a log-reduction value (LRV) of at least 1.0 against a virus. By way of example, but not limitation, the LRV can be at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.5, 5.0 or more. By way of further example, but not limitation, the LRV can be from about 1.0 to about 10.0, about 1.0 to about 10.0, about 2.0 to about 10.0, about 2.0 to about 10.0, about 3.0 to about 10.0, about 4.0 to about 10.0, about 5.0 to about 10.0, about 1.0 to about 5.0, about 2.0 to about 5.0, about 3.0 to about 5.0, about 4.0 to about 5.0, about 1.0 to about 4.0, about 2.0 to about 4.0, about 3.0 to about 4.0, about 1.0 to about 3.0, about 2.0 to about 3.0, or about 1.0 to about 2.0. It should be understood that the LRV can be as measured against an untreated virus control after growth for a suitable period of time in suitable cells, such as, by way of example but not limitation, MA-104 cells over 5 days.

Compositions of the present disclosure can be useful in antimicrobial applications. Thus, in some embodiments an antimicrobial composition can include an antimicrobial oil composition, composition thereof, or nanoemulsion composition of the present disclosure. Preferably, the nanoemulsion composition of the present disclosure is a nanoemulsion with droplets or particles having a Z average size of about 300 nm or less. In some embodiments, the composition can include droplets or particles with a Z average size of greater than about 300 nm or greater than 1000 nm as described in the present disclosure. The antimicrobial composition can include an effective amount of the antimicrobial oil composition of the present disclosure, where the effective amount is an amount sufficient to prevent growth, reduce growth or kill a microorganism, such as bacteria or fungi, or viruses.

In some embodiments, the antimicrobial composition which can be or contain the antimicrobial oil composition can be in the form of a cream, a lotion, an eye drop, an ear drop, a sinus rinse, a spray, such as nasal, oral, mucosal or for skin or feet, an ointment, a deodorant formulation, a body wash, a shampoo, a scalp treatment, a mouthwash, a toothpaste, a lozenge, a beverage, or a lubricant, such as vaginal lubricant. By way of example, but not limitation, the antimicrobial composition can be a cream such as for skin application, a lotion such as for skin application, an eye drop, an ear drop, a sinus rinse, a spray such as for nasal, oral mucosal, skin or foot treatment, an ointment, a deodorant, a body wash, a shampoo, a scalp treatment, a mouthwash, a toothpaste, a lozenge, a beverage, a lubricant, or a powder, such as a spray-dried nanoemulsion (or microemulsion). Methods for formulating such compositions are well-known in the art. It should be understood that for compositions in a cream, such compositions can also be formulated in a lotion.

It should be understood that compositions of the present disclosure and antimicrobial compositions thereof can be effective at preventing growth, reducing growth or killing bacteria, fungi and/or viruses. Thus, in some embodiments, an antimicrobial oil composition of the present disclosure, antimicrobial composition, or nanoemulsion composition is effective against bacteria to either prevent or reduce growth or to kill the bacteria. In some embodiments, a composition of the present disclosure or antimicrobial composition thereof is effective against fungi to either prevent or reduce growth or kill the fungi. In some embodiments, a composition of the present disclosure or antimicrobial composition thereof can be effective against viruses to reduce or prevent transmission of the virus or to kill the virus, e.g. to render the virus non-contagious or having reduced transmissibility. It should be understood that compositions of the present disclosure and antimicrobial compositions thereof can be effective against a single type of microbe, e.g. bacteria or fungi, or against viruses or against a combination of these.

It has surprisingly been found that certain combinations of EMO and FFA oils have superior antimicrobial properties against specific pathogens. In certain aspects, these antimicrobial properties include specificity for the target microbe while others, such as commensal bacteria, are either unaffected or affected to a lesser extent. It should be understood that the following disclosure represents preferred embodiments for specific target microbes but should not be construed as excluding other embodiments such as other combinations of EMO and FFA oil, or EMO or FFA oil alone.

Staphylococcus aureus

In any of the foregoing embodiments, the microbial infection can be caused by or the target for sterilizing or disinfecting can be Staphylococcus aureus. In such embodiments, the method of treating or preventing can be to treat or prevent a skin infection such as, by way of example, but not limitation, atopic dermatitis. In certain preferred embodiments, where the microbial infection is caused by S. aureus, the antimicrobial oil composition can include EMO derived from oat, macadamia nut, coconut oil, or a combination thereof, and FFA oil from flaxseed oil. Thus, in such embodiments, the MAGs can be derived from oat, macadamia nut, coconut oil, or combinations thereof, and the FFA can be derived from oat, macadamia nut, coconut oil, or combinations thereof (for the first portion of FFA) and flaxseed oil (for the second portion of FFA). In certain preferred embodiments, where the microbial infection is caused by or the target for sterilizing or disinfecting is S. aureus, the antimicrobial oil composition is a mixture of 25% oat EMO/75% flaxseed FFA oil, 25% macadamia nut EMO/75% flaxseed FFA oil, or 25% coconut EMO/75% flaxseed FFA oil, i.e. the first amount is 25% and the second amount is 75%. The expected fatty acid profile and MAG and FFA content are provided in the table below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| | | | | Flax- | | | |
|---|---|---|---|---|---|---|---|
| Fatty | Oat | Macadamia | Coconut | seed | Oa/ | Mac/ | Co/ |
| Acid | Oil | Nut Oil* | Oil* | Oil | Fx-25 | Fx-25 | Fx-25 |
| C6:0 | 0.0 | 0.0 | 0.0 | 0.0 | 0.0 | 0.0 | 0.0 |
| C8:0 | 0.0 | 0.0 | 0.0 | 0.0 | 0.0 | 0.0 | 0.0 |
| C10 | 0.0 | 0.0 | 6.0 | 0.0 | 0.0 | 0.0 | 1.5 |
| C12 | 0.0 | 0.0 | 47.0 | 0.0 | 0.0 | 0.0 | 11.8 |
| C14 | 0.0 | 1.2 | 18.0 | 0.0 | 0.0 | 0.3 | 4.5 |
| C16 | 11.4 | 41 | 9.0 | 5.2 | 6.8 | 14.2 | 6.2 |
| C18 | 4.3 | 5.4 | 3.0 | 3.3 | 3.6 | 3.8 | 3.2 |
| C18:1 | 22.5 | 41 | 2.0 | 15.4 | 17.2 | 21.8 | 12.0 |
| C18:2 | 53.8 | 2.7 | 2.0 | 16.0 | 25.4 | 12.7 | 12.5 |
| C18:3 | 7.2 | 1 | 0.0 | 59.7 | 46.6 | 45.0 | 44.8 |
| C20 | 0.8 | 6.5 | 0.0 | 0.4 | 0.5 | 1.9 | 0.3 |
| C22 | 0.0 | 1.1 | 0.0 | 0.0 | 0.0 | 0.3 | 0.0 |
| EMO | | | | | EMO/FFA Oil Blends | | |
| MAG | 65.9 | | 72 | 57.9 | 16.5 | | 18 |
| DAG | 9.4 | | 16 | 16.5 | 2.4 | | 4 |
| TAG | BLD | | BLD | BLD | BLD | | BLD |
| FFA | 13 | | 12 | 18 | 73.0 | | 72.8 |

| | | |
|---|---|---|
| MAG/FFA Ratio | 0.2 | 0.2 |

*Values from literature; other values from experimental data

In certain preferred embodiments, where the microbial infection is caused by or the target for sterilizing or disinfecting is S. aureus, the antimicrobial oil composition can include about 10% to about 30% MAGs and about 60% to about 80% FFA. In certain preferred embodiments, where the microbial infection is caused by or the target for sterilizing or disinfecting is S. aureus, the antimicrobial oil composition can have a fatty acid profile that comprises two or more (or three, four, or five) fatty acids selected from the group consisting of:

| Fatty Acid | Amount out of Total Fatty Acids |
|---|---|
| C10 | 0.1%-5% |
| C12 | 5%-15% |
| C14 | 0.1%-10% |
| C16 | 0.1%-20%; 0.1%-10%; 10%-20% |
| C18 | 0.1%-10% |
| C18:1 | 5%-30%; 15%-30% |
| C18:2 | 5%-35%; 20%-35% |
| C18:3 | 40%-55% |
| C20 | 0.1%-5% |
| C22 | 0.1%-5% |

In certain preferred embodiments, where the microbial infection is caused by or the target for sterilizing or disinfecting is S. aureus, the MAGs antimicrobial oil composition can have a fatty acid profile that comprises two or more fatty acids (or three, four, five, six, seven, eight, nine or ten) selected from the group consisting of:

| Fatty Acid | Amount out of Total Fatty Acids |
|---|---|
| C10 | 0.1%-10%; 5%-10% |
| C12 | 40%-55% |
| C14 | 0.1%-25%; 0.1%-10%, 10%-25% |
| C16 | 5%-50%; 5%-15%; 35%-50% |
| C18 | 0.1%-10% |
| C18:1 | 0.1%-50%; 0.1%-10%; 15%-30%; 35%- |
| | 50% |
| C18:2 | 0.1%-60%; 0.1%-10%; 45%-60% |
| C18:3 | 0.1%-15% |
| C20 | 0.1%-10% |
| C22 | 0.1%-5% |

In certain preferred embodiments, where the microbial infection is caused by or the target for sterilizing or disinfecting is S. aureus, the ratio of MAG/FFA in the antimicrobial oil composition is about 0.2.

It should be understood that the foregoing preferred embodiments for S. aureus can be utilized as the composition or part of the composition for treating or preventing a skin condition, such as atopic dermatitis.

Streptococcus pyogenes

In any of the foregoing embodiments, the microbial infection can be caused by or the target for sterilizing or disinfecting is Streptococcus pyogenes. In such embodiments, the method of treating or preventing can be to treat or prevent strep throat. In certain preferred embodiments, where the microbial infection is caused by or the target for sterilizing or disinfecting is S. pyogenes, the antimicrobial oil composition can include EMO derived from flaxseed oil, rosehip oil, or a combination thereof, and FFA oil from flaxseed oil, rosehip oil, coconut oil, or a combination thereof. Thus, in such embodiments, the MAGs can be derived from flaxseed oil, rosehip oil, or combinations thereof, and the FFA can be derived from flaxseed oil, rosehip oil, coconut oil, or combinations thereof (for the first portion of FFA) and flaxseed oil, rosehip oil, or combinations thereof (for the second portion of FFA). In certain preferred embodiments, where the microbial infection is caused by or the target for sterilizing or disinfecting is S. pyogenes, the antimicrobial oil composition is a mixture of 50% flaxseed EMO/50% rosehip FFA oil, 50% rosehip EMO/50% flaxseed FFA oil, or 25% rosehip EMO/75% rosehip FFA oil. The expected fatty acid profile and MAG and FFA content are provided in the Table below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| Fatty | Flaxseed | | | | |
|---|---|---|---|---|---|
| Acid | Oil | Rosehip Oil | Fx/R-50 | R/Fx-50 | R-25 |
| C6:0 | 0.0 | 0.0 | 0.0 | 0.0 | 0.0 |
| C8:0 | 0.0 | 0.0 | 0.0 | 0.0 | 0.0 |
| C10 | 0.0 | 0.0 | 0.0 | 0.0 | 0.0 |
| C12 | 0.0 | 0.0 | 0.0 | 0.0 | 0.0 |
| C14 | 0.0 | 0.0 | 0.0 | 0.0 | 0.0 |
| C16 | 5.2 | 4.5 | 4.8 | 4.8 | 4.5 |
| C18 | 3.3 | 2.2 | 2.8 | 2.8 | 2.2 |
| C18:1 | 15.4 | 19.9 | 17.6 | 17.6 | 19.9 |
| C18:2 | 16.0 | 50.8 | 33.4 | 33.4 | 50.8 |
| C18:3 | 59.7 | 20.7 | 40.2 | 40.2 | 20.7 |
| C20 | 0.4 | 1.9 | 1.2 | 1.2 | 1.9 |
| C22 | 0.0 | 0.0 | 0.0 | 0.0 | 0.0 |
| EMO | | | EMO/FFA Oil Blends | | |
| MAG | 57.9 | 54.6 | 29 | 27.3 | 13.7 |
| DAG | 16.5 | 16.5 | 8.25 | 8.25 | 4.1 |
| TAG | BLD | BLD | BLD | BLD | BLD |
| FFA | 18 | 16 | 55.5 | 55 | 27.25 |

| | | | |
|---|---|---|---|
| MAG/FFA Ratio | 0.5 | 0.5 | 0.5 |

*Values from literature; other values from experimental data

In certain preferred embodiments, where the microbial infection is caused by or the target for sterilizing or disinfecting is S. pyogenes, the antimicrobial oil composition can include about 10% to about 30% MAGs and about 25% to about 60% FFA. In certain preferred embodiments, where the microbial infection is caused by or the target for sterilizing or disinfecting is S. pyogenes, the antimicrobial oil composition can have a fatty acid profile that comprises two or more (or three, four, or five) fatty acids selected from the group consisting of:

| Fatty Acid | Amount out of Total Fatty Acids |
|---|---|
| C16 | 0.1%-10% |
| C18 | 0.1%-10% |
| C18:1 | 10%-25% |
| C18:2 | 25%-60% |
| C18:3 | 15%-50%; 35%-45% |
| C20 | 0.1%-10% |

In certain preferred embodiments, where the microbial infection is caused by or the target for sterilizing or disinfecting is S. pyogenes, the MAGs antimicrobial oil composition can have a fatty acid profile that comprises two or more fatty acids (or three, four, five, six, seven, eight, nine or ten) selected from the group consisting of:

| Fatty Acid | Amount out of Total Fatty Acids |
|---|---|
| C16 | 0.1%-10% |
| C18 | 0.1%-10% |
| C18:1 | 10%-25% |
| C18:2 | 10%-60%; 10%-25%; 45%-60% |
| C18:3 | 15%-65%; 15%-30%; 55%-65% |
| C20 | 0.1%-10% |

In certain preferred embodiments, where the microbial infection is caused by or the target for sterilizing or disinfecting is S. pyogenes, the ratio of MAG/FFA in the antimicrobial oil composition is about 0.5.

It should be understood that embodiments related to S. aureus can also apply to S. pseudintermedius and can, in certain instances, include treating canine ear infections.

Streptococcus mutans

In any of the foregoing embodiments, the microbial infection can be caused by or the target for sterilizing or disinfecting is Streptococcus mutans. In such embodiments, the method of treating or preventing can be to treat or prevent dental caries. In some embodiments, the composition administered can be a paste to the teeth. In certain preferred embodiments, where the microbial infection is caused by or the target for sterilizing or disinfecting is S. mutans, the antimicrobial oil composition can include EMO derived from flaxseed oil, oat oil, hemp seed oil, rosehip oil, pomegranate oil, or a combination thereof, and FFA oil from oat oil, hemp seed oil, rosehip oil, pomegranate oil, flaxseed oil, or a combination thereof. Thus, in such embodiments, the MAGs can be derived from flaxseed oil, oat oil, hemp seed oil, rosehip oil, pomegranate oil, or combinations thereof, and the FFA can be derived from flaxseed oil, oat oil, hemp seed oil, rosehip oil, pomegranate oil, or combinations thereof (for the first portion of FFA) and oat oil, hemp seed oil, rosehip oil, pomegranate oil, flaxseed oil, or combinations thereof (for the second portion of FFA). In certain preferred embodiments, where the microbial infection is caused by or the target for sterilizing or disinfecting is S. mutans, the antimicrobial oil composition is a mixture of 50% flaxseed EMO/50% rosehip FFA oil, 50% flaxseed EMO/50% hemp seed FFA oil, 50% rosehip EMO/50% flaxseed FFA oil, or 50% hemp seed EMO/50% flaxseed FFA oil. The expected fatty acid profile and MAG and FFA content are provided in the table below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| | Flax- | | | | | | |
|---|---|---|---|---|---|---|---|
| Fatty | seed | Rosehip | Hempseed | Fx/ | Fx/ | R/ | Hemp/ |
| Acid | Oil | Oil | Oil | R-50 | Hemp-50 | Fx-50 | Fx-50 |
| C6:0 | 0.0 | 0.0 | 0.0 | 0.0 | 0.0 | 0.0 | 0.0 |
| C8:0 | 0.0 | 0.0 | 0.0 | 0.0 | 0.0 | 0.0 | 0.0 |
| C10 | 0.0 | 0.0 | 0.0 | 0.0 | 0.0 | 0.0 | 0.0 |
| C12 | 0.0 | 0.0 | 0.0 | 0.0 | 0.0 | 0.0 | 0.0 |
| C14 | 0.0 | 0.0 | 0.0 | 0.0 | 0.0 | 0.0 | 0.0 |
| C16 | 5.2 | 4.5 | 6.6 | 4.8 | 5.9 | 4.8 | 5.9 |
| C18 | 3.3 | 2.2 | 2.6 | 2.8 | 3.0 | 2.8 | 3.0 |
| C18:1 | 15.4 | 19.9 | 11.5 | 17.6 | 13.4 | 17.6 | 13.4 |
| C18:2 | 16.0 | 50.8 | 58.6 | 33.4 | 37.3 | 33.4 | 37.3 |
| C18:3 | 59.7 | 20.7 | 19.2 | 40.2 | 39.4 | 40.2 | 39.4 |
| C20 | 0.4 | 1.9 | 1.5 | 1.2 | 1.0 | 1.2 | 1.0 |
| C22 | 0.0 | 0.0 | 0.0 | 0.0 | 0.0 | 0.0 | 0.0 |
| EMO | | | | EMO/FFA Oil Blends | | | |
| MAG | 57.9 | 54.6 | 69.9 | 29 | 29 | 27.3 | 35 |
| DAG | 16.5 | 16.5 | 11 | 8.25 | 8.25 | 8.25 | 2.75 |
| TAG | BLD | BLD | BLD | BLD | BLD | BLD | BLD |
| FFA | 18 | 16 | 9 | 56 | 56 | 55 | 52 |

| | | | | |
|---|---|---|---|---|
| MAG/FFA Ratio | 0.5 | 0.5 | 0.5 | 0.7 |

In certain preferred embodiments, where the microbial infection is caused by or the target for sterilizing or disinfecting is S. mutans, the antimicrobial oil composition can include about 10% to about 40% MAGs and about 50% to about 60% FFA. In certain preferred embodiments, where the microbial infection is caused by or the target for sterilizing or disinfecting is S. mutans, the antimicrobial oil composition can have a fatty acid profile that comprises two or more (or three, four, or five) fatty acids selected from the group consisting of:

| Fatty Acid | Amount out of Total Fatty Acids |
|---|---|
| C16 | 0.1%-10% |
| C18 | 0.1%-10% |
| C18:1 | 10%-25% |
| C18:2 | 25%-50% |
| C18:3 | 15%-50% |

In certain preferred embodiments, where the microbial infection is caused by or the target for sterilizing or disinfecting is S. mutans, the MAGs antimicrobial oil composition can have a fatty acid profile that comprises two or more fatty acids (or three, four, or five) selected from the group consisting of:

| Fatty Acid | Amount out of Total Fatty Acids |
|---|---|
| C16 | 0.1%-10% |
| C18 | 0.1%-10% |
| C18:1 | 5%-25% |
| C18:2 | 10%-65% |
| C18:3 | 15%-65% |

In certain preferred embodiments, where the microbial infection is caused by or the target for sterilizing or disinfecting is S. mutans, the ratio of MAG/FFA in the antimicrobial oil composition is about 0.5 to about 0.7.

Cutibacterium acnes

In any of the foregoing embodiments, the microbial infection can be caused by or the target for sterilizing or disinfecting is Cutibacterium acnes. In such embodiments, the method of treating or preventing can be to treat or prevent acne. In certain preferred embodiments, where the microbial infection is caused by C. acnes, the antimicrobial oil composition can include EMO derived from coconut oil, hemp seed oil, fish oil, flaxseed oil, MCT oil, rosehip oil, or a combination thereof, and FFA oil from coconut oil, hemp seed oil, flaxseed oil, fish oil, rosehip oil, or a combination thereof. Thus, in such embodiments, the MAGs can be derived from coconut oil, hemp seed oil, fish oil, flaxseed oil, MCT oil, rosehip oil, or a combination thereof, and the FFA can be derived from coconut oil, hemp seed oil, fish oil, flaxseed oil, MCT oil, rosehip oil, or a combination thereof (for the first portion of FFA) and coconut oil, hemp seed oil, flaxseed oil, fish oil, rosehip oil, or a combination thereof (for the second portion of FFA). In certain preferred embodiments, where the microbial infection is caused by or the target for sterilizing or disinfecting is C. acnes, the antimicrobial oil composition is a mixture of 50% hemp seed EMO/50% rosehip FFA oil, 50% rosehip EMO/50% hemp seed FFA oil, or 50% flaxseed EMO/50% coconut FFA oil. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| | Flax- | Rose- | Hemp- | | | | |
|---|---|---|---|---|---|---|---|
| Fatty | seed | hip | seed | Coconut | Hemp/ | R/ | Fx/ |
| Acid | Oil | Oil | Oil | Oil | R-50 | Hemp-50 | Co-50 |
| C6:0 | 0.0 | 0.0 | 0.0 | 0.0 | 0.0 | 0.0 | 0.0 |
| C8:0 | 0.0 | 0.0 | 0.0 | 0.0 | 0.0 | 0.0 | 0.0 |
| C10 | 0.0 | 0.0 | 0.0 | 6.0 | 0.0 | 0.0 | 3.0 |
| C12 | 0.0 | 0.0 | 0.0 | 47.0 | 0.0 | 0.0 | 23.5 |
| C14 | 0.0 | 0.0 | 0.0 | 18.0 | 0.0 | 0.0 | 9.0 |
| C16 | 5.2 | 4.5 | 6.6 | 9.0 | 4.5 | 4.5 | 7.1 |
| C18 | 3.3 | 2.2 | 2.6 | 3.0 | 2.2 | 2.2 | 3.1 |
| C18:1 | 15.4 | 19.9 | 11.5 | 2.0 | 19.9 | 19.9 | 8.7 |
| C18:2 | 16.0 | 50.8 | 58.6 | 2.0 | 50.8 | 50.8 | 9.0 |
| C18:3 | 59.7 | 20.7 | 19.2 | 0.0 | 20.7 | 20.7 | 29.8 |
| C20 | 0.4 | 1.9 | 1.5 | 0.0 | 1.9 | 1.9 | 0.2 |
| C22 | 0.0 | 0.0 | 0.0 | 0.0 | 0.0 | 0.0 | 0.0 |
| EMO | | | | | EMO/FFA Oil Blends | | |
| MAG | 57.9 | 54.6 | 69.9 | 72 | 35 | 27.3 | 29 |
| DAG | 16.5 | 16.5 | 11 | 16 | 2.75 | 8.25 | 8.25 |
| TAG | BLD | BLD | BLD | BLD | BLD | BLD | BLD |
| FFA | 18 | 16 | 9 | 12 | 52 | 55 | 56 |

| | | | |
|---|---|---|---|
| MAG/FFA Ratio | 0.7 | 0.5 | 0.5 |

In certain preferred embodiments, where the microbial infection is caused by or the target for sterilizing or disinfecting is C. acnes, the antimicrobial oil composition can include about 20% to about 40% MAGs and about 50% to about 60% FFA. In certain preferred embodiments, where the microbial infection is caused by or the target for sterilizing or disinfecting is C. acnes, the antimicrobial oil composition can have a fatty acid profile that comprises two or more (or three, four, or five) fatty acids selected from the group consisting of:

| Fatty Acid | Amount out of Total Fatty Acids |
|---|---|
| C16 | 0.1%-10% |
| C18 | 0.1%-10% |
| C18:1 | 5%-25% |
| C18:2 | 5%-60% |
| C18:3 | 15% -35% |

In certain preferred embodiments, where the microbial infection is caused by or the target for sterilizing or disinfecting is C. acnes, the MAGs antimicrobial oil composition can have a fatty acid profile that comprises two or more fatty acids (or three, four, or five) selected from the group consisting of:

| Fatty Acid | Amount out of Total Fatty Acids |
|---|---|
| C16 | 0.1%-10% |
| C18 | 0.1%-10% |
| C18:1 | 5%-25% |
| C18:2 | 10%-65% |
| C18:3 | 10%-65% |

In certain preferred embodiments, where the microbial infection is caused by or the target for sterilizing or disinfecting is C. acnes, the ratio of MAG/FFA in the antimicrobial oil composition is about 0.5 to about 0.7.

Helicobacter pylori

In any of the foregoing embodiments, the microbial infection can be caused by or the target for sterilizing or disinfecting is Helicobacter pylori. In such embodiments, the method of treating or preventing can be to treat or prevent ulcers. In certain preferred embodiments, where the microbial infection is caused by or the target for sterilizing or disinfecting is H. pylori, the antimicrobial oil composition can include EMO derived from fish oil, flaxseed oil, or a combination thereof, and FFA oil from coconut oil, fish oil, flaxseed oil, or a combination thereof. Thus, in such embodiments, the MAGs can be derived from fish oil, flaxseed oil, or a combination thereof, and the FFA can be derived from fish oil, flaxseed oil, or a combination thereof (for the first portion of FFA) and coconut oil, fish oil, flaxseed oil, or a combination thereof (for the second portion of FFA). In certain preferred embodiments, where the microbial infection is caused by or the target for sterilizing or disinfecting is H. pylori, the antimicrobial oil composition is a mixture of 50% flaxseed EMO/50% fish FFA oil, 50% fish EMO/50% flaxseed FFA oil, or 50% fish EMO/50% coconut FFA oil. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| Fatty | Flaxseed | Fish | Coconut | Fx/ | Fish/ | Fish/ |
|---|---|---|---|---|---|---|
| Acid | Oil | Oil | Oil* | Fish-50 | Fx-50 | Co-50 |
| C6:0 | 0.0 | 0.0 | 0.0 | 0.0 | 0.0 | 0.0 |
| C8:0 | 0.0 | 0.0 | 0.0 | 0.0 | 0.0 | 0.0 |
| C10 | 0.0 | 0.0 | 6.0 | 0.0 | 0.0 | 3.0 |
| C12 | 0.0 | 0.0 | 47.0 | 0.0 | 0.0 | 23.5 |
| C14 | 0.0 | 9.4 | 18.0 | 4.7 | 4.7 | 13.7 |
| C16 | 5.2 | 26.9 | 9.0 | 16.0 | 16.0 | 17.9 |
| C18 | 3.3 | 0.0 | 3.0 | 1.6 | 1.6 | 1.5 |
| C18:1 | 15.4 | 21.8 | 2.0 | 18.6 | 18.6 | 11.9 |
| C18:2 | 16.0 | 2.5 | 2.0 | 9.2 | 9.2 | 2.2 |
| C18:3 | 59.7 | 1.7 | 0.0 | 30.7 | 30.7 | 0.9 |
| C20 | 0.4 | 14.5 | 0.0 | 7.5 | 7.5 | 7.3 |
| C22 | 0.0 | 23.2 | 0.0 | 11.6 | 11.6 | 11.6 |
| EMO | | | | EMO/FFA Oil Blends | | |
| MAG | 57.9 | 56 | 72 | 29 | 28 | 28 |
| DAG | 16.5 | 10 | 16 | 13 | 13 | 13 |
| TAG | BLD | 16 | BLD | 8 | 8 | 8 |
| FFA | 18 | 12 | 12 | 48 | 53 | 53 |

| | | | |
|---|---|---|---|
| MAG/FFA Ratio | 0.6 | 0.5 | 0.5 |

*Values from literature

In certain preferred embodiments, where the microbial infection is caused by or the target for sterilizing or disinfecting is H. pylori, the antimicrobial oil composition can include about 20% to about 40% MAGs and about 40% to about 60% FFA. In certain preferred embodiments, where the microbial infection is caused by or the target for sterilizing or disinfecting is H. pylori, the antimicrobial oil composition can have a fatty acid profile that comprises two or more (or three, four, five, six, seven, or eight) fatty acids selected from the group consisting of:

| Fatty Acid | Amount out of Total Fatty Acids |
|---|---|
| C14 | 0.1%-20% |
| C16 | 10%-25% |
| C18 | 0.1%-5% |
| C18:1 | 10%-25% |
| C18:2 | 0.1%-15% |
| C18:3 | 0.1%-40% |
| C20 | 0.1%-15% |
| C22 | 5%-15% |

In certain preferred embodiments, where the microbial infection is caused by or the target for sterilizing or disinfecting is H. pylori, the MAGs antimicrobial oil composition can have a fatty acid profile that comprises two or more fatty acids (or three, four, five, six, seven, or eight) selected from the group consisting of:

| Fatty Acid | Amount out of Total Fatty Acids |
|---|---|
| C14 | 5%-25% |
| C16 | 0.1%-35% |
| C18 | 0.1%-10% |
| C18:1 | 0.1%-30% |
| C18:2 | 0.1%-25% |
| C18:3 | 0.1%-65% |
| C20 | 0.1%-20% |
| C22 | 20%-30% |

In certain preferred embodiments, where the microbial infection is caused by or the target for sterilizing or disinfecting is H. pylori, the ratio of MAG/FFA in the antimicrobial oil composition is about 0.5 to about 0.7.

Listeria monocytogenes

In any of the foregoing embodiments, the microbial infection can be caused by or the target for sterilizing or disinfecting is Listeria monocytogenes. In such embodiments, the method of treating or preventing can be to treat or prevent a foodborne infection. In certain preferred embodiments, where the microbial infection is caused by or the target for sterilizing or disinfecting is L. monocytogenes, the antimicrobial oil composition can include EMO derived from coconut oil, fish oil, flaxseed oil, or a combination thereof, and FFA oil from coconut oil, flaxseed oil, or a combination thereof. Thus, in such embodiments, the MAGs can be derived coconut oil, fish oil, flaxseed oil, or a combination thereof, and the FFA can be derived from coconut oil, fish oil, flaxseed oil, or a combination thereof (for the first portion of FFA) and coconut oil, flaxseed oil, or a combination thereof (for the second portion of FFA). In certain preferred embodiments, where the microbial infection is caused by or the target for sterilizing or disinfecting is L. monocytogenes, the antimicrobial oil composition is a mixture of 50% fish EMO/50% coconut FFA oil or 50% fish EMO/50% flaxseed FFA oil. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| Fatty | Flaxseed | | Coconut | Fish/ | Fish/ |
|---|---|---|---|---|---|
| Acid | Oil | Fish Oil* | Oil* | Fx-50 | Co-50 |
| C6:0 | 0.0 | 0.0 | 0.0 | 0.0 | 0.0 |
| C8:0 | 0.0 | 0.0 | 0.0 | 0.0 | 0.0 |
| C10 | 0.0 | 0.0 | 6.0 | 0.0 | 3.0 |
| C12 | 0.0 | 0.0 | 47.0 | 0.0 | 23.5 |
| C14 | 0.0 | 9.4 | 18.0 | 4.7 | 13.7 |
| C16 | 5.2 | 26.9 | 9.0 | 16.0 | 17.9 |
| C18 | 3.3 | 0.0 | 3.0 | 1.6 | 1.5 |
| C18:1 | 15.4 | 21.8 | 2.0 | 18.6 | 11.9 |
| C18:2 | 16.0 | 2.5 | 2.0 | 9.2 | 2.2 |
| C18:3 | 59.7 | 1.7 | 0.0 | 30.7 | 0.9 |
| C20 | 0.4 | 14.5 | 0.0 | 7.5 | 7.3 |
| C22 | 0.0 | 23.2 | 0.0 | 11.6 | 11.6 |
| | | | | EMO/FFA Oil | |
| EMO | | | | Blends | |
| MAG | 57.9 | 56 | 72 | 28 | 28 |
| DAG | 16.5 | | 16 | 7 | 7 |
| TAG | BLD | | BLD | 8 | 8 |
| FFA | 18 | 12 | 12 | 53 | 53 |

| | | |
|---|---|---|
| MAG/FFA Ratio | 0.5 | 0.5 |

*Values from literature

In certain preferred embodiments, where the microbial infection is caused by or the target for sterilizing or disinfecting is L. monocytogenes, the antimicrobial oil composition can include about 20% to about 40% MAGs and about 40% to about 60% FFA. In certain preferred embodiments, where the microbial infection is caused by or the target for sterilizing or disinfecting is L. monocytogenes, the antimicrobial oil composition can have a fatty acid profile that comprises two or more (or three, four, five, six, seven, eight, or nine) fatty acids selected from the group consisting of:

| Fatty Acid | Amount out of Total Fatty Acids |
|---|---|
| C12 | 20%-30% |
| C14 | 0.1%-20% |
| C16 | 10%-25% |
| C18 | 0.1%-5% |
| C18:1 | 5%-25% |
| C18:2 | 0.1%-15% |
| C18:3 | 0.1%-40% |
| C20 | 0.1%-15% |
| C22 | 5%-15% |

In certain preferred embodiments, where the microbial infection is caused by or the target for sterilizing or disinfecting is L. monocytogenes, the MAGs antimicrobial oil composition can have a fatty acid profile that comprises two or more fatty acids (or three, four, five, six, seven, or eight) selected from the group consisting of:

| Fatty Acid | Amount out of Total Fatty Acids |
|---|---|
| C14 | 5%-15% |
| C16 | 20%-35% |
| C18 | 0.1%-10% |
| C18:1 | 15-30% |
| C18:2 | 0.1%-10% |
| C18:3 | 0.1%-10% |
| C20 | 5%-20% |
| C22 | 15%-30% |

In certain preferred embodiments, where the microbial infection is caused by or the target for sterilizing or disinfecting is L. monocytogenes, the ratio of MAG/FFA in the antimicrobial oil composition is about 0.5.

Bacillus cereus

In any of the foregoing embodiments, the microbial infection can be caused by or the target for sterilizing or disinfecting is Bacillus cereus. In such embodiments, the method of treating or preventing can be to treat or prevent a foodborne infection. In certain preferred embodiments, where the microbial infection is caused by or the target for sterilizing or disinfecting is B. cereus, the antimicrobial oil composition can include EMO derived from coconut oil, fish oil, or a combination thereof, and FFA oil from coconut oil. Thus, in such embodiments, the MAGs can be derived coconut oil, fish oil, or a combination thereof, and the FFA can be derived from coconut oil, fish oil, or a combination thereof (for the first portion of FFA) and coconut oil (for the second portion of FFA). In certain preferred embodiments, where the microbial infection is caused by or the target for sterilizing or disinfecting is B. cereus, the antimicrobial oil composition is a mixture of 75% fish EMO/25% coconut FFA oil or 50% coconut EMO/50% coconut FFA oil. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| Fatty | | Coconut | Fish/ | |
|---|---|---|---|---|
| Acid | Fish Oil* | Oil* | Co-75 | Co50 |
| C6:0 | 0.0 | 0.0 | 0.0 | 0.0 |
| C8:0 | 0.0 | 0.0 | 0.0 | 0.0 |
| C10 | 0.0 | 6.0 | 1.5 | 6.0 |
| C12 | 0.0 | 47.0 | 11.8 | 47.0 |
| C14 | 9.4 | 18.0 | 11.6 | 18.0 |
| C16 | 26.9 | 9.0 | 22.4 | 9.0 |
| C18 | 0.0 | 3.0 | 0.8 | 3.0 |
| C18:1 | 21.8 | 2.0 | 16.8 | 2.0 |
| C18:2 | 2.5 | 2.0 | 2.4 | 2.0 |
| C18:3 | 1.7 | 0.0 | 1.3 | 0.0 |
| C20 | 14.5 | 0.0 | 10.9 | 0.0 |
| C22 | 23.2 | 0.0 | 17.4 | 0.0 |

| | EMO/FFA Oil |
|---|---|
| EMO | Blends |

| | | | | |
|---|---|---|---|---|
| MAG | 56 | 72 | 42 | 36 |
| DAG | | 16 | 10.5 | 8 |
| TAG | | BLD | 12 | BLD |
| FFA | 12 | 12 | 33 | 52.5 |

| | | |
|---|---|---|
| MAG/FFA Ratio | 1.3 | 0.7 |

*Values from literature

In certain preferred embodiments, where the microbial infection is caused by or the target for sterilizing or disinfecting is B. cereus, the antimicrobial oil composition can include about 30% to about 50% MAGs and about 25% to about 60% FFA. In certain preferred embodiments, where the microbial infection is caused by or the target for sterilizing or disinfecting is B. cereus, the antimicrobial oil composition can have a fatty acid profile that comprises two or more (or three, four, five, six, seven, eight, nine, or ten) fatty acids selected from the group consisting of:

| Fatty Acid | Amount out of Total Fatty Acids |
|---|---|
| C10 | 0.1%-15% |
| C12 | 5%-55% |
| C14 | 5%-25% |
| C16 | 5%-30% |
| C18 | 0.1%-10% |
| C18:1 | 0.1%-25% |
| C18:2 | 0.1%-10% |
| C18:3 | 0.1%-10% |
| C20 | 5%-15% |
| C22 | 10%-25% |

In certain preferred embodiments, where the microbial infection is caused by or the target for sterilizing or disinfecting is B. cereus, the MAGs antimicrobial oil composition can have a fatty acid profile that comprises two or more fatty acids (or three, four, five, six, seven, eight, nine or ten) selected from the group consisting of:

| Fatty Acid | Amount out of Total Fatty Acids |
|---|---|
| C10 | 0.1%-10% |
| C12 | 40%-55% |
| C14 | 5%-25% |
| C16 | 5%-35% |
| C18 | 0.1%-10% |
| C18:1 | 0.1%-30% |
| C18:2 | 0.1%-10% |
| C18:3 | 0.1%-10% |
| C20 | 10%-20% |
| C22 | 15%-30% |

In certain preferred embodiments, where the microbial infection is caused by or the target for sterilizing or disinfecting is B. cereus, the ratio of MAG/FFA in the antimicrobial oil composition is about 0.7 to about 1.3.

Streptococcus agalactiae

In any of the foregoing embodiments, the microbial infection can be caused by or the target for sterilizing or disinfecting is Streptococcus agalactiae. In such embodiments, the method of treating or preventing can be to treat or prevent a vaginal infection. In certain preferred embodiments, where the microbial infection is caused by or the target for sterilizing or disinfecting is S. agalactiae, the antimicrobial oil composition can include EMO derived from coconut oil, fish oil, or a combination thereof, and FFA oil from flaxseed oil. Thus, in such embodiments, the MAGs can be derived coconut oil, fish oil, or a combination thereof, and the FFA can be derived from coconut oil, fish oil, or a combination thereof (for the first portion of FFA) and flaxseed oil (for the second portion of FFA). In certain preferred embodiments, where the microbial infection is caused by or the target for sterilizing or disinfecting is S. agalactiae, the antimicrobial oil composition is a mixture of 50% fish EMO/50% flaxseed FFA oil or 50% coconut EMO/50% flaxseed FFA. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| Fatty | Flaxseed | | Coconut | Fish/ | Co/ |
|---|---|---|---|---|---|
| Acid | Oil | Fish Oil* | Oil* | Fx-50 | Fx-50 |
| C6:0 | 0.0 | 0.0 | 0.0 | 0.0 | 0.0 |
| C8:0 | 0.0 | 0.0 | 0.0 | 0.0 | 0.0 |
| C10 | 0.0 | 0.0 | 6.0 | 0.0 | 3.0 |
| C12 | 0.0 | 0.0 | 47.0 | 0.0 | 23.5 |
| C14 | 0.0 | 9.4 | 18.0 | 4.7 | 9.0 |
| C16 | 5.2 | 26.9 | 9.0 | 16.0 | 7.1 |
| C18 | 3.3 | 0.0 | 3.0 | 1.6 | 3.1 |
| C18:1 | 15.4 | 21.8 | 2.0 | 18.6 | 8.7 |
| C18:2 | 16.0 | 2.5 | 2.0 | 9.2 | 9.0 |
| C18:3 | 59.7 | 1.7 | 0.0 | 30.7 | 29.8 |
| C20 | 0.4 | 14.5 | 0.0 | 7.5 | 0.2 |
| C22 | 0.0 | 23.2 | 0.0 | 11.6 | 0.0 |
| | | | | EMO/FFA Oil | |
| EMO | | | | Blends | |
| MAG | 57.9 | 56 | 72 | 28 | 36 |
| DAG | 16.5 | 10 | 16 | 5 | 5 |
| TAG | BLD | 16 | BLD | 8 | BLD |
| FFA | 18 | 12 | 12 | 53 | 52.5 |

| | | |
|---|---|---|
| MAG/FFA Ratio | 0.5 | 0.7 |

*Values from literature

In certain preferred embodiments, where the microbial infection is caused by or the target for sterilizing or disinfecting is S. agalactiae, the antimicrobial oil composition can include about 20% to about 45% MAGs and about 45% to about 60% FFA. In certain preferred embodiments, where the microbial infection is caused by or the target for sterilizing or disinfecting is S. agalactiae, the antimicrobial oil composition can have a fatty acid profile that comprises two or more (or three, four, five, six, seven, eight, nine, or ten) fatty acids selected from the group consisting of:

| Fatty Acid | Amount out of Total Fatty Acids |
|---|---|
| C10 | 0.1%-10% |
| C12 | 15%-30% |
| C14 | 0.1%-15% |
| C16 | 0.1%-25% |
| C18 | 0.1%-10% |
| C18:1 | 0.1%-25% |
| C18:2 | 0.1%-15% |
| C18:3 | 20%-40% |
| C20 | 0.1%-15% |
| C22 | 5%-20% |

In certain preferred embodiments, where the microbial infection is caused by or the target for sterilizing or disinfecting is S. agalactiae, the MAGs antimicrobial oil composition can have a fatty acid profile that comprises two or more fatty acids (or three, four, five, six, seven, eight, nine or ten) selected from the group consisting of:

| Fatty Acid | Amount out of Total Fatty Acids |
|---|---|
| C10 | 0.1%-10% |
| C12 | 40%-55% |
| C14 | 5%-25% |
| C16 | 5%-35% |
| C18 | 0.1%-10% |
| C18:1 | 0.1%-30% |
| C18:2 | 0.1%-10% |
| C18:3 | 0.1%-10% |
| C20 | 10%-20% |
| C22 | 15%-30% |

In certain preferred embodiments, where the microbial infection is caused by or the target for sterilizing or disinfecting is S. agalactiae, the ratio of MAG/FFA in the antimicrobial oil composition is about 0.5 to about 0.7.

Enveloped Viruses

In certain preferred embodiments, where the microbial infection is caused by or the target for sterilizing or disinfecting is an enveloped virus such as, by way of example, but not limitation, a herpes simplex virus, a coronavirus, or an influenza virus, the antimicrobial oil composition can include EMO derived from coconut oil, fish oil, or a combination thereof, and FFA oil from flaxseed oil. Thus, in such embodiments, the MAGs can be derived coconut oil, fish oil, or a combination thereof, and the FFA can be derived from coconut oil, fish oil, or a combination thereof (for the first portion of FFA) and flaxseed oil (for the second portion of FFA). In certain preferred embodiments, where the microbial infection is caused by or the target for sterilizing or disinfecting is an enveloped virus, the antimicrobial oil composition is a mixture of 25% oat EMO/75% flaxseed FFA oil. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| | | | Flaxseed | |
|---|---|---|---|---|
| | Fatty Acid | Oat Oil | Oil | Oa/Fx-25 |
| | C6:0 | 0.0 | 0.0 | 0.0 |
| | C8:0 | 0.0 | 0.0 | 0.0 |
| | C10 | 0.0 | 0.0 | 0.0 |
| | C12 | 0.0 | 0.0 | 0.0 |
| | C14 | 0.0 | 0.0 | 0.0 |
| | C16 | 11.4 | 5.2 | 6.8 |
| | C18 | 4.3 | 3.3 | 3.6 |
| | C18:1 | 22.5 | 15.4 | 17.2 |
| | C18:2 | 53.8 | 16.0 | 25.4 |
| | C18:3 | 7.2 | 59.7 | 46.6 |
| | C20 | 0.8 | 0.4 | 0.5 |
| | C22 | 0.0 | 0.0 | 0.0 |
| | | | | EMO/FFA |
| EMO | | | | Oil Blends |
| | MAG | 65.9 | 57.9 | 16.5 |
| | DAG | 9.4 | 16.5 | 2.4 |
| | TAG | BLD | BLD | BLD |
| | FFA | 13 | 18 | 73.0 |

| | |
|---|---|
| MAG/FFA Ratio | 0.2 |

In certain preferred embodiments, where the microbial infection is caused by or the target for sterilizing or disinfecting is an enveloped virus, the antimicrobial oil composition can include about 20% to about 45% MAGs and about 45% to about 60% FFA. In certain preferred embodiments, where the microbial infection is caused by or the target for sterilizing or disinfecting is an enveloped virus, the antimicrobial oil composition can have a fatty acid profile that comprises two or more (or three, four, or five) fatty acids selected from the group consisting of:

| Fatty Acid | Amount out of Total Fatty Acids |
|---|---|
| C16 | 0.1%-10% |
| C18 | 0.1%-10% |
| C18:1 | 10%-20% |
| C18:2 | 10%-20% |
| C18:3 | 55-65% |

In certain preferred embodiments, where the microbial infection is caused by or the target for sterilizing or disinfecting is an enveloped virus, the MAGs antimicrobial oil composition can have a fatty acid profile that comprises two or more fatty acids (or three, four, five, or six) selected from the group consisting of:

| Fatty Acid | Amount out of Total Fatty Acids |
|---|---|
| C16 | 5%-20% |
| C18 | 0.1%-10% |
| C18:1 | 15%-30% |
| C18:2 | 45%-60% |
| C18:3 | 0.1%-15% |
| C20 | 0.1%-5% |

In certain preferred embodiments, where the microbial infection is caused by or the target for sterilizing or disinfecting is an enveloped virus, the ratio of MAG/FFA in the antimicrobial oil composition is about 0.2.

Respiratory Syncitial Virus

In certain preferred embodiments, where the microbial infection is caused by or the target for sterilizing or disinfecting is respiratory syncytial virus (RSV), the antimicrobial oil composition can include EMO derived from MCT oil, oat oil, or combinations thereof and FFA oil from flaxseed oil. Thus, in such embodiments, the MAGs can be derived MCT oil, oat oil, or combinations thereof and the FFA can be derived from MCT oil, oat oil, or combinations thereof (for the first portion of FFA) and flaxseed oil (for the second portion of FFA). In certain preferred embodiments, where the microbial infection is caused by or the target for sterilizing or disinfecting is respiratory syncytial virus, the antimicrobial oil composition is a mixture of 50% MCT EMO/50% flaxseed FFA oil or 25% oat EMO/75% flaxseed FFA oil. The expected fatty acid profile and MAG and FFA content are provided in The table below below. It should be understood that, where MAG, DAG, TAG and FFA values are provided, these are from experimental results but can be varied by the conditions of the processing to yield EMO. For the following tables, it is assumed that FFA oils contain 93% FFA, with the exception that fish oil is assumed to contain 77% FFA.

| Fatty | | Flaxseed | | | |
|---|---|---|---|---|---|
| Acid | MCT Oil | Oil | Oat Oil | MCT/Fx-50 | Oa/Fx-25 |
| C6:0 | 0.06 | 0.0 | 0.0 | 0.0 | 0.0 |
| C8:0 | 57.42 | 0.0 | 0.0 | 28.7 | 0.0 |
| C10 | 41.96 | 0.0 | 0.0 | 21.0 | 0.0 |
| C12 | 0.32 | 0.0 | 0.0 | 0.2 | 0.0 |
| C14 | 0.0 | 0.0 | 0.0 | 0.0 | 0.0 |
| C16 | 0.0 | 5.2 | 11.4 | 2.6 | 6.8 |
| C18 | 0.0 | 3.3 | 4.3 | 1.6 | 3.6 |
| C18:1 | 0.0 | 15.4 | 22.5 | 7.7 | 17.2 |
| C18:2 | 0.0 | 16.0 | 53.8 | 8.0 | 25.4 |
| C18:3 | 0.0 | 59.7 | 7.2 | 29.8 | 46.6 |
| C20 | 0.0 | 0.4 | 0.8 | 0.2 | 0.5 |
| C22 | 0.0 | 0.0 | 0.0 | 0.0 | 0.0 |
| EMO | | | | EMO/FFA Oil Blend | |
| MAG | Est. 64 | 57.9 | 65.9 | 32 | 16.5 |
| DAG | Est. 15 | 16.5 | 9.4 | 7.5 | 2.4 |
| TAG | BLD | BLD | BLD | BLD | BLD |
| FFA | 21 | 18 | 13 | 53 | 73.0 |

| | | |
|---|---|---|
| MAG/FFA Ratio | 0.6 | 0.2 |

In certain preferred embodiments, where the microbial infection is caused by or the target for sterilizing or disinfecting is RSV, the antimicrobial oil composition can include about 10% to about 40% MAGs and about 50% to about 80% FFA. In certain preferred embodiments, where the microbial infection is caused by or the target for sterilizing or disinfecting is RSV, the antimicrobial oil composition can have a fatty acid profile that comprises two or more (or three, four, five, six, or seven) fatty acids selected from the group consisting of:

| Fatty Acid | Amount out of Total Fatty Acids |
|---|---|
| C8:0 | 20%-35% |
| C10 | 15%-30% |
| C16 | 0.1%-10% |
| C18 | 0.1%-25% |
| C18:1 | 0.1%-30% |
| C18:2 | 10%-20% |
| C18:3 | 20-55% |

In certain preferred embodiments, where the microbial infection is caused by or the target for sterilizing or disinfecting is RSV, the MAGs antimicrobial oil composition can have a fatty acid profile that comprises two or more fatty acids (or three, four, five, six, or seven) selected from the group consisting of:

| Fatty Acid | Amount out of Total Fatty Acids |
|---|---|
| C8:0 | 50%-65% |
| C10 | 35%-50% |
| C16 | 5%-15% |
| C18 | 0.1%-10% |
| C18:1 | 15%-30% |
| C18:2 | 45%-60% |
| C18:3 | 0.1%-15% |

In certain preferred embodiments, where the microbial infection is caused by or the target for sterilizing or disinfecting is RSV, the ratio of MAG/FFA in the antimicrobial oil composition is about 0.2 to about 0.6.

It should also be understood that, in some embodiments, the methods of treating or preventing or of sanitizing or sterilizing can be used against Clostridium difficile, including to treat gastrointestinal infections.

Exemplary antimicrobial compositions for antimicrobial applications are provided in the tables below and include the fatty acid profile of the oil sources, of the blend of EMO/FFA oil, and the glyceride compositions as MAG, DAG, TAG and FFA. It should be understood that the fatty acid profile for each fatty acid can vary by as much as 3-5% due to outside factors such as growth conditions of the source of the oil. It should be further understood that the following compositions are merely exemplary, but represent preferred oil compositions that can be used in the antimicrobial compositions and nanoemulsions of the present disclosure. Other preferred EMO/FFA oil ratios and blends are provided in Tables the tables below. The fatty acid profile for a blend can be calculated based on the weight ratio of EMO to FFA oil for any combination. The tables below provide additional fatty acid profiles for other oil sources. It should be understood that the MAG/DAG/TAG/FFA amounts provided for for exemplary EMOs that were produced in the Examples, however, conditions of the enzymatic treatment can be adjusted to arrive at different values, if desired.

Compositions of the present disclosure and antimicrobial compositions thereof can also be used in various devices, such as in a nebulizer, a vaping device, a bandage, gauze, a suture, a plastic object such as a catheter or IV port, floss, or a condom, such as to prevent sexually transmitted disease transmission.

Methods of Treatment

Oil compositions and nanoemulsion compositions of the present disclosure and antimicrobial compositions thereof can be useful for the treatment of surfaces and systemic conditions including, but not limited to: skin (hands, feet, face, genitals, torso, scalp, and the like), mouth and teeth, ear, nose, throat, pulmonary system (e.g., lungs), gastrointestinal system such as stomach, small/large bowl, rectum, or anus, gallbladder, appendix, surgical incision sites, surgical incisions and internal tissue exposed during surgery or trauma, cuts and wounds, the circulatory system (e.g. blood), brain, food work surfaces, packaging equipment, biofilm eradication in oil and gas pipelines, packaging, and as disinfectant sprays and dips.

In some embodiments, a method for treating or preventing a microbial infection or disease or condition caused by a microbial infection in a subject in need thereof includes the step of administering an effective amount of an antimicrobial composition of the present disclosure to said patient to treat or prevent the microbial infection. In certain aspects, the oil composition or nanoemulsion composition of the present disclosure can be administered itself or as part of an antimicrobial composition.

Treatment of Skin Conditions

In some embodiments, a method for treating or preventing a skin condition in a subject in need thereof is provided that can include administering a therapeutically effective amount of an antimicrobial composition of the present disclosure, such as one containing an antimicrobial oil composition, to the skin of the subject. In some embodiments, the composition can be in the form of a cream, such as by way of example but not limitation, incorporated into an off-the-shelf cream. In some embodiments, the composition can be applied to a lesion or an adjacent region of the skin. In some embodiments, the method can include applying the composition to cover the skin. In some embodiments, the skin condition is atopic dermatitis. In some embodiments, the skin conditions can be psoriasis or icthyosis. It should be understood that the compositions of the present disclosure can be used to treat a skin condition such as, by way of example, but not limitation, atopic dermatitis or acne. In some embodiments, the antimicrobial oil composition can include 25% oat EMO/75% flaxseed FFA.

In any of the foregoing embodiments, the antimicrobial composition can be applied to the skin at any suitable frequency. By way of example, but not limitation, the antimicrobial composition can be applied to the skin daily, twice daily, three times daily, every 2 days, every 3 days, every 4 days, every 5 days, every 6 days, weekly, twice weekly, three times weekly, four times weekly, five times weekly, six times weekly, every two weeks, or monthly.

In any of the foregoing embodiments, the antimicrobial composition can be applied to the skin for at least a day, at least a week, at least 2 weeks, at least 3 weeks, at least a month, at least two months, or for any suitable period.

In any of the foregoing embodiments, the antimicrobial composition can be applied to the skin until a reduction in S. aureus strains meets Criteria A (a 2-log reduction in viable bacteria in 2 days, 3 log reduction in 7 days, and No Increase in 28 days; and a 2-log reduction in viable fungi in 14 days and No Increase in 28 days).

In any of the foregoing embodiments, the antimicrobial composition can reduce S. aureus strains while not reducing commensal strains such as, by way of example, but not limitation, S. hominis, S. capitis, and S. epidermidis. In certain aspects, any reduction in the commensal strains is less than the reduction in S. aureus. By way of example, but not limitation, the reduction S. aureus strains can meet Criteria A while any reduction in any of the commensal strains does not.

In some embodiments, a method is provided for treating or preventing acne in a subject in need thereof that can include administering a therapeutically effective amount of an antimicrobial composition of the present disclosure to the skin of the subject.

In some embodiments, a method for sanitizing or sterilizing an object or surface is provided that includes the step of applying an effective amount of a composition of the present disclosure to the object or surface.

In some embodiments, a method for biofilm eradication in gas or oil pipelines can include the step of flowing a composition of the present disclosure through the gas or oil pipeline.

In some embodiments, a method for inhibiting or reducing the growth of a microbial agent can include the step of applying an antimicrobial composition or nanoemulsion (or microemulsion) composition of the present disclosure to the microbial agent. It should be understood that the microbial agent can be bacterial, fungal or viral. In some embodiments, a method for killing a microbial agent can include the step of applying an antimicrobial composition or nanoemulsion (or microemulsion) composition of the present disclosure to the microbial agent. It should be understood that the microbial agent can be bacterial, fungal or viral. In the foregoing embodiments of the methods for inhibiting or reducing the growth of or killing a microbial agent, the composition or antimicrobial composition can be applied at an effective amount to inhibit or reduce growth or kill the microbial agent, respectively.

In some embodiments, a method for producing an antimicrobial composition of the present disclosure can include the step of combining a nanoemulsion (or microemulsion) of the present disclosure with a carrier formulation. Thus, while in some embodiments, the nanoemulsion (or microemulsion) of the present disclosure can not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts, the carrier formulation can include these agents. By way of example, but not limitation, the antimicrobial composition can be formulated as a cream which may require a thickener or other agent, however, the nanoemulsion (or microemulsion) component can, in some embodiments, not include such an agent. Examples of potential cream formulations are described in Example 19. In some embodiments, the nanoemulsions (or microemulsions) of the present disclosure are combined with an existing or off-the-shelf cream to provide antimicrobial properties to the cream or to enhance the antimicrobial properties of the cream. It should be understood that the EMO and FFA oil obtained from the enzymatic processes disclosed herein can be used without formulating into nanoemulsions or microemulsions. In some instances, the EMO or FFA oil, or both can be used such as, by way of example but not limitation, incorporation by homogenization into a pre-formulated cream, such as by way of example but not limitation an off-the-shelf cream. Thus, in any of the embodiments of the present disclosure, the composition can have all of the properties recited except for the size and other properties related to nanoemulsions and microemulsions. Alternatively, the EMO and FFA oil incorporated can be in the form of a nanoemulsion or microemulsion or later homogenized to form a nanoemulsion or microemulsion. It should be further understood that any of the compositions of the present disclosure can comprise FFA oil and not EMO or can include both in accordance with the present disclosure. The ratio of nanoemulsion (or composition) of the present disclosure to cream can be from about 1:1 to less than 1:20, from about 1:2 to about 1:9, from about 1:3 to about 1:8, from about 1:4 to about 1:7, from about 1:5 to about 1:6 and all intermediate ranges and values between the foregoing. By way of further example, the EMO and/or FFA oil can be present in the cream at about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.25%, 2.5%, 2.75%, 3%, 3.25%, 3.5%, 4%, 4.25%, 4.5%, 4.75%, 5%, 5.5%, 6%, 6.5%, 7%, 7.5%, 8%, 8.5%, 9%, 9.5%, 10%, 11%, 12%, 13%, 14%, 15%, 20%, 25%, 30%, 35%, 40%, 45%, 50% or any range or value therebetween. In some embodiments, the antimicrobial composition does not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts.

In some embodiments, the antimicrobial composition can include a first component that includes a nanoemulsion (or microemulsion) or EMO and/or FFA oil of the present disclosure, and a second component that includes a carrier formulation as described herein. Thus, while in some embodiments, the nanoemulsion (or microemulsion) or EMO and/or FFA oil of the present disclosure can not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts, the carrier formulation can include these agents. By way of example, but not limitation, the antimicrobial composition can be formulated as a cream which may require a thickener or other agent, however, the nanoemulsion (or microemulsion) component can, in some embodiments, not include such an agent. In some embodiments, the antimicrobial composition does not include any exogenous additive with surfactant and/or emulsifier properties that is not naturally present in the oil in an amount sufficient to enhance droplet or particle stability, any exogenous additive with thickening or crystallization inhibiting properties that is not naturally present in the oil in an amount sufficient to thicken the composition or inhibit crystal formation, respectively, a polyhydric alcohol, or bile salts.

Methods for Making Antimicrobial Oil Compositions

In some embodiments, a method for preparing an antimicrobial oil composition of the present disclosure is provided that can include combining a first amount of an enzyme-modified oil (EMO) derived from a first oil source and a second amount of a free fatty acid (FFA) oil derived from a second oil source to yield the antimicrobial oil composition. It should be understood that the EMO and the FFA oil can have any of the properties thereof disclosed in the present disclosure and in any of the foregoing embodiments.

In any of the foregoing methods for preparing the antimicrobial oil composition, the method can further include the step of compounding the antimicrobial oil composition to yield a cream, a lotion, a balm, an eye drop, an ear drop, a sinus rinse, a spray, such as nasal, oral, mucosal or for skin or feet, an ointment, a deodorant formulation, a body wash, a shampoo, a scalp treatment, a mouthwash, a toothpaste, a lozenge, a beverage, a paste or a lubricant, such as vaginal lubricant. In any of the foregoing methods for preparing the antimicrobial oil composition, the method can further include combining the antimicrobial oil composition with an aqueous phase and homogenizing to obtain a nanoemulsion or microemulsion. The nanoemulsion or microemulsion can have the properties of any of the nanoemulsion and microemulsion compositions disclosed herein.

In any of the foregoing embodiments, the first amount out of the total amount of the first amount and the second amount can be from about 0.1% to about 99.9%, about 0.1% to about 99.8%, about 0.1% to about 99.5%, about 0.1% to about 99%, about 0.1% to about 98%, about 0.1% to about 97%, about 0.1% to about 96%, about 0.1% to about 95%, about 0.1% to about 94%, about 0.1% to about 93%, about 0.1% to about 92%, about 0.1% to about 91%, about 0.1% to about 90%, about 0.1% to about 80%, about 0.1% to about 75%, about 0.1% to about 70%, about 0.1% to about 60%, about 0.1% to about 50%, about 0.1% to about 40%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, about 0.1% to about 1%, about 0.1% to about 0.5%, about 0.1% to about 0.2%, 0.2% to about 99.9%, about 0.2% to about 99.8%, about 0.2% to about 99.5%, about 0.2% to about 99%, about 0.2% to about 98%, about 0.2% to about 97%, about 0.2% to about 96%, about 0.2% to about 95%, about 0.2% to about 94%, about 0.2% to about 93%, about 0.2% to about 92%, about 0.2% to about 91%, about 0.2% to about 90%, about 0.2% to about 80%, about 0.2% to about 75%, about 0.2% to about 70%, about 0.2% to about 60%, about 0.2% to about 50%, about 0.2% to about 40%, about 0.2% to about 30%, about 0.2% to about 25%, about 0.2% to about 20%, about 0.2% to about 15%, about 0.2% to about 10%, about 0.2% to about 5%, about 0.2% to about 4%, about 0.2% to about 3%, about 0.2% to about 2%, about 0.2% to about 1%, about 0.2% to about 0.5%, 0.5% to about 99.9%, about 0.5% to about 99.8%, about 0.5% to about 99.5%, about 0.5% to about 99%, about 0.5% to about 98%, about 0.5% to about 97%, about 0.5% to about 96%, about 0.5% to about 95%, about 0.5% to about 94%, about 0.5% to about 93%, about 0.5% to about 92%, about 0.5% to about 91%, about 0.5% to about 90%, about 0.5% to about 80%, about 0.5% to about 75%, about 0.5% to about 70%, about 0.5% to about 60%, about 0.5% to about 50%, about 0.5% to about 40%, about 0.5% to about 30%, about 0.5% to about 25%, about 0.5% to about 20%, about 0.5% to about 15%, about 0.5% to about 10%, about 0.5% to about 5%, about 0.5% to about 4%, about 0.5% to about 3%, about 0.5% to about 2%, about 0.5% to about 1%, about 1% to about 99.9%, about 1% to about 99.8%, about 1% to about 99.5%, about 1% to about 99%, about 1% to about 98%, about 1% to about 97%, about 1% to about 96%, about 1% to about 95%, about 1% to about 94%, about 1% to about 93%, about 1% to about 92%, about 1% to about 91%, about 1% to about 90%, about 1% to about 80%, about 1% to about 75%, about 1% to about 70%, about 1% to about 60%, about 1% to about 50%, about 1% to about 40%, about 1% to about 30%, about 1% to about 25%, about 1% to about 20%, about 1% to about 15%, about 1% to about 10%, about 1% to about 5%, about 1% to about 4%, about 1% to about 3%, about 1% to about 2%, about 2% to about 99.9%, about 2% to about 99.8%, about 2% to about 99.5%, about 2% to about 99%, about 2% to about 98%, about 2% to about 97%, about 2% to about 96%, about 2% to about 95%, about 2% to about 94%, about 2% to about 93%, about 2% to about 92%, about 2% to about 91%, about 2% to about 90%, about 2% to about 80%, about 2% to about 75%, about 2% to about 70%, about 2% to about 60%, about 2% to about 50%, about 2% to about 40%, about 2% to about 30%, about 2% to about 25%, about 2% to about 20%, about 2% to about 10%, about 2% to about 5%, about 5% to about 99.9%, about 5% to about 99.8%, about 5% to about 99.5%, about 5% to about 99%, about 5% to about 98%, about 5% to about 97%, about 5% to about 96%, about 5% to about 95%, about 5% to about 94%, about 5% to about 93%, about 5% to about 92%, about 5% to about 91%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 10%, about 10% to about 99.9%, about 10% to about 99.8%, about 10% to about 99.5%, about 10% to about 99%, about 10% to about 98%, about 10% to about 97%, about 10% to about 96%, about 10% to about 95%, about 10% to about 94%, about 10% to about 93%, about 10% to about 92%, about 10% to about 91%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 20% to about 99.9%, about 20% to about 99.8%, about 20% to about 99.5%, about 20% to about 99%, about 20% to about 98%, about 20% to about 97%, about 20% to about 96%, about 20% to about 95%, about 20% to about 94%, about 20% to about 93%, about 20% to about 92%, about 20% to about 91%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 99.9%, about 25% to about 99.8%, about 25% to about 99.5%, about 25% to about 99%, about 25% to about 98%, about 25% to about 97%, about 25% to about 96%, about 25% to about 95%, about 25% to about 94%, about 25% to about 93%, about 25% to about 92%, about 25% to about 91%, about 25% to about 90%, about 25% to about 80%, about 25% to about 75%, about 25% to about 70%, about 25% to about 60%, about 25% to about 50%, about 25% to about 40%, about 25% to about 30%, about 30% to about 99.9%, about 30% to about 99.8%, about 30% to about 99.5%, about 30% to about 99%, about 30% to about 98%, about 30% to about 97%, about 30% to about 96%, about 30% to about 95%, about 30% to about 94%, about 30% to about 93%, about 30% to about 92%, about 30% to about 91%, about 30% to about 90%, about 30% to about 80%, about 30% to about 75%, about 30% to about 70%, about 30% to about 60%, about 30% to about 50%, about 30% to about 40%, about 40% to about 99.9%, about 40% to about 99.8%, about 40% to about 99.5%, about 40% to about 99%, about 40% to about 98%, about 40% to about 97%, about 40% to about 96%, about 40% to about 95%, about 40% to about 94%, about 40% to about 93%, about 40% to about 92%, about 40% to about 91%, about 40% to about 90%, about 40% to about 80%, about 40% to about 75%, about 40% to about 70%, about 40% to about 60%, about 40% to about 50%, about 50% to about 99.9%, about 50% to about 99.8%, about 50% to about 99.5%, about 50% to about 99%, about 50% to about 98%, about 50% to about 97%, about 50% to about 96%, about 50% to about 95%, about 50% to about 94%, about 50% to about 93%, about 50% to about 92%, about 50% to about 91%, about 50% to about 90%, about 50% to about 80%, about 50% to about 75%, about 50% to about 70%, about 50% to about 60%, about 60% to about 99.9%, about 60% to about 99.8%, about 60% to about 99.5%, about 60% to about 99%, about 60% to about 98%, about 60% to about 97%, about 60% to about 96%, about 60% to about 95%, about 60% to about 94%, about 60% to about 93%, about 60% to about 92%, about 60% to about 91%, about 60% to about 90%, about 60% to about 80%, about 60% to about 75%, about 60% to about 70%, about 70% to about 99.9%, about 70% to about 99.8%, about 70% to about 99.5%, about 70% to about 99%, about 70% to about 98%, about 70% to about 97%, about 70% to about 96%, about 70% to about 95%, about 70% to about 94%, about 70% to about 93%, about 70% to about 92%, about 70% to about 91%, about 70% to about 90%, about 70% to about 80%, about 80% to about 99.9%, about 80% to about 99.8%, about 80% to about 99.5%, about 80% to about 99%, about 80% to about 98%, about 80% to about 97%, about 80% to about 96%, about 80% to about 95%, about 80% to about 94%, about 80% to about 93%, about 80% to about 92%, about 80% to about 91%, about 80% to about 90%, about 90% to about 99.9%, about 90% to about 99.8%, about 90% to about 99.5%, about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 99.9%, about 91% to about 99.8%, about 91% to about 99.5%, about 91% to about 99%, about 91% to about 98%, about 91% to about 97%, about 91% to about 96%, about 91% to about 95%, about 91% to about 94%, about 91% to about 93%, about 91% to about 92%, about 92% to about 99.9%, about 92% to about 99.8%, about 92% to about 99.5%, about 92% to about 99%, about 92% to about 98%, about 92% to about 97%, about 92% to about 96%, about 92% to about 95%, about 92% to about 94%, about 92% to about 93%, about 93% to about 99.9%, about 93% to about 99.8%, about 93% to about 99.5%, about 93% to about 99%, about 93% to about 98%, about 93% to about 97%, about 93% to about 96%, about 93% to about 95%, about 93% to about 94%, about 94% to about 99.9%, about 94% to about 99.8%, about 94% to about 99.5%, about 94% to about 99%, about 94% to about 98%, about 94% to about 97%, about 94% to about 96%, about 94% to about 95%, about 95% to about 99.9%, about 95% to about 99.8%, about 95% to about 99.5%, about 95% to about 99%, about 95% to about 98%, about 95% to about 97%, about 95% to about 96%, about 96% to about 99.9%, about 96% to about 99.8%, about 96% to about 99.5%, about 96% to about 99%, about 96% to about 98%, about 96% to about 97%, about 97% to about 99.9%, about 97% to about 99.8%, about 97% to about 99.5%, about 97% to about 99%, about 97% to about 98%, about 98% to about 99.9%, about 98% to about 99.8%, about 98% to about 99.5%, about 98% to about 99%, about 99% to about 99.9%, about 99% to about 99.8%, about 99% to about 99.5%, about 99.5% to about 99.9%, about 99.5% to about 99.8%, about 99.8% to about 99.9%, or about 0.1%, 0.2%, 0.5%, 1%, 2%, 3%, 4%, 5%, 6%, 7%, 8%, 9%, 10%, 15%, 20%, 25%, 30%, 40%, 50%, 60%, 70%, 75%, 80%, 90%, 91%, 92%, 93%, 94%, 95%, 96%, 97%, 98%, 99%, 99.5%, 99.8%, or 99.9%. By way of still further example, the first amount as a percentage of the total of the first amount and the second amount can be from about 25% to about 75%.

It should be understood that the EMO and the FFA as disclosed herein, can have a fatty acid profile as described in the foregoing embodiments herein. It should be further understood that the antimicrobial oil composition can have a fatty acid profile as described in the foregoing embodiments herein.

In any of the foregoing embodiments, a method for preparing an antimicrobial composition can include combining an antimicrobial oil composition, nanoemulsion or microemulsion of any embodiments of the present disclosure with a cream, a lotion, a balm, an eye drop, an ear drop, a sinus rinse, a spray, such as nasal, oral, mucosal or for skin or feet, an ointment, a deodorant formulation, a body wash, a shampoo, a scalp treatment, a mouthwash, a toothpaste, a lozenge, a beverage, a paste or a lubricant, such as vaginal lubricant. It should be understood that the antimicrobial oil composition, nanoemulsion or microemulsion can be added to a cream, a lotion, a balm, an eye drop, an ear drop, a sinus rinse, a spray, such as nasal, oral, mucosal or for skin or feet, an ointment, a deodorant formulation, a body wash, a shampoo, a scalp treatment, a mouthwash, a toothpaste, a lozenge, a beverage, a paste or a lubricant, such as vaginal lubricant after either or both have been heated, by way of example, but not limitation to 60-80° C. It should be further understood that the antimicrobial oil composition, nanoemulsion or microemulsion can be added in separate parts as EMO and FFA and later homogenized to obtain the nanoemulsion or microemulsion. It should also be understood that the EMO and FFA can be added separately. In some embodiments, the antimicrobial composition can include a preservative selected from the group consisting of phenoxyethanol, potassium sorbate, EDTA and ethylhexylglycerin. In some embodiments, the preservative is present in an amount from about 0.1% to about 2% (w/w). In some embodiments, the antimicrobial composition can include a stabilizer selected from the group consisting of ceramide NP and xanthan gum. In some embodiments, the stabilizer is present in an amount from about 0.01% to about 0.5% (w/w). In any of the foregoing embodiments, the antimicrobial composition can not include quaternary amines.

In any of the foregoing embodiments, the antimicrobial composition can not include any exogenous additive with surfactant and/or emulsifier properties that is not naturally present in the oil in an amount sufficient to enhance droplet or particle stability. By way of example, but not limitation, the antimicrobial composition can not include polysorbates, phospholipids, sterols, cationic lipids, poloxamers, sorbitan esters, sugar esters, polyoxyethylene conjugates, ammonium phosphatidyl lapsidate, polyphenols, polyvinylalcohol, lecithin, ethanol, lysophospholipids, ceramides, Tweens, Spans, Kolliphor, propylene glycol, vitamin D, polyethylene glycol, polyethylene glycol derivatives, gum acacia, modified gum acacia, agar, ghatti gum, modified ghatti gum, pectin, carrageenan, xanthan gum, modified starches, modified alginate, fatty alcohols, ethoxylated polyols, and combinations thereof.

In any of the foregoing embodiments, the antimicrobial composition can not include any exogenous additive with thickening or crystallization inhibiting properties that is not naturally present in the oil in an amount sufficient to thicken the composition or inhibit crystal formation, respectively. By way of example, but not limitation, the antimicrobial composition can not include methyl cellulose, cornstarch, sodium alginate, or gelatin. By way of further example, but not limitation, the antimicrobial oil composition, nanoemulsion composition, or a composition comprising either can not include polyglycerol fatty acid esters, sucrose fatty acid esters, sorbitan fatty acid esters, and combinations thereof. In certain aspects, the composition can not include any exogenous additive with thickening properties that is not naturally present in the oil in an amount sufficient to thicken the composition. In certain aspects, the antimicrobial composition can not include any exogenous additive with crystallization inhibiting properties that is not naturally present in the oil in an amount sufficient to inhibit crystal formation. In other aspects, the antimicrobial oil composition, nanoemulsion composition, or a composition comprising either can not include any exogenous additive with thickening or crystallization inhibiting properties that is not naturally present in the oil in an amount sufficient to thicken the composition or inhibit crystal formation, respectively.

In any of the foregoing embodiments, the antimicrobial composition can not include a quaternary amine. It has surprisingly been found that the antimicrobial oil compositions of the present disclosure can avoid the need for harsh preservatives such as quaternary amines and can enable to the use of milder preservatives.

In any of the foregoing embodiments, the antimicrobial composition can not include a polyhydric alcohol.

In any of the foregoing embodiments, the antimicrobial composition can not include bile salts.

It should be understood that the disclosure herein is not limited to the use of EMO and FFA oils and that any oil may be used that meets the requirements of the present disclosure.

### EXAMPLES

It should understood that in the following examples, the prefix “n” before a type of oil, e.g. nOA/Fx-25, refers to a nanoemulsion having 25% Oat (OA) MAG EMO and 75% flaxseed FFA oil. For all examples, FFA oils were assumed to contain 93%+/−2% FFA with the exception of fish FFA oil which contains 77%+/−2% FFA and algal FFA oil which contains 61%+/−2% FFA. However, longer reaction times and different conditions can yield higher FFA if desired.

In the following examples, all percentages are (w/w) unless indicated otherwise or inconsistent with the standard interpretation of the percentage expressed.

Fatty Acid Oil Preparation

Fatty acid oils in the following examples (except Example 1) were prepared by the following procedure unless otherwise specified.

Fatty acid oils were prepared by the complete hydrolysis of the triacylglcyerides (TAGs) in the source oil to free fatty acids (FFA) and glycerol, followed by subsequent separation of the FFA and glycerol.

Free Fatty Acid (FFA) Oil Preparation

Free fatty acid oil was prepared by the complete hydrolysis of TAGs to FFA and glycerol followed by subsequent separation of the glycerol from the FFA oil. FFA oils were prepared in a 5 L stirred, jacketed glass reactor. Citric acid buffer (100 mM, pH 5.8) was prepared by combining 18.5 grams of food grade citric acid with 1.50 g of food grade sodium hydroxide in 963 mL of distilled de-ionized water. Amano Lipase AY (0.78 g) was dissolved in the solution which was then heated to 33° C. and agitated at 300 rpm. After a brief (10 min.) vacuum degassing, 780 g of starting oil was added to the reactor. The reaction volume was overlaid with nitrogen and the reaction proceeded for 24 hours until all of the TAGs had been converted to fatty acids and glycerol. Once the reaction was complete, the mixture was heated to 70° C. for 1 hour to inactivate the lipase. Agitation was stopped. After the two phases had separated, the upper phase (the free fatty acid oil) was removed and stored at 4° C. in opaque plastic bottles under nitrogen. Components of the oil samples were separated using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed. The intensity of the spots diminished after 30-60 minutes.

Enzyme-Modified Oil (EMO) Preparation

Enzyme-modified oil was produced from the FFA oil through the selective re-esterification of the FFA to glycerol to form MAG and (to a much lesser extent) DAG. FFA oil as described above was combined with 1,560 g of food grade glycerol and heated to 30° C. with agitation (300 rpm). Vacuum was applied (˜720 mmHg) to degas the material and remove residual water. Amano Lipase G (1.56 g in 50 mL DI water) was then added to the reactor and the vacuum was reapplied (˜740 mmHg). The reaction was allowed to proceed for 72 hours with progress monitored by TLC. Once the level of FFA was reduced to below 20%, the reaction was stopped by removing the vacuum, overlaying the reactor with nitrogen and heating the system to 70° C. for an hour to inactivate the enzyme. Fatty acid concentrations were measured by titrimetry. Samples were dissolved in a solvent (95% ethanol/diethyl ether, 1/1, v/v) and titrated to neutrality (indicated with phenolphthalein) with 0.1M KOH in ethanol. Table salt (sodium chloride) was then added to the reactor. Once the salt was suspended, the agitation was stopped. After the two phases had separated (˜3 hours), the upper phase (the EMO) was removed. Antioxidant was added (tocopherol, 200 ppm) and the material was stored at 4° C. in opaque plastic bottles under nitrogen.

Analysis of EMOs was performed using an HPLC method described previously (Bruno Lima dos Santos, Kissya Kropf da Silva, Amanda Pereira Franco dos Santos, Débora Franca de Andrade & Luiz Antonio d'Avila (2016) Simultaneous analysis of esters and acylglycerols in biodiesel by high-performance liquid chromatography with refractive index detection, Journal of Liquid Chromatography & Related Technologies, 39:13, 620-626, DOI: 10.1080/10826076.2016.1225221).

The HPLC system consisted of an LC-20AD pump, SIL-20AC HT autosampler, CTO-20A column oven, and RID-10A refractive index detector (Shimadzu Scientific Instruments, Inc.). HPLC conditions were: 20 uL injection volume, isocratic elution of mobile phase consisting of 85% methanol, 10% isopropanol, and 5% hexanes, flow rate of 0.800 mL/min, and column temperature of 40° C. Column used was a SUPELCOSIL LC-18 column (250 mm×4.6 mm; 5 um particle size). Samples were prepared in mobile phase. A mobile phase blank chromatogram was used to correct for background.

In general, the retention times were 3.4-4.0 min for glycerol, 4.0-6.2 min for MAGs and FFAs, 6.3-10.8 min for DAGs, and 11.0-50.0 min for TAGs.

Integration of these peaks provided relative ratios, or area-%, of glycerol, MAG+FFA, DAG, and TAG.

Since the MAG and FFA peaks overlapped, FFA content was determined by titrimetry. Samples were dissolved in a solvent (95% ethanol/diethyl ether, 1/1, v/v) and titrated to neutrality (indicated with phenolphthalein) with 0.1M KOH in ethanol. This value is reported as a weight percent.

MAG content was calculated by subtracting the FFA content from the MAG+FFA determined by HPLC.

Table 1 below provides the relative glycerol, MAG, DAG, TAG, and FFA content of the resulting EMOs which are used in the following Examples unless otherwise noted.

TABLE 1
MAG, DAG, TAG, and FFA Content of EMOs

| | MAG | DAG | TAG | FFA (wt- |
|---|---|---|---|---|
| EMO | (area-%) | (area-%) | (area-%) | %) |
| Sunflower | 58.3 | 25.5 | BLD | 9 |
| Canola | 60.9 | 16.3 | BLD | 13.9 |
| Almond | 74.9 | 12.5 | BLD | 10 |
| Flaxseed | 57.9 | 16.5 | BLD | 18 |
| Sesame | 62.5 | 17.9 | BLD | 12 |
| Olive | 55.9 | 14.4 | BLD | 20 |
| Rosehip | 54.6 | 16.5 | BLD | 16 |
| Hemp | 69 | 11 | 2.1 | 9 |
| Oat | 65.9 | 9.4 | BLD | 13 |
| Algae | 3.2 | 28 | 32.1 | 36.6 |

| | | | |
|---|---|---|---|
| Fish | 69* (estimated 56% | 15.7 | 12 |
| | MAG and 14% DAG) | | |
| MCT | 79 (estimated 64% MAG | BLD | 21 |
| | and 15% DAG) | | |
| Coconut | 88 (estimated 72% MAG | BLD | 12 |
| | and 16% DAG) | | |

BLD = below limit of detection
*MAG and DAG peaks overlapped

Nanoemulsion Preparation

Nanoemulsions were prepared from either EMOs or a combination of EMO and FFA oil(s). 1.5 g of total oils was weighed into a 50 mL metal beaker and heated to 60° C. A coarse emulsion was prepared by adding 13.5 mL water (pre-heated to 60° C.) to the oil. The mixture was subjected to high-sheer homogenizer for 30 seconds, resulting in a milky coarse emulsion. The coarse emulsion was then transferred to QSonica Q700CA sonicator with ½ inch probe. The emulsion was processed for 2 cycles (5 minutes sonication/2 minutes cooling) at 90% power. This process was used unless otherwise stated for all examples.

### Example 1: Production of Almond Fatty Acid Oil and Enzyme-Modified Oil (MO)

Fatty acid oil was prepared by complete hydrolysis of the TAGs to FFA and glycerol and their subsequent separation. Almond fatty acid oil was prepared in a 20 L stirred, jacketed glass reactor. Citric acid buffer (100 mM, pH 5.8) was prepared by combining 133 grams of food grade citric acid with 75 g of food grade sodium hydroxide in 7,000 mL of distilled de-ionized water. Amano Lipase AY (5.8 g) was dissolved in the solution which was then heated to 33° C. and agitated at 300 rpm. After a brief (10 min.) vacuum degassing, 5,600 g of almond oil was added to the reactor. The reaction volume was overlaid with nitrogen and the reaction proceeded for 12 hours until all of the TAGs had been converted to fatty acids and glycerol.

Once the reaction was complete, the mixture was heated to 70° C. for 1 hour to inactivate the lipase. Agitation was stopped. After the two phases had separated, the upper phase (the fatty acid oil) was removed and stored at room temperature in opaque plastic bottles under nitrogen.

Components of the oil samples were separated using TLC plates (Analtech Uniplate Silica Gel GHL with inorganic binder, 20×20 cm, 250 μm). The solvent was hexane:diethyl ether:acetic acid (70:30:1) solution. Typical sample sizes were 0.2 μL. After the solvent front ran to near the top of the plate (˜1 cm), plates were removed from the TLC tank and the solvent evaporated in a fume hood. The components were visualized with iodine vapors (at room temperature) in a TLC tank and relative intensities estimated by colorimetric imaging (Amersham 600 Imager). After 15 minutes in the tank, plates were removed and photographed. The intensity of the spots diminished after 30-60 minutes.

Enzyme-modified oil was produced from the fatty acid oil through the selective re-esterification of the FFA to glycerol to form MAG and (to a much lesser extent) DAG. Fatty acid oil as described above was combined with 10,260 g of food grade glycerol and heated to 30° C. with agitation (300 rpm). Vacuum was applied (˜720 mmHg) to degas the material and remove residual water. Amano Lipase G (5 g in 50 mL DI water) was then added to the reactor and the vacuum was reapplied (˜740 mmHg). The reaction was allowed to proceed for 72 hours with progress monitored by TLC.

Once the level of FFA was reduced to below 15%, the reaction was stopped by removing the vacuum, overlaying the reactor with nitrogen and heating the system to 70° C. for an hour to inactivate the enzyme.

Fatty acid concentrations were measured by titrimetry. Samples were dissolved in a solvent (95% ethanol/diethyl ether, 1/1, v/v) and titrated to neutrality (indicated with phenolphthalein) with 0.1M KOH in ethanol.

Table salt (sodium chloride) was then added to the reactor. Once the salt was suspended, the agitation was stopped. After the two phases had separated (3 hours), the upper phase (the acylglycerol oil) was removed. Antioxidant was added (tocopherol, 200 ppm) and the material was stored at room temperature in opaque plastic bottles under nitrogen.

Lipid components including C10:0 Capric Acid, C12:0 Lauric Acid, C14:0 Myristic Acid, C16:0 Palmitic Acid, C18:0 Stearic Acid, C18:1 Oleic Acid, C18:2 Linoleic Acid, and C18:3 Alpha Linolenic Acid were analyzed after derivatization as the fatty acid methyl esters and compared to standards. For the derivatization, a sample (500μl) was added to a 5-ml reaction tube containing 2 ml boron trifluoride solution (12% in methanol), 20 μl dimethoxypropane and 100 μl of a tridecanoic acid internal standard solution (10 mg/ml). The reaction tube was vortexed and incubated in a heating block at 60° C. for 30 minutes.

The reaction tube was removed from the heating block and allowed to cool for 15 minutes. Then, 1 ml of distilled water was added to quench the reaction, followed by 1 ml of hexane. The reaction tube was vortexed for 60 seconds and the phases were allowed to separate for 3 minutes. The top (hydrophobic) phase was removed to a 1.5-ml tube containing about 50 mg sodium sulfate (anhydrous). After vortexing for 60 seconds, the 1.5-ml tube was centrifuged and ˜500μl of the clarified, dried hydrophobic phase was transferred to a gas chromatography sample vial.

Samples were analyzed using an Agilent 7890A gas chromatograph with Flame ionization detector and Agilent Openlab CDS Chemstation software. GC Column: Omegawax 100 (15 m×0.1 mm×0.1 um) column. Results were converted to weight % by internal standard reference.

MCT, canola and almond oils were processed according to the manufacturing method of the present disclosure. Fatty acid profiles for each were measured in both the starting oil and the MAG oil as shown in Table 2 below.

As shown, both the starting oil and EMO contained substantially the same fatty acid profiles.

A separate experiment was run using almond and canola oil to obtain the data in Table 3 which show the same result for oleic, linoleic and linolenic acid.

TABLE 2
Conservation of Fatty acid profile

| | | MCT | | Canola | | Almond |
|---|---|---|---|---|---|---|
| Compound | MCT | MAG | Canola | MAG | Almond | MAG |
| C6:0 (Caproic acid) | 5.43% | 0.06% | | | | |
| C8:0 (Caprylic acid) | 58.41% | 57.42% | | | | |
| C10:0 (Capric acid) | 40.96% | 41.96% | | | | |
| C11:0 (Undecanoic acid) | 0.07% | 0.07% | | | | |
| C12:0 (Lauric Acid) | 0.32% | 0.32% | | | | |
| C14:0 (Myristic acid) | 0.03% | 0.03% | 0.05% | 0.06% | 0.06% | 0.06% |
| C15:0 (Palmitic Acid) | | | 4.06% | 4.06% | 6.61% | 6.65% |
| C15:1 Total (Palmitoleic Acid + isomers) | | | 0.30% | 0.30% | 0.57% | 0.57% |
| C17:0 (Margaric Acid) | | | 0.04% | 0.04% | 0.06% | 0.06% |
| C17:1 (Hepta decenoic Acid) | | | 0.05% | 0.06% | 0.11% | 0.11% |
| C18:0 (Stearic Acid) | | | 1.78% | 1.78% | 1.56% | 1.57% |
| C18:1 (Vaccenic acid) | | | 3.28% | 3.27% | 1.38% | 1.39% |
| C18:1. Total (Ofeic Acid + isomers) | 0.04% | 0.06% | 62.78% | 62.66% | 64.79% | 64.94% |
| C18:2. Total (Linoleic Acid + isomers) | 0.04% | 0.04% | 19.07% | 19.07% | 25.28% | 25.14% |
| C18:3. Total (Linolenic Acid + iso mers) | | | 8.26% | 8.22% | 0.29% | 0.28% |
| C18:4 Total (Octadecatetraenoic Acid) | | | 0.04% | 0.04% | 0.09% | 0.08% |
| C20:0 (Arachidic Acid) | | | 0.57% | 0.58% | 0.08% | 0.07% |
| C20:1 Total (Gondoic Acid + isomers) | | | 2.10% | 2.08% | 0.14% | 0.14% |
| C20:2 Total (Eicosadien oic Acid) | | | 0.11% | 0.18% | 0.12% | 0.14% |
| C22:0 (Behenic Acid) | | | 0.31% | 0.31% | 0.03% | 0.04% |
| C22:1 Total (Erucic Acid + isomers) | | | 0.02% | | | |
| C24:0 (Lignoceric Acid) | | | 0.15% | 0.16% | | |
| C24:1 Total (Nervonic Acid + isomers) | | | 0.16% | 0.16% | | |

TABLE 3
Conservation of poly unsaturated Fatty
acids (NOTE: MAG refers to the EMO)

| | | | Almond | Canola |
|---|---|---|---|---|
| FATTY ACID | Almond | Canola | MAG | MAG |
| C18:1cis (n-9) Oleic | 63.2 | 59.33 | 63.31 | 59.33 |
| C18:2cis (n-6) Linoleic | 25.28 | 19.07 | 25.14 | 19.07 |
| C18:3 (n-3) Linolenic | 0.29 | 8.26 | 0.2 | 8.22 |
| Oleic/Linoleic ratio | 2.5 | 3.1 | 2.5 | 3.1 |
| Oleic/Linolenic ratio | 218 | 7 | 317 | 7 |

These results demonstrate that the manufacturing process of the present disclosure is able to preserve the fatty acid profile of the starting oil in the EMO. This result is different than for conventional “distilled” oils whose fatty acid profile differs significantly from the starting oil based on the functional use of the distilled oil.

### Example 2: Preparation of Nanoemulsions from a Single Oil Source

Nanoemulsions were prepared from Medium Chain Triglyceride (MCT) enzyme-modified oil as described above in Tables 1-2. Briefly, MCT oil was subjected to complete enzymatic hydrolysis to yield a FFA oil containing free fatty acids and subsequently was further enzymatically processed in the presence of glycerol to re-esterify the FFA to MAGs and DAGs to yield an EMO containing 79% MAGs and DAGs and 21% FFA.

To prepare the first nanoemulsion, 1.5 g of the MCT EMO was weighed into a 50 mL beaker and heated to 60° C. A coarse emulsion was prepared by adding 13.5 mL of water (pre-heated to 60° C.) to the oil. The mixture was subjected to high sheer using a high-sheer homogenizer for 30 seconds, resulting in a milky coarse emulsion. The coarse emulsion was then transferred to a QSonica Q700CA sonicator with a ½ inch probe. The emulsion was processed for 2 cycles (5 minutes sonication/2 minutes cooling) at 90% power.

Additional nanoemulsions were prepared by combining the MCT EMO (including MAGs and DAGs) and the MCT FFA oil (˜93% FFA) yielded after enzymatic hydrolysis to achieve various ratios of MAG and DAG to FFA as shown in Table 4 below. These nanoemulsions were assessed for Z average size, polydispersity index and average zeta potential of the droplets using a Zetasizer Ultra (Malvern Panalytical) according to the manufacturer's instructions. Measurements were made in triplicate.

TABLE 4
Medium Chain Triglyceride (MCT) EMO Nanoemulsions (data
represented as average ± SD from at least three
measurements); MCT is representative of oils with medium
chain fats (C6, C8, C10, C12) and also for saturated fats

| | EMO (non- | | | | |
|---|---|---|---|---|---|
| | FFA com- | | | | Zeta |
| | ponent) | FFA | Z avg | | potential |
| Oil | % | % | (nm) | PDI | (mV) |
| MCT | 79 | 21 | 233 ± 5 | 0.252 ± 0.006 | −31.9 ± 12.5 |
| MCT | 50 | 50 | 191 ± 4 | 0.217 ± 0.007 | −35.5 ± 9.4 |
| MCT | 25 | 75 | 182 ± 5 | 0.213 ± 0.008 | −32.0 ± 0.5 |
| MCT | 10 | 90 | 205 ± 1 | 0.238 ± 0.019 | −34.9 ± 0.3 |
| MCT | 0 | 100 | 257 ± 4 | 0.290 ± 0.022 | −40.7 ± 4.2 |

A corresponding process was performed using olive oil with the resulting data shown in Table 5 below.

TABLE 5
Olive EMO Nanoemulsions (data represented as average ±
SD from at least three measurements); Olive oil is representative
for mono-unsaturated fats (high in oleic acid C18:1)

| | EMO (non- | | | | |
|---|---|---|---|---|---|
| | FFA com- | Total | | | Zeta |
| | ponent) | FFA | Z avg | | potential |
| Oil | % | % | (nm) | PDI | (mV) |
| Olive | 80 | 20 | 229 ± 5 | 0.187 ± 0.035 | −52.8 ± 0.8 |
| Olive | 50 | 50 | 225 ± 4 | 0.224 ± 0.018 | −63.3 ± 1.1 |
| Olive | 25 | 75 | 221 ± 2 | 0.220 ± 0.063 | −60.7 ± 1.1 |
| Olive | 10 | 90 | 237 ± 2 | 0.198 ± 0.019 | −57.7 ± 1.8 |
| Olive | 0 | 100 | 305 ± 6 | 0.169 ± 0.022 | −65.2 ± 1.1 |

A corresponding process was performed using flaxseed oil with the resulting data shown in Table 6 below.

TABLE 6
Flaxseed EMO Nanoemulsions (data represented as average ± SD
from at least three measurements); Flaxseed oil is representative
for poly-unsaturated fats (high in alpha-linoleic, C18:3)

| | EMO (non- | Total | | | Zeta |
|---|---|---|---|---|---|
| | FFA | FFA | Z avg | | potential |
| Oil | component) % | % | (nm) | PDI | (mV) |
| Flaxseed | 82 | 18 | 142 ± 3 | 0.205 ± 0.013 | −49.5 ± 1.0 |
| Flaxseed | 75 | 25 | 141 ± 3 | 0.208 ± 0.012 | −55.8 ± 0.8 |
| Flaxseed | 50 | 50 | 162 ± 5 | 0.179 ± 0.027 | −55.1 ± 3.8 |
| Flaxseed | 25 | 75 | 191 ± 3 | 0.192 ± 0.012 | −75.5 ± 0.9 |
| Flaxseed | 10 | 90 | 273 ± 3 | 0.28 ± 0.043 | −74.7 ± 1.5 |
| Flaxseed | 0 | 100 | 345 ± 12 | 0.232 ± 0.066 | −74.1 ± 1.8 |

A corresponding process was performed using sesame oil with the resulting data shown in Table 7 below.

TABLE 7
Sesame EMO Nanoemulsions (data represented as average ± SD
from at least three measurements); Sesame oil is representative for
unsaturated fats (high in linoleic acid (C18:2) and oleic acid (C18:1))

| | EMO (non- | Total | | | Zeta |
|---|---|---|---|---|---|
| | FFA | FFA | Z avg | | potential |
| Oil | component) % | % | (nm) | PDI | (mV) |
| Sesame | 88 | 12 | 181 ± 4 | 0.186 ± 0.015 | −61.4 ± 2.5 |
| Sesame | 75 | 25 | 180 ± 3 | 0.182 ± 0.023 | −64.0 ± 1.4 |
| Sesame | 50 | 50 | 180 ± 3 | 0.184 ± 0.025 | −72.4 ± 0.6 |
| Sesame | 25 | 75 | 203 ± 4 | 0.174 ± 0.022 | −66.1 ± 1.4 |
| Sesame | 10 | 90 | 250 ± 3 | 0.200 ± 0.027 | −66.2 ± 1.5 |
| Sesame | 0 | 100 | 392 ± 15 | 0.236 ± 0.088 | −70.1 ± 0.2 |

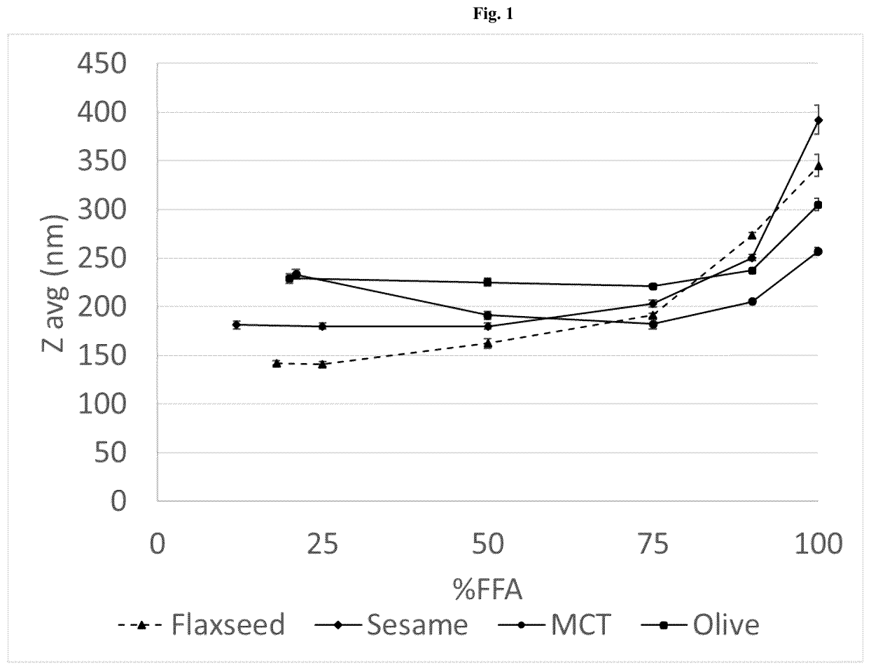

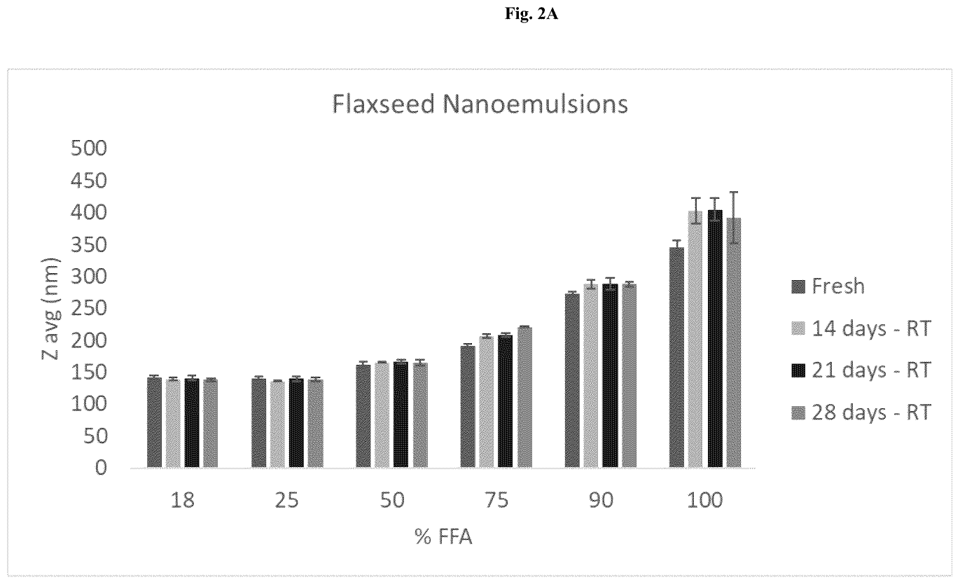

FIG. 1 depicts the Z average size of the droplets in the nanoemulsions produced from the four different starting oils as a function of % FFA.

A corresponding process was performed using rosehip oil with the resulting data shown in Table 8 below.

TABLE 8
Rosehip EMO Nanoemulsions (data represented as average ±
SD from at least three measurements); Rosehip oil is
representative for polyunsaturated fats (high in linoleic acid
(C18:2) and linolenic acid (C18:3))

| | EMO (non- | Total | | | Zeta |
|---|---|---|---|---|---|
| | FFA | FFA | Z avg | | potential |
| Oil | component) % | % | (nm) | PDI | (mV) |
| Rosehip | 75 | 25 | 203 ± 1 | 0.187 ± 0.019 | −53.3 ± 0.4 |
| Rosehip | 50 | 50 | 205 ± 3 | 0.174 ± 0.012 | −57.3 ± 1.7 |
| Rosehip | 25 | 75 | 204 ± 3 | 0.231 ± 0.026 | −54.5 ± 1.1 |
| Rosehip | 0 | 100 | 245 ± 3 | 0.200 ± 0.010 | −59.0 ± 0.3 |

A corresponding process was performed using hemp seed oil with the resulting data shown in Table 9 below.

TABLE 9
Hemp seed EMO Nanoemulsions (data represented as average ± SD
from at least three measurements); Hemp seed oil is representative for
unsaturated fats (high in linoleic acid (C18:2) and linolenic acid (C18:3))

| | EMO (non- | Total | | | Zeta |
|---|---|---|---|---|---|
| | FFA | FFA | Z avg | | potential |
| Oil | component) % | % | (nm) | PDI | (mV) |
| Hemp | 91 | 9 | 183 ± 4 | 0.215 ± 0.026 | −52.7 ± 0.6 |
| Hemp | 75 | 25 | 174 ± 1 | 0.200 ± 0.019 | −57.3 ± 1.1 |
| Hemp | 50 | 50 | 180 ± 2 | 0.199 ± 0.023 | −58.8 ± 1.9 |
| Hemp | 25 | 75 | 181 ± 2 | 0.180 ± 0.041 | −63.0 ± 1.4 |
| Hemp | 0 | 100 | 245 ± 1 | 0.197 ± 0.001 | −61.6 ± 0.4 |

A corresponding process was performed using sunflower oil with the resulting data shown in Table 10 below.

TABLE 10
Sunflower EMO Nanoemulsions (data represented as average ± SD
from at least three measurements); Sunflower oil is representative
for unsaturated fats (high in linoleic acid (C18:2))

| | EMO (non- | Total | | | Zeta |
|---|---|---|---|---|---|
| | FFA | FFA | Z avg | | potential |
| Oil | component) % | % | (nm) | PDI | (mV) |
| Sunflower | 75 | 25 | 194 ± 4 | 0.140 ± 0.016 | −59.4 ± 0.5 |
| Sunflower | 50 | 50 | 211 ± 2 | 0.158 ± 0.017 | −63.0 ± 0.2 |
| Sunflower | 25 | 75 | 228 ± 6 | 0.156 ± 0.023 | −64.4 ± 0.2 |
| Sunflower | 0 | 100 | 320 ± 9 | 0.181 ± 0.032 | −64.8 ± 1.3 |

A corresponding process was performed using oat oil with the resulting data shown in Table 11 below.

TABLE 11
Oat EMO Nanoemulsions (data represented as average ± SD from
at least three measurements); Oat oil is representative for unsaturated
fats (high in linoleic acid (C18:2) and oleic acid (C18:1))

| | EMO (non- | Total | | | Zeta |
|---|---|---|---|---|---|
| | FFA | FFA | Z avg | | potential |
| Oil | component) % | % | (nm) | PDI | (mV) |
| Oat | 87 | 13 | 166 ± 3 | 0.193 ± 0.021 | −61.6 ± 0.4 |
| Oat | 75 | 25 | 177 ± 5 | 0.184 ± 0.010 | −62.8 ± 0.2 |
| Oat | 50 | 50 | 197 ± 6 | 0.144 ± 0.041 | −62.9 ± 1.0 |
| Oat | 25 | 75 | 221 ± 5 | 0.187 ± 0.014 | −63.8 ± 1.3 |
| Oat | 0 | 100 | 361 ± 10 | 0.072 ± 0.051 | −63.7 ± 0.9 |

The foregoing results demonstrate that different ratios of MAGs and DAGs and FFAs can be utilized to prepare nanoemulsions from different starting oils. In some instances, the FFA oil alone can form a nanoemulsion without the addition of the enzyme-modified oil that includes MAGs, and to some extent, DAGs. In all cases the TAG content would be low or non-existent since the TAGs from the starting oil have been substantially or completely hydrolyzed in the first processing step. It should be understood that, where the oil in the nanoemulsion includes EMO, the EMO can be derived from a single starting oil source or from two, three or more starting oil sources. For example, the starting oil sources can be blended prior to enzymatic treatment to obtain the EMO.

### Example 3: Preparation of Nanoemulsions from Different Oil Sources

Nanoemulsions from combinations of EMOs and FFA oils from different oils at different ratios were prepared and analyzed as described in Example 2.

For the combination of coconut EMO and MCT FFA oil, the resulting data are shown in Table 12 below.

TABLE 12
Coconut EMO/MCT FFA Oil Nanoemulsions (data represented as
average ± SD from at least three measurements); This is a
representative blend for medium chain fatty acids and lauric acid

| | | | | | Zeta |
|---|---|---|---|---|---|
| | EMO | FFA | Z avg | | potential |
| EMO/FFA | % | oil % | (nm) | PDI | (mV) |
| Coconut | 100 | 0 | 138 ± 2 | 0.193 ± 0.020 | −38.6 ± 0.5 |
| Coconut/MCT | 90 | 10 | 147 ± 2 | 0.19 ± 0.012 | −30.4 ± 0.3 |
| Coconut/MCT | 75 | 25 | 154 ± 2 | 0.186 ± 0.019 | −30.8 ± 0.8 |
| Coconut/MCT | 50 | 50 | 163 ± 2 | 0.196 ± 0.015 | −31.1 ± 0.5 |
| Coconut/MCT | 25 | 75 | 184 ± 2 | 0.23 ± 0.018 | −32.6 ± 1.3 |
| Coconut/MCT | 10 | 90 | 224 ± 3 | 0.268 ± 0.012 | −37.9 ± 5.6 |
| MCT | 0 | 100 | 255 ± 4 | 0.296 ± 0.053 | −35.3 ± 0.4 |

For the combination of MCT EMO and Coconut FFA oil, the resulting data are shown in Table 13 below.

TABLE 13
MCT EMO/Coconut FFA Oil Nanoemulsions (data
represented as average ± SD from at least three
measurements); This is a representative blend for
medium chain fatty acids and lauric acid (C12)

| | | | | | Zeta |
|---|---|---|---|---|---|
| | EMO | FFA | Z avg | | potential |
| EMO/FFA | % | oil % | (nm) | PDI | (mV) |
| MCT | 100 | 0 | 272 ± 6 | 0.276 ± 0.028 | −30.2 ± 8.0 |
| MCT/Coconut | 90 | 10 | 226 ± 6 | 0.248 ± 0.014 | −37.6 ± 11.6 |
| MCT/Coconut | 75 | 25 | 162 ± 3 | 0.200 ± 0.020 | −32.8 ± 0.4 |
| MCT/Coconut | 50 | 50 | 135 ± 2 | 0.193 ± 0.017 | −37.3 ± 0.9 |
| MCT/Coconut | 25 | 75 | 147 ± 2 | 0.219 ± 0.013 | −41.6 ± 1.4 |
| MCT/Coconut | 10 | 90 | 173 ± 3 | 0.220 ± 0.017 | −46.3 ± 1.0 |
| Coconut | 0 | 100 | 215 ± 5 | 0.233 ± 0.019 | −51.3 ± 0.8 |

For the combination of fish EMO and flaxseed FFA oil, the resulting data are shown in Table 14 below.

TABLE 14
Fish EMO/Flaxseed FFA Oil Nanoemulsions (data represented as average #
SD from at least three measurements); This blend is representative for polyunsaturated
fatty acids, specifically alpha-linolenic acid (C18:3), EPA (C20:5) and DHA (C22:6)

| | | | | | Zeta |
|---|---|---|---|---|---|
| | EMO | FFA | Z avg | | potential |
| EMO/FFA | % | oil % | (nm) | PDI | (mV) |
| Fish/Flaxseed | 75 | 25 | 161 ± 4 | 0.233 ± 0.010 | −54.6 ± 0.3 |
| Fish/Flaxseed | 50 | 50 | 164 ± 3 | 0.199 ± 0.013 | −56.4 ± 1.6 |
| Fish/Flaxseed | 25 | 75 | 172 ± 3 | 0.197 ± 0.021 | −72.0 ± 4.0 |

For the combination of flaxseed EMO and fish FFA oil, the resulting data are shown in Table 15 below.

TABLE 15
Flaxseed EMO/Fish FFA Oil Nanoemulsions (data represented as average ±
SD from at least three measurements); This blend is representative for polyunsaturated
fatty acids, specifically alpha-linolenic acid (C18:3), EPA (C20:5) and DHA (C22:6)

| | | | | | Zeta |
|---|---|---|---|---|---|
| | EMO | FFA | Z avg | | potential |
| EMO/FFA | % | oil % | (nm) | PDI | (mV) |
| Flaxseed/Fish | 75 | 25 | 155 ± 2 | 0.215 ± 0.024 | −56.3 ± 1.2 |
| Flaxseed/Fish | 50 | 50 | 157 ± 2 | 0.213 ± 0.011 | −56.7 ± 1.7 |
| Flaxseed/Fish | 25 | 75 | 162 ± 3 | 0.229 ± 0.013 | −55.1 ± 0.7 |

For the combination of fish EMO and coconut FFA oil, the resulting data are shown in Table 16 below.

TABLE 16
Fish EMO/Coconut FFA Oil Nanoemulsions (data represented as average #
SD from at least three measurements); This blend is representative for polyunsaturated
fatty acids (specifically EPA (C20:5) and DHA (C22:6)) with medium chain fatty acids
(specifically lauric acid (C12))

| | | | | | Zeta |
|---|---|---|---|---|---|
| | EMO | FFA | Z avg | | potential |
| EMO/FFA | % | oil % | (nm) | PDI | (mV) |
| Fish/Coconut | 75 | 25 | 177 ± 4 | 0.237 ± 0.017 | −40.7 ± 0.6 |
| Fish/Coconut | 50 | 50 | 165 ± 4 | 0.230 ± 0.018 | −43.4 ± 0.7 |
| Fish/Coconut | 25 | 75 | 164 ± 3 | 0.232 ± 0.013 | −46.2 ± 1.0 |

For the combination of flaxseed EMO and coconut FFA oil, the resulting data are shown in Table 17 below.

TABLE 17
Flaxseed EMO/Coconut FFA Oil Nanoemulsions (data represented as average ± SD
from at least three measurements); This blend is representative for polyunsaturated
fatty acids (specifically alpha linolenic acid (C18:3)) with medium chain fatty acids
(specifically lauric acid (C12))

| | | | | | Zeta |
|---|---|---|---|---|---|
| | EMO | FFA | Z avg | | potential |
| EMO/FFA | % | oil % | (nm) | PDI | (mV) |
| Flaxseed/coconut | 75 | 25 | 179 ± 2 | 0.227 ± 0.010 | −45.7 ± 1.4 |
| Flaxseed/coconut | 50 | 50 | 182 ± 1 | 0.232 ± 0.017 | −50.1 ± 1.6 |
| Flaxseed/coconut | 25 | 75 | 184 ± 2 | 0.242 ± 0.009 | −48.0 ± 0.2 |

For the combination of coconut EMO and flaxseed FFA oil, the resulting data are shown in Table 18 below.

TABLE 18
Coconut EMO/Flaxseed FFA Oil Nanoemulsions (data represented as average ± SD
from at least three measurements); The blend is representative for polyunsaturated
fatty acids (specifically alpha linolenic acid (C18:3)) with medium chain fatty acids
(specifically lauric acid (C12))

| | | | | | Zeta |
|---|---|---|---|---|---|
| | EMO | FFA | Z avg | | potential |
| EMO/FFA | % | oil % | (nm) | PDI | (mV) |
| Coconut/flaxseed | 75 | 25 | 145 ± 3 | 0.228 ± 0.022 | −45.6 ± 1.9 |
| Coconut/flaxseed | 50 | 50 | 165 ± 2 | 0.167 ± 0.039 | −47.4 ± 1.9 |
| Coconut/flaxseed | 25 | 75 | 188 ± 2 | 0.163 ± 0.006 | −50.7 ± 1.1 |

For the combination of coconut EMO and rosehip FFA oil, the resulting data are shown in Table 19 below.

TABLE 19
Coconut EMO/Rosehip FFA Oil Nanoemulsions (data represented as
average ± SD from at least three measurements); The blend is representative for
polyunsaturated fats (high in linoleic acid (C18:2) and linolenic acid (C18:3)) with
medium chain fatty acids (specifically lauric acid (C12))

| | | | | | Zeta |
|---|---|---|---|---|---|
| | EMO | FFA | Z avg | | potential |
| EMO/FFA | % | oil % | (nm) | PDI | (mV) |
| Coconut/rosehip | 75 | 25 | 153 ± 1 | 0.228 ± 0.024 | −44.3 ± 1.3 |
| Coconut/rosehip | 50 | 50 | 165 ± 4 | 0.208 ± 0.031 | −46.7 ± 0.7 |
| Coconut/rosehip | 25 | 75 | 190 ± 3 | 0.227 ± 0.024 | −52.2 ± 0.9 |

For the combination of rosehip EMO and coconut FFA oil, the resulting data are shown in Table 20 below.

TABLE 20
Rosehip EMO/coconut FFA Oil Nanoemulsions (data represented as
average ± SD from at least three measurements); The blend is representative for
polyunsaturated fats (high in linoleic acid (C18:2) and linolenic acid (C18:3)) with
medium chain fatty acids (specifically lauric acid (C12))

| | | | | | Zeta |
|---|---|---|---|---|---|
| | EMO | FFA | Z avg | | potential |
| EMO/FFA | % | oil % | (nm) | PDI | (mV) |
| Rosehip/coconut | 75 | 25 | 210 ± 3 | 0.260 ± 0.007 | −46.9 ± 0.9 |
| Rosehip/coconut | 50 | 50 | 195 ± 2 | 0.244 ± 0.014 | −46.7 ± 0.8 |
| Rosehip/coconut | 25 | 75 | 191 ± 2 | 0.224 ± 0.011 | −48.0 ± 0.5 |

For the combination of rosehip EMO and flaxseed FFA oil, the resulting data are shown in Table 21 below.

TABLE 21
Rosehip EMO/flaxseed FFA Oil Nanoemulsions (data represented as average ± SD
from at least three measurements); The blend is representative for unsaturated fats

| | | | | | Zeta |
|---|---|---|---|---|---|
| | | FFA | Z avg | | potential |
| EMO/FFA | EMO % | oil % | (nm) | PDI | (mV) |
| Rosehip/flaxseed | 75 | 25 | 189 ± 3 | 0.184 ± 0.032 | −52.2 ± 0.6 |
| Rosehip/flaxseed | 50 | 50 | 193 ± 3 | 0.177 ± 0.007 | −58.4 ± 0.6 |
| Rosehip/flaxseed | 25 | 75 | 205 ± 2 | 0.203 ± 0.025 | −55.6 ± 2.6 |

For the combination of flaxseed EMO and rosehip FFA oil, the resulting data are shown in Table 22 below.

TABLE 22
Flaxseed EMO/rosehip FFA Oil Nanoemulsions (data represented as average ± SD
from at least three measurements); The blend is representative for unsaturated fats

| | | | | | Zeta |
|---|---|---|---|---|---|
| | | FFA | Z avg | | potential |
| EMO/FFA | EMO % | oil % | (nm) | PDI | (mV) |
| Flaxseed/rosehip | 75 | 25 | 170 ± 4 | 0.179 ± 0.040 | −60.4 ± 2.2 |
| Flaxseed/rosehip | 50 | 50 | 186 ± 6 | 0.187 ± 0.010 | −55.0 ± 0.7 |
| Flaxseed/rosehip | 25 | 75 | 197 ± 2 | 0.208 ± 0.017 | −61.7 ± 0.4 |

For the combination of algae EMO and flaxseed FFA oil, the resulting data are shown in Table 23 below.

TABLE 23
Algae EMO/flaxseed FFA Oil Nanoemulsions (data represented as
average ± SD from at least three measurements); The blend
is representative for polyunsaturated fatty acids, specifically
alpha-linolenic acid (C18:3), EPA (C20:5) and DHA (C22:6)

| | | | | | Zeta |
|---|---|---|---|---|---|
| | | FFA | Z avg | | potential |
| EMO/FFA | EMO % | oil % | (nm) | PDI | (mV) |
| Algae/flaxseed | 75 | 25 | 186 ± 4 | 0.197 ± 0.016 | Ppt before |
| | | | | | measuring |
| Algae/flaxseed | 50 | 50 | 187 ± 4 | 0.180 ± 0.029 | −47.0 ± 1.7 |
| Algae/flaxseed | 25 | 75 | 198 ± 1 | 0.187 ± 0.034 | −42.9 ± 1.0 |

For the combination of algae EMO and MCT FFA oil, the resulting data are shown in Table 24 below.

TABLE 24
Algae EMO/MCT FFA Oil Nanoemulsions (data represented as average ±
SD from at least three measurements); The blend is representative for
polyunsaturated fatty acids (specifically EPA (C20:5) and DHA (C22:6))
in combination with medium chain fatty acids.

| | | | | | Zeta |
|---|---|---|---|---|---|
| | | FFA | Z avg | | potential |
| EMO/FFA | EMO % | oil % | (nm) | PDI | (mV) |
| Algae/MCT | 75 | 25 | 452 ± 49 | 0.504 ± 0.445 | −45.8 ± 1.1 |
| Algae/MCT | 50 | 50 | 317 ± 17 | 0.077 ± 0.097 | −40.2 ± 0.6 |
| Algae/MCT | 25 | 75 | 270 ± 5 | 0.244 ± 0.044 | −39.5 ± 1.0 |

For the combination of Oat EMO and flaxseed FFA oil, the resulting data are shown in Table 25 below.

TABLE 25
Oat EMO/Flaxseed FFA Oil Nanoemulsions (data represented
as average ± SD from at least three measurements).

| | | | | | Zeta |
|---|---|---|---|---|---|
| | | FFA | Z avg | | potential |
| EMO/FFA | EMO % | oil % | (nm) | PDI | (mV) |
| Oat/Flaxseed | 75 | 25 | 182 ± 2 | 0.152 ± 0.021 | −62.4 ± 1.3 |
| Oat/Flaxseed | 50 | 50 | 186 ± 5 | 0.179 ± 0.008 | −62.3 ± 1.2 |
| Oat/Flaxseed | 25 | 75 | 195 ± 2 | 0.172 ± 0.035 | −62.6 ± 0.4 |

For the combination of Flaxseed EMO and Oat FFA oil, the resulting data are shown in Table 26 below.

TABLE 26
Flaxseed EMO/Oat FFA Oil Nanoemulsions (data represented
as average ± SD from at least three measurements).

| | | | | | Zeta |
|---|---|---|---|---|---|
| | | FFA | Z avg | | potential |
| EMO/FFA | EMO % | oil % | (nm) | PDI | (mV) |
| Flaxseed/Oat | 75 | 25 | 172 ± 2 | 0.191 ± 0.022 | −62.1 ± 1.3 |
| Flaxseed/Oat | 50 | 50 | 189 ± 1 | 0.181 ± 0.005 | −63.6 ± 0.9 |
| Flaxseed/Oat | 25 | 75 | 215 ± 7 | 0.183 ± 0.018 | −64.1 ± 1.8 |

### Example 4: Stability of Nanoemulsions

Nanoemulsions prepared in Examples 2 and 3 were stored at room temperature or refrigerated (4° C.). At various timepoints, as indicated in the tables below, the samples were re-tested for Z average particle size, polydispersity index and zeta potential (“Fresh” indicates the starting nanoemulsion).

TABLE 27
Stability of Coconut EMO/MCT FFA Oil Nanoemulsions stored at room temperature
(22° C.) (data represented as average ± SD from at least three
measurements; Ppt denotes visible precipitation of the formulation)

| | | | Fresh | | 18 days | | 24 days | | 31 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Coconut | 100 | 0 | 137 ± | 0.193 ± | 243 ± | 0.549 ± | Ppt | Ppt | Ppt | Ppt |
| | | | 2 | 0.020 | 66 | 0.096 | | | | |
| Coconut/MCT | 90 | 10 | 147 ± | 0.190 ± | 147 ± | 0.193 ± | 149 ± | 0.208 ± | 148 ± | 0.185 ± |
| | | | 2 | 0.012 | 4 | 0.018 | 2 | 0.012 | 2 | 0.004 |
| Coconut/MCT | 75 | 25 | 154 ± | 0.186 ± | 159 ± | 0.183 ± | 160 ± | 0.199 ± | 163 ± | 0.178 ± |
| | | | 2 | 0.019 | 5 | 0.013 | 1 | 0.016 | 2 | 0.023 |
| Coconut/MCT | 50 | 50 | 163 ± | 0.196 ± | 173 ± | 0.191 ± | 175 ± | 0.202 ± | 177 ± | 0.189 ± |
| | | | 2 | 0.015 | 4 | 0.018 | 3 | 0.012 | 3 | 0.013 |
| Coconut/MCT | 25 | 75 | 184 ± | 0.230 ± | 202 ± | 0.182 ± | 204 ± | 0.191 ± | 208 ± | 0.214 ± |
| | | | 2 | 0.018 | 4 | 0.027 | 4 | 0.009 | 3 | 0.018 |
| Coconut/MCT | 10 | 90 | 224 ± | 0.268 ± | 245 ± | 0.239 ± | 249 ± | 0.216 ± | 252 ± | 0.200 ± |
| | | | 3 | 0.012 | 5 | 0.023 | 5 | 0.001 | 3 | 0.023 |
| MCT | 0 | 100 | 255 ± | 0.296 ± | 276 ± | 0.235 ± | 278 ± | 0.186 ± | 277 ± | 0.205 ± |
| | | | 4 | 0.053 | 3 | 0.064 | 3 | 0.063 | 2 | 0.013 |

TABLE 28
Stability of Coconut EMO/MCT FFA Oil Nanoemulsions stored at 4°
C. (data represented as average ± SD from at least three measurements;
Ppt denotes visible precipitation of the formulation)

| | | | Fresh | | 18 days | | 24 days | | 31 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Coconut | 100 | 0 | 137 ± | 0.193 ± | 947 ± | 0.964 ± | Ppt | Ppt | Ppt | Ppt |
| | | | 2 | 0.020 | 526 | 0.062 | | | | |
| Coconut/MCT | 90 | 10 | 147 ± | 0.190 ± | 620 ± | 0.932 ± | 149 ± | 0.185 ± | Ppt | Ppt |
| | | | 2 | 0.012 | 132 | 0.118 | 3 | 0.043 | | |
| Coconut/MCT | 75 | 25 | 154 ± | 0.186 ± | 278 ± | 0.560 ± | 182 ± | 0.410 ± | 157 ± | 0.305 ± |
| | | | 2 | 0.019 | 118 | 0.134 | 15 | 0.032 | 6 | 0.074 |
| Coconut/MCT | 50 | 50 | 163 ± | 0.196 ± | 172 ± | 0.197 ± | 173 ± | 0.231 ± | 175 ± | 0.204 ± |
| | | | 2 | 0.015 | 7 | 0.022 | 5 | 0.009 | 2 | 0.009 |
| Coconut/MCT | 25 | 75 | 184 ± | 0.230 ± | 198 ± | 0.192 ± | 197 ± | 0.231 ± | 202 ± | 0.203 ± |
| | | | 2 | 0.018 | 5 | 0.017 | 5 | 0.015 | 2 | 0.032 |
| Coconut/MCT | 10 | 90 | 224 ± | 0.268 ± | 236 ± | 0.209 ± | 242 ± | 0.216 ± | 246 ± | 0.213 ± |
| | | | 3 | 0.012 | 3 | 0.045 | 4 | 0.049 | 7 | 0.031 |
| MCT | 0 | 100 | 255 ± | 0.296 ± | 274 ± | 0.241 ± | 265 ± | 0.166 ± | 267 ± | 0.264 ± |
| | | | 4 | 0.053 | 3 | 0.049 | 4 | 0.033 | 5 | 0.027 |

TABLE 29
Stability of MCT EMO/Coconut FFA Oil Nanoemulsions stored at room
temperature (data represented as average ± SD from at least
three measurements; Ppt denotes visible precipitation of the formulation)

| | | | Fresh | | 16 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| MCT | 100 | 0 | 272 ± | 0.276 ± | 271 ± | 0.256 ± | 274 ± | 0.220 ± | 284 ± | 0.233 ± |
| | | | 6 | 0.028 | 4 | 0.040 | 3 | 0.011 | 3 | 0.014 |
| MCT/Coconut | 90 | 10 | 226 ± | 0.248 ± | 237 ± | 0.228 ± | 236 ± | 0.195 ± | 238 ± | 0.195 ± |
| | | | 6 | 0.014 | 2 | 0.014 | 3 | 0.019 | 6 | 0.007 |
| MCT/Coconut | 75 | 25 | 162 ± | 0.200 ± | 175 ± | 0.168 ± | 177 ± | 0.168 ± | 177 ± | 0.200 ± |
| | | | 3 | 0.020 | 3 | 0.027 | 2 | 0.013 | 3 | 0.026 |
| MCT/Coconut | 50 | 50 | 135 ± | 0.193 ± | 150 ± | 0.201 ± | 151 ± | 0.180 ± | 151 ± | 0.190 ± |
| | | | 2 | 0.017 | 2 | 0.004 | 2 | 0.014 | 2 | 0.027 |
| MCT/Coconut | 25 | 75 | 147 ± | 0.219 ± | 162 ± | 0.212 ± | Ppt | Ppt | Ppt | Ppt |
| | | | 2 | 0.013 | 3 | 0.011 | | | | |
| MCT/Coconut | 10 | 90 | 173 ± | 0.220 ± | 174 ± | 0.238 ± | Ppt | Ppt | Ppt | Ppt |
| | | | 3 | 0.017 | 4 | 0.020 | | | | |
| Coconut | 0 | 100 | 214 ± | 0.233 ± | 205 ± | 0.217 ± | Ppt | Ppt | Ppt | Ppt |
| | | | 5 | 0.019 | 4 | 0.008 | | | | |

TABLE 30
Stability of MCT EMO/Coconut FFA Oil Nanoemulsions stored at 4°
C. (data represented as average ± SD from at least three measurements;
Ppt denotes visible precipitation of the formulation)

| | | | Fresh | | 16 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| MCT | 100 | 0 | 272 ± | 0.276 ± | 251 ± | 0.230 ± | 250 ± | 0.210 ± | 248 ± | 0.196 ± |
| | | | 6 | 0.028 | 7 | 0.009 | 3 | 0.018 | 2 | 0.011 |
| MCT/Coconut | 90 | 10 | 226 ± | 0.248 ± | 229 ± | 0.229 ± | 232 ± | 0.232 ± | 227 ± | 0.222 ± |
| | | | 6 | 0.014 | 7 | 0.033 | 3 | 0.016 | 5 | 0.013 |
| MCT/Coconut | 75 | 25 | 162 ± | 0.200 ± | 170 ± | 0.189 ± | 169 ± | 0.189 ± | 172 ± | 0.204 ± |
| | | | 3 | 0.020 | 2 | 0.025 | 4 | 0.018 | 1 | 0.026 |
| MCT/Coconut | 50 | 50 | 135 ± | 0.193 ± | 131 ± | 0.267 ± | Ppt | Ppt | Ppt | Ppt |
| | | | 2 | 0.017 | 1 | 0.025 | | | | |
| MCT/Coconut | 25 | 75 | 147 ± | 0.219 ± | 139 ± | 0.329 ± | Ppt | Ppt | Ppt | Ppt |
| | | | 2 | 0.013 | 16 | 0.083 | | | | |
| MCT/Coconut | 10 | 90 | 173 ± | 0.220 ± | 141 ± | 0.245 ± | Ppt | Ppt | Ppt | Ppt |
| | | | 3 | 0.017 | 1 | 0.010 | | | | |
| Coconut | 0 | 100 | 214 ± | 0.233 ± | 177 ± | 0.225 ± | Ppt | Ppt | Ppt | Ppt |
| | | | 5 | 0.019 | 3 | 0.026 | | | | |

TABLE 31
Stability of Flaxseed and Sesame EMO Nanoemulsions stored at room
temperature (data represented as average ± SD from at least
three measurements; Ppt denotes visible precipitation of the formulation)

| | EMO | | Fresh | | 14 days | | 21 days | | 28 days | |
|---|---|---|---|---|---|---|---|---|---|---|
| | (non-FFA | Total | Z | | Z | | Z | | Z | |
| | component) | FFA | avg | | avg | | avg | | avg | |
| Oil | % | % | (nm) | PDI | (nm) | PDI | (nm) | PDI | (nm) | PDI |
| Flaxseed | 82 | 18 | 142 ± | 0.205± | 139± | 0.208 ± | 141 ± | 0.212 ± | 138 ± | 0.207 ± |
| | | | 3 | 0.013 | 2 | 0.013 | 4 | 0.012 | 3 | 0.007 |
| Flaxseed | 75 | 25 | 141 ± | 0.208 ± | 136 ± | 0.192 ± | 141 ± | 0.206 ± | 139 ± | 0.190 ± |
| | | | 3 | 0.012 | 1 | 0.009 | 4 | 0.028 | 3 | 0.021 |
| Flaxseed | 50 | 50 | 162 ± | 0.179 ± | 166 ± | 0.196 ± | 166 ± | 0.210 ± | 165 ± | 0.180 ± |
| | | | 5 | 0.027 | 1 | 0.014 | 3 | 0.036 | 4 | 0.018 |
| Flaxseed | 25 | 75 | 191 ± | 0.192 ± | 206 ± | 0.222 ± | 208 ± | 0.209 ± | 221 ± | 0.220 ± |
| | | | 3 | 0.012 | 3 | 0.014 | 3 | 0.032 | 1 | 0.037 |
| Flaxseed | 10 | 90 | 273 ± | 0.281 ± | 288 ± | 0.211 ± | 289 ± | 0.160 ± | 287 ± | 0.167 ± |
| | | | 2 | 0.043 | 7 | 0.064 | 9 | 0.025 | 4 | 0.051 |
| Flaxseed | 0 | 100 | 345 ± | 0.232 ± | 403 ± | 0.227 ± | 405 ± | 0.142 ± | 392 ± | 0.488 ± |
| | | | 12 | 0.066 | 20 | 0.066 | 18 | 0.030 | 40 | 0.290 |
| Sesame | 88 | 12 | 181 ± | 0.186 ± | 183 ± | 0.179 ± | 179 ± | 0.195 ± | Ppt | Ppt |
| | | | 4 | 0.015 | 2 | 0.012 | 4 | 0.015 | | |
| Sesame | 75 | 25 | 180 ± | 0.182 ± | 186 ± | 0.193 ± | 180 ± | 0.167 ± | 187 ± | 0.178 ± |
| | | | 3 | 0.023 | 4 | 0.017 | 3 | 0.012 | 4 | 0.004 |
| Sesame | 50 | 50 | 180 ± | 0.184 ± | 184 ± | 0.187 ± | 188 ± | 0.211 ± | 188 ± | 0.195 ± |
| | | | 3 | 0.025 | 5 | 0.007 | 3 | 0.018 | 3 | 0.018 |
| Sesame | 25 | 75 | 203 ± | 0.174 ± | 211 ± | 0.177 ± | 210 ± | 0.169 ± | 216 ± | 0.209 ± |
| | | | 4 | 0.022 | 2 | 0.010 | 6 | 0.023 | 4 | 0.031 |
| Sesame | 10 | 90 | 248 ± | 0.200 ± | 400 ± | 0.111 ± | 447 ± | 0.092 ± | Ppt | Ppt |
| | | | 3 | 0.027 | 9 | 0.130 | 5 | 0.092 | | |
| Sesame | 0 | 100 | 392 ± | 0.236 ± | 444 ± | 0.595 ± | Ppt | Ppt | Ppt | Ppt |
| | | | 15 | 0.088 | 16 | 0.315 | | | | |

TABLE 32
Stability of Flaxseed and Sesame EMO Nanoemulsions stored at 4°
C. (data represented as average ± SD from at least three measurements;
Ppt denotes visible precipitation of the formulation)

| | EMO | | Fresh | | 14 days | | 21 days | | 28 days | |
|---|---|---|---|---|---|---|---|---|---|---|
| | (non-FFA | Total | Z | | Z | | Z | | Z | |
| | component) | FFA | avg | | avg | | avg | | avg | |
| Oil | % | % | (nm) | PDI | (nm) | PDI | (nm) | PDI | (nm) | PDI |
| Flaxseed | 82 | 18 | 142 ± | 0.205 ± | 141 ± | 0.234 ± | 170 ± | 0.204 ± | Ppt | Ppt |
| | | | 3 | 0.013 | 3 | 0.009 | 1 | 0.013 | | |
| Flaxseed | 75 | 25 | 141 ± | 0.208 ± | 142 ± | 0.205 ± | 144 ± | 0.210 ± | 141 ± | 0.206 ± |
| | | | 3 | 0.012 | 4 | 0.010 | 1 | 0.006 | 1 | 0.022 |
| Flaxseed | 50 | 50 | 162 ± | 0.179 ± | 165 ± | 0.208 ± | 171 ± | 0.223 ± | Ppt | Ppt |
| | | | 5 | 0.027 | 1 | 0.017 | 3 | 0.019 | | |
| Flaxseed | 25 | 75 | 191 ± | 0.192 ± | 200 ± | 0.216 ± | Ppt | Ppt | Ppt | Ppt |
| | | | 3 | 0.012 | 1 | 0.032 | | | | |
| Flaxseed | 10 | 90 | 273 ± | 0.281 ± | 258 ± | 0.246 ± | Ppt | Ppt | Ppt | Ppt |
| | | | 2 | 0.043 | 1 | 0.020 | | | | |
| Flaxseed | 0 | 100 | 345 ± | 0.232 ± | 284 ± | 0.185 ± | Ppt | Ppt | Ppt | Ppt |
| | | | 12 | 0.066 | 11 | 0.075 | | | | |
| Sesame | 88 | 12 | 181 ± | 0.186 ± | Ppt | Ppt | Ppt | Ppt | Ppt | Ppt |
| | | | 4 | 0.015 | | | | | | |
| Sesame | 75 | 25 | 180 ± | 0.182 ± | Ppt | Ppt | Ppt | Ppt | Ppt | Ppt |
| | | | 3 | 0.023 | | | | | | |
| Sesame | 50 | 50 | 180 ± | 0.184 ± | Ppt | Ppt | Ppt | Ppt | Ppt | Ppt |
| | | | 3 | 0.025 | | | | | | |
| Sesame | 25 | 75 | 203 ± | 0.174 ± | Ppt | Ppt | Ppt | Ppt | Ppt | Ppt |
| | | | 4 | 0.022 | | | | | | |
| Sesame | 10 | 90 | 248 ± | 0.200 ± | Ppt | Ppt | Ppt | Ppt | Ppt | Ppt |
| | | | 3 | 0.027 | | | | | | |
| Sesame | 0 | 100 | 392 ± | 0.236 ± | Ppt | Ppt | Ppt | Ppt | Ppt | Ppt |
| | | | 15 | 0.088 | | | | | | |

TABLE 33
Stability of Rosehip and Hemp seed EMO Nanoemulsions stored at room
temperature (data represented as average ± SD from at least three
measurements; Ppt denotes visible precipitation of the formulation)

| | EMO | | Fresh | | 14 days | | 21 days | | 28 days | |
|---|---|---|---|---|---|---|---|---|---|---|
| | (non-FFA | Total | Z | | Z | | Z | | Z | |
| | component) | FFA | avg | | avg | | avg | | avg | |
| Oil | % | % | (nm) | PDI | (nm) | PDI | (nm) | PDI | (nm) | PDI |
| Rosehip | 75 | 25 | 203 ± | 0.187 ± | 211 ± | 0.163 ± | 210 ± | 0.209 ± | 215 ± | 0.205 ± |
| | | | 1 | 0.019 | 8 | 0.021 | 4 | 0.015 | 3 | 0.016 |
| Rosehip | 50 | 50 | 205 ± | 0.174 ± | 212 ± | 0.186 ± | 207 ± | 0.191 ± | 210 ± | 0.203 ± |
| | | | 3 | 0.012 | 7 | 0.021 | 1 | 0.038 | 2 | 0.032 |
| Rosehip | 25 | 75 | 204 ± | 0.231 ± | 216 ± | 0.217 ± | 212 ± | 0.216 ± | 215 ± | 0.218 ± |
| | | | 3 | 0.026 | 2 | 0.023 | 2 | 0.032 | 5 | 0.013 |
| Rosehip | 0 | 100 | 245 ± | 0.200 ± | 312 ± | 0.142 ± | 339 ± | 0.234 ± | 327 ± | 0.162 ± |
| | | | 3 | 0.010 | 26 | 0.024 | 3 | 0.051 | 4 | 0.082 |
| Hemp seed | 91 | 9 | 183 ± | 0.215 ± | 191 ± | 0.242 ± | 197 ± | 0.249 ± | 201 ± | 0.256 ± |
| | | | 4 | 0.026 | 3 | 0.011 | 3 | 0.016 | 3 | 0.009 |
| Hemp seed | 75 | 25 | 174 ± | 0.200 ± | 183 ± | 0.201 ± | 185 ± | 0.226 ± | 190 ± | 0.233 ± |
| | | | 1 | 0.019 | 3 | 0.022 | 4 | 0.005 | 3 | 0.023 |
| Hemp seed | 50 | 50 | 180 ± | 0.199 ± | 183 ± | 0.205 ± | 187 ± | 0.192 ± | 185 ± | 0.182 ± |
| | | | 2 | 0.023 | 1 | 0.024 | 3 | 0.025 | 2 | 0.016 |
| Hemp seed | 25 | 75 | 181 ± | 0.180 ± | 186 ± | 0.207 ± | 191 ± | 0.181 ± | 192 ± | 0.194 ± |
| | | | 2 | 0.041 | 1 | 0.018 | 4 | 0.036 | 1 | 0.016 |
| Hemp seed | 0 | 100 | 245 ± | 0.197 ± | 327 ± | 0.110 ± | 315 ± | 0.143 ± | 313 ± | 0.100 ± |
| | | | 1 | 0.001 | 7 | 0.086 | 4 | 0.093 | 9 | 0.089 |

TABLE 34
Stability of MCT EMO Nanoemulsions stored at room temperature
(data represented as average ± SD from at least three measurements;
Ppt denotes visible precipitation of the formulation)

| | EMO | | Fresh | | 14 days | | 21 days | | 28 days | |
|---|---|---|---|---|---|---|---|---|---|---|
| | (non-FFA | Total | Z | | Z | | Z | | Z | |
| | component) | FFA | avg | | avg | | avg | | avg | |
| Oil | % | % | (nm) | PDI | (nm) | PDI | (nm) | PDI | (nm) | PDI |
| MCT | 79 | 21 | 233 ± | 0.252 ± | 243 ± | 0.231 ± | 254 ± | 0.232 ± | 255 ± | 0.208 ± |
| | | | 5 | 0.006 | 3 | 0.005 | 5 | 0.026 | 9 | 0.010 |
| MCT | 50 | 50 | 191 ± | 0.217 ± | 207 ± | 0.176 ± | 209 ± | 0.165 ± | 215 ± | 0.156 ± |
| | | | 4 | 0.007 | 4 | 0.051 | 1 | 0.049 | 3 | 0.052 |
| MCT | 25 | 75 | 182 ± | 0.213 ± | 201 ± | 0.182 ± | 201 ± | 0.288 ± | 211 ± | 0.143 ± |
| | | | 5 | 0.008 | 6 | 0.028 | 11 | 0.081 | 6 | 0.010 |
| MCT | 10 | 90 | 205 ± | 0.238 ± | 221 ± | 0.219 ± | 233 ± | 0.186 ± | 232 ± | 0.201 ± |
| | | | 1 | 0.019 | 2 | 0.027 | 4 | 0.025 | 2 | 0.023 |
| MCT | 0 | 100 | 257 ± | 0.290 ± | 258 ± | 0.224 ± | 272 ± | 0.198 ± | 269 ± | 0.181 ± |
| | | | 4 | 0.022 | 4 | 0.043 | 7 | 0.042 | 3 | 0.029 |

TABLE 35
Stability of MCT EMO Nanoemulsions stored at 4° C. (data represented as average ±
SD from at least three measurements; Ppt denotes visible precipitation of the formulation)

| | EMO | | Fresh | | 14 days | | 21 days | | 28 days | |
|---|---|---|---|---|---|---|---|---|---|---|
| | (non-FFA | Total | Z | | Z | | Z | | Z | |
| | component) | FFA | avg | | avg | | avg | | avg | |
| Oil | % | % | (nm) | PDI | (nm) | PDI | (nm) | PDI | (nm) | PDI |
| MCT | 79 | 21 | 233 ± | 0.252 ± | 228 ± | 0.240 ± | 224 ± | 0.241 ± | 236 ± | 0.327 ± |
| | | | 5 | 0.006 | 2 | 0.017 | 2 | 0.006 | 4 | 0.038 |
| MCT | 50 | 50 | 191 ± | 0.217 ± | 199 ± | 0.174 ± | 200 ± | 0.187 ± | 204 ± | 0.199 ± |
| | | | 4 | 0.007 | 2 | 0.045 | 1 | 0.021 | 1 | 0.033 |
| MCT | 25 | 75 | 182 ± | 0.213 ± | 194 ± | 0.188 ± | 196 ± | 0.174 ± | 200 ± | 0.149 ± |
| | | | 5 | 0.008 | 2 | 0.009 | 3 | 0.017 | 1 | 0.049 |
| MCT | 10 | 90 | 205 ± | 0.238 ± | 214 ± | 0.239 ± | 197 ± | 0.215 ± | 226 ± | 0.189 ± |
| | | | 1 | 0.019 | 7 | 0.014 | 2 | 0.024 | 3 | 0.010 |
| MCT | 0 | 100 | 257 ± | 0.290 ± | 260 ± | 0.242 ± | 257 ± | 0.220 ± | 276 ± | 0.219 ± |
| | | | 4 | 0.022 | 3 | 0.037 | 4 | 0.038 | 1 | 0.005 |

TABLE 36
Stability of Nanoemulsions from Flaxseed EMO and FFAs of fish, coconut, and rosehip
stored at room temperature (data represented as average ± SD from at least
three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Flaxseed/Fish | 75 | 25 | 155 ± | 0.215 ± | 154 ± | 0.207 ± | 157 ± | 0.207 ± | 149 ± | 0.218 ± |
| | | | 2 | 0.024 | 1 | 0.013 | 3 | 0.019 | 3 | 0.028 |
| Flaxseed/Fish | 50 | 50 | 157 ± | 0.214 ± | 154 ± | 0.195 ± | Ppt | Ppt | Ppt | Ppt |
| | | | 2 | 0.011 | 1 | 0.020 | | | | |
| Flaxseed/Fish | 25 | 75 | 162 ± | 0.229 ± | 165 ± | 0.235 ± | Ppt | Ppt | Ppt | Ppt |
| | | | 3 | 0.013 | 3 | 0.024 | | | | |
| Flaxseed/Coconut | 75 | 25 | 179 ± | 0.227 ± | 180 ± | 0.227 ± | 182 ± | 0.217 ± | 186 ± | 0.222 ± |
| | | | 2 | 0.010 | 3 | 0.005 | 1 | 0.016 | 3 | 0.006 |
| Flaxseed/Coconut | 50 | 50 | 182 ± | 0.232 ± | 187 ± | 0.232 ± | 187 ± | 0.231 ± | 189 ± | 0.227 ± |
| | | | 1 | 0.017 | 6 | 0.010 | 4 | 0.018 | 3 | 0.011 |
| Flaxseed/Coconut | 25 | 75 | 184 ± | 0.242 ± | Ppt | Ppt | Ppt | Ppt | Ppt | Ppt |
| | | | 2 | 0.009 | | | | | | |
| Flaxseed/rosehip | 75 | 25 | 170 ± | 0.179 ± | 171 ± | 0.155 ± | 168 ± | 0.175 ± | 178 ± | 0.173 ± |
| | | | 4 | 0.040 | 7 | 0.021 | 2 | 0.020 | 5 | 0.027 |
| Flaxseed/rosehip | 50 | 50 | 186 ± | 0.187 ± | 185 ± | 0.173 ± | 183 ± | 0.172 ± | 185 ± | 0.221 ± |
| | | | 6 | 0.010 | 4 | 0.005 | 3 | 0.032 | 7 | 0.035 |
| Flaxseed/rosehip | 25 | 75 | 197 ± | 0.208 ± | 213 ± | 0.197 ± | 222 ± | 0.240 ± | 223 ± | 0.274 ± |
| | | | 2 | 0.017 | 1 | 0.011 | 3 | 0.021 | 4 | 0.074 |

TABLE 37
Stability of Nanoemulsions from Flaxseed EMO and FFAs of fish, coconut, and
rosehip stored at 4° C. (data represented as average ± SD from
at least three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Flaxseed/Fish | 75 | 25 | 155 ± | 0.215 ± | 163 ± | 0.292 ± | Ppt | Ppt | Ppt | Ppt |
| | | | 2 | 0.024 | 7 | 0.065 | | | | |
| Flaxseed/Fish | 50 | 50 | 157 ± | 0.214 ± | 176 ± | 0.386 ± | Ppt | Ppt | Ppt | Ppt |
| | | | 2 | 0.011 | 11 | 0.030 | | | | |
| Flaxseed/Fish | 25 | 75 | 162 ± | 0.229 ± | 243 ± | 0.493 ± | Ppt | Ppt | Ppt | Ppt |
| | | | 3 | 0.013 | 22 | 0.059 | | | | |
| Flaxseed/Coconut | 75 | 25 | 179 ± | 0.227 ± | Ppt | Ppt | Ppt | Ppt | Ppt | Ppt |
| | | | 2 | 0.010 | | | | | | |
| Flaxseed/Coconut | 50 | 50 | 182 ± | 0.232 ± | Ppt | Ppt | Ppt | Ppt | Ppt | Ppt |
| | | | 1 | 0.017 | | | | | | |
| Flaxseed/Coconut | 25 | 75 | 184 ± | 0.242 ± | Ppt | Ppt | Ppt | Ppt | Ppt | Ppt |
| | | | 2 | 0.009 | | | | | | |
| Flaxseed/rosehip | 75 | 25 | 170 ± | 0.179 ± | Ppt | Ppt | Ppt | Ppt | Ppt | Ppt |
| | | | 4 | 0.040 | | | | | | |
| Flaxseed/rosehip | 50 | 50 | 186 ± | 0.187 ± | Ppt | Ppt | Ppt | Ppt | Ppt | Ppt |
| | | | 6 | 0.010 | | | | | | |
| Flaxseed/rosehip | 25 | 75 | 197 ± | 0.208 ± | Ppt | Ppt | Ppt | Ppt | Ppt | Ppt |
| | | | 2 | 0.017 | | | | | | |

TABLE 38
Stability of Nanoemulsions from Fish EMO and FFAs of flaxseed and coconut
stored at room temperature (data represented as average ± SD from at
least three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Fish/Flaxseed | 75 | 25 | 161 ± | 0.233 ± | 153 ± | 0.238 ± | Ppt | Ppt | Ppt | Ppt |
| | | | 4 | 0.010 | 4 | 0.006 | | | | |
| Fish/Flaxseed | 50 | 50 | 164 ± | 0.199 ± | 160 ± | 0.189 ± | Ppt | Ppt | Ppt | Ppt |
| | | | 3 | 0.013 | 3 | 0.014 | | | | |
| Fish/Flaxseed | 25 | 75 | 172 ± | 0.197 ± | 171 ± | 0.210 ± | 170 ± | 0.209 ± | 141 ± | 0.206 ± |
| | | | 3 | 0.021 | 1 | 0.007 | 2 | 0.015 | 1 | 0.022 |
| Fish/Coconut | 75 | 25 | 177 ± | 0.237 ± | Ppt | Ppt | Ppt | Ppt | Ppt | Ppt |
| | | | 4 | 0.017 | | | | | | |
| Fish/Coconut | 50 | 50 | 165 ± | 0.230 ± | 168 ± | 0.228 ± | 170 ± | 0.209 ± | 171 ± | 0.210 ± |
| | | | 4 | 0.018 | 3 | 0.006 | 6 | 0.016 | 2 | 0.013 |
| Fish/Coconut | 25 | 75 | 164 ± | 0.232 ± | Ppt | Ppt | Ppt | Ppt | Ppt | Ppt |
| | | | 3 | 0.013 | | | | | | |

TABLE 39
Stability of Nanoemulsions from Fish EMO and FFAs of flaxseed and coconut
stored at 4° C. (data represented as average ± SD from at least
three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Fish/Flaxseed | 75 | 25 | 161 ± | 0.233 ± | 158 ± | 0.263 ± | Ppt | Ppt | Ppt | Ppt |
| | | | 4 | 0.010 | 1 | 0.023 | | | | |
| Fish/Flaxseed | 50 | 50 | 164 ± | 0.199 ± | 165 ± | 0.258 ± | Ppt | Ppt | Ppt | Ppt |
| | | | 3 | 0.013 | 3 | 0.046 | | | | |
| Fish/Flaxseed | 25 | 75 | 172 ± | 0.197 ± | 169 ± | 0.202 ± | Ppt | Ppt | Ppt | Ppt |
| | | | 3 | 0.021 | 3 | 0.029 | | | | |
| Fish/Coconut | 75 | 25 | 177 ± | 0.237 ± | Ppt | Ppt | Ppt | Ppt | Ppt | Ppt |
| | | | 4 | 0.017 | | | | | | |
| Fish/Coconut | 50 | 50 | 165 ± | 0.230 ± | Ppt | Ppt | Ppt | Ppt | Ppt | Ppt |
| | | | 4 | 0.018 | | | | | | |
| Fish/Coconut | 25 | 75 | 164 ± | 0.232 ± | Ppt | Ppt | Ppt | Ppt | Ppt | Ppt |
| | | | 3 | 0.013 | | | | | | |

TABLE 40
Stability of Nanoemulsions from Coconut EMO and FFAs of flaxseed and rosehip
stored at room temperature (data represented as average ± SD from at
least three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Coconut/Flaxseed | 75 | 25 | 145 ± | 0.228 ± | 158 ± | 0.228 ± | 158 ± | 0.227 ± | 165 ± | 0.214 ± |
| | | | 3 | 0.022 | 1 | 0.017 | 4 | 0.012 | 4 | 0.012 |
| Coconut/Flaxseed | 50 | 50 | 165 ± | 0.167 ± | 188 ± | 0.232 ± | 194 ± | 0.221 ± | 197 ± | 0.249 ± |
| | | | 2 | 0.039 | 4 | 0.016 | 3 | 0.009 | 6 | 0.005 |
| Coconut/Flaxseed | 25 | 75 | 188 ± | 0.163 ± | 203 ± | 0.208 ± | 212 ± | 0.191 ± | 229 ± | 0.233 ± |
| | | | 2 | 0.006 | 3 | 0.023 | 4 | 0.035 | 1 | 0.018 |
| Coconut/rosehip | 75 | 25 | 153 ± | 0.228 ± | 165 ± | 0.225 ± | 164 ± | 0.236 ± | 171 ± | 0.213 ± |
| | | | 1 | 0.024 | 4 | 0.017 | 2 | 0.015 | 1 | 0.012 |
| Coconut/rosehip | 50 | 50 | 165 ± | 0.208 ± | 181 ± | 0.214 ± | 186 ± | 0.250 ± | 195 ± | 0.257 ± |
| | | | 4 | 0.031 | 2 | 0.004 | 3 | 0.014 | 1 | 0.008 |
| Coconut/rosehip | 25 | 75 | 190 ± | 0.227 ± | 210 ± | 0.189 ± | 211 ± | 0.195 ± | 218 ± | 0.222 ± |
| | | | 3 | 0.024 | 6 | 0.039 | 1 | 0.018 | 3 | 0.019 |

TABLE 41
Stability of Nanoemulsions from Coconut EMO and FFAs of flaxseed and rosehip
stored at 4° C. (data represented as average ± SD from at least
three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Coconut/Flaxseed | 75 | 25 | 145 ± | 0.228 ± | Ppt | Ppt | Ppt | Ppt | Ppt | Ppt |
| | | | 3 | 0.022 | | | | | | |
| Coconut/Flaxseed | 50 | 50 | 165 ± | 0.167 ± | 184 ± | 0.289 ± | Ppt | Ppt | Ppt | Ppt |
| | | | 2 | 0.039 | 1 | 0.027 | | | | |
| Coconut/Flaxseed | 25 | 75 | 188 ± | 0.163 ± | Ppt | Ppt | Ppt | Ppt | Ppt | Ppt |
| | | | 2 | 0.006 | | | | | | |
| Coconut/rosehip | 75 | 25 | 153 ± | 0.228 ± | Ppt | Ppt | Ppt | Ppt | Ppt | Ppt |
| | | | 1 | 0.024 | | | | | | |
| Coconut/rosehip | 50 | 50 | 165 ± | 0.208 ± | Ppt | Ppt | Ppt | Ppt | Ppt | Ppt |
| | | | 4 | 0.031 | | | | | | |
| Coconut/rosehip | 25 | 75 | 190 ± | 0.227 ± | Ppt | Ppt | Ppt | Ppt | Ppt | Ppt |
| | | | 3 | 0.024 | | | | | | |

TABLE 42
Stability of Nanoemulsions from Rosehip EMO and coconut FFA stored at
room temperature (data represented as average ± SD from at least
three measurements; Ppt denotes visible precipitation of the formulation)

| | | | Fresh | | 14 days | | 21 days | | 39 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Rosehip/coconut | 75 | 25 | 210 ± | 0.260 ± | 220 ± | 0.248 ± | 225 ± | 0.257 ± | 227 ± | 0.260 ± |
| | | | 3 | 0.007 | 5 | 0.028 | 3 | 0.011 | 3 | 0.041 |
| Rosehip/coconut | 50 | 50 | 195 ± | 0.244 ± | 194 ± | 0.288 ± | 195 ± | 0.254 ± | 199 ± | 0.250 ± |
| | | | 2 | 0.014 | 9 | 0.033 | 2 | 0.021 | 3 | 0.007 |
| Rosehip/coconut | 25 | 75 | 191 ± | 0.224 ± | Ppt | Ppt | Ppt | Ppt | Ppt | Ppt |
| | | | 2 | 0.011 | | | | | | |

TABLE 43
Stability of Nanoemulsions from Rosehip EMO and flaxseed FFA stored at room temperature (data represented
as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Rosehip/ | 75 | 25 | 189 ± 3 | 0.184 ± 0.032 | 200 ± 3 | 0.205 ± 0.004 | 200 ± 4 | 0.221 ± 0.014 | 208 ± 3 | 0.253 ± 0.007 |
| flaxseed | | | | | | | | | | |
| Rosehip/ | 50 | 50 | 193 ± 3 | 0.17 ± 0.007 | 199 ± 5 | 0.205 ± 0.035 | 233 ± 2 | 0.260 ± 0.026 | 220 ± 7 | 0.203 ± 0.027 |
| flaxseed | | | | | | | | | | |
| Rosehip/ | 25 | 75 | 205 ± 2 | 0.203 ± 0.025 | 237 ± 3 | 0.186 ± 0.053 | 287 ± 3 | 0.311 ± 0.052 | 276 ± 10 | 0.229 ± 0.087 |
| flaxseed | | | | | | | | | | |

TABLE 44
Stability of Nanoemulsions from Algae EMO and FFAs of flaxseed and MCT stored at room temperature (data represented
as average ± SD from at least three measurements; Ppt denotes visible precipitation of the formulation)

| | | | Fresh | | 14 days | | 21 days | | 28 days | |
|---|---|---|---|---|---|---|---|---|---|---|
| | | FFA | Z | | Z | | Z | | Z | |
| | EMO | oil | avg | | avg | | avg | | avg | |
| EMO/FFA | % | % | (nm) | PDI | (nm) | PDI | (nm) | PDI | (nm) | PDI |
| Algae/ | 75 | 25 | 186 ± 4 | 0.197 ± 0.016 | Ppt | Ppt | Ppt | Ppt | Ppt | Ppt |
| flaxseed | | | | | | | | | | |
| Algae/ | 50 | 50 | 187 ± 4 | 0.180 ± 0.029 | Ppt | Ppt | Ppt | Ppt | Ppt | Ppt |
| flaxseed | | | | | | | | | | |
| Algae/ | 25 | 75 | 198 ± 1 | 0.187 ± 0.034 | 214 ± 2 | 0.199 ± 0.004 | 214 ± 1 | 0.195 ± 0.025 | 216 ± 3 | 0.163 ± 0.012 |
| flaxseed | | | | | | | | | | |
| Algae/MCT | 75 | 25 | 452 ± 49 | 0.504 ± 0.445 | Ppt | Ppt | Ppt | Ppt | Ppt | Ppt |
| Algae/MCT | 50 | 50 | 317 ± 17 | 0.077 ± 0.097 | 324 ± 7 | 0.135 ± 0.109 | 318 ± 13 | 0.316 ± 0.165 | Ppt | Ppt |
| Algae/MCT | 25 | 75 | 270 ± 5 | 0.244 ± 0.044 | 289 ± 4 | 0.282 ± 0.014 | 304 ± 9 | 0.160 ± 0.121 | 259 ± 6 | 0.213 ± 0.059 |

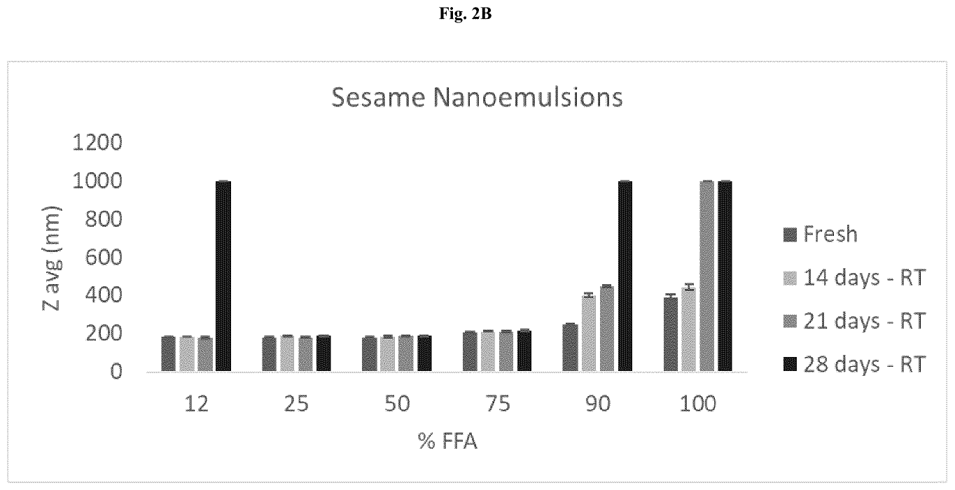

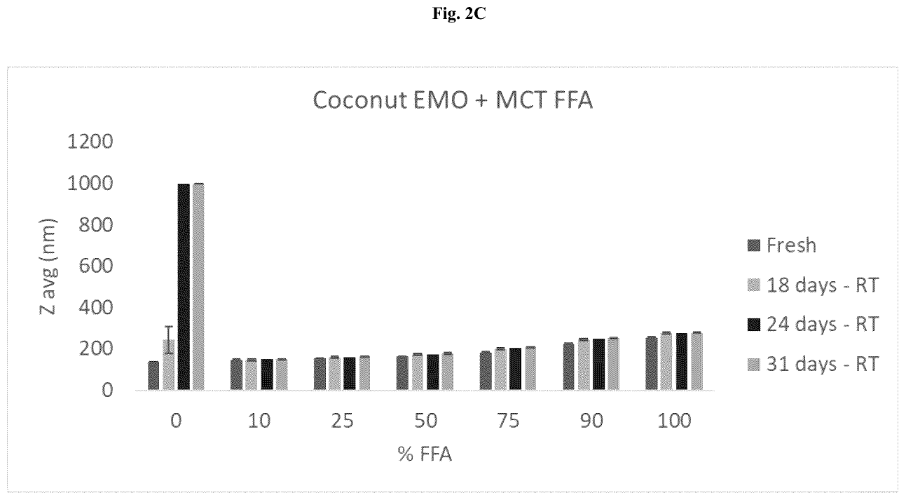

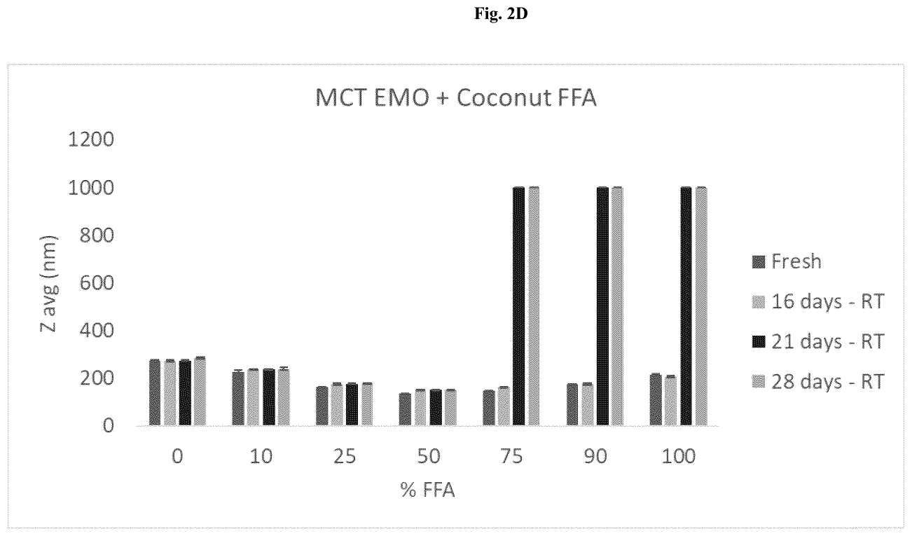

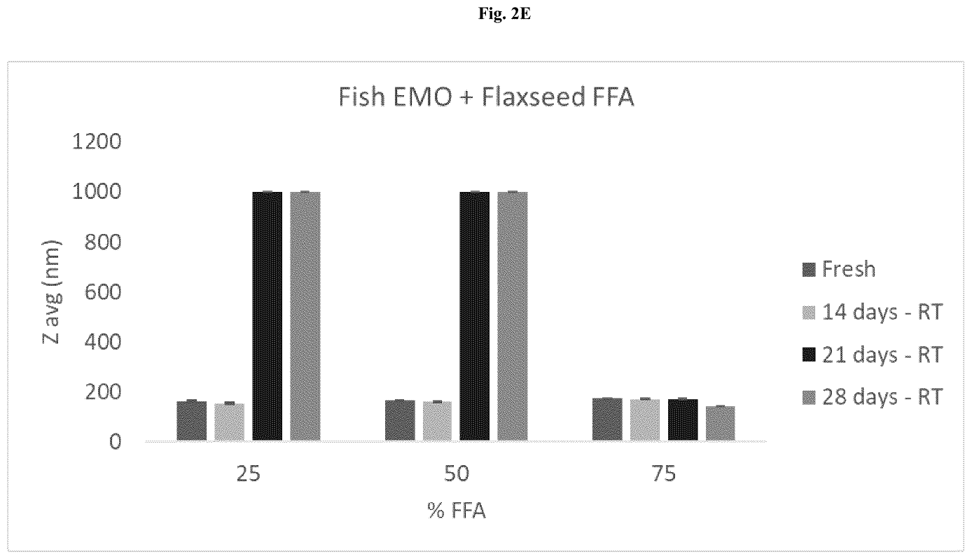

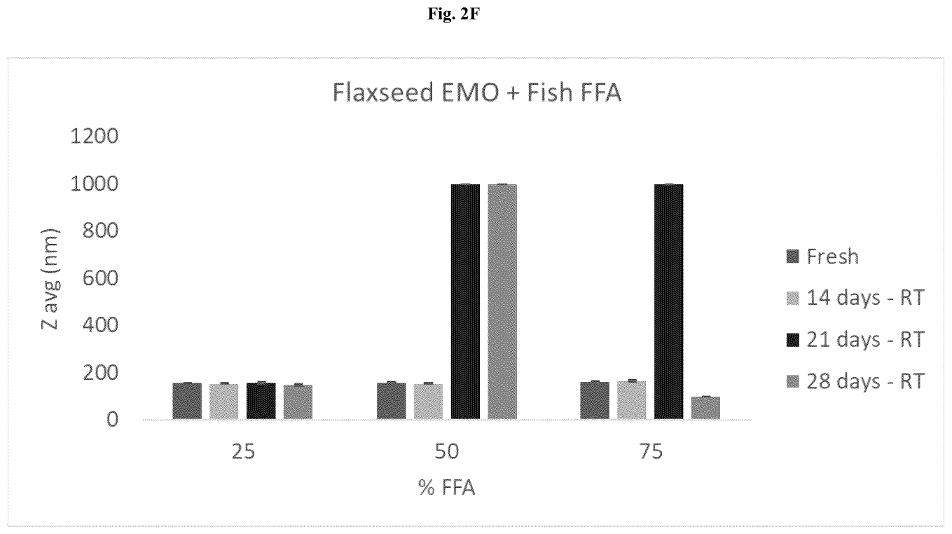

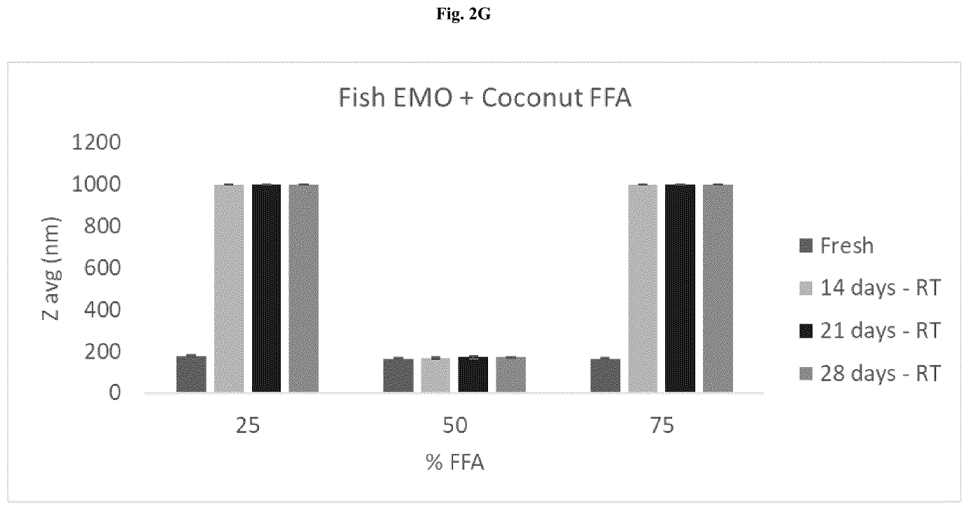

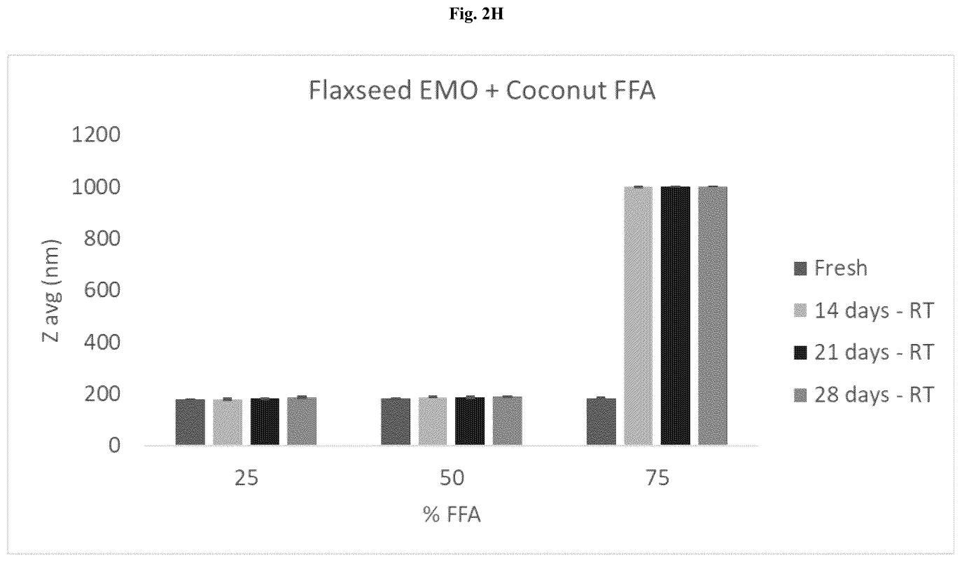

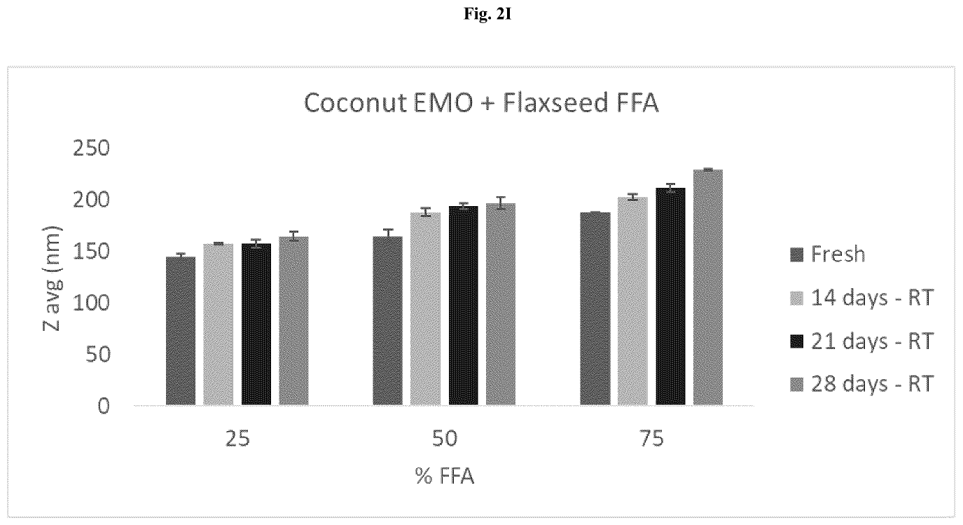

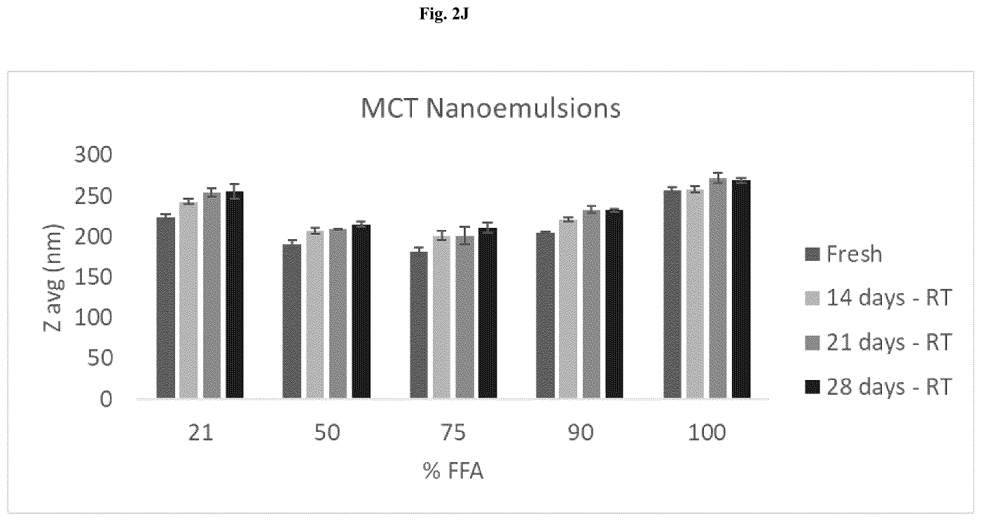

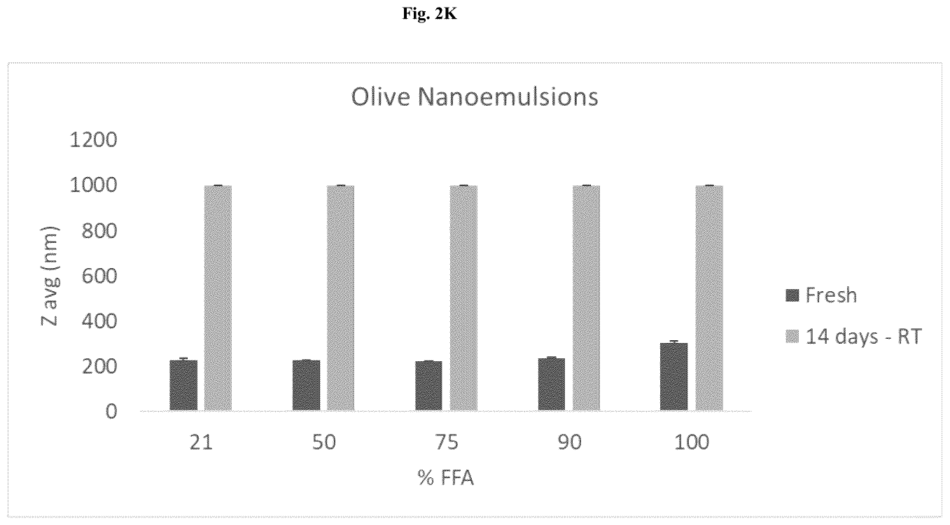

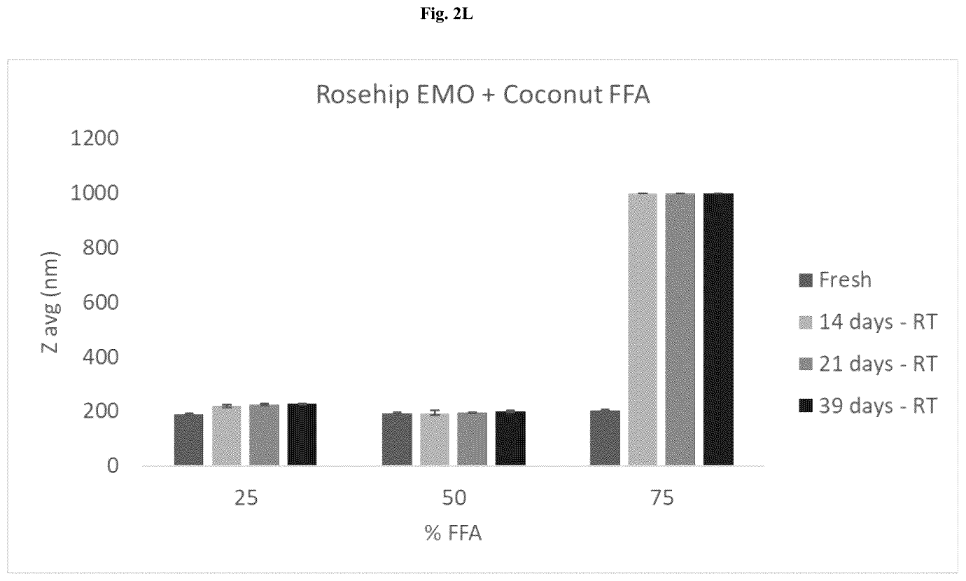

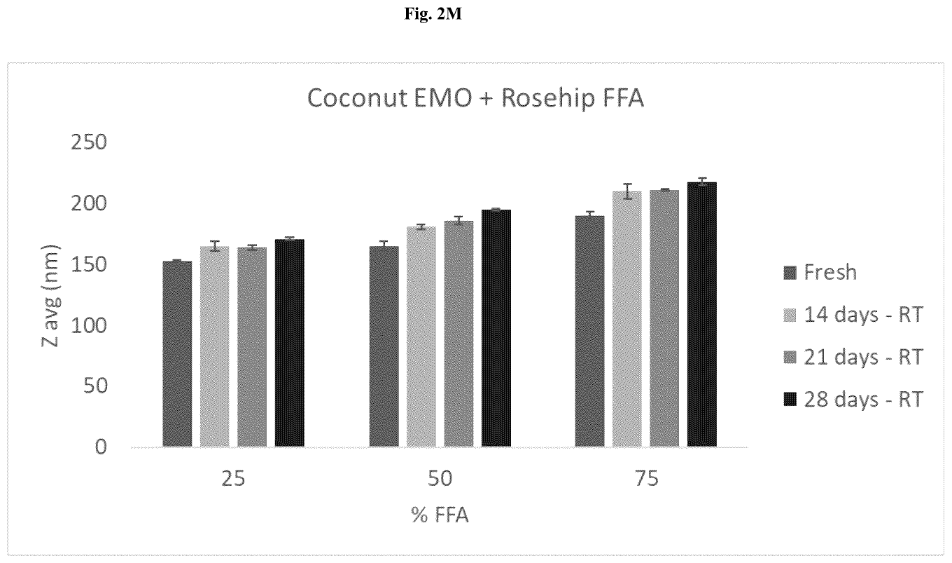

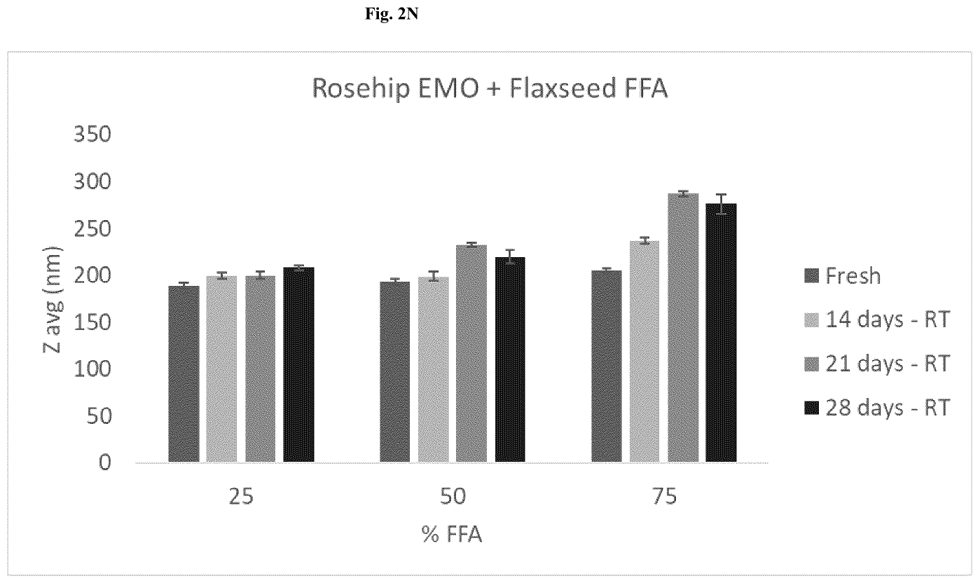

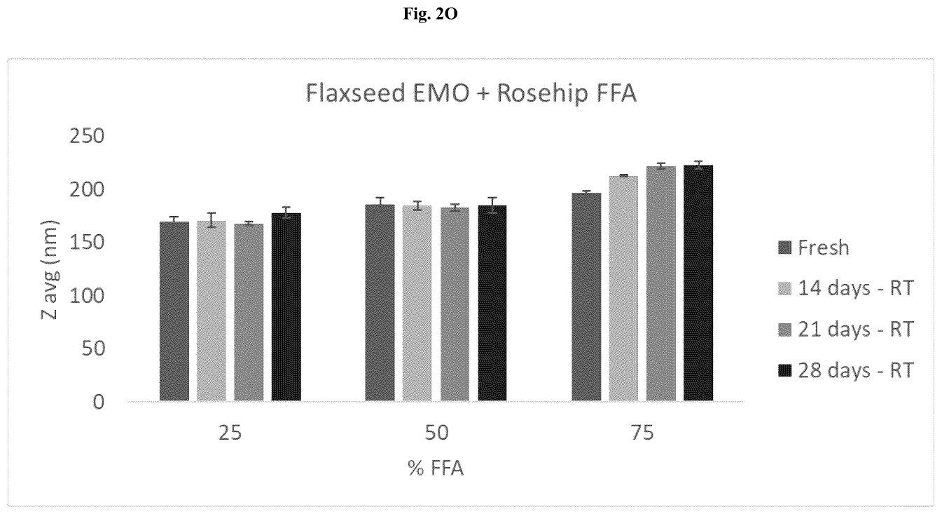

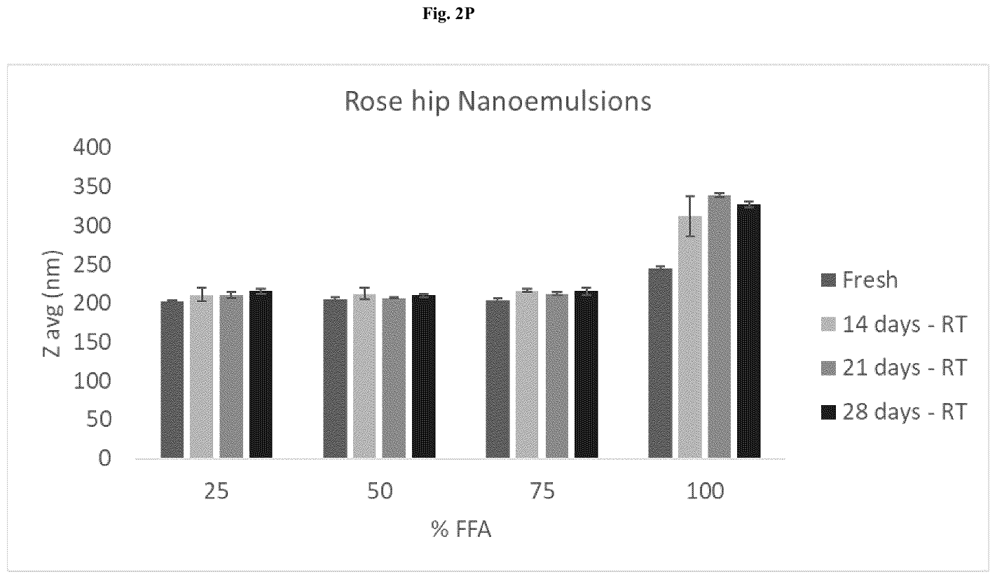

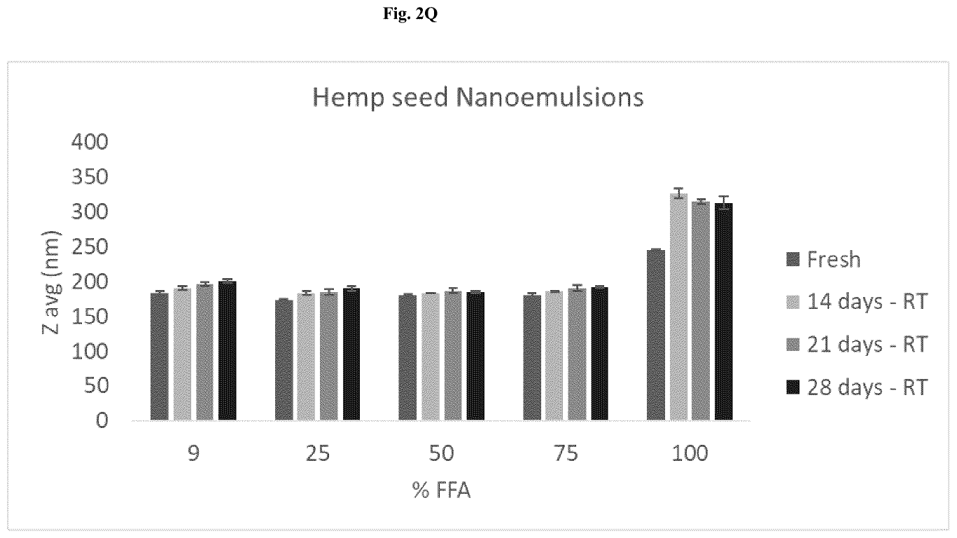

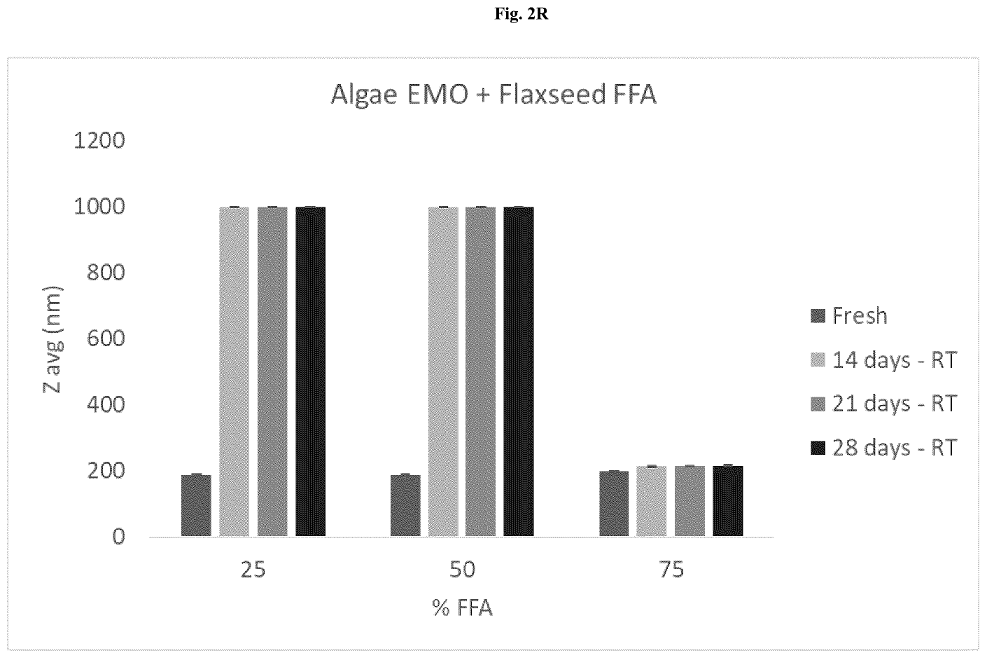

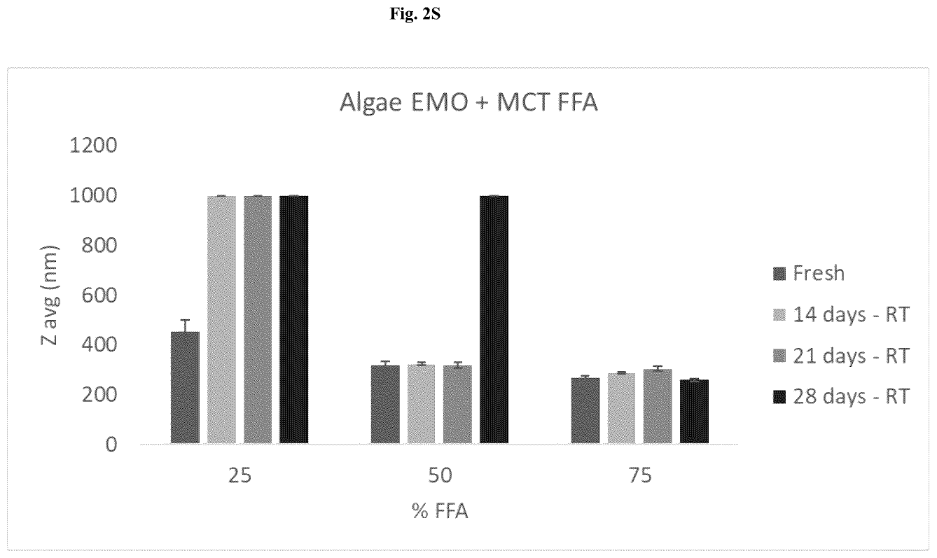

FIGS. 2A-2S show the Z average particle size for the nanoemulsions in Tables 27-44.

### Example 5: Preparation of Nanoemulsions from Unmodified Oils

In order to assess the effect of TAG content on making nanoemulsions of the present disclosure, olive oil (unmodified, TAG form) was mixed with olive FFA oil prepared as described above at different ratios, and the mixtures were prepared as nanoemulsions according to the procedure described above. Z average particle size and polydispersity index were measured for these samples and the results are provided below in Table 45 below.

TABLE 45
Particle size analysis of olive oil and olive FFA oil
nanoemulsions by dynamic light scattering (data represented
as average ± SD from at least three measurements)

| Oil % | FFA oil % | Z avg (nm) | PDI |
|---|---|---|---|
| 100 | 0 | 2 layers | |
| 75 | 25 | 399 ± 46 | 0.349 ± 0.388 |
| 70 | 30 | 349 ± 29 | 0.211 ± 0.203 |
| 60 | 40 | 343 ± 10 | 0.101 ± 0.088 |
| 50 | 50 | 248 ± 5 | 0.199 ± 0.021 |
| 25 | 75 | 219 ± 3 | 0.189 ± 0.030 |

As shown in Table 45, olive oil alone, which is essentially TAGs, was unable to form a nanoemulsion as it resulted in the formation of two separate layers. In addition, the size of the droplets decreased with increasing FFA content and decreasing TAG content. Specifically, up to 50% olive oil (essentially TAGs) still yielded nanoemulsions with a Z average size of less than 300 nm, while 60% olive oil yielded larger particles and the 100% TAG sample quickly separated into 2 layers.

Olive oil (unmodified, TAG form) was also blended with olive EMO, which includes MAGs, some DAGs and FFAs, prepared as described above at various ratios and nanoemulsions prepared therefrom as described above. Z average particle size and polydispersity index were measured for these samples and the results are provided in Table 46 below.

TABLE 46
Particle size analysis of olive oil and olive EMO
nanoemulsions by dynamic light scattering (data represented
as average ± SD from at least three measurements)

| Oil % | EMO % | FFA oil % | Z avg (nm) | PDI |
|---|---|---|---|---|
| 90 | 8 | 2 | 496 ± 47 | 0.520 ± 0.443 |
| 80 | 16 | 4 | 293 ± 13 | 0.130 ± 0.091 |
| 75 | 20 | 5 | 243 ± 5 | 0.228 ± 0.010 |
| 50 | 40 | 10 | 178 ± 3 | 0.215 ± 0.009 |
| 25 | 60 | 15 | 183 ± 4 | 0.201 ± 0.018 |

As shown in Table 46, up to 80% TAG was still able to yield Z average particle sizes below 300 nm.

The same procedure was performed using olive oil (unmodified, TAG form) blended with either oleic acid or glyceryl oleate at ratios of 75/25, 50/50 and 25/75. In no cases did the blend form a nanoemulsion, rather, 2 layers were obtained.

### Example 6: Preparation of Nanoemulsions Using Off-the-Shelf Ingredients

To assess nanoemulsions prepared from off-the-shelf ingredients, nanoemulsions were prepared from glyceryl oleate (MakingCosmetics, part #EMF-GLYOL-01) and/or oleic acid (Beantown Chemical, part #126125) at various ratios. Z average size and polydispersity index were measured as described above. The results are shown in Table 47 below.

TABLE 47
Particle size analysis of glyceryl oleate/oleic acid
nanoemulsions by dynamic light scattering (data represented
as average ± SD from at least three measurements)

| Glyceryl oleate % | Oleic acid % | Z avg (nm) | PDI |
|---|---|---|---|
| 100 | 0 | 629 ± 42 | 0.227 ± 0.047 |
| 75 | 25 | 627 ± 42 | 0.250 ± 0.021 |
| 50 | 50 | 330 ± 10* | 0.323 ± 0.030* |
| 25 | 75 | ** | ** |
| 0 | 100 | 572 ± 100 | 0.132 ± 0.119 |

*Analysis from liquid layer
** Forms a cream

These results can be compared to those in Table 5 for olive EMO nanoemulsions. In contrast, the glyceryl oleate/oleic acid nanoemulsions did not form nanoemulsions of less than 300 nm Z average droplet size. Both the 50/50 and 25/75 blends yielded creams and the other samples yielded large droplet sizes of about 600 nm or larger. Thus, as performed in this Example, off-the-shelf ingredients did not replicate the results with EMO.

### Example 7: Nanoemulsions from Multiple Oil Sources

Nanoemulsions were formed from EMOs and FFAs of rosehip, flaxseed and MCT oils as described in the previous examples. The size, PDI and zeta potential data are shown in Table 48 below.

TABLE 48
Nanoemulsions from EMOs and FFAs of rosehip, flaxseed, and
MCT oils (data represented as average ± SD from at
least three measurements); This is a representative blend
for saturated and unsaturated fats from three different sources

| | | | | | Zeta |
|---|---|---|---|---|---|
| | | FFA | Z avg | | potential |
| Oils | EMO % | oil % | (nm) | PDI | (mV) |
| Rosehip | 100 | 0 | 193 ± 2 | 0.228 ± 0.025 | −29.2 ± 0.4 |
| Flaxseed | | | | | |
| MCT | | | | | |
| Rosehip | 50 | 50 | 213 ± 5 | 0.288 ± 0.050 | −42.3 ± 0.4 |
| Flaxseed | | | | | |
| MCT | | | | | |

Nanoemulsions containing three different EMOs or three different EMOs plus their corresponding FFA oil at 50/50 ratios achieved particle sizes less than 300 nm and PDI less than 0.300.

### Example 8: Amount of Oil in Nanoemulsion

Nanoemulsions were prepared from rosehip EMO at various % oil out of the total weight of the nanoemulsion. Table 49 below provides the size and PDI data.

TABLE 49
Nanoemulsions from rosehip EMO at various % oil compositions (data
represented as average ± SD from at least three measurements)

| | EMO (% wt | | |
|---|---|---|---|
| | of the total | | |
| EMO | composition) | Z avg (nm) | PDI |
| Rosehip | 1 | 178 ± 4 | 0.215 ± 0.018 |
| Rosehip | 5 | 180 ± 3 | 0.180 ± 0.004 |
| Rosehip | 10 | 199 ± 1 | 0.144 ± 0.014 |
| Rosehip | 20 | 255 ± 3 | 0.239 ± 0.005 |
| Rosehip | 30 | 270 ± 5 | 0.277 ± 0.018 |
| Rosehip | 50 | Formed a cream | |
| Rosehip | 70 | Formed a cream | |

Rosehip EMO nanoemulsions could be formed when the percent weight of the oil was 1% to 30% of the total weight of the formulation. At 50% oil weight or greater, a cream was formed.

### Example 9: Minimum Inhibitory Concentration (MIC) Assays

MIC measurements reflect the drug concentration that inhibits cellular growth (i.e., bacteriostatic). The MIC is the lowest concentration of an antimicrobial agent that prevents the visible growth of bacteria. We are measuring not by visible growth but by OD600 on a plate reader, which is more sensitive. So we are defining MIC as the lowest concentration that does not allow an exponential growth phase during the time course of 16 hours. The drug does not necessarily kill the microorganism, but rather restricts further growth. In the case of infection this is often sufficient, allowing elements of the immune system to kill any bacteria that persist. Beta-lactam antibiotics (e.g., penicillin) are bacteriostatic.

Nanoemulsions were prepared as previously described and as listed in Table 50 below as 10% (w/w) oil in water nanoemulsions (this same ratio was used in all subsequent Examples). It should be understood that as referred to herein, such stocks of MAG refer to EMO that contain MAG and that FFA refers to FFA oil, such that MAG/FFA refers to a combination of EMO/FFA oil. Stocks of “MAG” solution and “FFA” solution were kept in a 4° C. refrigerator (at which temperature the oils solidify), in amber bottles topped with nitrogen to prevent exposure to light and oxygen. Prior to use, aliquots of oil solutions were placed in a 60° C. water bath to melt, and kept at 60° C. To a 100 mL stainless-steel cup, 13.5 g of water was added and warmed in the 60° C. water bath. 1.5 g of total oil was added to the water (for example, 0.75 g MAG, 0.75 FFA for 50/50 MAG/FFA ratio) and immediately homogenized using a Brinkmann 110V homogenizer for 1 minute with manual rotation to ensure evenness. The samples were then sonicated using a QSonica at 90% amplitude with 5 min On, 1 min Off, three times for a total of 15 min On. Aliquots of each 5 min are diluted 1:1000 in water and analyzed on a Malvern Zetasizer for Z-avg, PDI, and zeta potential. These properties are provided in the preceding tables. Analysis was performed with three consecutive reading replicates conducted. Nanoemulsions were aliquoted and stored both at room temperature in the dark, and in a 4° C. refrigerator in the dark.

TABLE 50
List of nanoemulsions used for bacterial studies.
MAG = EMO; FFA = FFA oil.

| Number | MAG | FFA | Ratio |
|---|---|---|---|
| 1 | Almond | Almond | 50/50 |
| 2 | Coconut | Coconut | 50/50 |
| 3 | Coconut | Coconut | 25/75 |
| 4 | Fish | Fish | 50/50 |
| 5 | Flaxseed | Flaxseed | 75/25 |
| 6 | Flaxseed | Flaxseed | 50/50 |
| 7 | Flaxseed | Flaxseed | 25/75 |
| 8 | Hemp | Hemp | 50/50 |
| 9 | MCT | MCT | 50/50 |
| 10 | Oat | Oat | 50/50 |
| 11 | Rosehip | Rosehip | 50/50 |
| 12 | Sesame | Sesame | 50/50 |
| 13 | Algae | MCT | 50/50 |
| 14 | Algae | MCT | 25/75 |
| 15 | Coconut | Fish | 50/50 |
| 16 | Coconut | Flaxseed | 50/50 |
| 17 | Coconut | Oat | 50/50 |
| 18 | Coconut | MCT | 50/50 |
| 19 | Coconut | Rosehip | 50/50 |
| 20 | Fish | Almond | 50/50 |
| 21 | Fish | Coconut | 50/50 |
| 22 | Fish | Flaxseed | 50/50 |
| 23 | Flaxseed | Coconut | 50/50 |
| 24 | Flaxseed | Fish | 75/25 |
| 25 | Flaxseed | Fish | 50/50 |
| 26 | Flaxseed | Oat | 50/50 |
| 27 | Flaxseed | Fish | 25/75 |
| 28 | Flaxseed | Rosehip | 50/50 |
| 29 | MCT | Coconut | 50/50 |
| 30 | Oat | Flaxseed | 25/75 |
| 31 | Rosehip | Coconut | 50/50 |
| 32 | Rosehip | Flaxseed | 50/50 |
| 33 | Algae | Flaxseed | 25/75 |

Abbreviations for each source oil as follows: Algae: Ag; Almond: Al; Coconut: Co; Fish: F; Flaxseed: Fx; Hempseed: H; MCT: M; Oat: Oa; Olive: O; Rosehip: R; Sesame: S

Each compound used in the following examples is named using the following nomenclature scheme. For each compound, the name is n(MAG)/(FFA)-MAG %, with the FFA term omitted if same as MAG (MAG as used in this instance refers to the EMO). For example: a nanoemulsion of 50% Flaxseed EMO and 50% Flaxseed FFA oil would be nFx-50; a nanoemulsion of 25% Coconut EMO and 75% Rosehip FFA oil would be nCo/R-25.

The list of bacteria used in this assay are provided in Table 51 below.

TABLE 51
List of bacteria used in this study.

| Species | Strain |
|---|---|
| Acinetobacter baumannii | ATCC 19606 |
| Bacillus cereus | ATCC 11778 |
| Corynebacterium xerosis** | ATCC 373 |
| Cutibacterium acnes† | ATCC 6919 |
| Enterococcus faecalis | ATCC 29212 |
| Escherichia coli | ATCC 25922 |
| Helicobacter pylori† | ATCC 43504 |
| Klebsiella pneumoniae | ATCC 13883 |
| Listeria monocytogenes | ATCC 19115 |
| Micrococcus luteus** | ATCC 4698 |
| Pseudomonas aeruginosa | ATCC 27853 |
| Staphylococcus aureus | ATCC 25923 |
| Staphylococcus epidermidis | ATCC 12228 |
| Staphylococcus capitis** | ATCC 35661 |
| Streptococcus agalactiae | ATCC 13813 |
| Streptococcus mutans | ATCC 35668 |
| Streptococcus pyogenes | ATCC 12344 |

**Bacteria that were used for non-inhibitory experiments
†Required anaerobic or microaerophilic growth conditions not amenable to standard plate-reader growth experiments

C. acnes (ATCC 6919), S. epidermidis (ATCC 12228), S. aureus (ATCC 25923), L. monocytogenes (ATCC 19115), B. cereus (ATCC 11778), S. agalactiae (ATCC 13813), S. pyogenes (ATCC 12344), E. faecalis (ATCC 29212), S. mutans (ATCC 35668), E. coli (ATCC 25922), P. aeruginosa (ATCC 27853), A. baumannii (ATCC 19606), K. pneumoniae (ATCC 13883), P. gingivalis (ATCC 33277), and H. pylori (ATCC 43504) were cultured according to the ATCC recommendations.

C. acnes and P. gingivalis were cultured under anaerobic conditions and H. pylori was cultured under microaerophilic conditions using Gas-Pak (BD) pouches.

Single colonies were used to inoculate the recommended broth and shaken at 200 rpm at 37° C. until reaching an OD₆₀₀ of near 1.0 as measured by a uv spectrophotometer blanked with the corresponding broth. For non-fastidious bacteria, 25% glycerol stocks were made by a 1:1 dilution of bacterial culture to a previously autoclaved solution of 50% glycerol in water, and frozen and stored in a −80° C. freezer.

Fastidious bacteria were kept on sealed agar plates at 4° C. and periodically re-streaked on fresh plates.

Note: Nanoemulsions were diluted as needed for experiments, since solutions that were diluted more than 8-fold did not exhibit the same long-term stability as the original nanoemulsion solution.

Concentrations of 1.25 mg/mL, 0.625 mg/mL, 0.31 mg/mL, and 0.156 mg/mL of each nanoemulsion were prepared in DI water, serving as 12.5×stocks (final concentrations of 100 μg/mL, 50 μg/mL, 25 μg/mL, and 12.5 μg/mL respectively). To a 96-well non-tissue culture treated clear flat bottom polystyrene plate, 16μL of each solution was pipetted per well, in duplicate. 16 μL of DI water was added to the non-treated control wells. In a laminar flow hood, 25μL of a bacterial glycerol stock (frozen 1:1 with 50% glycerol at an initial OD₆₀₀ of ˜1) was added to 25 mL of either Mueller-Hinton broth, Tryptic Soy broth, Reinforced Clostridial broth, or Brain-Heart Infusion broth. Aerobic bacterial solutions were vortexed and shaken at 200 rpm for 5 minutes, anaerobic or microaerophilic bacterial solutions were placed in GasPak pouches and shaken at 200 rpm for 5 minutes to mix. The bacterial solution was then poured into a 100 mL multichannel reservoir and 184 μL was immediately pipetted into each well of the nanoemulsion-containing 96-well plate; to give a 12.5× dilution of each nanoemulsion.

A Biotek Cytation 5 plate reader was used for overnight OD₆₀₀ scans of aerobic bacteria. The plate reader was preheated to 37° C., and a method was made to shake the plate orbitally at 200 rpm and 37° C. with the lid on, and measure absorbance at 600 nm every 30 min for 16 hr. For anaerobic and microaerophilic bacteria, the 96-well plates were shaken at 120 rpm inside GasPak pouches taped to the bottom of a shaking incubator at 37° C., quickly removed at determined time points for OD₆₀₀ scans on the plate reader, and immediately replaced in the Gaspak pouch.

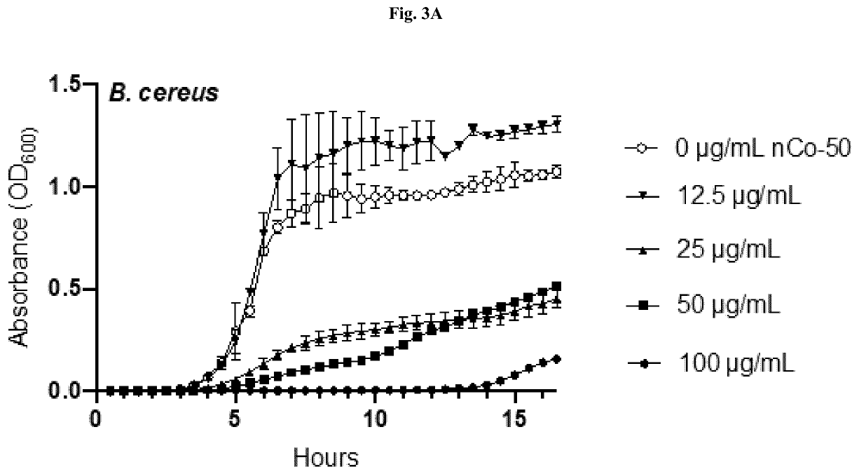

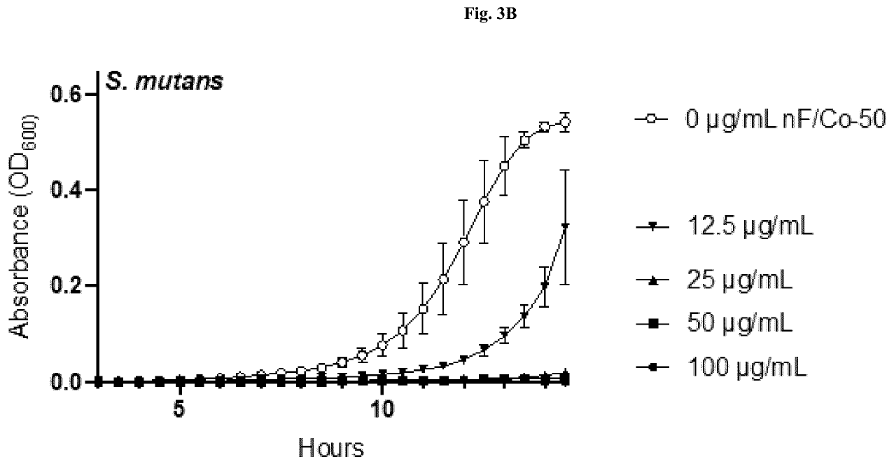

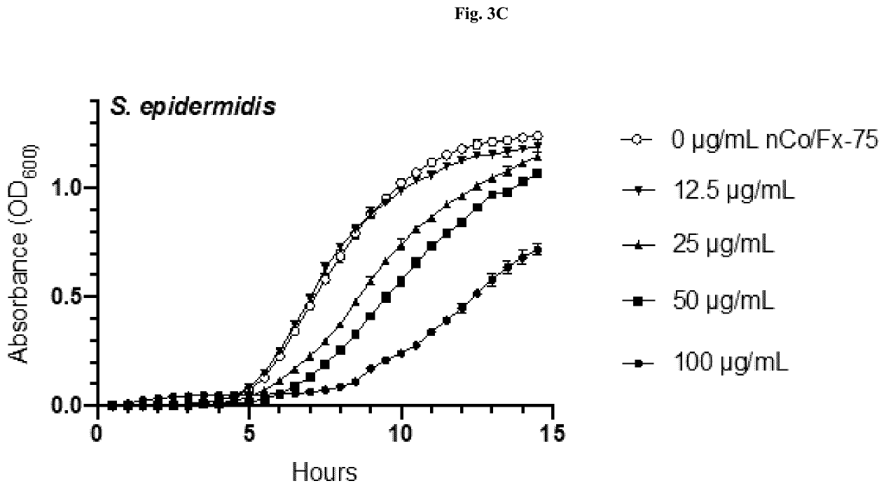

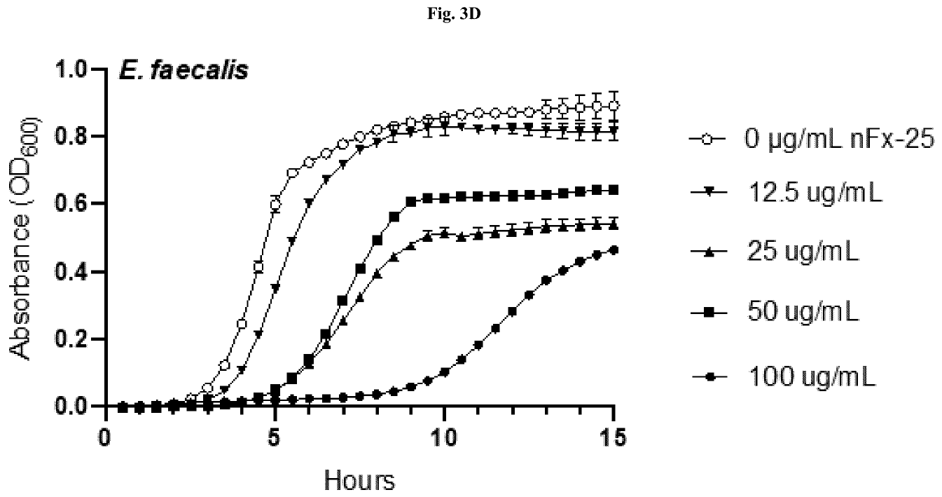

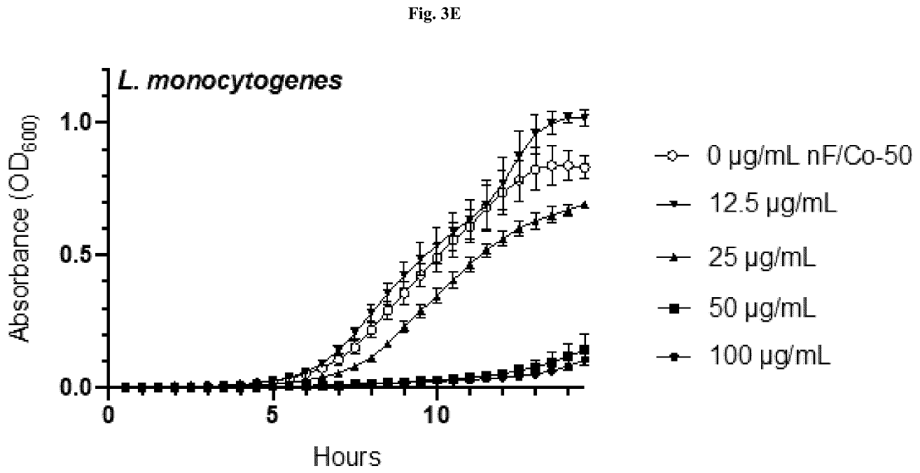

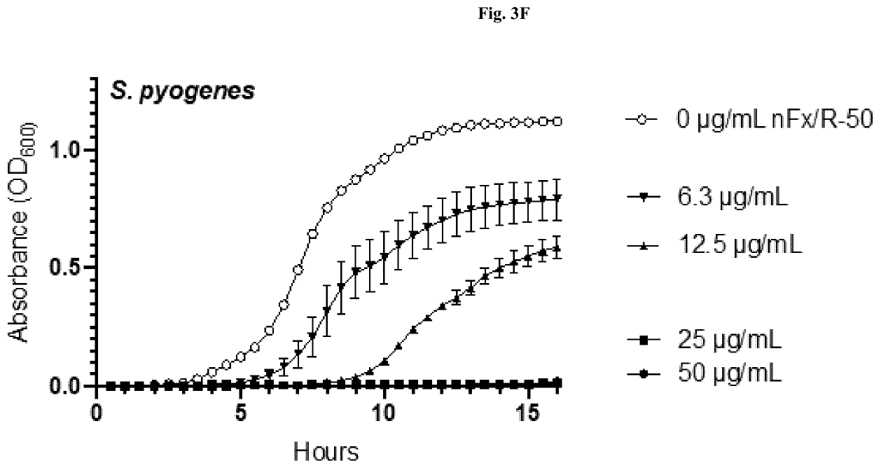

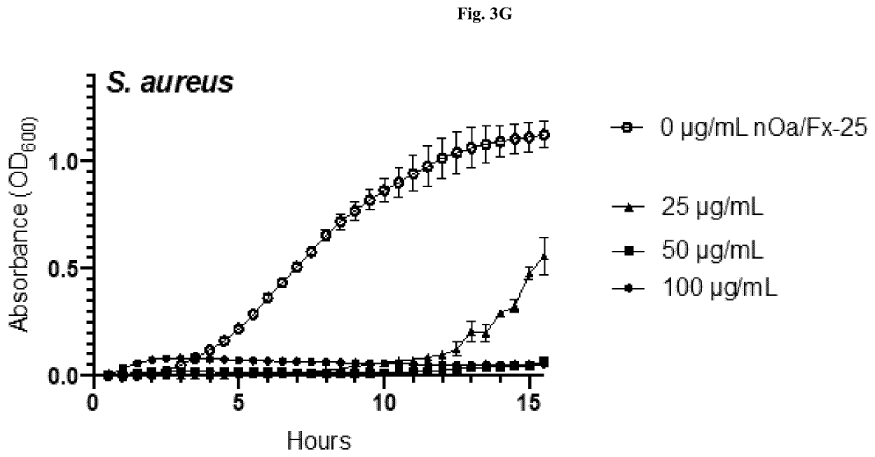

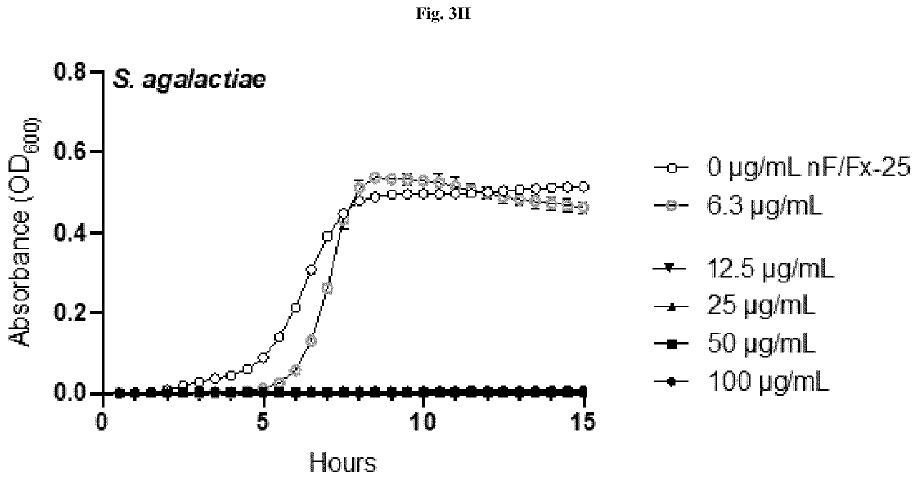

Resulting growth curves for certain bacterial cultures are provided in FIGS. 3A-3H.

FIGS. 3A-3H highlight the most effective nanoemulsions and their corresponding MICs, which was defined as a >90% reduction in bacterial growth throughout the log phase in reference to the untreated bacteria.

Initial screens were first conducted with the complete or incomplete nanoemulsion library at 100 μg/mL against the bacteria in Table 50. Concentrations above 100 μg/mL were not routinely tested due to the larger background absorbance associated with poor solubility at higher concentrations.

The lowest MIC values for the nanoemulsions against specific bacterial strains are provided in Table 52.

TABLE 52
Minimum Inhibitory Concentrations for Certain
Nanoemulsions and Bacterial Strains.

| Bacteria | Nanoemulsion | MIC | |
|---|---|---|---|
| B. cereus | nCo-50 | 50 | μg/mL |
| S. mutans | nFx/R-50 | 10 | μg/mL |
| S. epidermidis | nCo/Fx-75 | 100 | μg/mL |
| E. faecalis | nFx-25 | 100 | μg/mL |
| L. monocytogenes | nF/Co-50 | 50 | μg/mL |
| S. pyogenes | nFx/R-50 | 6.3 | μg/mL |
| S. aureus | nOa/Fx-25 | 25 | μg/mL |
| S. agalactiae | nF/Fx-25 | 12.5 | μg/mL |
| H. pylori | nFx/F-50 | 50 | μg/mL |
| C. acnes | nCo-Fx-25 | 50 | μg/mL |

### Example 10: Bactericidal Assays

Minimum Bactericidal Concentration (MBC) measurements reflect the drug concentration that actively kills the target organisms (bactericidal). This is a much more stringent standard.

| Bacteria | Nanoemulsion | MBC |
|---|---|---|
| S. aureus | nOa/Fx-25 | 50 μg/mL |
| S. pyogenes | nFx/R-50 | 60 μg/mL |
| S. mutans | nFx/R-50 | 25 μg/mL |
| C. acnes | nCo/Fx-25 | 50 μg/mL |

One measure of bactericidal activity is the reduction of viable cells as measured by subsequent growth (following dilution) on appropriate nutrient agar plates. The reduction of colony forming units (CFU) is recorded.

Following a modified procedure from Nakatsuji et. al., a 5 mL overnight culture of S. pyogenes in Tryptic Soy Broth with an OD₆₀₀ of 0.92 (0.9×10\hat{ }7 CFU/mL) was pelleted at 5,000×g for 5 minutes and the broth was removed. The bacterial pellet was resuspended in equal volume of PBS, and 184 μL was added to microtiter plate wells containing 16 μL of nanoemulsion in water. Plates were shaken at 130 rpm in a 37° C. incubator for 5 hours, and reactions were then diluted 1:10-1:10⁶ with PBS. 100 μL of each dilution was added to a Tryptic Soy Broth with 5% defibrinated sheep blood plate and spread with a T-shape spreader. Plates were incubated for 24 hours at 37° C. and CFUs were determined by counting plates that fell in between standard TNTC (too numerous to count) or TFTC (too few to count) range, typically 20-150 CFUs, and multiplying by the dilution factor of the counted plate to determine the undiluted concentration. The same procedure was applied for S. aureus and C. acnes, adjusting for OD to CFU correlations of each species. C. acnes was exclusively grown and treated in an anaerobic GasPak pouch.

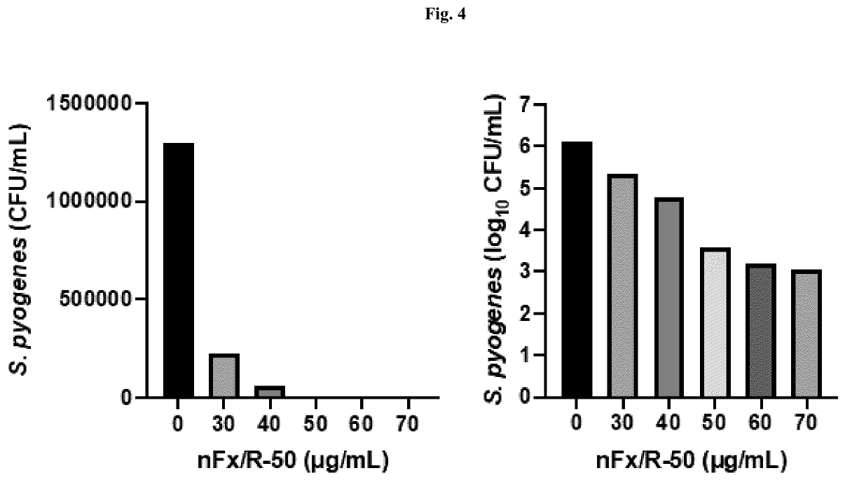

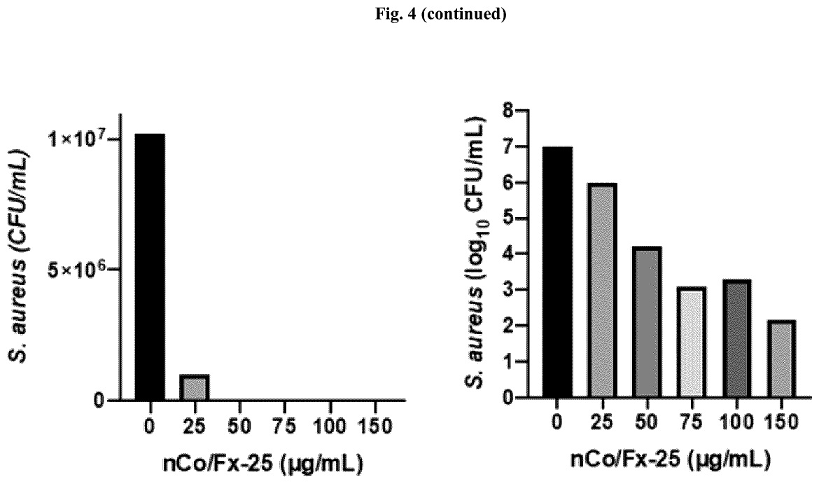

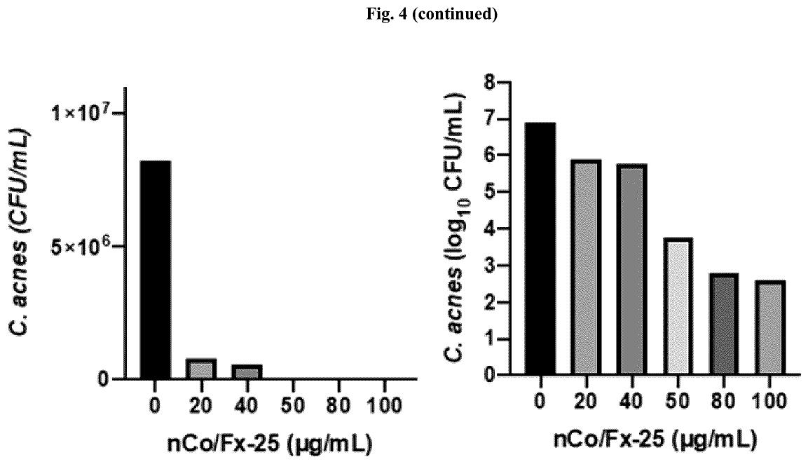

FIG. 4 provides the MBC data. Bacteria were incubated with 0, 25, 50, 75, or 150 μg/mL of nanoemulsions in PBS for 5 hours. Suspensions were diluted 1:10-1:10⁶ with PBS, 100 μL was plated and spread on Tryptic Soy Agar plates with 5% defibrinated sheep blood, incubated at 37° C. for 24 hours, and CFUs were counted. Error bars indicate mean±SD, n=2. Graph shows numerical values on the left-hand side and log values of the same results on the right-hand side.

### Example 11: Microbial Cell Viability Assay

Another method to evaluate bactericidal effects is to measure the amount of a key intracellular metabolite following lysis of the bacteria. The ATP content of cells is very highly correlated with their viability. A cell with little or no ATP is non-viable.

The BacTiter-Glo™ Microbial Cell Viability kit (Promega G8230) was used to determine reduction in viable cells after exposure to nanoemulsions based on quantification of ATP. An overnight culture of S. aureus in Mueller-Hinton broth was diluted 1:100 in fresh Mueller-Hinton broth and added to a 96-well plate containing nCo/Fx-25 at a final concentration of 0, 25, 50 or 100 ug/mL in triplicate. The plate was shaken at 150 rpm in a 37° C. incubator for 5 hours, then 75 μL of each well was transferred to a new well and 75 μL of Reagent (Reagent: BacTiter-Glo Substrate+BacTiter-Glo Buffer, aliquoted and stored at −80 C) was added. The plate was shaken at 150 rpm in a 37 C incubator for 5 minutes, and immediately placed on a Biotek Cytation 5 plate reader for luminescence reading.

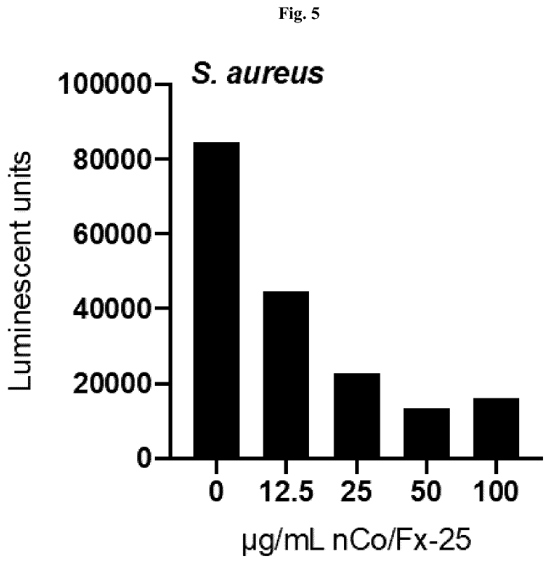

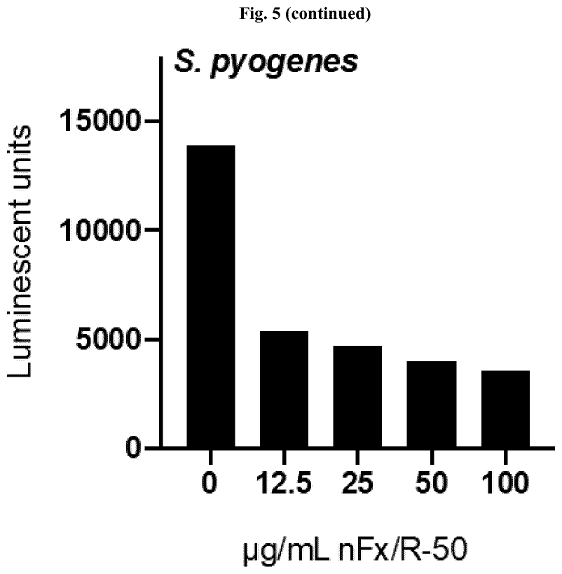

FIG. 5 shows the bactericidal effects of nanoemulsions on cell viability (ATP). 0.9×10⁷ CFU/mL of S. aureus or S. pyogenes incubated with 0, 12.5, 25, 50, or 100 μg/mL of nanoemulsion in broth for 5 hours, followed by addition of BacTiter-Glo reagent and luminescence reading.

The effect of select nanoemulsions on cell viability via cellular ATP measurement provides an additional means of evaluation for antibacterial efficacy, and is useful for quick, high-throughput screening of large libraries of materials without the large consumption of agar plates. S. aureus appears to have significant cell viability reduction at a concentration of 25 μg/mL nCo/Fx-25 and is near the values seen in alternative experiments.

MBC were determined based on the concentration at which a 2-log-fold reduction was observed in reference to the untreated sample. This value happened to be 50 μg/mL for all experiments: nFx/R-50 against S. pyogenes, nCo/Fx-25 against S. aureus, and nCo/Fx-25 against C. acnes.

### Example 12: Asymmetric Bactericidal Effects: Co-cultured Staphylococcus Species-Specific Bactericidal Assay

The compositions of the present disclosure can provide the opportunity to prepare nanoemulsions (and microemulsions) that selectively inhibit the growth of certain target bacteria (e.g., pathogenic S. aurous) while limiting the effects on other bacteria present in the environment.

Overnight cultures of three Staphylococcus species: S. aureus, S. epidermidis, and S. capitis were diluted to OD₆₀₀=1, and combined into a single microcentrifuge tube. 50 μL of the combined culture was added to 5 mL culture tubes containing 0, 50 or 100 μg/mL nanoemulsion nCo/Fx-25 in Mueller-Hinton broth and shaken at 200 rpm in a 37 C incubator for 5 hrs. The cultures were then 10-fold serially diluted 1:10-1:10⁶, and 100 μL was plated on Tryptic Soy Broth plates with 5% defibrinated sheep blood and evenly spread with a sterile T-shaped spreader. Repeat experiments only plated 1:10⁴-1:10⁶ due to consistent too-numerous-to-count growth in less diluted samples.

Plates were incubated at 37° C. overnight, colonies were counted, and S. aureus vs. non-S. aureus were differentiated based on visible rings of beta-hemolysis around S. aureus colonies. On plates with 10-50 non-S. aureus colonies, individual not-overlapping colonies were dabbed with a sterile pipet tip and spotted on Mannitol Salt Agar plates. Yellow S. capitis colonies was clearly differentiated from pinkish-red S. epidermidis colonies due to consumption of the sugar mannitol and release of acidic fermentation byproducts that lower the pH of the media, turning the phenol red indicator yellow.

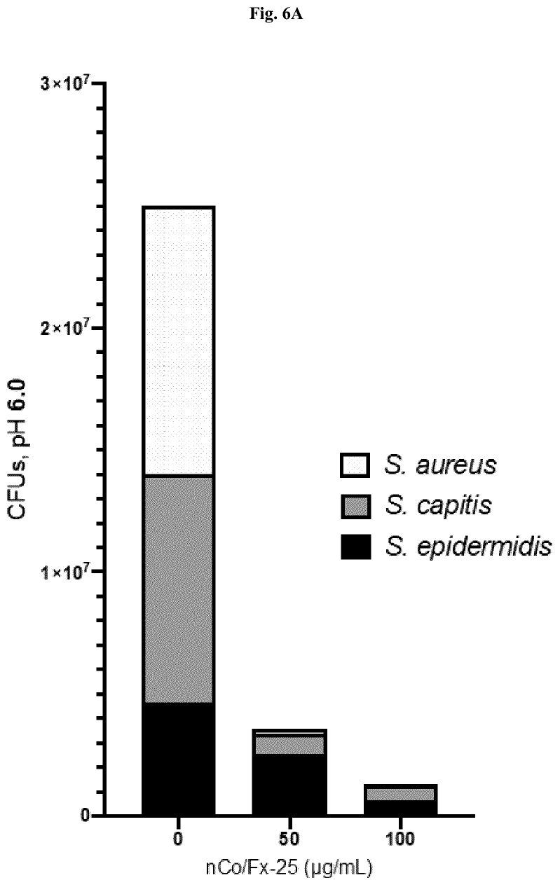

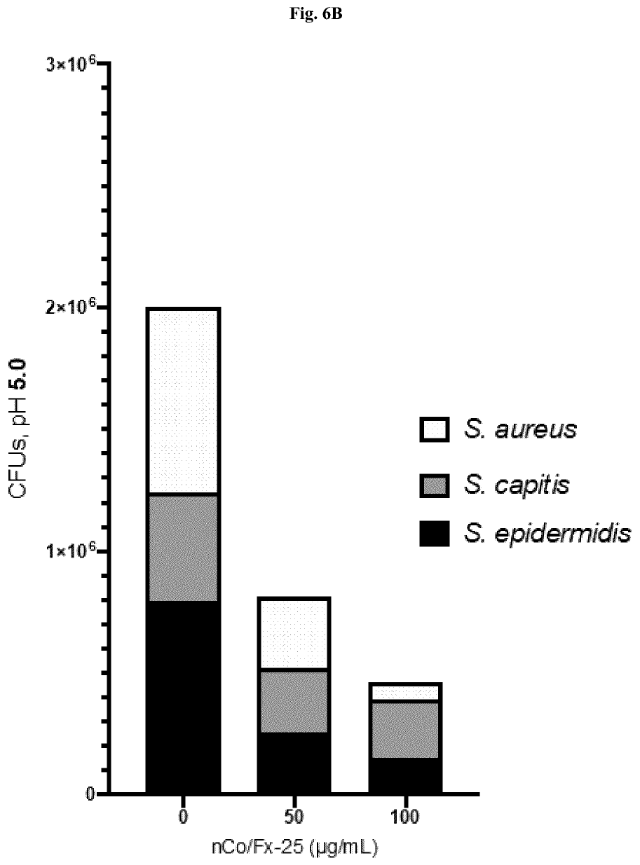

FIGS. 6A-6B show the bactericidal effects of nanoemulsions on three co-cultured bacteria. S. aureus, S. epidermidis, and S. capitis co-cultured and treated with nCo/Fx-25 for 5 hrs at either pH 6 (FIG. 6A) or pH 5 (FIG. 6B).

A significant percentage of the skin microbiome consists of Staphylococcus bacteria, which can range from 35% of the total in children to 80+% in young adults, and the species also greatly vary with age, with S. aureus comprising a much higher percentage in children than in adults. The goal of this experiment was to disproportionately reduce the colonies of S. aureus in a mixed population of three bacteria, while not greatly reducing colonies of S. epidermidis and S. capitis, two of the most abundant commensal skin bacteria. S. epidermidis can comprise up to 30% of the Staphylococcus species present in the skin, and S. capitis up to 40%, depending on body location, age, and overall health. Application of this nanoemulsion formulation provided asymmetric reduction of the target species.

FIGS. 6A-6B also demonstrate the effect of pH on the bactericidal activity of the nanoemulsion. At pH 6.0 (FIG. 6A) there is an overall ˜5× decrease in all bacteria after treatment with 50 μg/mL nanoemulsion, which decreases even further at 100 μg/mL.

At pH 5.0, FIG. 6B shows an overall ˜2× decrease in all bacteria after treatment with 50 μg/mL nanoemulsion, with another 2× decrease at 100 μg/mL, with a degree of S. aureus reduction that is less than the effect seen at higher pHs.

### Example 13: Biofilm Inhibition Assays

Biofilm formation is an important mechanism of resistance for bacteria on skin and other surfaces. Antimicrobials that disrupt these films offer special advantages in infection prevention and control. The presence and cellular activity of biofilm formation in select bacteria was assessed colorimetrically by the Crystal Violet (CV) and XTT assays in 96-well plates.

Following a procedure by Hobby et. al., bacteria were grown overnight in Mueller-Hinton broth, and diluted into 96-well plates that contained each nanoemulsion dissolved in Tryptic Soy Broth. Plates were incubated for 24 h at 37° C., planktonic cells were removed by pipetting, and wells were gently washed 3× with DI water.

For the CV assay, 125 μL of sterile filtered 2% CV in water solution was added to each well, plates were incubated at RT for 15 min, washed 3× with DI water, and set aside to dry for 3 hr. 125 μL of 30% acetic acid was added to each well and incubated for 15 min, then the well contents were transferred to a fresh 96-well plate for reading. Absorbance at 570 nm was measured with a Biotek Cytation 5 plate reader. In the alternative XTT assay, the reagent was prepared at 0.5 g/L in phosphate-buffered saline, and menadione was prepared as a 10 mM stock in acetone. Before use, menadione was added to the XTT solution at a final concentration of 1 μM and stored covered with foil. 100 μL of XTT reagent was added to pre-washed wells, the plate was covered with foil and incubated at 37° C. for 3 hours, and absorbance read on a plate reader at 490 nm. XTT was also used as a means of measuring biofilm formation and has been shown to be useful for compound comparison within a particular strain or species, but comparisons between strains cannot be accurately made because of unequal metabolism of XTT.

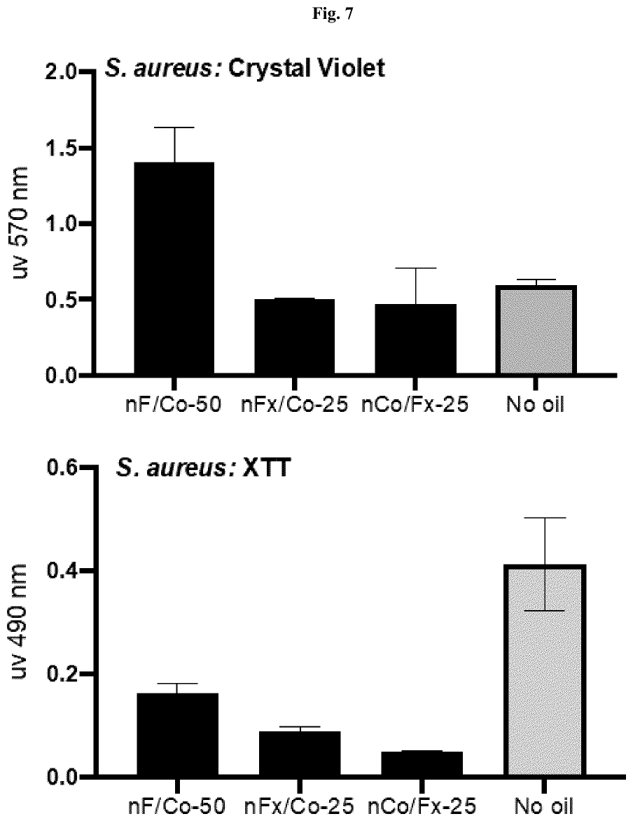

Results of the CV and XTT assays are shown in FIG. 7.

FIG. 7 illustrates the results of the two methods of measuring biofilm formation in the presence or absence of select nanoemulsions.

### Example 14: Membrane Permeability and Depolarization Assays

Disruption of bacterial membrane integrity leads to increase in membrane permeability, leakage of cellular components into the medium, disruption of membrane polarization and other bacteriostatic and bactericidal effects. Changes in the membrane permeability and depolarization of S. aureus by nanoemulsions was assayed using modified procedures from Blaskovich et. al.

The membrane-impermeable fluorescent DNA intercalating dye Propidium iodide was used to assay the effect of nanoemulsion nCo/Fx-25. An overnight culture of S. aureus was pelleted, washed twice with buffer (10 mM HEPES buffer, pH 7.4, 50 μg/mL CaCl₂), 5 mM glucose), resuspended in buffer, and added to a 96-well plate containing nCo/Fx-25 at a final concentration of 0, 25, 50 or 100 ug/mL in triplicate.

The plate was incubated at 37° C. for 1 hr, then 5 ug/mL of Propidium iodide was added and immediately put on a Biotek Cytation 5 plate reader to read fluorescence every minute for 90 minutes (ex/em: 535/620 nm). Background fluorescence from untreated wells was subtracted from all readings.

The effects of membrane disruption can also be evaluated by directly assessing the polarization of the membrane. The membrane potential-sensitive fluorescent dye 3,3-dipropylthiacarbocyanide DiSC3(5) was used to assay the effect of nanoemulsion nCo/Fx-25. An overnight culture tube of S. aureus was pelleted, washed once with buffer (5 mM HEPES pH 7.2, 100 mM KCl, 20 mM glucose), resuspended in buffer to an OD₆₀₀=0.1, and DiSC3(5) was added to the tube at a final concentration of 100 nM. The tube was wrapped in foil and shaken at 220 rpm in a 37° C. incubator for 30 minutes. In a 96-well black polystyrene plate, 16 μL of nanoemulsion or water, and 184 μL of dye-saturated cells were added to make final nanoemulsion concentrations of 0, 25, 50 or 100 ug/mL in triplicate. The plate was immediately placed on a Biotek Cytation 5 plate reader to read fluorescence every 10 s for 60 min (ex/em: 622/670 nm).

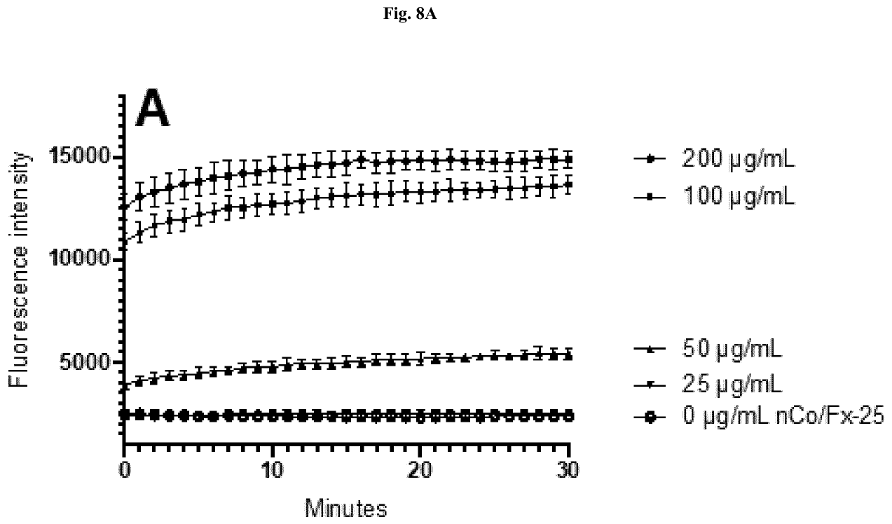

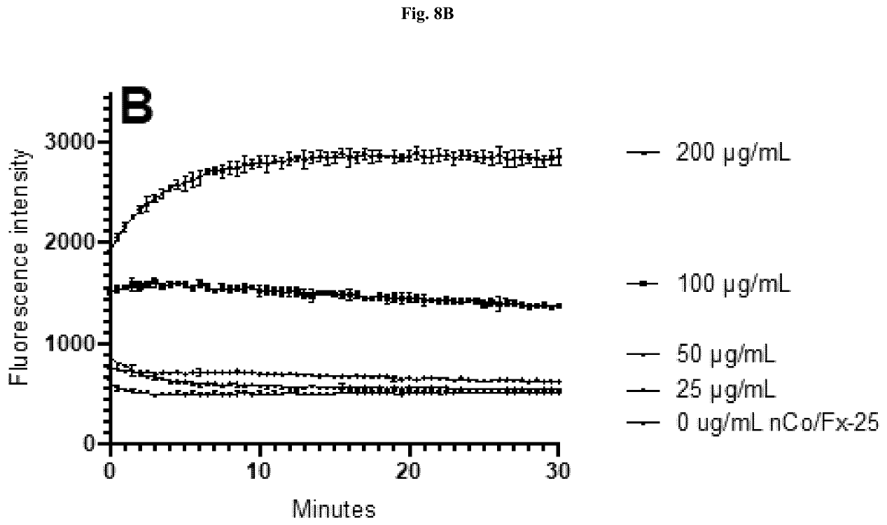

FIGS. 8A-8B show the effect of nCo/Fx-25 on membrane permeability and membrane depolarization in S. auereus.

FIG. 8A: Membrane permeability assay on S. aureus with Propidium iodide dye, with increasing concentrations of nanoemulsion nCo/Fx-25; fluorescence excitation 535 nm, emission 620 nm. FIG. 8B Membrane depolarization assay on S. aureus with DiSC3(5) dye, with increasing concentrations of nanoemulsion nCo/Fx-25; fluorescence excitation 622 nm, emission 670 nm.

FIG. 8A shows clear S. aureus dose-response to treatment with nanoemulsion nCo/Fx-25, with no increase in permeability at 25 μg/mL, a small increase at 50 μg/mL, and large sustained increases at 100 and 200 μg/mL. This provides evidence towards a mechanism of action in which select monoglycerides/fatty acid nanoemulsions directly impact the permeability of bacterial membranes, which results in release of cellular components and hinders cell viability. FIG. 8B shows membrane depolarization of S. aureus in response to nanoemulsion nCo/Fx-25. The DiSC3(5) dye signal stabilizes over the course of the 30 min pre-incubation (not shown) and following addition of the nanoemulsion at time=0 the untreated bacteria show a slow consistent decrease in signal. This also occurs with bacteria treated with 25 μg/mL nanoemulsion, but higher fluorescent values are sustained with 50, 100 and 200 μg/mL.

### Example 15: Comparison of Triglyceride Nanoemulsions, MAG/FFA Microemulsions, and MAG/FFA Nanoemulsions

The observed long-term stability of many nanoemulsions and the lack of necessity to add extra emulsifiers and co-surfactants is an important factor towards manufacturing a consistent and desirable product, but certain means of application may dictate conditions in which the triglyceride starting oil or a crude emulsification method is more practical. To this end, comparisons have been made from an antibacterial standpoint to evaluate differences of activity. Because of the quick phase separation that occurs in triglyceride/water emulsifications or in EMO/FFA oil in water homogenizations, materials were prepared and plated immediately with bacteria before significant separation could occur. The procedure was identical to MIC assays used to generate FIGS. 3A-3H.

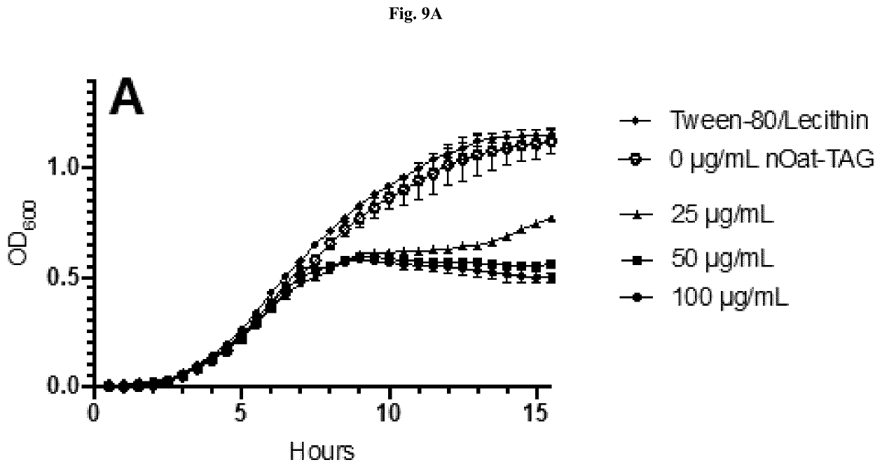

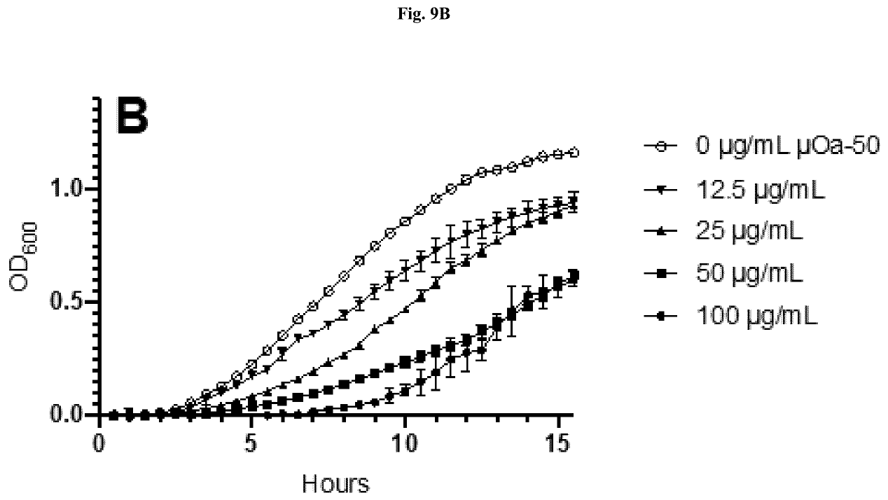

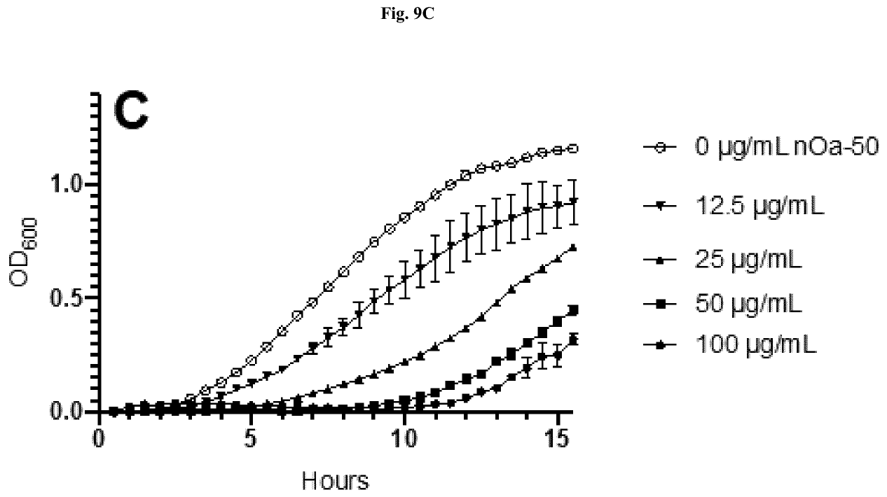

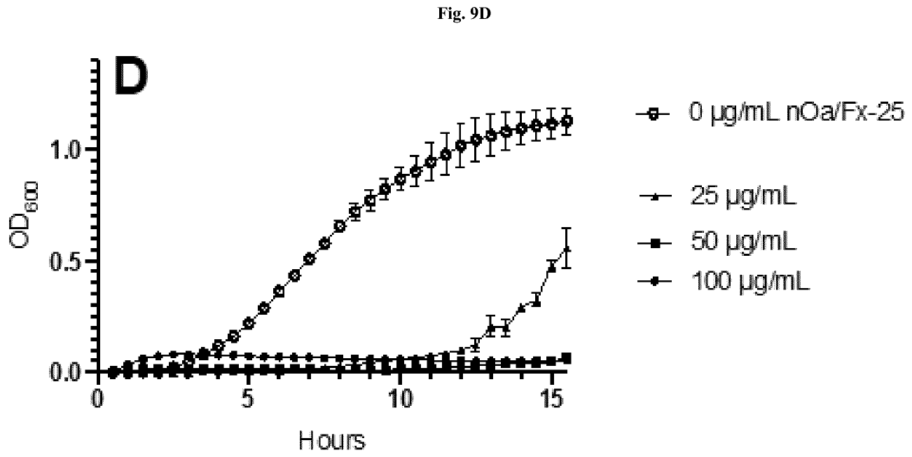

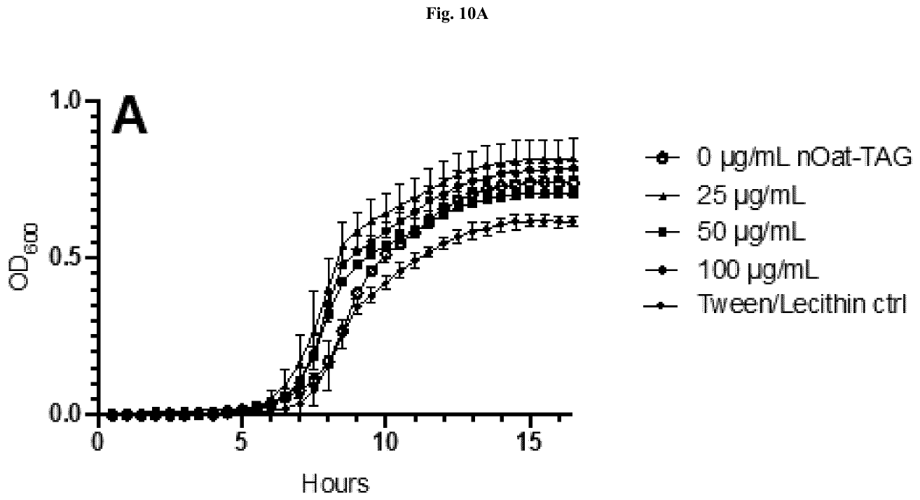

FIGS. 9A-9D demonstrates a graph-to-graph increase in S. aureus inhibition when incubated with A) an Oat Oil nanoemulsion containing Tween-80/Lecithin; B) 50/50 EMO/FFA Oat Oil microemulsion; C) 50/50 EMO/FFA Oat Oil nanoemulsion, and D) 25/75 EMO/FFA Oat/Flaxseed nanoemulsion. In 9A, a vehicle control was measured of Tween-80/Lecithin, showing no growth inhibition. The inhibition that does occur in oil-treated bacteria at the 8-hour mark is likely contributed by bacterial manipulation of the triglyceride, either through the secretion of glycerol ester hydrolases (lipases), metabolic byproducts such as lactic acid, or changes in media composition that contribute to a delayed degree of inhibition. The difference between 9B (oat microemulsion) and 9C (oat nanoemulsion) is not as dramatic, but inhibition continues for 3-4 hours longer in the nanoemulsion conditions. 9D demonstrates the ability of distinct oil combinations to have a significantly lower MIC, albeit adding complexity to the product development process.

1:9 oil in water emulsions prepared immediately before use and diluted into 96 wells plates followed by addition of diluted bacteria. Plates were placed on a microplate shaker for 16 hours at 200 rpm and 37° C., measuring OD₆₀₀ at 30 min intervals. A) Nanoemulsion made with Oat oil (mainly triglycerides), 5% Tween-80 and 1% Lecithin (340±5 nm, PDI 0.241±0.140); B) Microemulsion of 50/50 Oat-EMO/Oat-FFA oil, 1:9 oil in water (821±117 nm, PDI 0.201±0.106); C) Nanoemulsion nOa-50 (197±nm; PDI 0.172±0.035); D) Best combo nanoemulsion nOa/Fx-25 (195±2 nm, PDI 0.172±0.035).

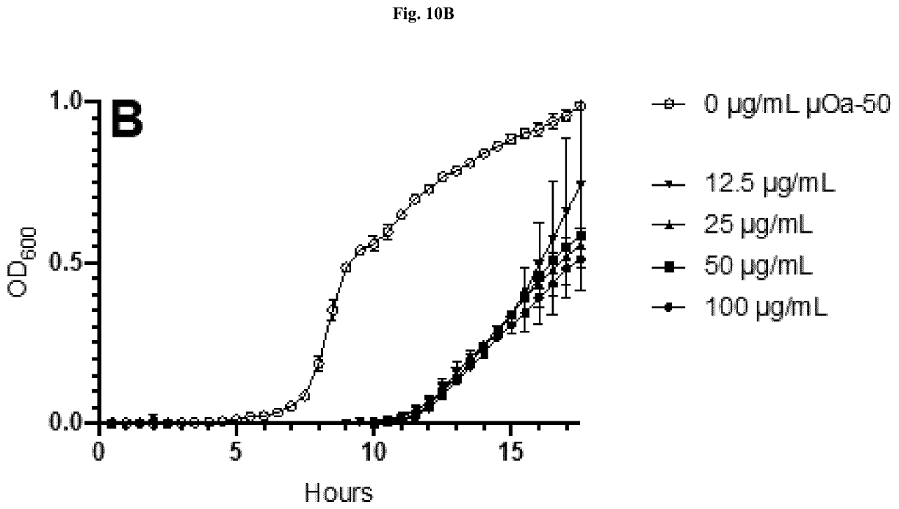

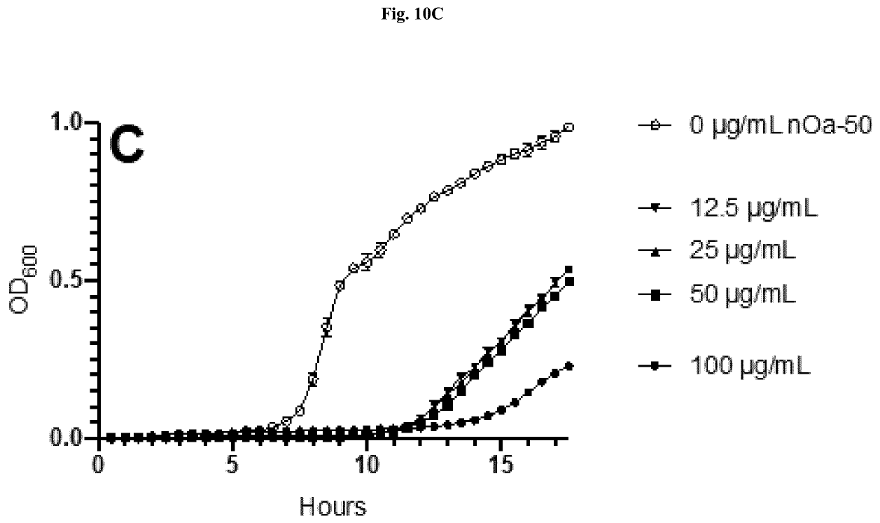

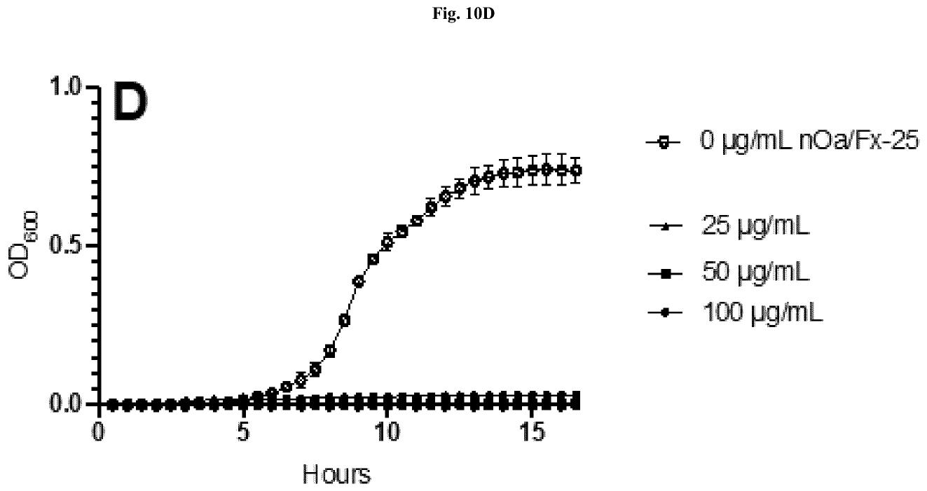

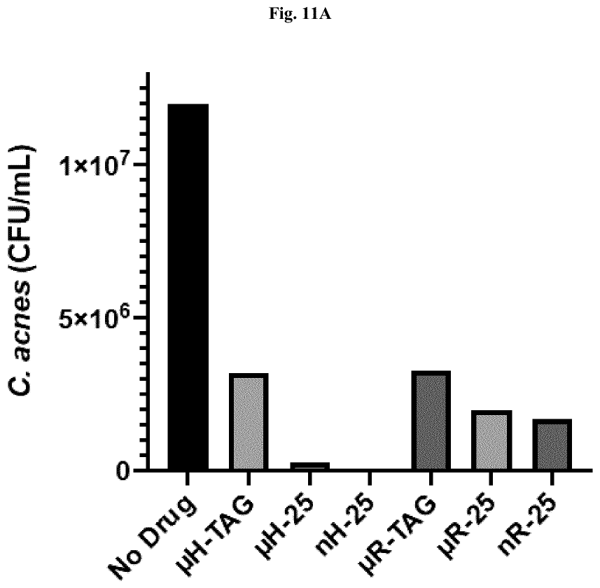

The same trend held for growth inhibition of S. pyogenes as shown in FIGS. 10A-10D, which is a more fastidious bacteria that was inhibited at overall much lower concentrations of nanoemulsions in this study. S. pyogenes is unaffected by the unprocessed Oat oil (10A), is moderately inhibited by either the microemulsion or nanoemulsion form of Oat MAG/FFA (10B and 10C), and completely inhibited at all measured concentrations of an Oat/Flaxseed EMO/FFA oil (10D).

FIGS. 10A-10D: 1:9 oil in water emulsions prepared immediately before use and diluted into 96 wells plates followed by addition of diluted bacteria. Plates were placed on a microplate shaker for 16 hours at 200 rpm and 37° C., measuring OD₆₀₀ at 30 min intervals. A) Nanoemulsion made with Oat oil (mainly triglycerides), 5% Tween-80 and Lecithin; B) Microemulsion of 50/50 Oat-EMO/Oat-FFA oil, 1:9 oil in water; C) Nanoemulsion nOa-50; D) Best combo nanoemulsion nOa/Fx-25.

In a bactericidal experiment using the anaerobic bacteria C. acnes, TAG emulsions, microemulsions and nanoemulsions of Hemp seed oil and Rosehip oil were compared. These oils were chosen as prime formulation reagents for facial skin care due to their non-comedogenicity, as well as many other beneficial properties. Cis-unsaturated lipids are such as oleic acid and linoleic acid are often used in skin cosmetic formulations due to their skin penetration enhancement, and the majority component of both hemp seed and rose hip oil is triglycerides of linoleic acid.

In this experiment, μH-TAG and μR-TAG were made as 1:9 oil in water microemulsions of Hemp seed oil or Rose hip oil, without adding any emulsifiers/surfactants, and used immediately before separation could occur. Since bactericidal experiments using anaerobic bacteria is more time sensitive and space limited, this change removed the necessity for extra control experiments with emulsifiers/surfactants.

Methods used were the same as those to generate MBC data in FIG. 4, except a single 1-hr incubation time point was gathered for dilutions and plating. All treated bacteria had a decrease in colony forming units, as shown in FIGS. 10A (CFUs/mL) and 10B (log₁₀ CFU/mL), however no Rose hip oil construct gave a 1-log decrease. The clearest distinctions can be drawn from the Hemp seed oil, in which the TAG microemulsion gave a minor reduction, the EMO/FFA oil microemulsion gave a nearly 2-log reduction, and the EMO/FFA oil nanoemulsion giving a greater than 2-log reduction in colonies.

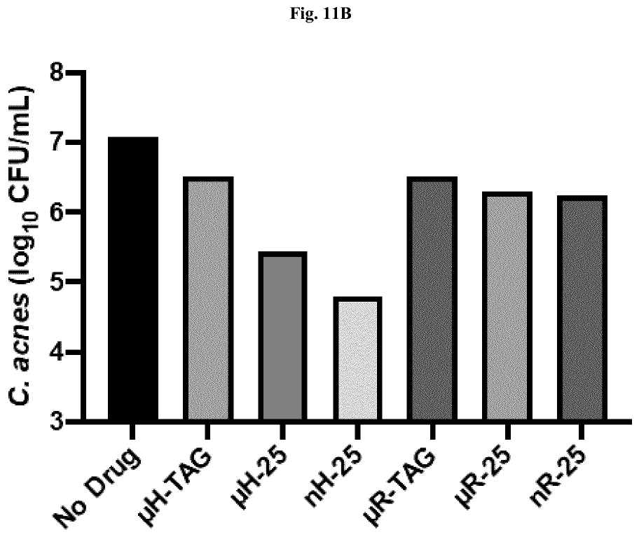

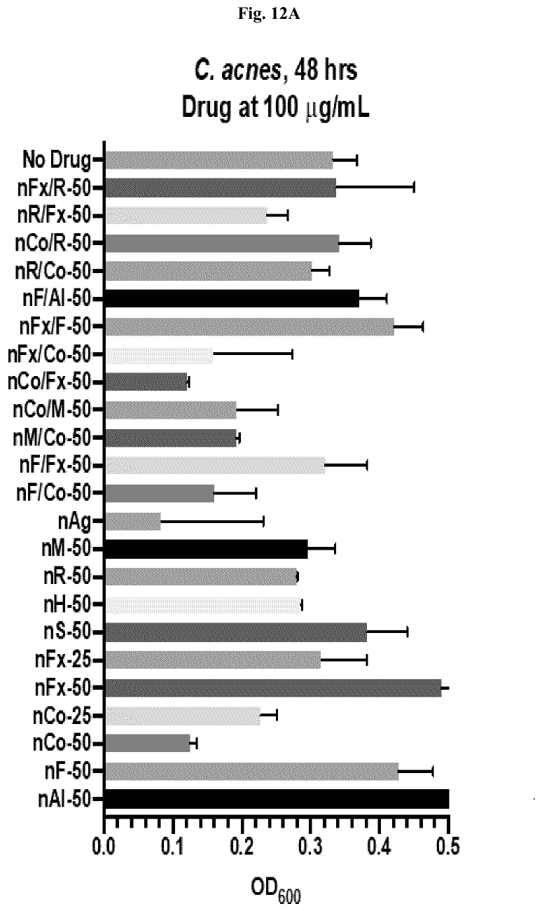

FIGS. 11A-11B show the results of the assay. FIG. 11A: CFUs/mL and FIG. 11B: log₁₀ CFUs/mL after 1 hr incubation with oil emulsions, plating on Tryptic soy broth agar plates with 5% defibrinated sheep blood, and incubation in anaerobic pouches for 3 days.

### Example 16: Library Screen of Nanoemulsions Against C. acnes and H. pylori

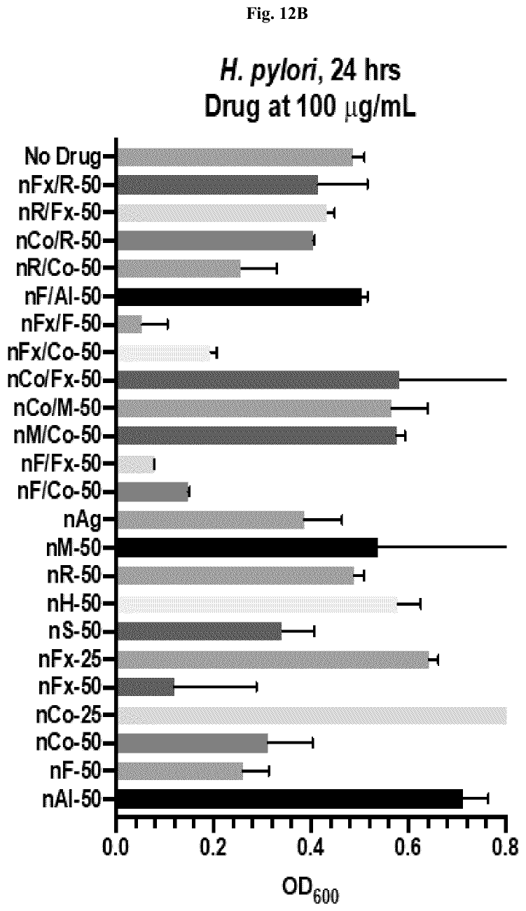

A general screen was conducted with all the nanoemulsions against C. acnes and H. pylori. FIG. 12A provides the screening results for C. acnes. Combinations containing coconut EMO were superior growth inhibitors.

Bacteria were grown in Gaspak pouches with anaerobic or microaerophilic catalysts in 96 well plates. Plates were removed at fixed time points for absorbance reading at 600 nm on a Biotek Cytation 5 plate reader.

The same screen was conducted with H. pylori in FIG. 12B, with nanoemulsion combos of Flaxseed and Fish standing out as superior growth inhibitors. This is probably due to the known effect of PUFAs on H. pylori, and will be further explored.

Results are shown in FIGS. 12A-12B.

### Example 17: Screen of Nanoemulsions Against P. aeruginosa

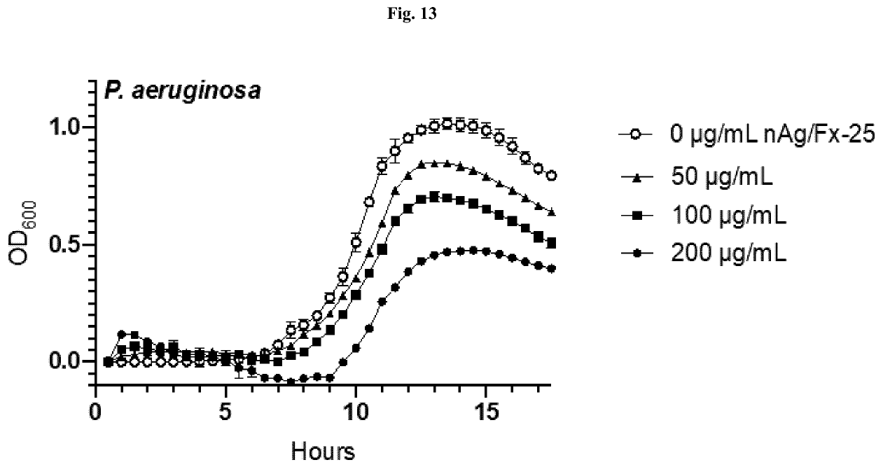

P. aeruginosa was also screened against nAg/Fx-25 at varying concentration with the results shown in FIG. 13 which shows inhibition of P. aeruginosa by the Algae-EMO Flaxseed-FFA oil nanoemulsion nAg/Fx-25 at higher concentrations.

### Example 18: Screen of Nanoemulsions Against Micrococcus luteus and Corynebacterium xerosis

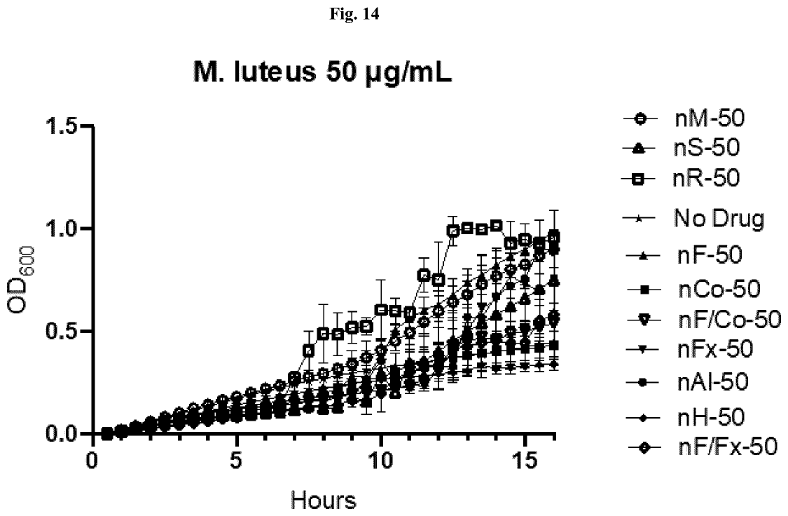

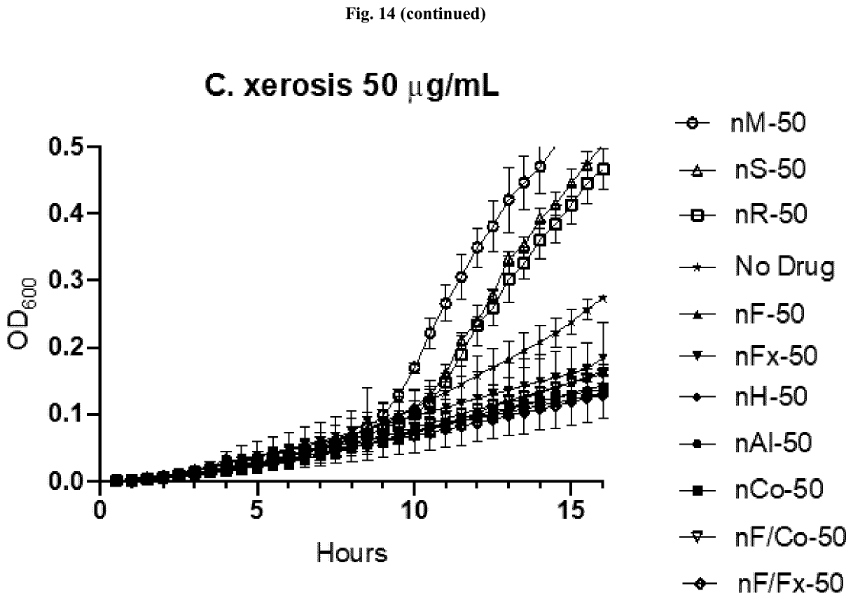

FIG. 14 shows a screen of several nanoemulsions against the often commensal skin bacteria Micrococcus luteus and Corynebacterium xerosis.

### Example 19: Cream Formulations Incorporating the Present Nanoemulsions

Various base cream formulations (BC_v1-BC_v17) were prepared with the following: (1) Glycerin 5%; (2) Petrolatum 3%; (3) Dimethicone 1%; (4) Cetyl Alcohol 2.5%; (5) Colloidal Oatmeal 1%; (6) variable ingredients listed in Table 53; and (7) q.s. with DI water to 100%. The base cream formulations are combined with any one of the nanoemulsions of the present disclosure (as described in Examples 2-4, and in preferred embodiments, nOa/Fx-25) with the nanoemulsion being added at an about from about 1% to about 5%, about 2% to about 4% and more preferably about 2.5%. It should be noted that the EMO and FFA oil listed under the “Low HLB Emulsifier” of Table 53 is separate from the nanoemulsion component of the formulation. It should be understood that the ingredients of the base cream formulations of this Example 19 could be substituted with other ingredients and this should be taken as exemplary and not limiting as to the base cream formulation suitable for use in the present disclosure.

To generate the creams, water and water soluble ingredients were combined separately into Phase A, oil soluble ingredients were combined separately into Phase B, and the potentially heat-sensitive ingredients: Colloidal Oatmeal and Ceramide NP (see Table 53) were combined separately into Phase C. Phases A and B are heated at 70 C, and Phase B is added to Phase A in a stainless steel vessel. Ingredients are homogenized together at 5000-8000 rpm until solidification begins, pausing every 5 minutes to avoid overheating. When thickening occurs, Phase C ingredients are added and homogenized until uniform. The cream is then transferred to an appropriate storage vessel for testing.

TABLE 53
Variable Ingredients of Based Cream Formulations.

| | | Low HLB | High HLB | Skin |
|---|---|---|---|---|
| | Emollient | Emulsifier | Emulsifier | Conditioning |
| BC_v1 | Isopropyl palmitate 3% | Steareth-2 2% | Steareth-20 1% | — |
| BC_v2 | Isopropyl palmitate 3% | Steareth-2 2% | Tween-20 1% | — |
| BC_v3 | — | Steareth-2 2% | Tween-20 1% | — |
| BC_v4 | Isopropyl palmitate 3% | Steareth-2 2% | — | — |
| BC_v5 | — | Steareth-2 2% | — | — |
| BC_v6 | Isopropyl palmitate 3% | Steareth-2 2% | Tween-80 1% | — |
| BC_v7 | — | Steareth-2 2% | Tween-80 1% | — |
| BC_v8 | — | Steareth-2 1% | Tween-80 1% | — |
| | | Oat EMO 1% | | |
| BC_v9 | — | Oat EMO 2% | Tween-80 1% | — |
| BC_v10 | — | Steareth-2 1% | — | — |
| | | Oat EMO 3% | | |
| BC_v11 | — | Steareth-2 1% | — | — |
| | | Oat EMO 3% | | |
| | | Flaxseed FFA 2% | | |
| BC_v12 | — | Oat EMO 4% | — | — |
| | | Flaxseed FFA 2% | | |
| BC_v13 | — | Glyceryl | — | — |
| | | Stearate SE 2% | | |
| | | Oat EMO 3% | | |
| | | Flaxseed FFA 1% | | |
| BC_v14 | — | Oat EMO 3% | Steareth-20 | — |
| | | Flaxseed FFA 3% | 0.25% | |
| BC_v15 | Isopropyl palmitate 3% | Steareth-2 2% | Steareth-20 1% | Panthenol 2% |
| | | | | Ceramide NP 0.1% |
| BC_v16 | — | Oat EMO 3% | Steareth-20 | Panthenol 2% |
| | | Flaxseed FFA 3% | 0.25% | Ceramide NP 0.1% |
| BC_v17 | Avena Sativa | Oat EMO 3% | Steareth-20 | Panthenol 2% |
| | Oat Extract 1% | Flaxseed FFA 3% | 0.25% | |

Design considerations for the base cream formulations are as follows: 1) the cream contains sufficient emulsifiers to form a semi-solid cream, i.e., slow movement out of a conical tube when held upside down; 2) is smooth and homogenous in texture, without granulation or separation; 3) does not separate into phases when centrifuged; 4) is not strongly odorous or discolored; and 5) does not interfere with the ability of the nEMO nOa/Fx-25 to reduce S. aureus CFU/mL by more than 2-log in a typical 3-hour bactericidal experiment.

The bactericidal experiments can be performed as follows. The cream formulations are transferred in a laminar flow hood to a 10 mL culture tube using a sterile pipet tip and weighed until the desired weight is added. Tubes will be centrifuged at 1,000×g for 30 seconds to pool the cream at the bottom of the tube. 4.5 mL of sterile PBS is added to each tube using a 10 mL serological pipet, nEMO is pipetted into the solution (unless mixed with base formulation beforehand), and tubes are vortexed until the cream is evenly distributed. Control tubes are prepared without Basic Cream for comparison. A 5 mL overnight culture of S. aureus in Mueller-Hinton Broth with an OD₆₀₀ of ˜3.0 is pelleted at 4,000×g for 3 minutes and the broth is removed. The bacterial pellet is resuspended in equal volume of PBS, and 500 μL is added to each culture tube for a final volume of 5 mL. Tubes are shaken at 200 rpm in a 37° C. incubator for 5 hours, and reactions are then diluted 1:10-1:10⁶ with PBS. 100 μL of each dilution is added to a Tryptic Soy Broth with 5% defibrinated sheep blood plate and spread with a T-shape spreader. Plates are incubated for 24 hours at 37° C. and CFUs are determined by counting plates that fell in between standard TNTC (too numerous to count) or TFTC (too few to count) range, typically 20-150 CFUs, and multiplying by the dilution factor of the counted plate to determine the undiluted concentration. This method is used in further examples for bactericidal analysis unless otherwise specified, with the exception that reaction time may be varied and starting bacterial concentrations can be between 10⁵-10⁶ unless otherwise specified.

### Example 20. Selective Bactericidal nEA10-Cream in Culture Experiments

To assess the ability of nOa/Fx-25 nanoemulsion to function not only as a potent bactericidal compound against S. aureus when combined with the base creams of Example 19, but to also not significantly kill commensal bacteria, a co-culture experiment is provided using S. epidermidis and S. hominis, two generally harmless Staphylococcal species that make up a majority of the skin microbiome population.

The bactericidal experimental design described in Example 19 is followed with one key exception. OD600 readings of overnight cultures of S. aureus, S. epidermidis, and S. hominis are taken and after pelleted, the cultures are resuspended in a volume of PBS to make the three cultures equal in concentration. Microcentrifuge tubes are then prepared containing either S. aureus and S. epidermidis (SA/SE), S. aureus and S. hominis (SA/SH), or S. aureus, S. epidermidis, and S. hominis (SA/SE/SH).

### Example 21. Clinical Research Program

A clinical research program to evaluate an enzyme-modified oil formulation for the management of atopic dermatitis (AD) flare-ups will be performed. The study will be a single-center, randomized, blinded, controlled trial assessing a topical formulation containing enzyme modified oil (“activated oil”) and colloidal oatmeal, compared to a standard-of-care formulation.

It is expected that the enzyme-modified oil-supplemented formulation will perform better than the standard of care formulation (Control) in re-establishing a healthy microbiome on AD lesion skin, and in particular will reduce the absolute abundance and population density of S. aureus during recovery from the AD flare-up.

The study will determine if the activated oil-supplemented formulation will selectively inhibit the outgrowth of S. aureus while enriching the population of other commensal skin microbial flora and accelerating the return of a healthy microbiome and healthy skin.

60 patients, aged 18 an over will be enrolled and will include male or female patients who have a recent atopic dermatitis itch flare-up and/or pre-flare-up symptoms at screening as determined by visual analog scale (VAS), diagnosis of atopic dermatitis with mild to moderate eczema (grade 3.0-7.5 on the Rajka-Langeland severity index), having a target lesion with an ADSI score of 6-12 and an erythema and pruritis subscore of ≥2 (moderate), a baseline score of ≥4 on the itch assessment VAS, generally good health based on reported history, ability to discontinue all topical emollients, moisturizers and/or other skin barrier lotions, emulsion treatments for eczema at the targeted lesion areas and surrounding areas, females of child-bearing potential should agree to continue using a medically acceptable method of birth control throughout the study and for 30 days immediately after the last dose of study drug, ability to administer topical medication and willing to adhere to the study interventions, and agreement to adhere to Lifestyle Consideration throughout the duration of the study.

Patients will be excluded for pregnancy or lactation, any type of malignancy involving the skin in the last 5 years, known allergy to hydrocortisone or topical antibiotic, topical or oral use of an antibiotic within the last 7 days, bleach bathing in the last 7 days, suspected non-compliance or non-cooperation, intake of experimental drugs or experimental topical skin treatments within 30 days prior to study start, mental disability or any other lack of fitness, or diagnosis of human immunodeficiency virus in the medical history.

In the trial, patients with an escalated eczema lesion will be provided with a blinded study material, either the colloidal oatmeal and activated-oil topical lotion (Protocol Product) or the colloidal oatmeal topical standard of care lotion (Control Product). Patients will be instructed to consistently treat the lesion with the study material twice per day for 14 days, followed by a 7-day regression period during which the test products are not used to assess durability of the response.

Patients will also be provided with a standard mild moisturizing body wash to use in place of normal body wash and/or soap during their usual daily cleaning routine (i.e. daily shower) for the 21-day trial period. Alternatively, the patient will be instructed to use their normal body wash and/or soap during their usual daily cleaning routine.

Patient assessments will be conducted during clinical visits at baseline, day 1, day 3, day 7, day 14 and day 21.

After enrollment, this study will commence with patients coming to the clinic for baseline measurements and instruction. A target lesion will be identified for at home treatment by the patient or guardian or clinician. Patients will be given a 14-day supply of the topical treatments for the study lesion and surrounding area. Patients will also be provided with a standard mild moisturizing body wash to use in place of normal body wash and/or soap during their usual daily cleansing routine (i.e., daily shower) for the 21-day trial period. Alternatively, the patient will not be provided with the standard mild moisturizing body wash.

During the 14-day at home treatment patients will be randomly assigned to apply the blinded product to the target lesion and surrounding areas twice per day (once in the morning and once in the evening). Patients will be instructed to apply the blinded products to the target lesion and surrounding area approximately one inch of normal skin extending beyond the lesion. The patients will return to the clinic on day 1, day 3, day 7, and day 14 for study measurements and clinical assessment.

After day 14, patients will be instructed to follow a 7-day regression period during which time the test products are not administered. The patients will return to the clinic on day 21 to assess durability, response to the treatments, and final measurements.

At baseline, patients will undergo a physical examination, vital signs check, and measurement of height and weight. At baseline, the target lesion and surrounding area will be identified, and the lesion and surrounding areas will be sampled for a microbiome baseline using sterile polyester-tipped swabs, baseline EASI (Eczema Area and Severity Score) and ADSI (Atopic Dermatitis Severity Index) scores, skin pH measurement, baseline TEWL (Trans Epidermal Water Loss), VAS (Visual Analogue Scale) measurements, skin surface conductance and QOL (Quality of Life) measurements.

Patients will administer the labeled lotion to the entire study lesion and approximately an inch of normal skin surrounding the lesion to treat the non-lesion skin with the lotion and will use a mild moisturizing body wash in place of normal body wash and/or soap during their usual cleaning routine during the 21-day trial period.

Patients will be instructed to apply the test products on all areas of the body affected by eczema or with eczema-prone skin, as directed (this includes consistently treating the clinically identified target lesion and non-lesion sites) twice per day for 14 days, followed by a 7-day regression durability period during which the test products are not used. Such application can include covering the areas of the body affected by eczema or with eczema-prone skin.

The Protocol formulation is a standard-of-care (SOC) product (Off-the-Shelf eczema relief lotion) amended with Activated Oil (AO) and water. The control has a similar base but is amended only with water. The off-the-shelf eczema relief lotion contains 2% colloidal oatmeal in addition to aloe barbadensis leaf juice, Butyrospermum parkii (Shea) butter extract, Ceramide NG, Palmitoyl hexapeptide-12, C10-C30 cholesterol/lanosterol esters and bisabolol. To prepare 400 g clinical sample prepare 110% of required amount to compensate for material lost during production and packaging:

### Protocol Formulation.

Heat and Homogenize:
35.0 g Oat EMO
105.0 g Flax FFA oil prepared using Step 1 of the EMO process
700 g Sterile Distilled Deionized Water

Add:
2660 g Off-the-Shelf Lotion

Homogenize
Total: 3500 g Prepared Lotion

Transfer exactly 90 g to clinical packing for blinding and randomization.

### Control Formulation:

Heat:
840 g sterile Distilled Deionized Water

Add:
2660 g Off-the-Shelf Lotion (pre-heated to 70° C.)

Homogenize
Total: 3500 g Prepared Lotion

Transfer exactly 90 g to clinical packing for blinding and randomization.

On days 1, 3, 7 and 14, the patients will be assessed at the target lesion and surrounding areas by sampling for microbiome baseline using sterile polyester-tipped swabs, EASI and ADSI scores, skin pH measurement, TWEL, VAS measurements, skin surface conductance and QOL measurements.

At day 21, the patients will undergo a physical examination, vital signs and weight check and will be assessed at the target lesion and surrounding areas by sampling for microbiome baseline using sterile polyester-tipped swabs, EASI and ADSI scores, skin pH measurement, TWEL, VAS measurements, skin surface conductance and QOL measurements.

Primary endpoints for the study will include: metagenomics analysis of the entire skin microbiome, including measurement of the relative abundance of S. aureus on the lesion sites and non-lesion (control) sites, measurement of microbiome alpha diversity by the Shannon Diversity Index at lesion and non-lesion sites, and absolute abundance of S. aureus, S. capitis, S. hominis, and S. epidermidis on lesion sites and non-lesion sites, including all measurements versus baseline measurement and between lesion and non-lesion sites.

Secondary endpoints for the study will include: disease severity as measured by difference in mean EASI and ADSI scores from baseline by the end of the 14-day treatment period and 7-day regression period, skin pH at the lesion site and adjacent non-lesion site by the end of the 14-day treatment period and 7-day regression period, skin barrier as measured by difference in TEWL from baseline at lesion site and adjacent non-lesion site by the 14-day treatment period, skin hydration by the difference in SKICON (Skin Conductance) from baseline at the lesion site and adjacent non-lesion site by the 14-day treatment period, Visual Analog Scale (VAS) by the difference in VAS from baseline at the lesion site and adjacent non-lesion site by the 14-day treatment period, and Quality of Life by the difference in QOL from baseline at the lesion site and adjacent non-lesion site by the 14-day treatment. It is expected, without being bound to theory that the Protocol Product will result in improved microbial diversity, reduced population density and relative abundance of S. aureus, increased population density of “healthy” microbial flora, and improved general skin condition.

### Example 22. Preservative Efficacy Testing

Five formulations were prepared with a nanoemulsion of the present disclosure in colloidal oatmeal according to ACv14 from Example 23 with the exception that the EMO and FFA oil were homogenized into the cream to form microemulsions with the preservative combinations added to the water phase before emulsification. Preservative Formulation 1 (Preserv. 1) included 0.5% Phenoxyethanol and 0.1% potassium sorbate at pH 5.4. Preservative Formulation 2 (Preserv. 2) included 0.5% Phenoxyethanol, 0.05% EDTA and 0.1% potassium sorbate at pH 5.4. Preservative Formulation 3 (Preserv. 3) included 0.5% phenoxyethanol, 0.055% ethylhexylglycerin and 0.1% potassium sorbate at pH 5.4. Preservative Formulation 4 included 0.75% phenoxyethanol and 0.1% potassium sorbate at pH 5.4. Preservative Formulation 5 included 1% phenoxyethanol and 0.1% potassium sorbate at pH 5.4. All formulations were prepared with distearyldimonium chloride (quaternary amine) and benzalkonium chloride (quaternary amine) removed with other ingredients reduced or removed to facilitate better nEMO action. The model cream upon which the formulations were based was a commercial, off-the-shelf eczema therapy cream.

Criteria A of the European Pharmacopeia from Table 5.1.3.-2 provides requirements for log-reduction in viable bacteria over a period of time (a 2-log reduction in viable bacteria in 2 days, 3 log reduction in 7 days, and No Increase in 28 days; and a 2-log reduction in viable fungi in 14 days and No Increase in 28 days) where the assay is conducted at 20-25 degrees Celsius with a maximum of 1% water addition while adding 10\hat{ }5 to 10\hat{ }6 bacteria to the formulation. Criteria B provides a 3-log reduction in bacteria in 14 days and No Increase in 28 days; 1 log reduction in Fungi in 14 days and No Increase in 28 days.

Table 54 below provides the results of the preservative evaluation over a few weeks.

TABLE 54
Log reduction of bacterial strains (starting bacteria at 10⁵-10⁶ CFUs/mL)

| | Preserv. | Preserv. | Preserv. | Preserv. | Preserv. |
|---|---|---|---|---|---|
| Bacteria | 1 | 2 | 3 | 4 | 5 |
| E. coli | 0.9 | 1.0 | 1.0 | 2.3 | >5 |
| (ATCC | | | | | |
| 8739) | | | | | |
| P. aeruginosa | 1.1 | 1.7 | 1.9 | 2.2 | >5 |
| (ATCC | | | | | |
| 9027) | | | | | |
| S. aureus | 1.8 | >5 | 2.0 | 2.0 | >5 |
| (ATCC | | | | | |
| 6538) | | | | | |

From these results, it is expected without being bound to theory that an acceptable preservative is phenoxyethanol and that a concentration between 0.5% and 1%, preferably 0.75% will yield a formulation with the desired antimicrobial properties.

### Example 23. Preparation of a Colloidal Oat Cream with Nanoemulsions

For formulations ACv10-15, nanoemulsions of the present disclosure were used.

To produce Acv10-15, the following procedure was followed:
1. Combine water soluble (Phase A) ingredients, heat to 70° C. Examples of water soluble ingredients: water, glycerin, panthenol, Avena sativa Oat Meal Extract, Phenoxyethanol, Potassium sorbate, xanthan gum.
2. Combine oil soluble (Phase B) ingredients, heat to 70° C. Examples of oil soluble ingredients: Petrolatum, Dimethicone, Steareth-2, Steareth-20, Cetyl alcohol, EMO/FFA*.
3. Add Phase B to Phase A, homogenize at 13,000 rpm to form emulsion. *EMO and FFA components can be omitted from Phase B and homogenized into the pre-formed Step 3 emulsion for better antibacterial results.
4. Add Citric Acid solution to adjust pH (5.4 preferred).
5. Combine sensitive ingredients (e.g. Colloidal Oatmeal, Ceramide NP), mixing to make uniform. Slowly add to Step 3 emulsion. Homogenize to make uniform.
6. Bottle and store in the dark.

ACv13 was prepared to contain Water, Glycerin, Panthenol, Avena sativa Oat Extract, Petrolatum, Dimethicone, 2% Oat EMO, 2% Flax FFA, 0.3% S-2, 0.3% S-20, Cetyl Alcohol, 1% Colloidal Oatmeal, 0.5% Phenoxyethanol, 0.01% Citric Acid, 0.1% Potassium Sorbate.

ACv15 was prepared to contain Water, Glycerin, Panthenol, Avena sativa Oat Extract, Petrolatum, Dimethicone, 4% Flax FFA, 0.1% Monolaurin, 0.3% S-20, Cetyl Alcohol, 1% Colloidal Oatmeal, 0.5% Phenoxyethanol, 0.01% Citric Acid, 0.1% Potassium Sorbate.

Other formulations included ACv10: Water, Glycerin, Panthenol, Avena Sativa Oat Extract, Petrolatum, Dimethicone, 2% Oat EMO, 2% Flax FFA, 0.3% S-20, Cetyl Alcohol, 2% Colloidal Oatmeal, 0.5% Phenoxyethanol, 0.01% Citric Acid, 0.1% Potassium Sorbate; ACv11: Water, Glycerin, Panthenol, Avena sativa Oat Extract, Petrolatum, Dimethicone, 2% Oat EMO, 2% Flax FFA, 0.3% S-2, 0.3% S-20, Cetyl Alcohol, 2% Colloidal Oatmeal, 0.5% Phenoxyethanol, 0.01% Citric Acid, 0.1% Potassium Sorbate; ACv12: Water, Glycerin, Panthenol, Avena sativa Oat Extract, Petrolatum, Dimethicone, 2% Oat EMO, 2% Flax FFA, 0.6% S-2, 0.3% S-20, Cetyl Alcohol, 2% Colloidal Oatmeal, 0.5% Phenoxyethanol, 0.01% Citric Acid, 0.1% Potassium Sorbate.

ACv14 was prepared to contain Water, Glycerin, Panthenol, Avena sativa Oat Extract, Petrolatum, Dimethicone, 4% Flax FFA, 0.1% S-2, 0.3% S-20, Cetyl Alcohol, 1% Colloidal Oatmeal, 0.5% Phenoxyethanol, 0.01% Citric Acid, 0.1% Potassium Sorbate, the formulation (oil phase) which is shown in part below (as relevant to HLB). ACv15 had the same oil phase.

TABLE 55
Exemplary compositional components and HLB values.

| | % of total | % of oil phase | Required HLB (rHLB) | rHLB of oil percentage |
|---|---|---|---|---|
| Petrolatum | 3% | 28.6 | 7 | 2.00 |
| Dimethicone | 1% | 9.5 | 5 | 0.48 |
| Oat EMO | 0% | 0.0 | 7 | 0.00 |
| Flaxseed FFA | 4% | 38.1 | 17 | 6.48 |
| Cetyl Alcohol | 2.50% | 23.8 | 15.5 | 3.69 |
| Total | 10.5% | | | 12.64 |

Exemplary HLB values are provided below. HLB is typically considered when planning oil-in-water cream formulations according to the requirements of the cream.

| | | | | |
|---|---|---|---|---|
| | HLB | 50/50 | Steareth-2/Steareth-20 | 10.1 |
| Steareth-2 | 4.9 | 45/55 | Steareth-2/Steareth-20 | 10.62 |
| Steareth-20 | 15.3 | 40/60 | Steareth-2/Steareth-20 | 11.14 |
| Glyceryl Stearate SE | 5.8 | 35/65 | Steareth-2/Steareth-20 | 11.66 |
| Glyceryl Steareate | 3.8 | 25/75 | Steareth-2/Steareth-20 | 12.7 |
| Glyceryl Laurate | 5.2 | 40/60 | Glyceryl Laurate/Steareth-20 | 11.26 |
| | | 35/65 | Glyceryl Laurate/Steareth-20 | 11.765 |
| | | 30/70 | Glyceryl Laurate/Steareth-20 | 12.27 |
| | | 25/75 | Glyceryl Laurate/Steareth-20 | 12.775 |

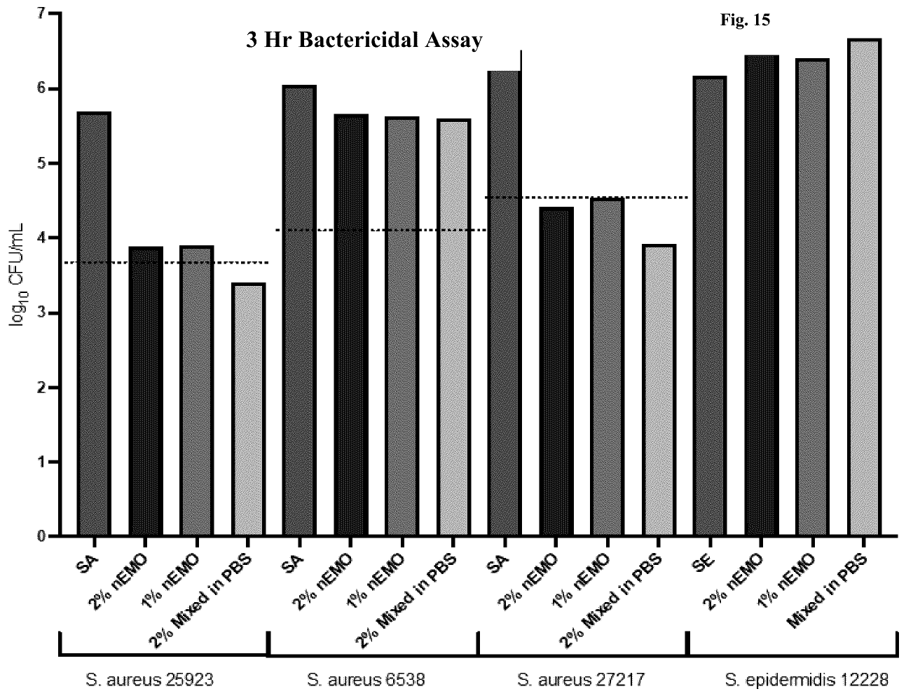

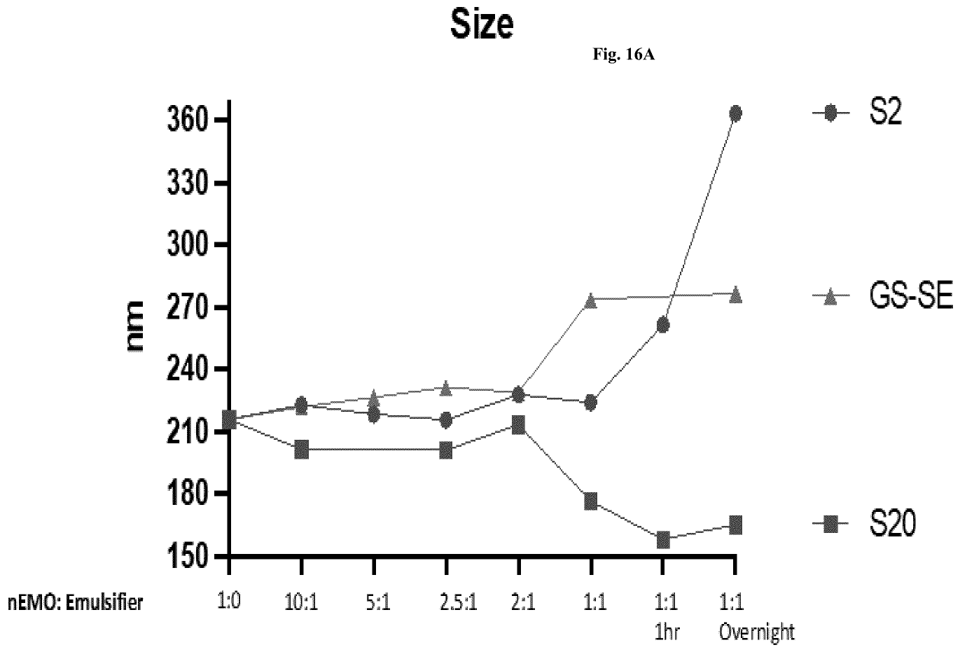

When centrifuged, the cream (ACv14) was stable. As shown in FIG. 15, S. aureus 25923 and 27217 were reduced by 2 logs in 3 hours, and S. epidermidis was unaffected. Performance against S. aureus 6538 was considered an outlier due to unique issues with this strain. 1% and 2% nEMO in the cream were statistically similar with 2% being slightly improved. The assay was the same as that used in Example 19 except for 3 hours instead of 5 hours.

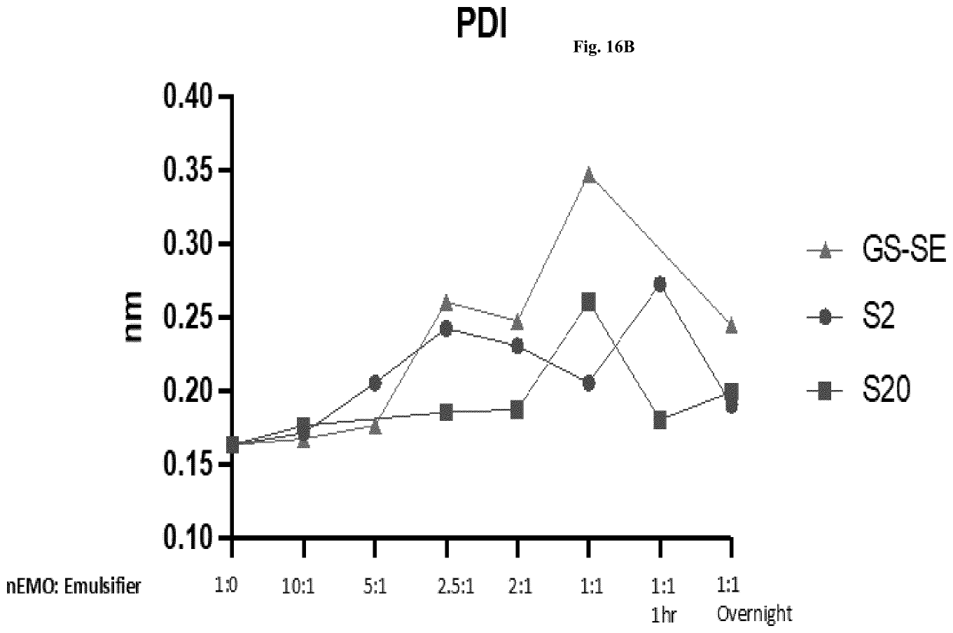

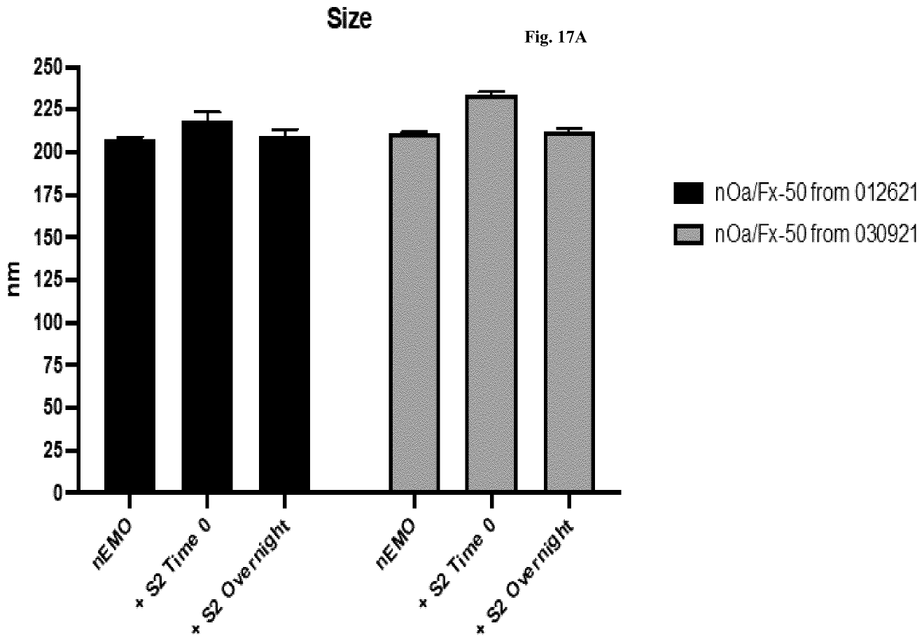

The effect of S-2, S-20 or Glyceryl Stearate incubated with nEMO nOA/Fx-25 (25% Oat MAG (EMO), 75% Flax FFA oil) were also evaluated at ratios of 1:0, 10:1, 5:1, 2.5:1, 2:1 or 1:1 (nEMO to emulsifier) and incubated for period of 1 hour or overnight and size and PDI measured by DLS as shown in FIGS. 16A-16B.

The most severe divergence from normal only occurred at the 1:1 ratio and at prolonged time periods. S-20 provided additional stability to the nEMO, lowering the size from 210 nm to 150 nm. GS-SE caused a moderate increase, while S-2 caused a near-doubling in size. PDI for all combinations fluctuated with an overall increasing trend at short time points and a reduction at the overnight time point.

If the Oat EMO is omitted and the Flax FFA oil raised, then a higher rHLB is required, meaning that a larger percentage of the emulsifier can be high HLB (S-20). This 0% Oat MAG (EMO) and 4% Flaxseed FFA oil combo gives an rHLB value of 12.64, which would coincide with a 25/75 ratio of S-2/S-20 or Monolaurin/S-20. This would minimize the amount of S-2 which caused larger coalescence at a 1:1 ratio overnight.

### Example 24. Use of Low HLB Emulsifiers

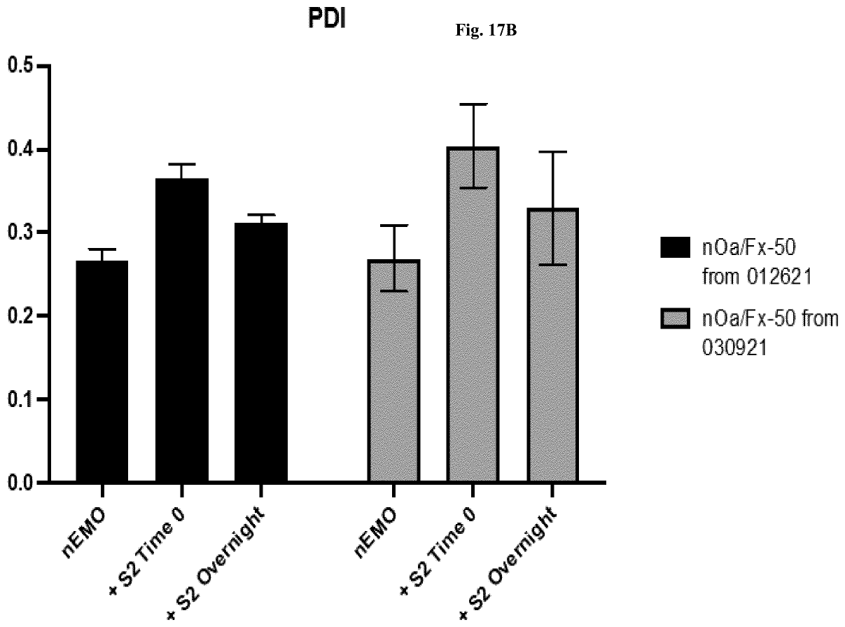

The preferred Anti-Staphylococcal nEMO nOa/Fx-25 (25% Oat MAG (EMO), 75% Flax FFA oil) has been observed to have some phase separation after 1 month of storage with oil bubbles floating on top of the emulsion, while the very similar nOa/Fx-50 (50% Oat MAG (EMO), 50% Flax FFA oil) did not show this after a time of 8+ months. There was only a modest increase in size from 170 nm to 210 nm during this time. A more robust nEMO could allow for an increased amount of S-2 emulsifier, thereby giving a more stable cream while keeping a stable and effective nEMO. A DLS experiment was performed by combining the nanoemulsions with the emulsifier at varying ratios and incubating together, using only S-2 and measuring right after addition and after overnight incubation. A slight increase in size at the initial addition time was then brought back to untreated size overnight as shown in FIGS. 17A-17B.

### Example 25. Bactericidal Effect of nOa/Fx-25 Against S. aureus (ACv14)

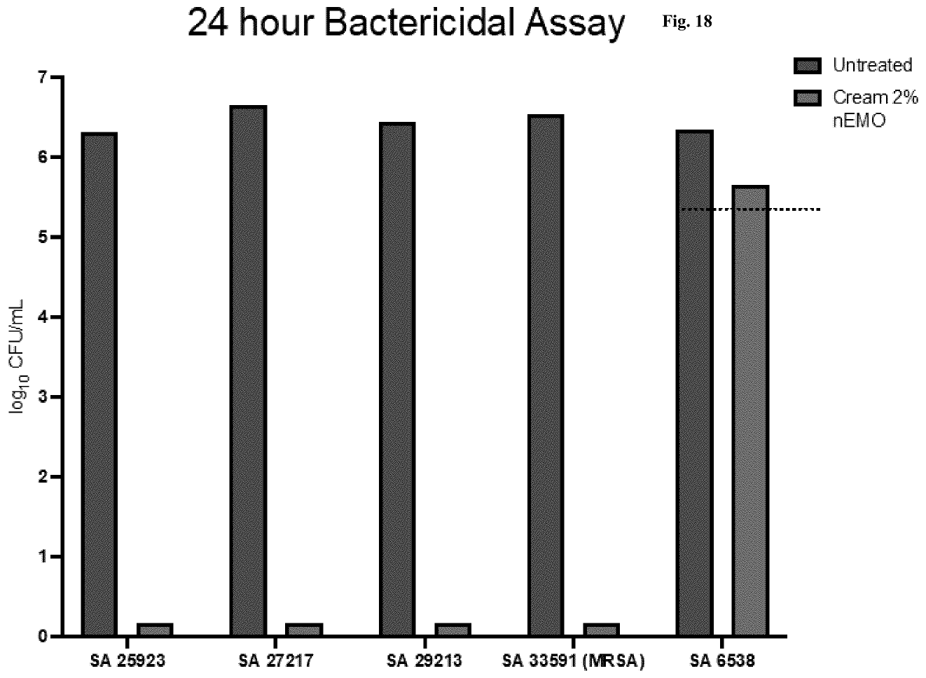

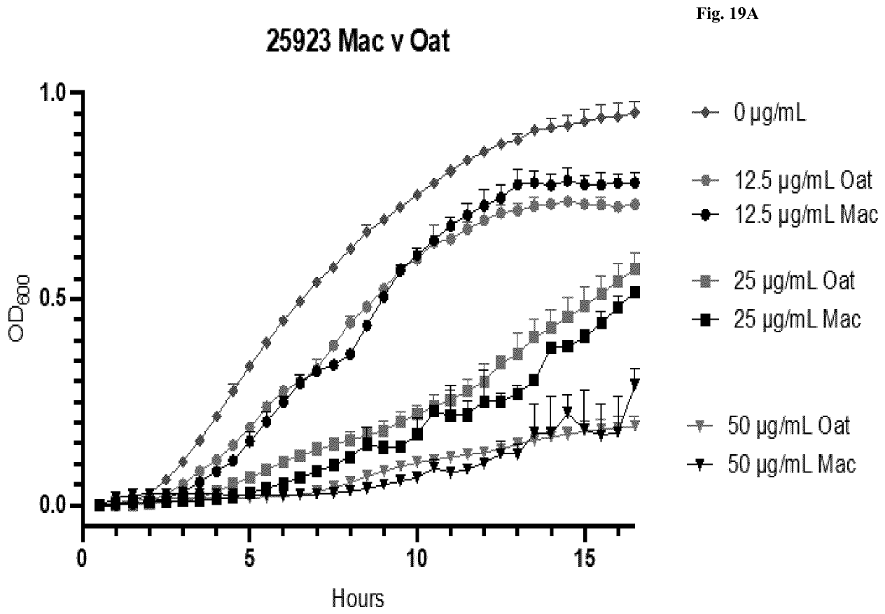

Bactericidal effect was measured against different S. aureus strains to establish S. aureus 6538 as an outlier. All experimental conditions were the same for the five shown strains in FIG. 18, using the culture tube-grown overnight suspension. They demonstrate a complete reduction in all bacteria over 24 hours with 2% nEMO-Cream of 25923, 27217, 29213, and 33591 strains, yet less than 1-log reduction in the 6538 strain.

### Example 26. Preparation of Macadamia Nut/Flaxseed Nanoemulsion

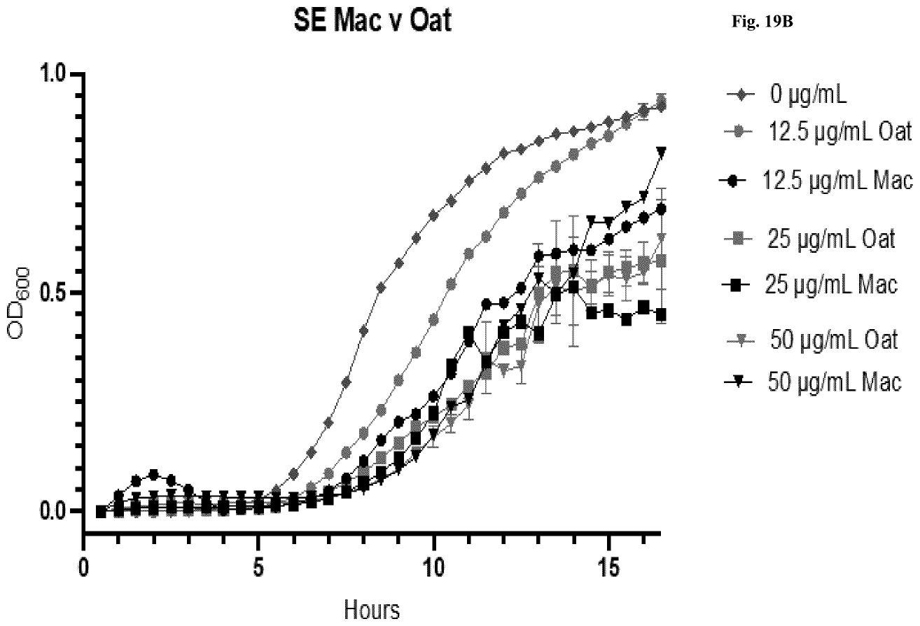

Macadamia nut oil was processed as described in previous examples to yield an enzyme modified oil with MAGs and FFA oil and was used to make nMac/Fx-25 (25% Mac MAG (EMO), 75% Flax FFA oil) and nMac/Fx-50 (50% Mac MAG (EMO), 50% Flax FFA oil). nOa/Fx (25 and 50) and nMac/Fx (25 and 50) were compared in 16 hr plate reader growth inhibition experiments using S. aureus 25923 and S. epidermidis ATCC 12228, with nOa/Fx-25 and nMac/Fx-25 giving superior results overall. FIGS. 19A-19B (which show nOa/Fx-25 and nMac/Fx-25) overlay those two results. It is notable that macadamia nut oil has 18% palmitoleic acid as opposed to typically 0-1% in other plant oils.

No significant difference was observed between the two formulations at several different concentrations, indicating that the Oat MAG could be substituted for the considerably less expensive Macadamia nut MAG.

### Example 27. Evaluation of the Order-of-Addition in Homogenization on a Microemulsified MAG/FFA Cream

For the following experiments, a new naming for each new cream formulation was established to distinguish them from previous formulations: “OL” refers to Oatmeal Lotion.

Each formula consists of: Water, Glycerin, Panthenol, Avena sativa Oat Extract, Petrolatum, Dimethicone, 1% Oat MAG (EMO), 3% Flax FFA oil, Cetyl Alcohol, 1% Colloidal Oatmeal, 0.5% Phenoxyethanol, 0.01% Citric Acid, 0.1% Potassium Sorbate, as well as the emulsifiers and additives listed in Table 55 (GS=glyceryl stearate; S-2=steareth-2; S-20=steareth-20).

TABLE 55
Summary of Formulations

| | OL1 | OL2 | OL3 | OL4 | OL5 |
|---|---|---|---|---|---|
| Emulsifiers | 0.2% S2 | 0.2% S2 | 0.2% S2 | 0.2% GS | 0.2% S2 |
| | 0.4% S20 | 0.4% 520 | 0.4% S20 | 0.4% 520 | 0.4% S20 |
| Additives | — | 0. 1% Ceramide NF | 0.1% Xanthan Gum | — | 0.1% Ceramide NE |
| | | | | | 0.1% Xanthan Gum |
| SA 25923 log reduction | 1.4 log | 0.78 log | 1.6 log | 1.7 log | — |
| w/o nEMO | | | | | |
| 3000 rpm, 40° C., 30 min | Unstable | Less Stable | Stable | Unstable | — |

The preparation of each OL involves adding all the normally heated Water phase A and Oil phase B ingredients for homogenization except the Oat MAG EMO and Flax FFA oil (both obtained by the enzymatic processes disclosed herein), forming a lotion, and then stirring in the Oat MAG (EMO)/Flax FFA oil and re-homogenizing the lotion. OL1 was the base formula for this strategy, with OL2, OL3 and OL4 modifying this. OL2 contains 0.1% Ceramide NP which was considered to be an essential ingredient for barrier/inflammation control. OL3 contains 0.1% Xanthan gum which is shown to stabilize oil/water emulsions as well as nanoemulsions. OL4 was investigated as a means of replacing S-2 with Glyceryl Stearate.

The addition of xanthan gum provided the greatest stability to a typical cosmetic industry stability test, which is the application of 3000 rpm centrifugation at 40° C. for 30 minutes. OL1 and OL4 separated very quickly, while OL2 experienced separation over the course of 30 minutes. OL3 did not indicate any separation. OL5 was then formulated as a way to combine the 0.1% Ceramide NP with 0.1% Xanthan Gum to give a more stabilized ceramide-containing lotion.

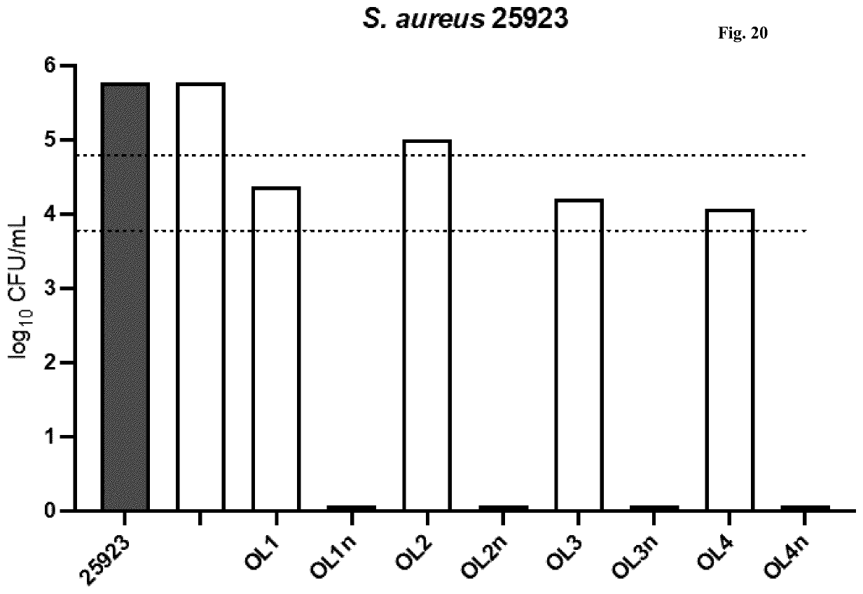

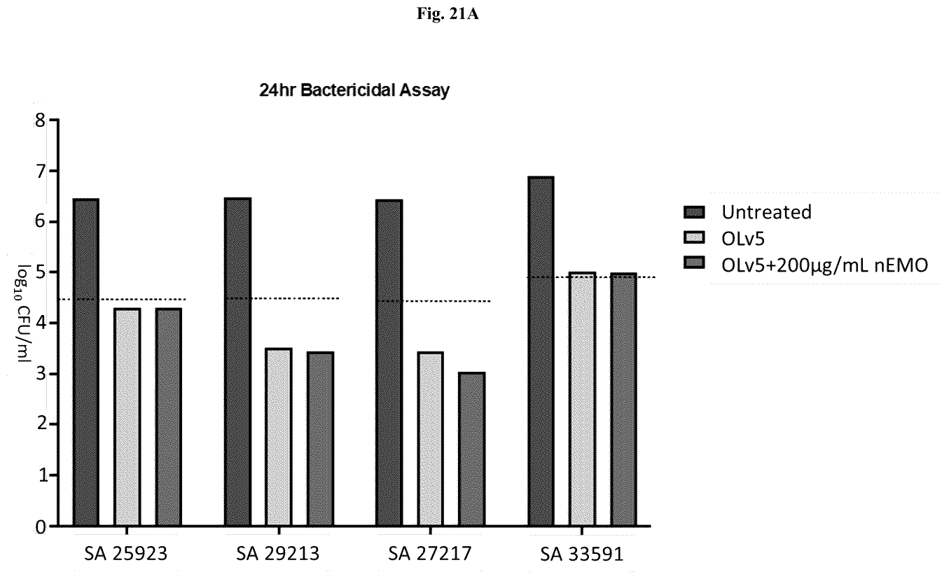

Table 55 also lists the performance of each OL against S. aureus 25923, in which antimicrobial results were obtained in a 3 hr bactericidal assay without the presence of nEMO. These results are shown in FIG. 20 compared to OL1 through OL4 with 800 μg/mL nEMO added.

It was found that pre-forming the lotion without the addition of MAG (EMO)/FFA oil, followed by addition and re-homogenization gives a lotion that has antibacterial action although not to the extent that is given when nEMO is added (formulations with suffix “n”). The addition of Ceramide NP reduces the bactericidal effect. The addition of xanthan gum greatly enhances the lotion stability to survive an industry standard stability test. A control off-the-shelf cream was not antibacterial in this assay (second column from the left).

### Example 28. Bactericidal Selectivity Assay

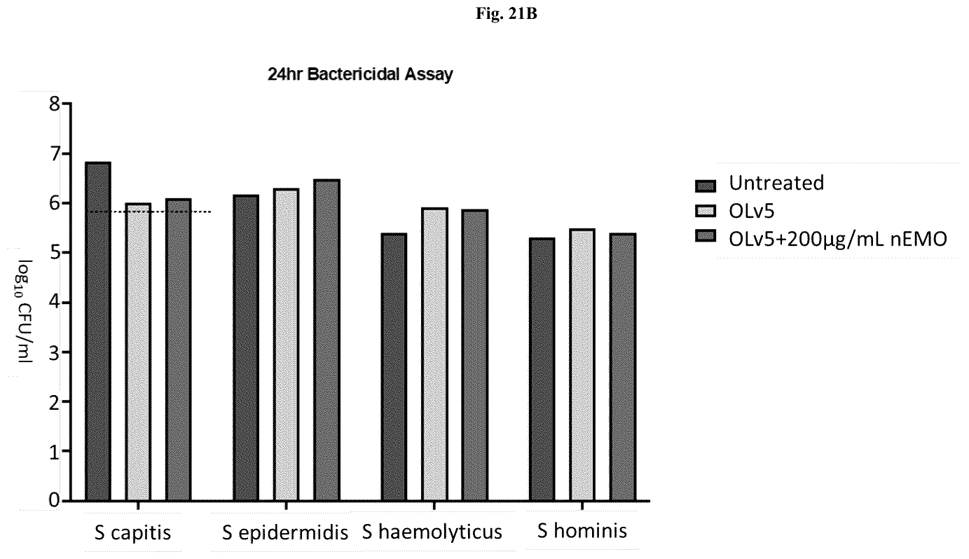

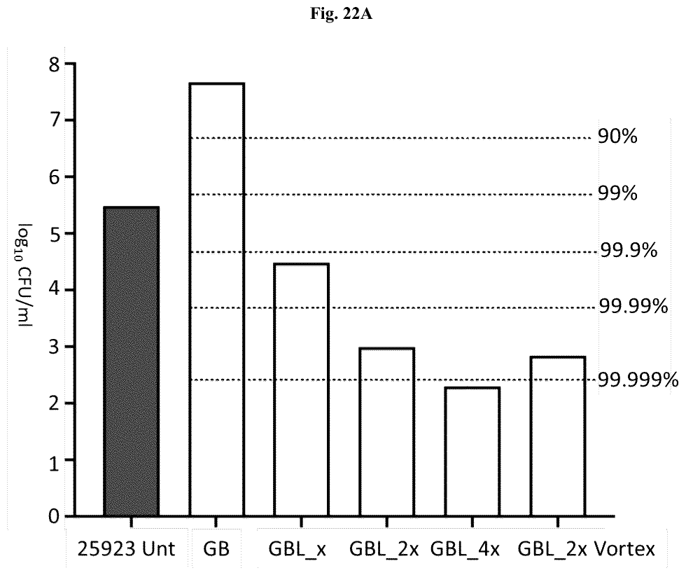

A full screen of our 4 major S. aureus strains and 4 major commensal Staphylococcus strains was conducted over a 24 hr incubation. OL5 was used and was also tested against the addition of a smaller quantity of nEMO, 200 μg/mL nOa/Fx-25 (25% Oat MAG (EMO), 75% Flax FFA oil), rather than the larger amount of 800 μg/mL as shown in FIGS. 21A-21B.

OL5 that had MAG/FFA homogenized in after lotion formation was able to reduce 4 strains of S. aureus by 2-3 logs over 24 hours and did not reduce any of the 4 commensal Staphylococcus strains by even a log after 24 hours. The addition of 200 μg/mL nOa/Fx-25 did not provide a significant benefit in this assay.

### Example 29. Incorporation of Enzyme-Modified MAG/FFA into a Commercial Off-the-Shelf Eczema Relief Cream/Lotion

The following experiments refer to creams/lotions (terms used interchangeably) in which the model is an off-the shelf eczema relief cream/lotion. The difference between the two commercially sold products, cream and lotion are unclear from a texture perspective, and may be a small adjustment for packaging purposes. The cream is in a squeezable tube while the lotion is in a pumpable bottle. The off-the-shelf eczema relief lotion contains 2% colloidal oatmeal in addition to aloe barbadensis leaf juice, Butyrospermum parkii (Shea) butter extract, Ceramide NG, Palmitoyl hexapeptide-12, C10-C30 cholesterol/lanosterol esters and bisabolol. The following new lotions were made by preheating the oil ingredients and water at 70° C., weighing all ingredients into a stainless-steel beaker, and then homogenizing at 10,000 rpm until fully incorporated.

Table 56 below provides the formulations that were prepared from MAGs obtained as enzyme-modified oils and FFA oils in previous examples.

TABLE 56
Formulations

| | Off-the-Shelf | Oat MAG | | |
|---|---|---|---|---|
| | Cream | EMO | Flax FFA oil | Water |
| GBL_x | 16 g | 0.2 g | 0.6 g | 16.8 g |
| GBL_2x | 16 g | 0.4 g | 1.2 g | 17.6 g |
| GBL_4x | 16 g | 0.8 g | 2.4 g | 19.2 g |

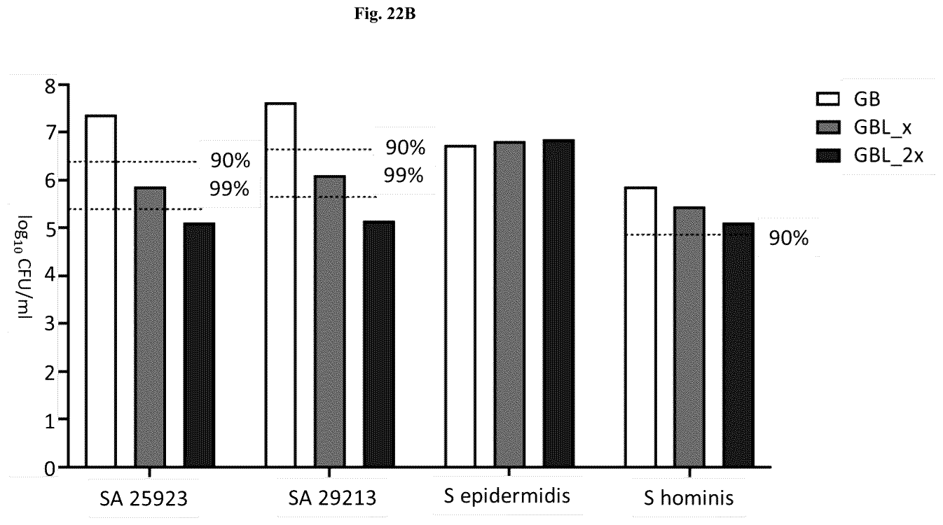

These formulations were tested against S. aureus 25923, in a 24 hr bactericidal assay as shown in FIG. 22A. The off-the-shelf cream was used as a control, and GBL_2x was also made as a vortexed mix rather than homogenization to compare the impact of homogenization.

The off-the-shelf treated bacteria actually increased in number by about 2 logs over 24 hours. GBL_x, GBL_2x, GBL_2x vortexed and GBL 4x all reduce the bacteria by 3-5 logs compared to the cell concentration in the ‘GB” sample (the “off-the-shelf” starting point). There was not a clear difference between the homogenized and vortexed GBL_2x, which indicates that the homogenization procedure is not detrimental to the formulation.

As shown in FIG. 22B, these formulations (GBL_x and GBL_2x) were further tested in a bactericidal assay against 2 S. aureus strains and commensal strains (S. epidermidis and S. hominis), the results of which are shown in the figure below. GBL_x reduced S. aureus CFUs by about 1.5 log, while the GBL_2x exceeded 2 logs. There was no decline in S. epidermidis and less than a log of decline in S. hominis.

### Example 30. Antiviral Assay Against Respiratory Syncitial Virus

Respiratory syncytial virus (RSV, A2) stock was prepared by growing virus in MA-104 cells. Test media used was MEM supplemented with 5% FBS and 50 μg/mL gentamicin.

nMCT/Fx-50 (50% MCT MAG (EMO)/50% flax FFA oil) and nOa/Fx-25, as previously described, were formulated as 100 mg/mL solutions. Samples were diluted in water to test concentrations of 50, 10, and 5 mg/mL. RSV virus stock was added to each prepared concentration in triplicate so that there was 10% virus solution by volume and 90% prepared sample. Media only was also added to prepared sample to serve as cytotoxicity controls. Ethanol was tested in parallel as a positive control and water only to serve as the virus control.

Compound and virus were incubated at 37° C. for a contact time of 2 minutes. Following the contact period, the solutions were neutralized by a 1/10 dilution in test media.

Surviving virus was quantified by standard end-point dilution assay. Neutralized samples were combined for quantification for the average of triplicate tests. Samples were serially diluted using eight 10-fold dilutions in test medium. Each dilution was added to 4 wells of a 96-well plate with 80-100% confluent cells. The toxicity controls were added to an additional 4 wells and 2 of these wells were infected with virus to serve as neutralization controls, ensuring that residual sample in the titer assay plated did not inhibit growth and detection of surviving virus.

Plates were incubated at 37±2° C. with 5% CO2. On day 5 post-infection plates were scored for presence or absence of viral cytopathic effect (CPE). The Reed-Muench method was used to determine end-point titers (50% cell culture infectious dose, CCID50) of the samples, and the log reduction value (LRV) of the compound compared to the negative (water) control was calculated.

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

Virus titer and log reduction value (LRV) for samples tested against RSV are shown in Tables 57A and 57B.

The virus control titer was 3.3 log CCID50 per 0.1 mL and this was used for comparison of all test sample titers to determine LRV. Samples with <1 log reduction are not considered active for virucidal activity. The limit of detection of virus for samples that did not exhibit cytotoxicity when plated for endpoint dilution assay was 0.7 log CCID50 per 0.1 mL.

When tested for a 2-minute contact time at 37° C., nMCT/Fx-50 exhibited virucidal activity against RSV, reducing virus titer below the limit of detection (LRV>2.6, >99%). nOa/Fx-25 also exhibited virucidal activity against RSV (LRV>1.6, >90%). Neutralization controls demonstrated that residual sample did not inhibit virus growth and detection in the endpoint titer assays in wells that did not have full cytotoxicity. Positive controls performed as expected.

Virus titer and log reduction value (LRV) for samples tested against RSV are shown in Table 57A (low concentrations) and 57B (follow up concentrations).

TABLE 57A
Virucidal activity against RSV after incubation with virus at 37 ± 2° C.

| | | | Contact | | Neut. | Virus | VC | |
|---|---|---|---|---|---|---|---|---|
| Compound | Concentration | | Time | Toxicity^{a} | Control^{b} | Titer^{c} | Titer^{c} | LRV^{d} |
| nMCT/Fx-50 | 1 | mg/mL | 2 min | None | None | 2.0 | 3.3 | 1.3 |
| nMCT/Fx-50 | 0.2 | mg/mL | 2 min | None | None | 2.5 | 3.3 | 0.8 |
| nMCT/Fx-50 | 0.04 | mg/mL | 2 min | None | None | 2.5 | 3.3 | 0.8 |
| nOa/Fx-25 | 1 | mg/mL | 2 min | None | None | 2.5 | 3.3 | 0.8 |
| nOa/Fx-25 | 0.2 | mg/mL | 2 min | None | None | 2.5 | 3.3 | 0.8 |
| nOa/Fx-25 | 1 | mg/mL | 2 min | None | None | 3.0 | 3.3 | 0.3 |

| | | | | | | | |
|---|---|---|---|---|---|---|---|
| Ethanol | 70% | 2 min | None | None | <0.7 | 3.3 | >2.6 |

TABLE 57B
Virucidal activity against RSV after incubation with virus at 37 ± 2° C.

| | | Contact | | Neut. | Virus | VC | |
|---|---|---|---|---|---|---|---|
| Compound | Concentration | Time | Toxicity^{a} | Control^{b} | Titer^{c} | Titer^{c} | LRV^{d} |
| nMCT/Fx-50 | 50 mg/mL | 2 min | 1/10 | None | <1.7 | 3.3 | >1.6 |
| nMCT/Fx-50 | 10 mg/mL | 2 min | 1/10 | None | <1.7 | 3.3 | >1.6 |
| nMCT/Fx-50 | 5 mg/mL | 2 min | None | None | <0.7 | 3.3 | >2.6 |
| nOa/Fx-25 | 50 mg/mL | 2 min | 1/100 | None | <2.7 | 3.3 | >0.6 |
| nOa/Fx-25 | 10 mg/mL | 2 min | 1/10 | None | <1.7 | 3.3 | >1.6 |
| nOa/Fx-25 | 5 mg/mL | 2 min | 1/10 | None | <1.7 | 3.3 | >1.6 |
| Ethanol | 70% | 2 min | None | None | <0.7 | 3.3 | >2.6 |

^{a}Cytotoxicity indicates the highest dilution of the endpoint titer where full (80-100%) cytotoxicity was observed
^{b}Neutralization control indicates the highest dilution of the endpoint titer where compound inhibited virus CPE in wells after neutralization
^{c}Virus titer of test sample or virus control (VC) in log10 CCID50 of virus per 0.1 mL
^{d}LRV (log reduction value) is the reduction of virus in test sample compared to the virus control

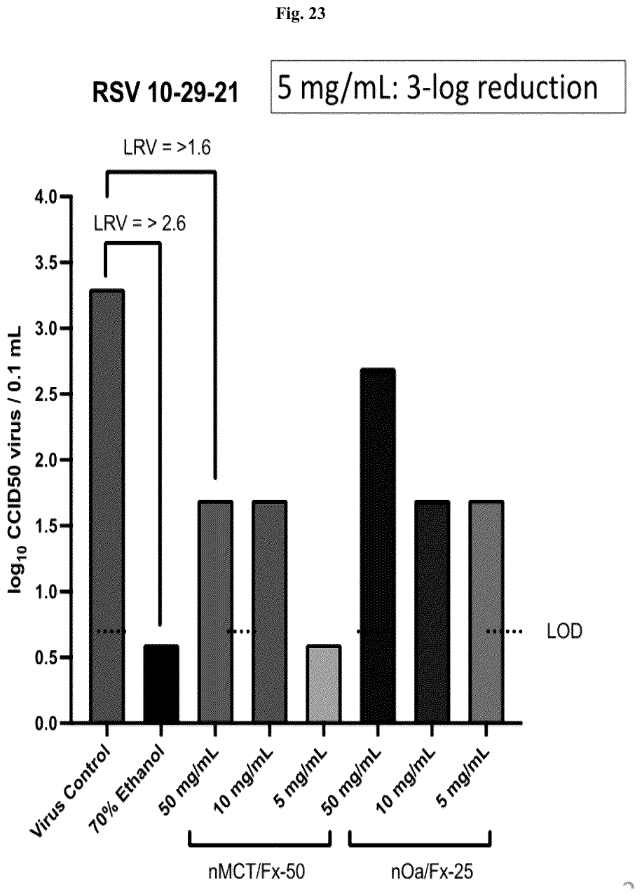

Resulting reduction in RSV is shown in FIG. 23.

### Example 31. Antiviral Assay Against Influenza Virus

Influenza A(H1N1)pdm09 (California/07/09) virus stock was prepared by growing virus in MDCK cells. Test media used was MEM supplemented with 10 U/mL trypsin, 1 μg/mL EDTA, and 50 μg/mL gentamicin.

Sample was prepared as a 100 mg/mL solution. Sample was diluted in water to test concentrations of 50, 10, and 5 mg/mL. Influenza A(H1N1)pdm09 virus stock was added to each prepared concentration in triplicate so that there was 10% virus solution by volume and 90% prepared sample. Media only was also added to prepared sample to serve as cytotoxicity controls. Ethanol was tested in parallel as a positive control and water only to serve as the virus control.

Compound and virus were incubated at 37° C. for a contact time of 2 minutes. Following the contact period, the solutions were neutralized by a 1/10 dilution in test media.

Surviving virus was quantified by standard end-point dilution assay. Neutralized samples were combined for quantification for the average of triplicate tests. Samples were serially diluted using eight 10-fold dilutions in test medium. Each dilution was added to 4 wells of a 96-well plate with 80-100% confluent Vero E6 cells. The toxicity controls were added to an additional 4 wells and 2 of these wells were infected with virus to serve as neutralization controls, ensuring that residual sample in the titer assay plated did not inhibit growth and detection of surviving virus.

Plates were incubated at 37±2° C. with 5% CO₂. On day 5 post-infection plates were scored for presence or absence of viral cytopathic effect (CPE). The Reed-Muench method was used to determine end-point titers (50% cell culture infectious dose, CCID50) of the samples, and the log reduction value (LRV) of the compound compared to the negative (water) control was calculated.

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

Virus titer and log reduction value (LRV) for samples tested against influenza A(H1N1)pdm09 are shown in Table 2B. The virus control titer was 5.7 log CCID50 per 0.1 mL and this was used for comparison of all test sample titers to determine LRV. Samples with <1 log reduction are not considered active for virucidal activity. The limit of detection of virus for samples that did not exhibit cytotoxicity when plated for endpoint dilution assay was 0.7 log CCID50 per 0.1 mL.

When tested for a 2-minute contact time at 37° C., nOa/Fx-25 exhibited virucidal activity against influenza A(H1N1)pdm09, reducing virus titer below the limit of detection (LRV>4.0, >99.99%). Neutralization controls demonstrated that residual sample did not inhibit virus growth and detection in the endpoint titer assays in wells that did not have full cytotoxicity. Positive controls performed as expected.

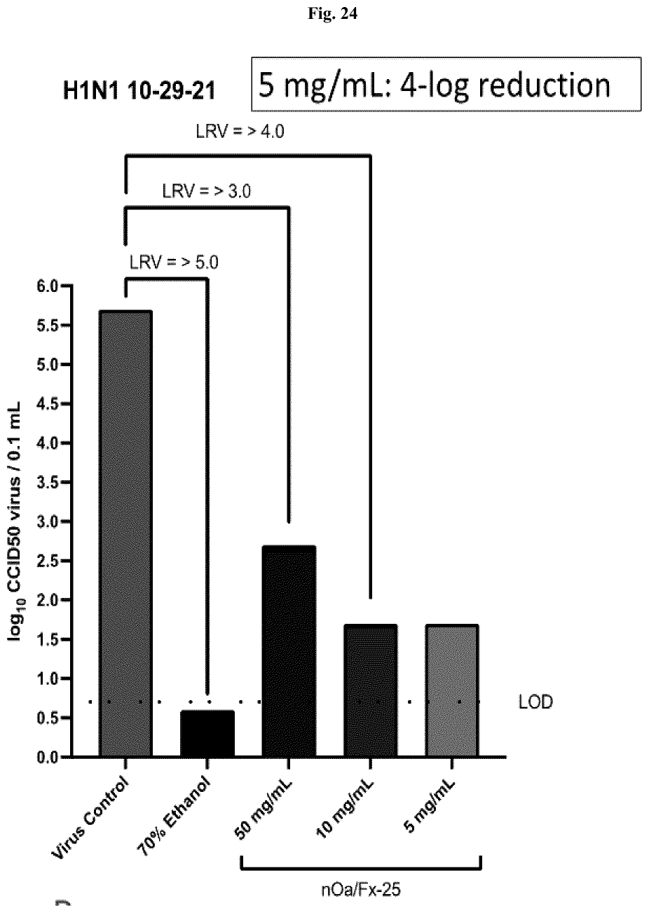

Virus titer and log reduction value (LRV) for samples tested against influenza A(H1N1)pdm09 are shown in Table 58A and 58B and FIG. 24.

TABLE 58A
Virucidal activity against influenza A(H1N1)pdm09 against H1N1
after incubation with virus at 37 ± 2° C.

| | | | Contact | | Neut. | Virus | VC | |
|---|---|---|---|---|---|---|---|---|
| Compound | Concentration | | Time | Toxicity^{a} | Control^{b} | Titer^{c} | Titer^{c} | LRV^{d} |
| nOa/Fx-25 | 1 | mg/mL | 2 min | None | None | 4.3 | 5.7 | 1.4 |
| nOa/Fx-25 | 0.2 | mg/mL | 2 min | None | None | 5.5 | 5.7 | 0.2 |
| nOa/Fx-25 | 0.04 | mg/mL | 2 min | None | None | 5.5 | 5.7 | 0.2 |

| | | | | | | | |
|---|---|---|---|---|---|---|---|
| Ethanol | 70% | 2 min | None | None | <0.7 | 5.7 | >5.0 |

TABLE 58B
Virucidal activity against influenza A(H1N1)pdm09 after
incubation with virus at 37 ± 2° C.

| | | Contact | | Neut. | Virus | VC | |
|---|---|---|---|---|---|---|---|
| Compound | Concentration | Time | Toxicity^{a} | Control^{b} | Titer^{c} | Titer^{c} | LRV^{d} |
| nOa/Fx-25 | 50 mg/mL | 2 min | 1/100 | None | <2.7 | 5.7 | >3.0 |
| nOa/Fx-25 | 10 mg/mL | 2 min | 1/10 | None | <1.7 | 5.7 | >4.0 |
| nOa/Fx-25 | 5 mg/mL | 2 min | 1/10 | None | <1.7 | 5.7 | >4.0 |
| Ethanol | 70% | 2 min | None | None | <0.7 | 5.7 | >5.0 |

^{a}Cytotoxicity indicates the highest dilution of the endpoint titer where full (80-100%) cytotoxicity was observed
^{b}Neutralization control indicates the highest dilution of the endpoint titer where compound inhibited virus CPE in wells after neutralization
^{c}Virus titer of test sample or virus control (VC) in log10 CCID50 of virus per 0.1 mL
^{d}LRV (log reduction value) is the reduction of virus in test sample compared to the virus control

### Example 32. Antiviral Assay Against SARS-CoV-2

SARS-CoV-2, USA_WA1/2020, virus stock was prepared by growing virus in Vero 76 cells. Test media used was MEM supplemented with 2% FBS and 50 μg/mL gentamicin.

nOa/Fx-25 was prepared as a 100 mg/mL solution. Sample was diluted in water to a test concentration of 50, 10, and 5 mg/mL. Virus stock was added to each prepared concentration in triplicate so that there was 10% virus solution by volume and 90% prepared sample. Media only was also added to prepared sample to serve as cytotoxicity controls.

Ethanol was tested in parallel as a positive control and water only to serve as the virus control.

Compound and virus were incubated at 37° C. for a contact time of 2 minutes. Following the contact period, the solutions were neutralized by a 1/10 dilution in test media.

Surviving virus was quantified by standard end-point dilution assay. Neutralized samples were combined for quantification for the average of triplicate tests. Samples were serially diluted using eight 10-fold dilutions in test medium. Each dilution was added to 4 wells of a 96-well plate with 80-100% confluent Vero E6 cells. The toxicity controls were added to an additional 4 wells and 2 of these wells were infected with virus to serve as neutralization controls, ensuring that residual sample in the titer assay plated did not inhibit growth and detection of surviving virus.

Plates were incubated at 37±2° C. with 5% CO2. On day 5 post-infection plates were scored for presence or absence of viral cytopathic effect (CPE). The Reed-Muench method was used to determine end-point titers (50% cell culture infectious dose, CCID50) of the samples, and the log reduction value (LRV) of the compound compared to the negative (water) control was calculated.

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

Virus titer and log reduction value (LRV) for samples tested against SARS-CoV-2 are shown in Table 59A (low concentrations) and 59B (follow up concentrations).

TABLE 59A
Virucidal activity against SARS-CoV-2 (USA_WA1/2020) after
incubation with virus at 37 ± 2° C.

| | | | Contact | | Neut. | Virus | VC | |
|---|---|---|---|---|---|---|---|---|
| Compound | Concentration | | Time | Toxicity^{a} | Control^{b} | Titer^{c} | Titer^{c} | LRV^{d} |
| nOa/Fx-25 | 1 | mg/mL | 2 min | None | None | 3.5 | 5.3 | 1.8 |
| nOa/Fx-25 | 0.2 | mg/mL | 2 min | None | None | 3.5 | 5.3 | 1.8 |
| nOa/Fx-25 | 0.04 | mg/mL | 2 min | None | None | 4.5 | 5.3 | 0.8 |

| | | | | | | | |
|---|---|---|---|---|---|---|---|
| Ethanol | 70% | 2 min | None | None | <0.7 | 5.3 | >4.6 |

TABLE 59B
Virucidal activity against SARS-CoV-2 after incubation with virus at 37 ± 2° C.

| | | Contact | | Neut. | Virus | VC | |
|---|---|---|---|---|---|---|---|
| Compound | Concentration | Time | Toxicity^{a} | Control^{b} | Titer^{c} | Titer^{c} | LRV^{d} |
| nOa/Fx-25 | 50 mg/mL | 2 min | 1/100 | None | <2.7 | 5.0 | >2.3 |
| nOa/Fx-25 | 10 mg/mL | 2 min | 1/10 | None | <1.7 | 5.0 | >3.3 |
| nOa/Fx-25 | 5 mg/mL | 2 min | 1/10 | None | 1.7 | 5.0 | 3.3 |
| Ethanol | 70% | 2 min | None | None | <0.7 | 5.0 | >4.3 |

^{a}Cytotoxicity indicates the highest dilution of the endpoint titer where full (80-100%) cytotoxicity was observed
^{b}Neutralization control indicates the highest dilution of the endpoint titer where compound inhibited virus CPE in wells after neutralization
^{c}Virus titer of test sample or virus control (VC) in log10 CCID50 of virus per 0.1 mL
^{d}LRV (log reduction value) is the reduction of virus in test sample compared to the virus control

### Example 33. Antiviral Assay Against HSV-1

Herpes simplex-1 (HSV-1, H1KOS S1) virus stock was prepared by growing virus in Vero 76 cells. Test media used was MEM supplemented with 2% FBS and 50 μg/mL gentamicin.

nOa/Fx-25 was prepared as a 100 mg/mL solution. Sample was diluted in water to a test concentration of 50, 10, and 5 mg/mL. HSV-1 virus stock was added to each prepared concentration in triplicate so that there was 10% virus solution by volume and 90% prepared sample. Media only was also added to prepared sample to serve as cytotoxicity controls. Ethanol was tested in parallel as a positive control and water only to serve as the virus control.

Compound and virus were incubated at 37° C. for a contact time of 2 minutes. Following the contact period, the solutions were neutralized by a 1/10 dilution in test media.

Surviving virus was quantified by standard end-point dilution assay. Neutralized samples were combined for quantification for the average of triplicate tests. Samples were serially diluted using eight 10-fold dilutions in test medium. Each dilution was added to 4 wells of a 96-well plate with 80-100% confluent cells. The toxicity controls were added to an additional 4 wells and 2 of these wells were infected with virus to serve as neutralization controls, ensuring that residual sample in the titer assay plated did not inhibit growth and detection of surviving virus.

Plates were incubated at 37±2° C. with 5% CO2. On day 5 post-infection plates were scored for presence or absence of viral cytopathic effect (CPE). The Reed-Muench method was used to determine end-point titers (50% cell culture infectious dose, CCID50) of the samples, and the log reduction value (LRV) of the compound compared to the negative (water) control was calculated.

Virus controls were tested in water and the reduction of virus in test wells compared to virus controls was calculated as the log reduction value (LRV). Toxicity controls were tested with media not containing virus to see if the samples were toxic to cells. Neutralization controls were tested to ensure that virus inactivation did not continue after the specified contact time, and that residual sample in the titer assay plates did not inhibit growth and detection of surviving virus. This was done by adding toxicity samples to titer test plates then spiking each well with a low amount of virus that would produce an observable amount of CPE during the incubation period.

Virus titer and log reduction value (LRV) for samples tested against HSV-1 are shown in Table 60A and 60B.

The virus control titer was 5.0 log CCID50 per 0.1 mL and this was used for comparison of all test sample titers to determine LRV. Samples with <1 log reduction are not considered active for virucidal activity. The limit of detection of virus for samples that did not exhibit cytotoxicity when plated for endpoint dilution assay was 0.7 log CCID50 per 0.1 mL.

nOa/Fx-25 exhibited virucidal activity against HSV-1, reducing virus titer below the limit of detection (LRV>3.3, >99.9%).

Neutralization controls demonstrated that residual sample did not inhibit virus growth and detection in the endpoint titer assays in wells that did not have full cytotoxicity. Positive controls performed as expected. Herpes simplex-1 (HSV-1, H1KOS S1) virus stock was prepared by growing virus in Vero 76 cells. Test media used was MEM supplemented with 2% FBS and 50 μg/mL gentamicin.

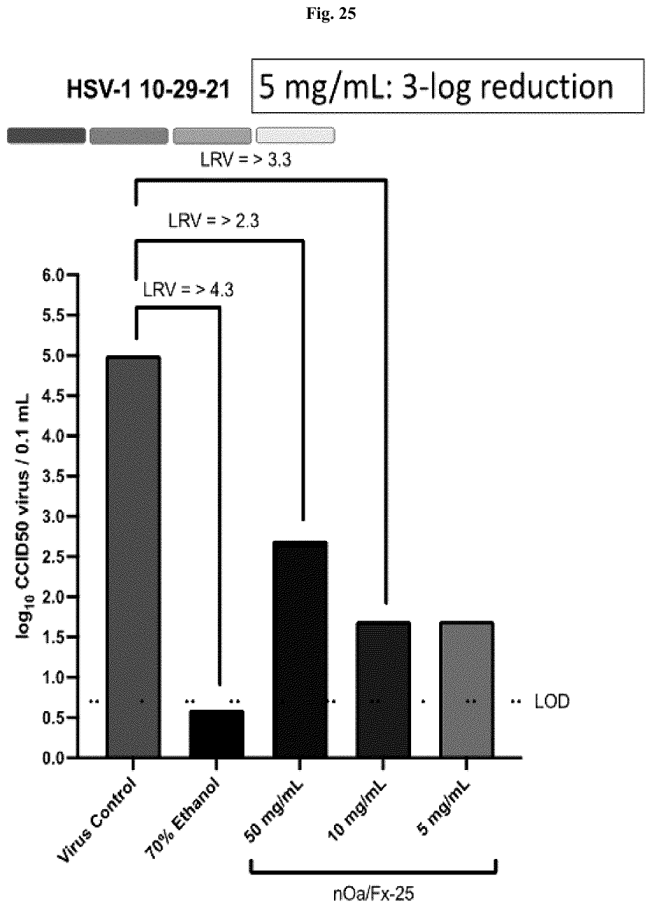

Virus titer and log reduction value (LRV) for samples tested against HSV-1 are shown in Table 60A (low concentrations) and 60B (follow up concentrations) and FIG. 25.

TABLE 60A
Virucidal activity against HSV-1 after incubation with virus at 37 ± 2° C.

| | | | Contact | Toxicity | Neut. | Virus | VC | |
|---|---|---|---|---|---|---|---|---|
| Compound | Concentration | | Time | a | Control^{b} | Titer^{c} | Titer^{c} | LRV^{d} |
| nOa/Fx-25 | 1 | mg/mL | 2 min | None | None | 1.7 | 4.5 | 2.8 |
| nOa/Fx-25 | 0.2 | mg/mL | 2 min | None | None | 4.0 | 4.5 | 0.5 |
| nOa/Fx-25 | 0.04 | mg/ml | 2 min | None | None | 4.5 | 4.5 | 0 |

| | | | | | | | |
|---|---|---|---|---|---|---|---|
| Ethanol | 70% | 2 min | None | None | <0.7 | 4.5 | >3.8 |

TABLE 60B
Virucidal activity against HSV-1 after incubation with virus at 37 ± 2° C.

| | | Contact | | Neut. | Virus | VC | |
|---|---|---|---|---|---|---|---|
| Compound | Concentration | Time | Toxicity^{a} | Control^{b} | Titer^{c} | Titer^{c} | LRV^{d} |
| nOa/Fx-25 | 50 mg/mL | 2 min | 1/100 | None | <2.7 | 5.0 | >2.3 |
| nOa/Fx-25 | 10 mg/mL | 2 min | 1/10 | None | <1.7 | 5.0 | >3.3 |
| nOa/Fx-25 | 5 mg/mL | 2 min | 1/10 | None | <1.7 | 5.0 | >3.3 |
| Ethanol | 70% | 2 min | None | None | <0.7 | 5.0 | >4.3 |

^{a}Cytotoxicity indicates the highest dilution of the endpoint titer where full (80-100%) cytotoxicity was observed
^{b}Neutralization control indicates the highest dilution of the endpoint titer where compound inhibited virus CPE in wells after neutralization
^{c}Virus titer of test sample or virus control (VC) in log10 CCID50 of virus per 0.1 mL
^{d}LRV (log reduction value) is the reduction of virus in test sample compared to the virus control

Follow-up experiments using 70% ethanol and a control, showed that nanoemulsions of the present disclosure demonstrated excellent virucidal activity (at amounts of 0.5 to 50 mg/mL) as shown in Table 61.

TABLE 61
Virucidal Activity

| Virus | Control* | nEMO | EtOH |
|---|---|---|---|
| RSV | 3.3 | >90% | >99% |
| H1N1 | 5.7 | >99.99% | >99.99% |
| SARS-COV-2 | 5 | >99.9% | >99.99% |
| HSV | 5 | >99.9% | >99.99% |

*Log10 of initial virus concentration.Initial

### Example 34. Evaluation of S. aureus 6538

There are experimental conditions in which our nEMO-containing creams are effective against all measured S. aureus strains, such as the growth inhibition (bacteriostatic) assays, where nEMOs in a very diluted cream are very effective against all tested strains. The limitation of this assay is that increased concentration of cream interferes with the OD600 reading, giving too much background absorbance. However, in the 3-hr bactericidal assay strain 6538 stands out as an exception.
ATCC 25923: common QC strain
ATCC 6538: common QC strain, used for preservative testing
ATCC 29213: common QC strain
ATCC 27217: tetracycline-resistant, non-invasive on skin
ATCC 33591: methicillin-resistant (MRSA), used in testing antimicrobial handwashing formulations

An observation was made that overnight S. aureus cultures shaken at 200 rpm at 37° C. in Mueller-Hinton Broth typically produce a homogenous suspension, while S. aureus 6538 formed a non-homogenous suspension with a sedimentary pellet (prior to any centrifugation or settling time). 6538 is exceptionally known for its biofilm-formation, which could play a role in reduced sensitivity to lipid-caused disruption through extracellular structure building.

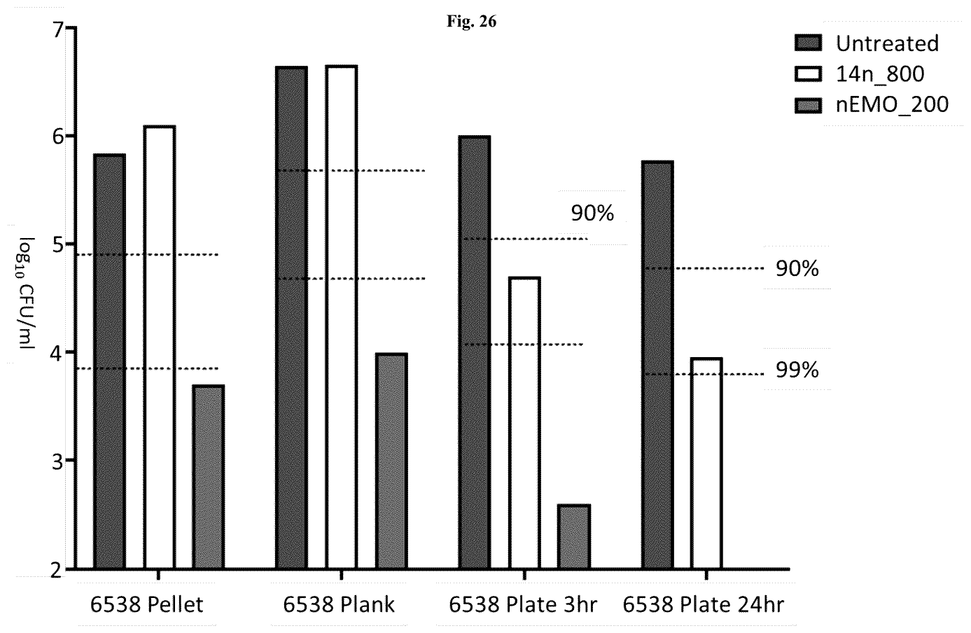

FIG. 26 compares the separated Planktonic culture growth (Plank), the Sedimentary culture growth (Pellet), and PBS-Suspended Plate-grown colonies (Plate) against ACv14 containing 800μg/mL nOa/Fx-25 (grey bars) and just the nOa/Fx-25 nEMO at 200 μg/mL (blue bars).

The first three sets of data shown are at the 3 hr time point, with the 4^{th} being extended to 24 hr. What this demonstrated was that the overnight culture in Mueller-Hinton Broth gave way to two populations of bacteria that were both equally resistant to nEMO-Cream, while neither was resistant to nEMO by itself. In contrast, the PBS-suspended Plate-grown colonies (Plate) were much more susceptible to nEMO-Cream which gave a >1-log reduction in 3 hr and gave a nearly 2-log reduction at 24 hr. The nEMO itself was very potent at both time points against the plated-isolated colonies.

### Example 35. Assessment of Antimicrobial Effect on Isolated Bacteria Versus Cultured Bacteria

OL5, as described previously, contains 79.5% water, 5% glycerin, 2% panthenol, 0.5% Avena sativa oat extract, 3% petrolatum, 1% dimethicone, 1% oat EMO, 3% flaxseed FFA oil, 2.5% cetyl alcohol, 1% colloidal oatmeal, 0.5% phenoxyethanol, 0.01% citric acid, 0.1% potassium sorbate, 0.2% steareth-2, 0.4% steareth-20, 0.1% ceramide NP, and 0.1% xanthan gum.

S. aureus is known to change its membrane fatty acid composition in response to the broth or serum it is grown in. S. aureus can use an oleate hydratase to reduce unsaturated fatty acids containing cis-9 double bonds, such as those found in palmitoleic acid.

Two isolates were obtained, one from PathEx, who specializes in capturing bacterial pathogens from blood to prevent sepsis, and the other from a nasal swab of a volunteer participant at GlycosBio.

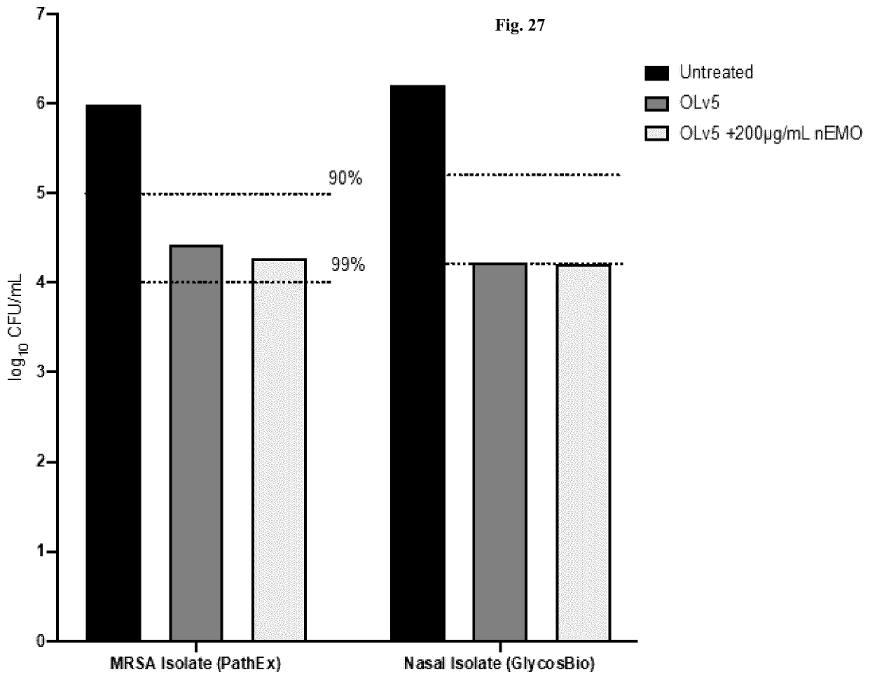

A standard bactericidal assay was performed and the results are shown in FIG. 27. Minimum Bactericidal Concentration (MBC) assay. A single colony from an agar plate of bacteria was used to inoculate 5 mL of nutrient-rich broth (generally Mueller-Hinton for Staphylococcus, Tryptic Soy for Streptococcus, based on ATCC recommendations) in a culture tube, and was shaken at 200 rpm at 37° C. overnight. The OD600 was measured using a spectrophotometer, the culture tube was pelleted at 4,000×g for 5 minutes, and the broth was removed. The bacterial pellet was resuspended in PBS pH 7.4 to a CFU/mL concentration that is 10× the desired CFU/mL for the reaction. In a separate culture tube the antibacterial compound (nEMO, lotion, etc) is placed into the tube and diluted with 4.5 mL PBS. 500 μL of the 10× bacterial stock is then added, vortexed, and placed in a 37° C. incubator with shaking at 200 rpm. Reaction time varies from 5 minutes to 24 hours, depending on the timing required. Reactions were then diluted 1×10\hat{ }1-1×10\hat{ }6 with PBS. 100 μL of each dilution was added to a Tryptic Soy Agar plate with 5% defibrinated sheep blood plate and spread with a T-shape spreader. Plates were incubated for 24 hours at 37° C. and CFUs were determined by counting plates that fell in between standard TNTC (too numerous to count) or TFTC (too few to count) range, typically 20-150 CFUs, and multiplying by the dilution factor of the counted plate to determine the undiluted concentration. C. acnes and other anaerobic bacteria were exclusively grown and treated in an anerobic GasPak pouch, and bacteria requiring 5% CO2 were treated in CO2-enriched GasPak pouches. OL5 (OLv5) was used as well as OLv5 spiked with 200 μg/mL nOa/Fx-25, with untreated bacteria as the control. As shown in FIG. 27, in the standard 5 hour timeframe, the MRSA isolate from PathEx was reduced nearly 2-logs with slight improvement from the addition of nEMO, while the nasal isolate from the volunteer participant had identical 2-log reductions from the OLv5 lotion with or without nEMO supplementation. This further supports the broad effectiveness of EMOs and FFA oils through 4 commercial strains (ATCC25923, ATCC29213, ATCC27217, and ATCC33591) and 2 isolated strains.

### Example 36. Optimization of Off-the-Shelf Eczema Relief Lotion

The off-the-shelf eczema relief lotion used in Example 29 was used for the following experimental procedures (labeled as Commercial Lotion in the Table 62 below, used in all further examples where an off-the-shelf eczema relief lotion is referenced). EMO and FFA oil were homogenized into the lotion and the control sample was diluted with 24% water and homogenized. Table 62 below shows a few of the combinations that narrowed down the constraints of homogenizing AO and water into the off-the-shelf lotion. Lotions that were immediately discarded are not shown, as well as those that contained additional additives such as xanthan gum, or those tried with other lotions. Two of the entries, 110921.1 and 110921.2 used the macadamia nut MAG oil (EMO) instead of Oat, to see if this could be substituted in the future to reduce the cost of procuring the rather expensive oat oil. Ratios are expressed to the EMO and FFA oil combined.

TABLE 62
Off-the-shelf Eczema Relief Lotion Formulations

| | Commercial | | Flax | | | | |
|---|---|---|---|---|---|---|---|
| | Lotion (CL) | | FFA | H2O | | | |
| Exp | (g) | Oat EMO (g) | oil (g) | (g) | H2O/EMO | CL/H2O | CL/EMO |
| 102521.1 | 16.0 | 0.2 | 0.6 | 16.8 | 21 to 1 | 1 to 1 | 20 to 1 |
| 102521.2 | 16.0 | 0.4 | 1.2 | 17.6 | 11 to 1 | 0.9 to 1 | 10 to 1 |
| 102521.3 | 16.0 | 0.8 | 2.4 | 19.2 | 6 to 1 | 0.8 to 1 | 5 to 1 |
| 102921.1 | 39.2 | 0.2 | 0.6 | 0 | — | — | 49 to 1 |
| 102921.2 | 38.4 | 0.4 | 1.2 | 0 | — | — | 24 to 1 |
| 102921.3 | 38.4 | 0.4 | 1.2 | 6.4 | 4 to 1 | 6 to 1 | 24 to 1 |
| 110821.1 | 38.4 | 0.46 | 1.37 | 6.4 | 3.5 to 1 | 6 to 1 | 21 to 1 |
| 110821.2 | 38.4 | 0.53 | 1.6 | 6.4 | 3 to 1 | 6 to 1 | 18 to 1 |
| 110821.3 | 38.4 | 0.4 | 1.2 | 3.84 | 2.4 to 1 | 10 to 1 | 24 to 1 |
| 110821.4 | 38.4 | 0.46 | 1.37 | 3.84 | 2.1 to 1 | 10 to 1 | 21 to 1 |
| 110821.5 | 38.4 | 0.53 | 1.6 | 3.84 | 1.8 to 1 | 10 to 1 | 18 to 1 |
| 110821.2 | 38.4 | 0.53 | 1.6 | 3.84 | 1.8 to 1 | 10 to 1 | 18 to 1 |
| 110821.5 | 38.4 | 0.53 | 1.6 | 6.4 | 3 to 1 | 6 to 1 | 18 to 1 |
| 110921.1 | 38.4 | 0.53 | 1.6 | 6.4 | 3 to 1 | 6 to 1 | 18 to 1 |
| | | (maca- | | | | | |
| | | damia) | | | | | |
| 110921.2 | 38.4 | 0.53 | 1.6 | 3.84 | 1.8 to 1 | 10 to 1 | 18 to 1 |
| | | (maca- | | | | | |
| | | damia) | | | | | |
| 111521.1 | 38.4 | 0.64 | 1.92 | 3.84 | 1.5 to 1 | 10 to 1 | 15 to 1 |
| 111521.2 | 38.4 | 0.64 | 1.92 | 9.6 | 3.8 to 1 | 4 to 1 | 15 to 1 |

Standard bactericidal assays were performed which obtained the following results: (1) CL/EMO ratios of greater than 15 to 1 resulted in suboptimal bactericidal action, while lower than 15 to 1 resulted in instability in the formulation, visible the next day by a layer of unincorporated oil on top of the lotion; (2) the first three entries in which either a large excess of water was used resulted in lotions that were very physically flowy and very bactericidal but diluted the colloidal oat and preservatives to an undesired level; (3) the next two entries with no water were difficult to mix, the oil did not readily incorporate, and thus no bactericidal action was observed; (4) most of the following entries had too little EMO or too little water yielding physically stable lotions that were insufficiently bactericidal; (5) no significant differences were observed between oat EMO and macadamia EMO; and (6) the last entry 111521.2, contained a 15:1 CL:EMO ratio and a higher water content of 4:1 CL/H2O, which provided the best physical stability and best bactericidal action out of similarly assembled formulations. This last entry corresponds to 5% EMO and FFA oil (1.25% EMO, 3.75% FFA oil) and 19% water. Minimum Bactericidal Concentration (MBC) assay. A single colony from an agar plate of bacteria was used to inoculate 5 mL of nutrient-rich broth (generally Mueller-Hinton for Staphylococcus, Tryptic Soy for Streptococcus, based on ATCC recommendations) in a culture tube, and was shaken at 200 rpm at 37° C. overnight. The OD600 was measured using a spectrophotometer, the culture tube was pelleted at 4,000×g for 5 minutes, and the broth was removed. The bacterial pellet was resuspended in PBS pH 7.4 to a CFU/mL concentration that is 10× the desired CFU/mL for the reaction. In a separate culture tube the antibacterial compound (nEMO, lotion, etc) is placed into the tube and diluted with 4.5 mL PBS. 500 μL of the 10× bacterial stock is then added, vortexed, and placed in a 37° C. incubator with shaking at 200 rpm. Reaction time varies from 5 minutes to 24 hours, depending on the timing required. Reactions were then diluted 1×10\hat{ }1-1×10\hat{ }6 with PBS. 100 μL of each dilution was added to a Tryptic Soy Agar plate with 5% defibrinated sheep blood plate and spread with a T-shape spreader. Plates were incubated for 24 hours at 37° C. and CFUs were determined by counting plates that fell in between standard TNTC (too numerous to count) or TFTC (too few to count) range, typically 20-150 CFUs, and multiplying by the dilution factor of the counted plate to determine the undiluted concentration. C. acnes and other anaerobic bacteria were exclusively grown and treated in an anerobic GasPak pouch, and bacteria requiring 5% CO2 were treated in CO2-enriched GasPak pouches.

### Example 37. Co-Cultured Bactericidal Assay with Repeat Exposure

This experiment was performed to assess whether a bacterial tolerance would develop in which recovery of S. aureus in a complex mixture of species would occur after repeated exposure to the EMO/FFA oil-supplemented off-the-shelf eczema relief lotion of Example 29.

Roughly equal amounts (based on OD₆₀₀) of S. aureus ATCC 29213, S. epidermidis ATCC 12228, and S. hominis ATCC 27844 were treated in combination in 5 mL of PBS alone, with the off-the-shelf eczema relief lotion of Example 29, or with the EMO/FFA oil-supplemented off-the-shelf eczema relief lotion (111521.2 from Table 62). All incubation steps were conducted at 37° C. with shaking at 200 rpm. After 24 hours, samples were taken for plating, the reaction tubes were centrifuged at 4000×g for 4 minutes to pellet the bacteria and the supernatant, which contains the lotion, was decanted. Fresh PBS was added, and the off-the-shelf eczema relief lotion and EMO/FFA oil-supplemented off-the-shelf eczema relief lotion were added to the previously treated tubes. This process was repeated at 48 hours, and a third sample was taken at 72 hours.

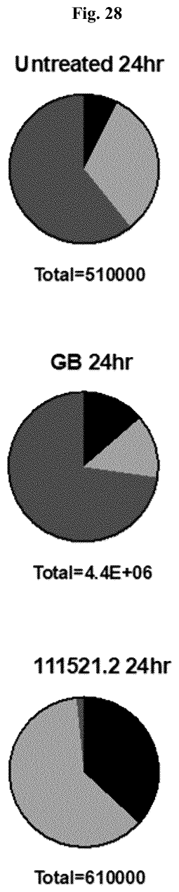

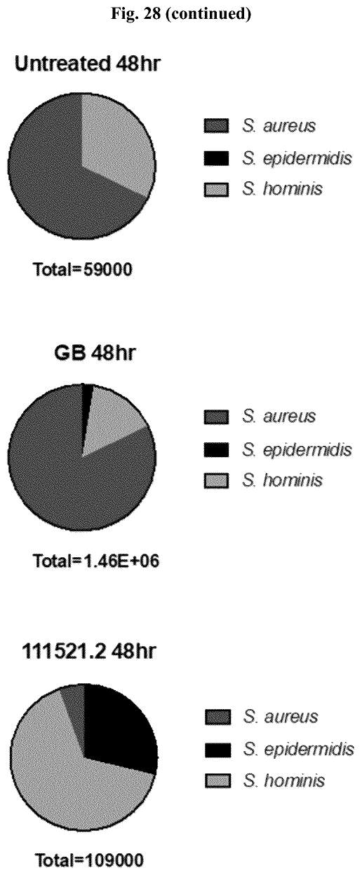

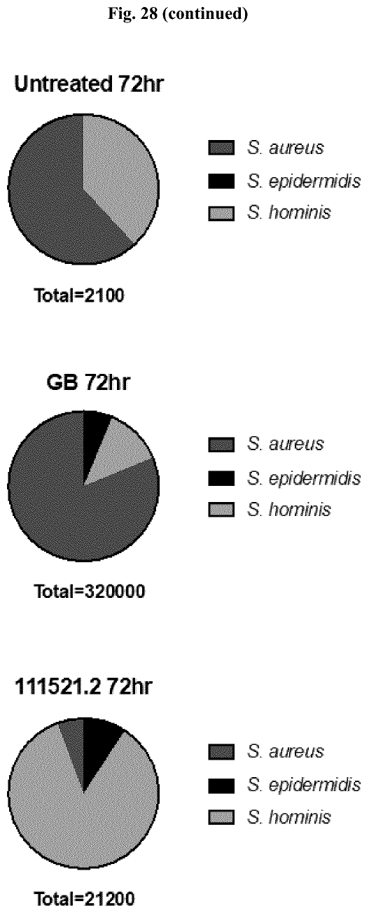

FIG. 28 shows the bacterial species composition for each sample and the total number of all species detected by plate counting for each experiment n cfu/mL. The untreated (PBS only), GB (off-the-shelf eczema relief lotion) and 111521.2 samples showed different effects on the bacterial mixture.

The untreated bacterial mixture yielded previously seen results where S. aureus preferentially grows in the presence of S. epidermidis and S. hominis, however the overall abundance went from 5×10\hat{ }5 (510,000) CFU/mL to 6×10\hat{ }4 to 2×10\hat{ }3 over the 24, 48, and 72 hr time frames, roughly a loss of a log unit per day.

As previously observed, the GB (unsupplemented off-the-shelf eczema relief lotion) treated samples experienced a growth in overall bacteria, and S. aureus grows the best in this nutrient and carbohydrate rich environment. The representation of species did not significantly change at the 48 and 72 hr times, and the overall abundance went from 4×10\hat{ }6 to 3×10\hat{ }5, about a half-log loss per day.

The 111521.2 treated samples had a near-total loss of S. aureus at 24 hr, which did not change at 48 and 72 hrs. As with the other samples, S. hominis survived better overall after prolonged incubation in PBS, becoming the majority of the population when not in competition with S. aureus. Total bacteria went from 6×10\hat{ }5 to 1×10\hat{ }5 to 2×10\hat{ }4 CFU/mL, a slower reduction than the untreated sample.

These results demonstrate both that S. aureus doesn't build resistance to the EMO/FFA oil-supplemented lotion, and that the nutrient-supplying formulation of of the off-the-shelf eczema relief lotion extends to the EMO/FFA oil-supplemented lotion with the added benefit of eliminating the S. aureus population.

### Example 38. Preservative Challenge Testing of Diluted Off-the-Shelf Eczema Relief Lotion

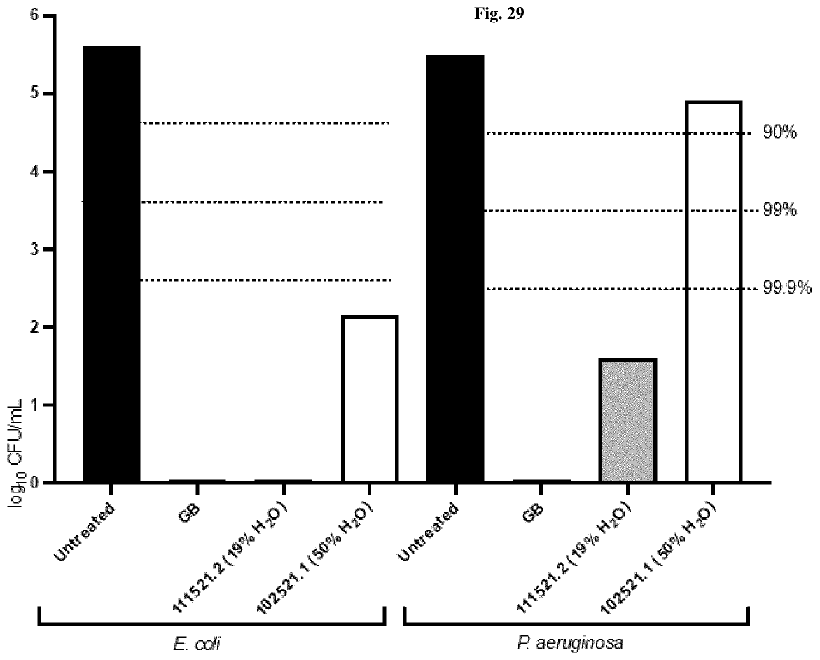

To assess whether diluting the off-the-shelf eczema relief lotion preservatives affects their effectiveness, the EMO/FFA oil-supplemented off-the-shelf eczema relief lotion formulations were challenged with two Gram negative bacteria: E. coli and P. aeruginosa to evaluate the effectiveness in a 2-day exposure with 10\hat{ }6 CFU/mL as shown in FIG. 29 (GB=off-the-shelf eczema relief lotion).

This was done following the European Phamacopoeia 5.1.3 Efficacy of Antimicrobial Preservation but does not include S. aureus (which is known to be strongly affected by the EMO) or fungi which are not being tested since the 2-day point is the critical gatekeeper for passing the test. Final formulations will undergo traditional 2-week PET testing.

In contrast to the bactericidal assays in which lotions are diluted in PBS before addition of bacteria, this test involves directly inoculating lotions with the bacteria at no more than 1% liquid addition. The preservatives in the off-the-shelf eczema relief lotion include: propanediol, ethylhexylglycerin, EDTA, phenoxyethanol, and methylparaben.

Unmodified off-the-shelf eczema relief lotion completely kills both bacteria added while the EMO/FFA oil-supplemented lotion that is 19% diluted with water and 5% diluted with EMO/FFA oil (111521.2) completely kills E. coli and nearly completely reduces P. aeruginosa by 4 logs. EMO/FFA oil-supplemented lotion that is diluted 50% with water and 8% with EMO (102521.1) kills E. coli by more than 3 logs, but P. aeruginosa is nearly unaffected by this formulation. This is a critical reason to avoid diluting the off-the-shelf eczema relief lotion too much, which could render it useless towards Pseudomonas contamination. Minimum Bactericidal Concentration (MBC) assay. A single colony from an agar plate of bacteria was used to inoculate 5 mL of nutrient-rich broth (generally Mueller-Hinton for Staphylococcus, Tryptic Soy for Streptococcus, based on ATCC recommendations) in a culture tube, and was shaken at 200 rpm at 37° C. overnight. The OD600 was measured using a spectrophotometer, the culture tube was pelleted at 4,000×g for 5 minutes, and the broth was removed. The bacterial pellet was resuspended in PBS pH 7.4 to a CFU/mL concentration that is 10× the desired CFU/mL for the reaction. In a separate culture tube the antibacterial compound (nEMO, lotion, etc) is placed into the tube and diluted with 4.5 mL PBS. 500 μL of the 10× bacterial stock is then added, vortexed, and placed in a 37° C. incubator with shaking at 200 rpm. Reaction time varies from 5 minutes to 24 hours, depending on the timing required. Reactions were then diluted 1×10\hat{ }1-1×10\hat{ }6 with PBS. 100 μL of each dilution was added to a Tryptic Soy Agar plate with 5% defibrinated sheep blood plate and spread with a T-shape spreader. Plates were incubated for 24 hours at 37° C. and CFUs were determined by counting plates that fell in between standard TNTC (too numerous to count) or TFTC (too few to count) range, typically 20-150 CFUs, and multiplying by the dilution factor of the counted plate to determine the undiluted concentration. C. acnes and other anaerobic bacteria were exclusively grown and treated in an anerobic GasPak pouch, and bacteria requiring 5% CO2 were treated in CO2-enriched GasPak pouches.

### Example 39. Antimicrobial Efficacy Test of EMO/FFA Oil-Supplemented Off-the-Shelf Eczema Relief Lotions at Extended Time Points

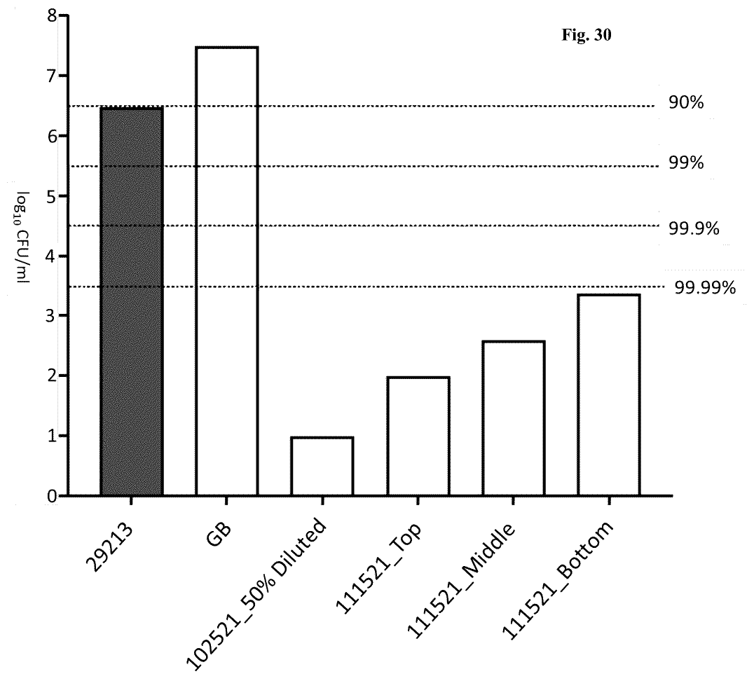

At a 10-week timepoint post-formulation the 19% water, 5% EMO formulation was subjected to a quality control evaluation for anti-S. aureus activity. Using a metal spatula, one-third of the material was scooped off and placed in a separated tube labeled “top,” followed by the middle section and the bottom section. The consistently potent formulation made on Oct. 25, 2021 (50% water, 10% EMO) was used as a comparator, and S. aureus strain ATCC 29213 was grown and treated in the typical bactericidal experiment for 24 h. As shown in FIG. 30, unmodified off-the-shelf eczema relief lotion increased bacterial growth 1-log over untreated, and all formulated-treated experiments resulted in exceptional potency. However, more than a log of activity separated the Top (most potent) fraction from the bottom (least potent).

### Example 40. Pre-Clinical Study In Vivo Assessment of EMO/FFA Oil-Supplemented Off-the-Shelf Eczema Relief Lotion

Trial 1

4 subjects, male, ages 35-65 were screened for normal skin on forearms. They were instructed to refrain from using antibacterial soaps and medicated lotions the night before the experiment, and to wear short sleeves and bring a loose-fitting jacket if needed.

Test materials are as shown in Tables 63-64 below.

TABLE 63
Test Formulations (GB = off-the-shelf eczema relief lotion)

| Name | Base | H2O | Oat EMO | Flax FFA |
|---|---|---|---|---|
| GB | 38.4 g (75.9%) | 12.2 g (24.1%) | — | — |
| GB+(2.5%) | 39.7 g (78.5%) | 9.6 g (19%) | 0.32 g (0.6%) | 0.96 g |
| | | | | (1.9%) |
| GB+(5%) | 38.4 g (75.9%) | 9.6 g (19%) | 0.64 g (1.3%) | 1.92 g |
| | | | | (3.8%) |
| GB+(10%) | 30.5 g (60.3%) | 15.0 g (29.6%) | 1.28 g (2.5%) | 3.84 g |
| | | | | (7.6%) |

TABLE 64
Test bacteria.

| Species | Strain | Traits | Sensitivity | CFU/mL |
|---|---|---|---|---|
| S. aureus | ATCC | Non-invasive | Penicillin, 50 μg | 10\hat{ }4 |
| | 27217 | | Tetracycline | |
| S. | ATCC | Non-biofilm | Vancomycin | 10\hat{ }4 |
| epidermidis | 12228 | forming | | |

Participants were free to shower the night before and were asked to not use medicated products or hand sanitizers on their arms. No participants were taking antibiotics at the time. Three circular test sites per arm were marked using a pen by tracing a 25 mm Hill Top Chamber, evenly spaced along the midsection of each forearm. Wrist and elbow areas were avoided. In plastic weigh boats 150 mg of each test lotion was weighed, and participants rubbed the material into the designated test site using a different gloved finger for each lotion. 10 minutes were permitted for the lotion to dissolve/evaporate, and then each site was inoculated with a pre-mixed 50/50 mixture of S. aureus ATCC 27217 and S. epidermidis ATCC 12228, which were grown overnight in Mueller-Hinton broth, pelleted, resuspended in PBS pH 7.4, and diluted to OD600's that would yield a plated CFU/mL of ˜1×10\hat{ }4, as determined by previously conducted plate counts. 10 μL of this mixture was pipetted into the center of the circled area, and spread using a sterile inoculating loop, within the center of the circle, avoiding the pen mark. Each site was immediately covered with a new 25 mm Hill Top Chamber without the absorbent pad and was secured to the site with a strip of Durapore Surgical Medical Tape (3M). Participants were allowed to continue daily activities for 3 hours, with caution of not disrupting the taped site.

After the three-hour period, the chambers were removed, and the sawed-off center of a 50 mL conical tube was used as an extraction chamber and pressed against the skin firmly. 1 mL of the extraction solution (75 mM sodium phosphate buffer pH 7.9, 0.1% Triton X-100, autoclaved) was pipetted into the extraction chamber, and the skin was rubbed for 10 seconds using a rubber policeman, and the solution from each site was pipetted off into a separate 2.0 mL microcentrifuge tube. Samples were 10× diluted in PBS, plated on Tryptic Soy Agar plates with 5% defibrinated sheep blood, and incubated for 24 hours at 37° C.

The table below provides the % recovery and % S. aureus in relation to the total count of S. aureus and S. epidermidis in each sample.

TABLE 65
Bacterial Harvesting Data.

| | Untreated | | GB | | GB+(2.5%) | | GB+(5%) | | GB+(10%) | |
|---|---|---|---|---|---|---|---|---|---|---|
| Partic- | % | % | % | % | % | % | % | % | % | % |
| ipant | Rec. | SA | Rec. | SA | Rec. | SA | Rec. | SA | Rec. | SA |
| 1 | 8 | 49 | 100 | 78 | 61 | 64 | 0 | 0 | 2 | 0 |
| 2 | 22 | 36 | 18 | 55 | 0 | 0 | 0 | 0 | 8 | 4 |
| 3 | 56 | 55 | 93 | 82 | 1 | 0 | 10 | 3 | 6 | 0 |
| 4 | 25 | 73 | 36 | 67 | 3 | 0 | 2 | 0 | 1 | 50 |
| Average | 28 | 53 | 62 | 71 | 16 | 16 | 3 | 1 | 4 | 14 |

In this initial experiment a few clear results stood out, despite a few high and low values that highly influenced the averages. This is an inherent limitation of a study in which a dynamic skin environment is being used on very different types of participants and not restricting movement and daily activities during the three-hour course.

The % S. aureus retrieved from the Untreated and off-the-shelf (GB) treated controls are in line with in vitro experimental models, where a slight increase in viable S. aureus occurs. The % recovery and % S. aureus from the three GB+(EMO/FFA oil-supplemented) treated samples were all significantly lower, with data points from single individuals preventing the % SA values from being essentially zero.

Trial 2

The materials and procedure were similar to Trial 1, except only GB, GB+(2.5%) and GB+(5.0%) were used, the order of addition was different on the right arm of participants, and only 5 μL inoculation volumes were applied.

5 μL of the inoculate mixture was applied to the center of the marked circles on the right forearm, and all evenly spread within the circles using a single sterile inoculating loop. The right arm samples were held horizontally flat and facing up for 15 minutes while the liquid evaporated. At the 10-minute mark, a separate volunteer treated the three left arm circles with GB, GB+(2.5%) or GB+(5.0%) using a gloved finger and left to dissolve/evaporate for 10 minutes. At the 15-minute mark the right arm inoculation sites were treated with GB, GB+(2.5%) or GB+(5.0%) and left to dissolve/evaporate for 10 minutes. At the 20-minute mark the left arm sites were inoculated with 5 μL of the bacterial mixture, spread using separate sterile inoculating loops for each sample, covered with a Hill Top Chamber, and secured with a strip of surgical tape. At the 25-minute mark the right arm sites were covered with a Hill Top Chamber and secured with a strip of surgical tape. After 3 hours the Bacterial Harvesting step was conducted identically to that described above.

TABLE 66
Procedure.

| Time | Action |
|---|---|
| 0 min | Inoculate Right sites, keep flat |
| 10 min | Apply lotion to Left sites |
| 15 min | Apply lotion to Right sites |
| 20 min | Inoculate Left sites, cover & secure |
| 25 min | Cover and secure Right sites |
| 3 hours | Bacterial Harvesting |

TABLE 67
Bacterial Harvesting Data.

| | a) Lotion-then-Bacteria | | | | | |
|---|---|---|---|---|---|---|
| | GB | | GB+(2.5%) | | GB+(5%) | |
| | % Re- | % S. | % Re- | % S. | % Re- | % S. |
| Participant | covered | aureus | covered | aureus | covered | aureus |
| 1 | 6 | 37 | 4 | 0 | 0 | 0 |
| 2 | 10 | 44 | 0 | 0 | 0 | 0 |
| 3 | 13 | 38 | 20 | 1 | 0 | 0 |
| 4 | 5 | 17 | 55 | 19 | 55 | 15 |
| 5 | 100 | 40 | 74 | 3 | 2 | 0 |
| Average | 27 | 34 | 31 | 5 | 11 | 3 |
| | a) Bacteria-then-Lotion | | | | | |
| | Gold Bond | | GB+(2.5%) | | GB+(5%) | |
| | % Re- | % S. | % Re- | % S. | % Re- | % S. |
| Participant | covered | aureus | covered | aureus | covered | aureus |
| 1 | 1 | 83 | 21 | 5 | 30 | 1 |
| 2 | 0 | 100 | 0 | 0 | 0 | 0 |
| 3 | 0 | 0 | 2 | 0 | 0 | 0 |
| 4 | 0 | 0 | 5 | 36 | 14 | 6 |
| 5 | 2 | 55 | 19 | 0 | 4 | 0 |
| Average | 1 | 46 | 9 | 8 | 10 | 1 |

Following these results, it was evident that overall recovery was significantly decreased in the Bacteria-then-Lotion (BTL) experiment, particularly with the off-the-shelf (GB) control where almost no recovery occurred. This is possibly due to the preservatives in the off-the-shelf product having direct contact with the bacteria from above, rather than the Lotion-then-Bacteria (LTB) experiment where the preservatives have time to be diluted in skin's water content and disperse. Percent recovery of S. aureus was fortunately very low once again in the GB+(2.5%) and GB+(5.0%) samples, and a clear distinction in the LTB experiment can be made between the % Recovered values between GB+(2.5%) and GB+(5.0%). The 31% total bacteria recovered in the 2.5% formulation more closely resembles the control, while the 11% in the 5.0% formulation represents a significant decrease in overall abundance and serves as a caution towards using this high of an Activated Oil concentration.

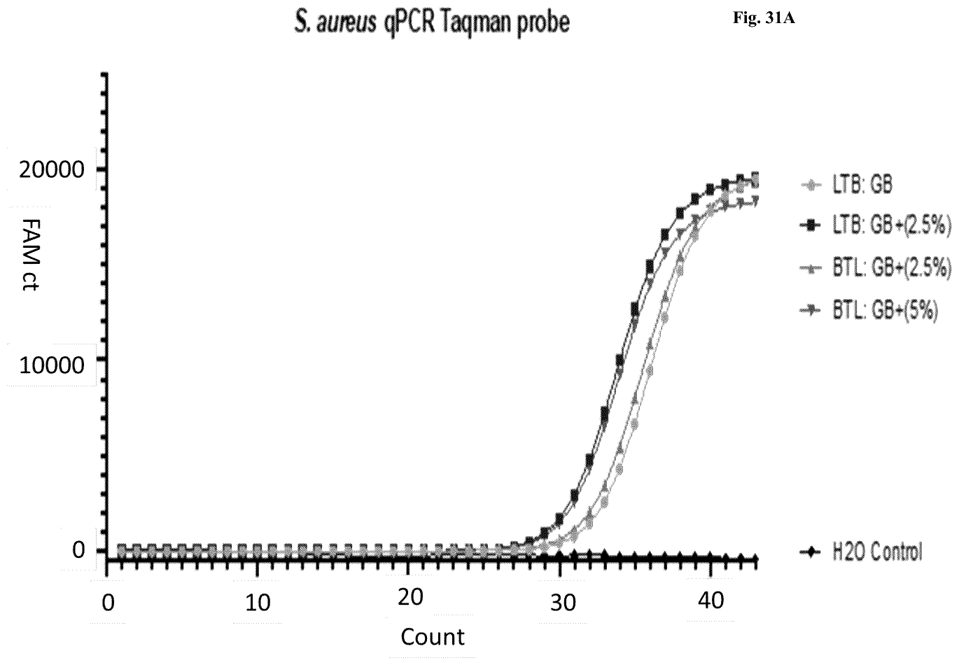

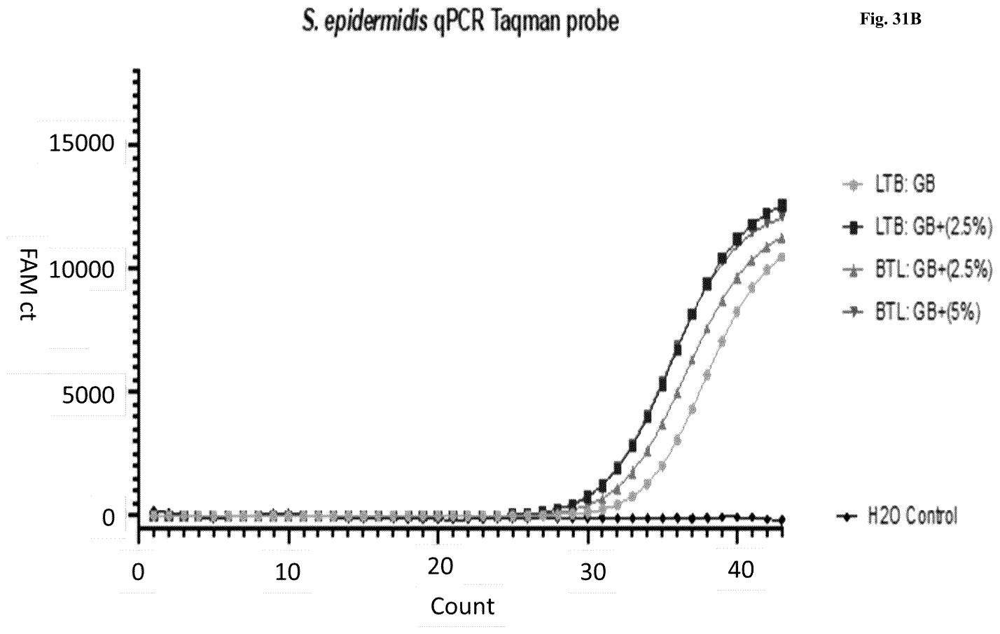

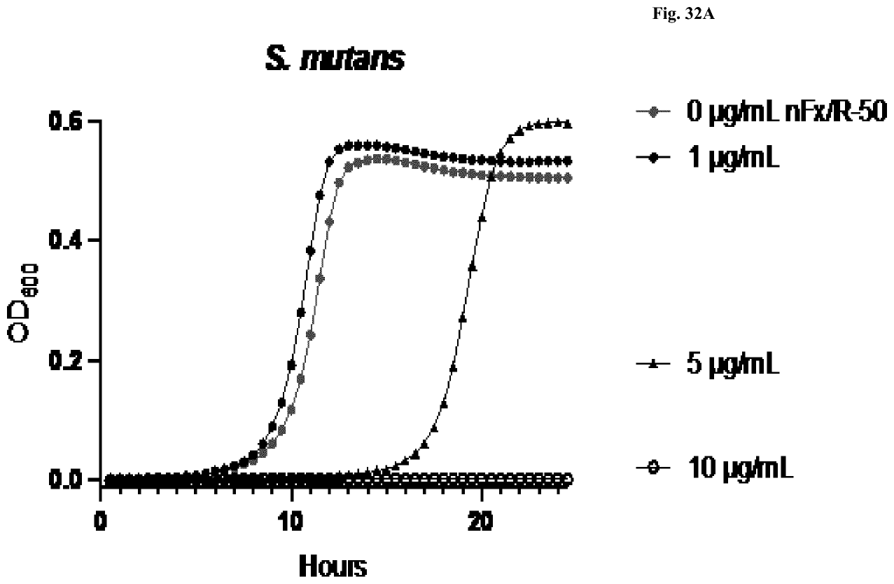

Bacterial samples retrieved from Trial #2 were extracted for DNA using the ZYMObiomics DNA extraction kit and qPCR was conducted using either a S. aureus or S. epidermidis Taqman probe following standard conditions, with results shown in FIGS. 31A-31B.

As expected, no clear differences were found between the DNA collected from samples, which reflects the short reaction time and Hilltop Chamber isolation which prevents the removal of dead bacteria from the skin through shedding and normal hygiene. Since the number of bacteria retrieved is very small, indicated by the 30+ cycles needed to begin measurable amplification, these samples would not be good candidates for PMA treatment and photolysis to differentiate live and dead bacteria. In the actual clinical study, there will be several days between data gathering which will allow time for dead bacteria to be removed from the skin through normal processes.

### Example 41. Reformulation of Eczema Relief Lotion

As shown in Table 68 below, additional formulations of EMO/FFA oil-supplemented off-the-shelf eczema relief lotion were prepared.

TABLE 68
Formulations.

| Experiment # | Name | Base Lotion | H2O | Oat EMO | Flax FFA |
|---|---|---|---|---|---|
| 022822.1 | GB+(4.0%) | 152 g (76%) | 40 g (20%) | 2.0 g (1%) | 6.0 g (3%) |
| 022822.2 | GB+(3.0%) | 154 g (77%) | 40 g (20%) | 1.5 g (0.75%) | 4.5 g (2.25%) |
| 022822.3 | GB+(1.0%) | 156 g (78%) | 42 g (21%) | 0.5 g (0.25%) | 1.5 g (0.75%) |

Formulated lotions were aliquoted into 25 g portions in 50 mL conical tubes, labeled with either #1 (Red Caps, 022822.1), #2 (Blue Caps, 022822.2), or #3 (Green Caps, 022822.3), and given to members of the GlycosBio team as a set with a blank evaluation sheet. The averages of all evaluations are shown in Table 69, with comments listed below the table.

TABLE 69
Comments on Formulae.

| | Formula | Formula | Formula |
|---|---|---|---|
| | 1 | 2 | 3 |
| Texture Ranking: 1 best, 3-worst | 2.13 | 2.50 | 1.63 |
| Texture Comment: | | | |
| Feel/Consistency, Oiliness, | | | |
| Shininess, Runny, Sticky | | | |
| Aesthetic Ranking: 1 best, 3-worst | 2.43 | 2.14 | 1.29 |
| Aesthetic Comment: | | | |
| Smell: strong, nutty, bitter, rich, | | | |
| floral, vanilla, etc. | | | |
| Color: White, Ivory, Off-white, | | | |
| Yellow, Orange, Pink | | | |
| Effect Ranking: 1 best, 3-worst | 2.33 | 1.83 | 1.33 |
| Effect Comment: | | | |
| Hydrating, Dryness/Itch Relief, | | | |
| Soothing, Softening, Irritating | | | |
| Overall Score | 2.30 | 2.16 | 1.41 |

The 4% AO formulation received the following comments: Texture: Shiny×2, Little oily, too dry, more oily, slightly oily; Aesthetic: Bitter, Nutty, mild, fishy, worse smell, less pleasant odor; Effect: Really dry, soft feeling, Nice, not slick, hydrating. The 3% AO: Texture: Sticky, little oily, less oily, well absorbed; Aesthetic: vanilla, mild scent, milder smell; Effect: Relief/Soothing, Slight hydration, best hydration, better absorption. The 1% AO: Texture: Really sticky/shiny, less oily ×2, little oily, shiny, wet, slightly oily; Aesthetic: Floral, mild scent, whiter, medium smelly, less pleasant odor; Effect: hydrating, hydrating/softening, absorbed.

Participants were asked to use the lotions at least once to comment on most of the Texture/Aesthetic/Effect features, with some of the Effect features likely needing multiple applications. The comments reveal some trends towards the 1% AO formulation being less oily and smelling better, with the 4% AO formulation being the more oily and worse smelling. This is reflected in the overall score, in which less AO ranked more positively (score of 1 being the best), but without a significant difference between the 4% and 3% AO.

Table 70 demonstrates the large impact of AO composition in the off-the-shelf eczema relief lotion formulations, where similar quantities of overall recoverable bacteria were cultured from the 4% AO and 3% AO formulations but with a 90% difference between the 4% and 3% AO in terms of % S. aureus upon plating. The 1% AO formulation is mostly consistent with off-the-shelf eczema relief lotion controls, where there is bacterial growth of all species and not a significant impact on the overall composition. 400 mg of lotion in 5 mL PBS with 1×10\hat{ }6 bacteria added with an initial composition of 80% S. aureus, 20% S. epidermidis. 200 mg lotion and 600 mg lotion in 5 mL PBS were also tested, with similar results (not shown).

TABLE 70
Selective Bactericidal Effect of Reformulations.

| | % Bacteria | % |
|---|---|---|
| Lotion | recovered | S. aureus |
| 022822.1: 4% AO | 56 | 1 |
| 022822.2: 3% AO | 62 | 10 |
| 022822.3: 1% AO | 336 | 58 |

The combination of Table 67 (in vivo comparison of 2.5% and 5% AO), 69 (peer evaluations) and 70 (in vitro bactericidal effect), make the case for a 4% AO formulation as the final selection. This is compounded by visual inspection of lotions in which 5% AO results in an oil layer deposition after a prolonged time resulting in a lack of homogeneity, which was not seen in the <5% AO formulations. This 5% AO heterogeneity was demonstrated in FIG. 30, in which the top, middle and bottom portions of the lotion demonstrated different bactericidal effects. In an effort to avoid this phenomenon, particularly in a clinical study in which long-term stability might be tested up to 1 year in length, it is strongly advised to maintain a maximum AO concentration of 4%.

### Example 42. Compounding

Product stability testing will be performed by measuring antimicrobial product activity of top, middle and bottom portions of the compounded formulation, by total aerobic plate counting, and by HPLC.

Tables 71 below provides the formulations that will be compounded for a clinical study.

TABLE 71
Compounding.

| Name | # Of Bottles | Total Volume | Base | | AO #1 | AO #2 | Water |
|---|---|---|---|---|---|---|---|
| Total Needed | | | | | | | |
| GB+ Active | 35 × 100 g | 3500 g | 2660 | g | 35 g | 105 g | 700 g |
| GB+ Control | 35 × 100 g | 3500 g | 2660 | g | 0 | 0 | 840 g |
| GB | 65 × 250 g | 16,250 g | 16,250 | g | 0 | 0 | 0 |
| Minimum needed for 10 patients | | | | | | | |
| GB+ Active | 11 × 100 g | 1100 g | 836 | g | 11 g | 33 g | 220 g |
| GB+ Control | 11 × 100 g | 1100 g | 836 | g | 0 | 0 | 264 g |
| GB | 11 × 250 g | 2750 g | 2750 | g | 0 | 0 | 0 |

GB+ Active from Table 71 was produced in a Sterilite polypropylene bucket that holds 6.6 L. The dimensions were 14⅜″ L×8¼ “W×6” H, weighing 300 g. In a plastic bottle, 700 g of Milli-Q water was heated to 70° C. in the water bath. Full 125 g amber HDPE bottles of AO #1 (Oat EMO) and AO #2 (Flaxseed FFA) were also heated to 70° C. and separated from the bulk inventory of EMO to prevent reuse. The water and oils were poured into the bucket on a scale and homogenized for 1 minute with tilting to insure even dispersion.

Pumps were removed from eight 396 g lotion bottles and the contents were squeezed into the bucket. Only about 325 g of lotion could be retrieved from each bottom by squeezing with force. This process took considerable time, may be advisable to squeeze into a separate bucket followed by addition of the still hot homogenized oil/water mixture.

The mixture was stirred briefly with a large spatula to disperse the oil/water mixture, and the bucket was then homogenized at 15,000 rpm with manual movement of the bucket back and forth while further whipping/dispersing the lotion with a spatula. The lotion as homogenized for 5 minutes, the machine was then allowed to cool for 5 minutes to prevent overheating, followed by 5 additional minutes of homogenization.

No visible oil pockets were observed after this time, so the mixture was bottled into 50 mL conical tubes by scooping with a spatula. The top, middle, and bottom of the bucket mixture were roughly placed into different colored tubes (yellow cap: top, blue cap: middle, green cap: bottom).

Small amounts of oil were noticed to be unincorporated in the thin grooves that line the bottom of the bucket and may account for 2 mL of the total 140 mL of oil (a 1.4% loss). This can be avoided by using a vessel without such grooves.

After 66 hours of settling, the conical tubes were centrifuged at 500×g for 30 seconds to provide a clear delineation at the top of the lotion tube to check for any layers of oil. None were found, indicating excellent incorporation. These samples were be analyzed by HPLC and for antibacterial activity and found to be consistent with antibacterial activity based on the peaks.

### Example 43. Dental Caries Intervention

Traditional oral biocides such as chlorhexidine are in need of replacement due to strong adverse effects and the development of bacterial resistance to these treatments. S. mutans plays a central role in cavity formation through excess production of acid and extracellular polysaccharides, which contribute to enhanced tooth adhesion and biofilm formation. Targeting S. mutans can have an effect on caries incidence in humans, which is applicable to many life stages. By 24 months of age, 84% of children harbor S. mutans, and it has been shown that 52% of children that carried S. mutans at age 3 had caries, while only 3% of children with measurable S. mutans levels had caries at the same age.

Among the many commensal oral bacteria are several Streptococcus species which can play a role in the inhibition of S. mutans over-colonization and infection. For example, S. thermophilus can inhibit biofilm formation of S. mutans UA159 through production of a bacteriocin thermophilin 110. S. salivarius can produce bacteriocins such as enocin, streptin, salivaricin, etc. to inhibited S. pyogenes among others.

Minimal Intervention Paste (MI Paste), a commercial product by GC America, contains casein phosphopeptides which release calcium and phosphate ions to improve remineralization of teeth. A clinical study suggests that caries detection may not be significantly lowered through the use of the product, but that higher salivary fluoride levels suggest that fluoride is being more effectively delivered.

From our initial screen of the nanoemulsion library in which we looked for strong growth inhibitors of S. mutans while not inhibiting the commensal Streptococcus strains, there were not significant trends toward success, with the minor exception of S. salivarius shown in FIG. 9. This growth assay was performed using the commensal strains S. mitts NCIMB 13770, S. orails ATCC 6249, S. salivarius ATCC 13419, S. sanguinis ATCC 10556, and S. thermophilus ATCC 19258, and S. mutans ATCC 35668.

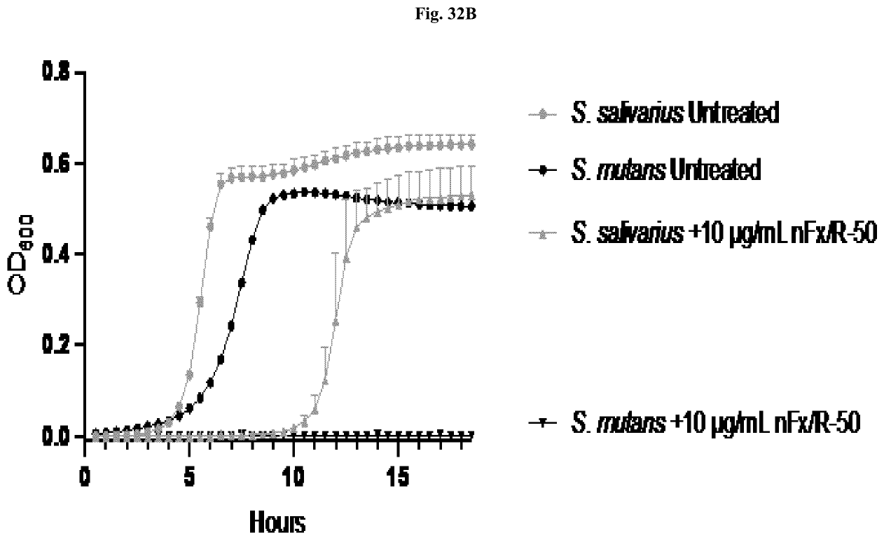

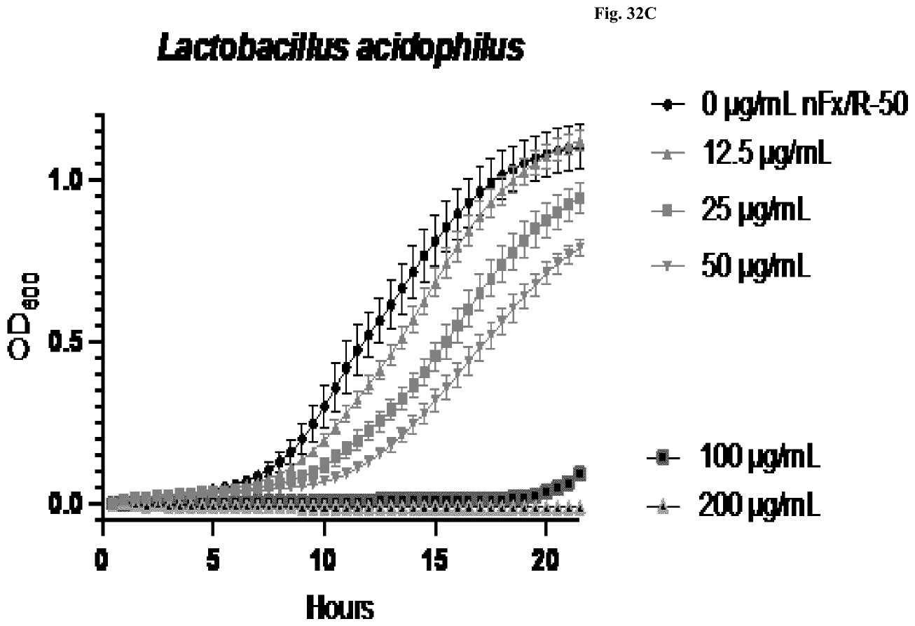

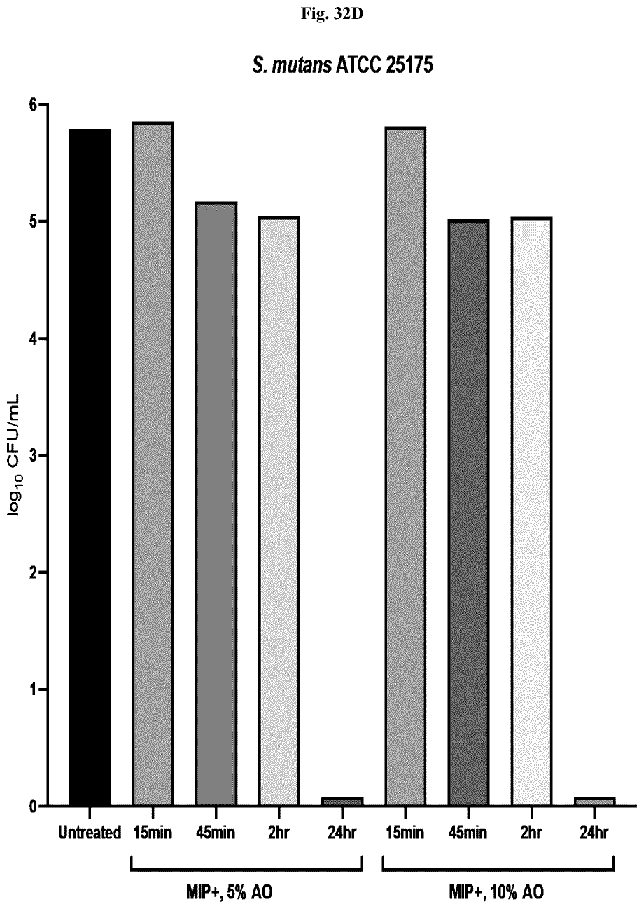

FIGS. 32A-32B clearly show an MIC for S. mutans with nanoemulsion nFx/R-50 of 10 μg/mL, with 5 μg/mL only delaying the growth by 8 hours. FIG. 32B is an overlay of the S. mutans MIC with that of S. salivarius, where 10 μg/mL nFx/R-50 delays the growth by 6 hours, relative to the same concentration completely inhibiting all growth of S. mutans over the entire experiment. Since this window of selective concentrations is very small, this methodology was not further developed. However, based on this result, this oil combination became the basis for prototype development.

To further enhance the features of potential anti-caries paste, the acid-producing species Lactobacillus acidophilus was investigated. Lactobacilli are known to be associated with progression of lesions in dental decay, where the lower pH caused by eating fermentable foods can select for aciduric organisms such as S. mutans and Lactobacillus acidophilus. These bacteria then secrete polysaccharides and acid long after food has been swallowed.

As shown in FIG. 32C, growth inhibition assays in Lactobacilli MRS broth yielded an MIC of 100 μg/mL. This is a 10-fold increase over the MIC for S. mutans, but still within a normal product range, and could potentially have a synergistic effect in vivo or at least be a desired secondary endpoint.

MI Paste prototypes containing Activated Oils were formulated as shown in Table 72, in which AO #1 is Flaxseed MAG (EMO) and AO #2 is Rosehip FFA oil. The 5% AO paste is named MIP+_5% AO, and the 10% AO paste is MIP+_10% AO. The water content needed compared to the GB lotions is much lower, since mixing proceeded with ease. The prototypes generated were surprisingly stable, without any sign of non-incorporated oil after prolonged settling tests.

TABLE 72
MI Paste Prototype Formulations.

| # | INCI Name | Supplier | Function | % | Grams |
|---|---|---|---|---|---|
| 1 | MI Paste | GC America | Base Paste | 85.0% | 17.0 |
| 2 | Activated Oil #1 | GlycosBio | Active | 2.5% | 0.5 |
| 3 | Activated Oil #2 | GlycosBio | Active | 2.5% | 0.5 |
| 4 | Milli-Q Water | JLabs Facility | Solvent | 10% | 2.0 |
| | | | | 100% | 20.0 |
| 1 | MI Paste | GC America | Base Paste | 70.0% | 14.0 |
| 2 | Activated Oil #1 | GlycosBio | Active | 5.0% | 1.0 |
| 3 | Activated Oil #2 | GlycosBio | Active | 5.0% | 1.0 |
| 4 | Milli-Q Water | JLabs Facility | Solvent | 20% | 4.0 |
| | | | | 100% | 20.0 |

5% and 10% AO prototypes were then tested against S. mutans ATCC 25175 in the usual bactericidal assay, with earlier time points taken to assess the speed of action as shown in FIG. 32D.

Both formulations showed a delayed bacterial killing, with only about 1-log reduction after 2 hours. Both formulations had complete elimination of bacteria at 24 hr, indicating a real time-of-action between these two measurements.

Since the MI Paste will be left on the tooth in vivo with a lengthy time of action, this slow bactericidal effect will likely be sufficient to influence the oral microbiota. This compounds with the positive impact that the MI Paste product makes on remineralization of the tooth, allowing fewer weakened areas for bacterial deposition and decay progression.

### Example 44. Biofilm Inhibition of S. mutans

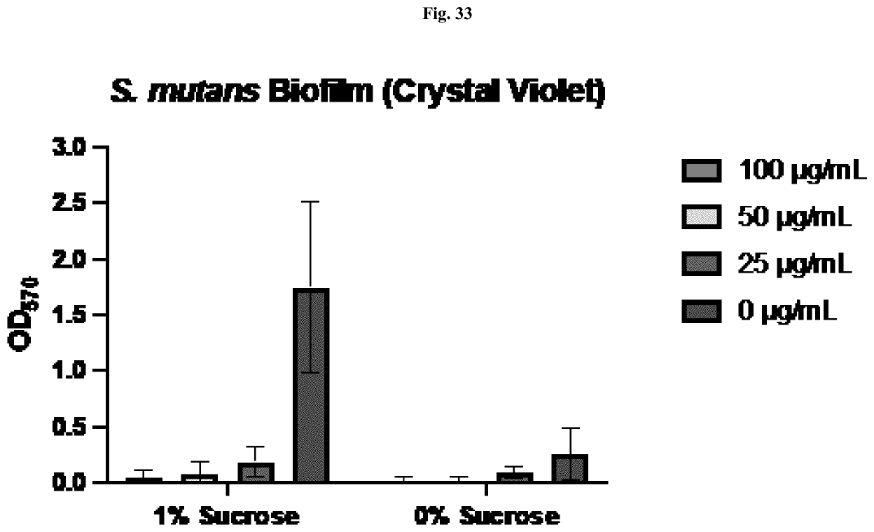

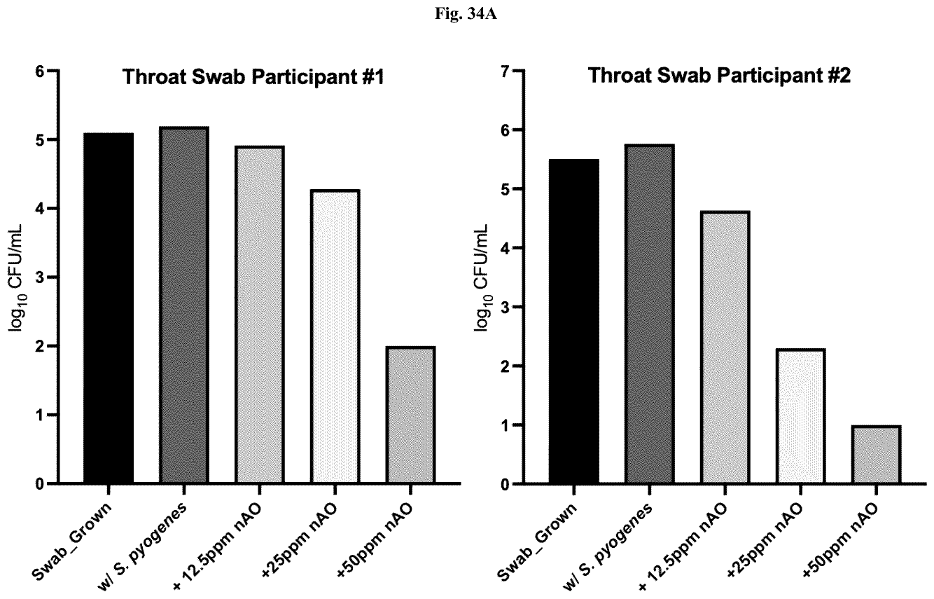

Since most of the bacterial population in tooth decay resides in biofilms and can be difficult to disrupt by antibacterial methods, the ability to nanoemulsion nFx/R-50 to disrupt biofilm formation of S. mutans ATCC 35668 was investigated. Several other bacteria and conditions were tested, but FIG. 33 shows the most informative result which shows a Crystal Violet biofilm assay with absorbance reading at 570 nm, with or without sucrose addition to the growth media, in the presence or absence of nFx/R-50 concentrations as describe above in Example 13.

Without sucrose there is not a significant enough biofilm formation to inhibit, but with sucrose the OD was sufficiently high to draw conclusions. nFx/R-50 has an MBC of 25 μg/mL in this assay, which is only slightly higher than the MIC of 10 μg/mL.

### Example 45. Strep Throat Intervention

A robust nanoemulsion, nFx/R-50 was previously shown in our lab to quickly inhibit and kill S. pyogenes, the causative agent of bacterial pharyngitis. Development of a prototype has been stalled because a clear model system has yet to be identified. Some progress has been made towards developing a model system, shown below.

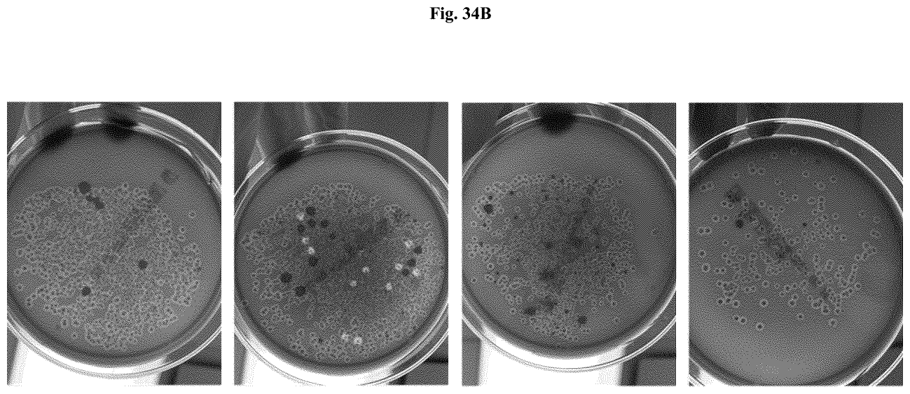

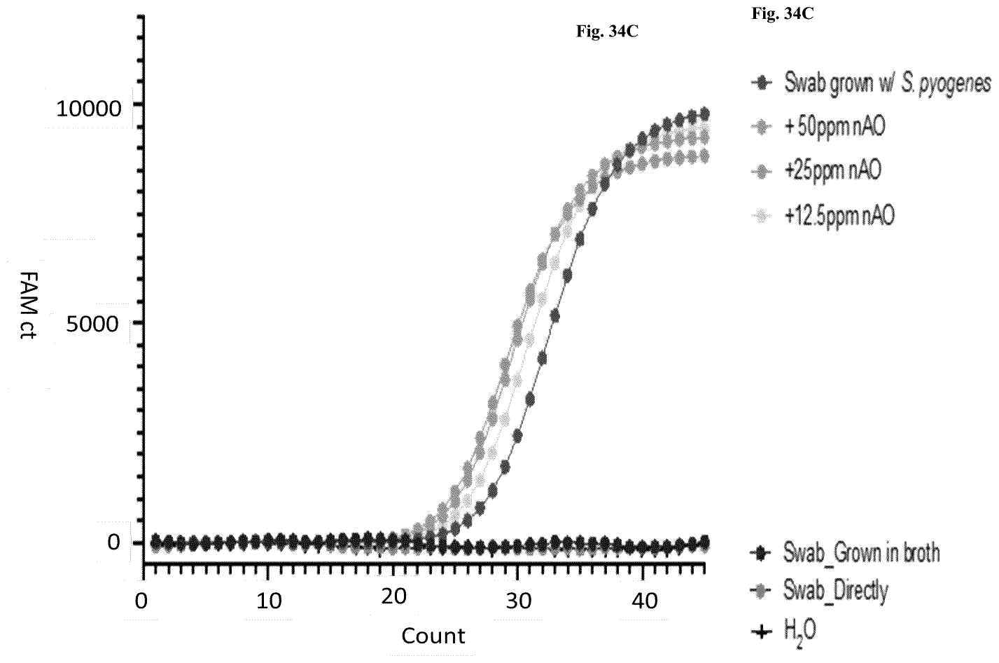

As show in FIGS. 34A-34B, throat swabs were collected from two Glycosbio participants and were grown in tryptic soy broth as a control. Treated samples were spiked with S. pyogenes, and 12.5, 25, or 50 μg/mL of nOa/Fx-25 was added to evaluate the reduction of S. pyogenes in the context of commensal throat species. FIG. 34B shows, from left to right, Swab, Swab with spiked S. pyogenes, Spiked swab treated with 12.5 μg/mL nFx/R-50, Spiked swab treated with 25 μg/mL nFx/R-50.

Participant #1 and #2 both had minimal reductions in overall bacterial population at 12.5 μg/mL, with much more precipitous drop-off for Participant #2 with 25 μg/mL of a nearly 4-log reduction. Participant #1's swab sample had this drop-off at 50 μg/mL. Below the graphs are a visual representation of the data, in which a mixed population is visible in the untreated swab, clear beta-hemolytic circles from spiked-in S. pyogenes can be observed in the 2^{nd} picture, and a disappearance of the beta-hemolytic colonies with very little loss of overall bacteria in the 3^{rd} picture treated with 12.5 μg/mL nAO. The 4^{th} picture shows the significant decrease in viable bacteria with 25 μg/mL nAO.

As with previous results of trying to monitor bactericidal assays by qPCR, the live and dead bacteria could not be differentiated when the lysis step to retrieve gDNA is done directly following the assay. FIG. 34C shows that all the treated and untreated samples overlap in amplification, with no amplification from the swab or broth-grown swab.

To simulate a bacterial pharyngitis environment in vitro, bacteria were chosen based on their known prevalence in normal human samples. Corynebacterium xerosis, Haemophilus parainfluenzae, and Neisseria lactamica were used based on their common detection in throat swabs.

An additional goal is to more effectively culture the species Streptococcus pneumoniae, with the hopes of having a surrogate commensal bacteria that has many of the same properties and disease relation as S. pyogenes but is present in healthy human throats. This could be the basis of a preclinical human model if a robust assay can be developed.

So far it has been easy to grow S. pneumoniae on blood agar plates, but not yet to grow a suitable culture in liquid broth. A Taqman qPCR probe has also been obtained for this species to track multi-day use of a formulation that is applied to the throat.

### Example 46. Broad-Spectrum Antibacterial nEMO for Gram-Negative Bacteria

Table 73 below provides formulations of a nEMO sanitizer with 0.5% nEMO, 29.5% glycerol, and 70% water. This corresponds to 5 mg/mL nEMO, which was effective in our viral screen at killing the following enveloped viruses: Respiratory Syncytial Virus (RSV), Herpes Simplex Virus (HSV), H1N1 flu virus, and SARS-CoV-2 in a 2-minute time period.

TABLE 73
nEMO Sanitizer Formulations.

| # | Ingredient | Supplier | Function | % | Grams |
|---|---|---|---|---|---|
| 1 | nOa/Fx-25 | GlycosBio Inc | Antibacterial | 0.5 | 0.25 |
| 2 | Glycerol | USP Humco | Humectant | 29.5 | 14.75 |
| 3 | Milli-Q Water | Jlabs | Solvent | 70 | 35 |

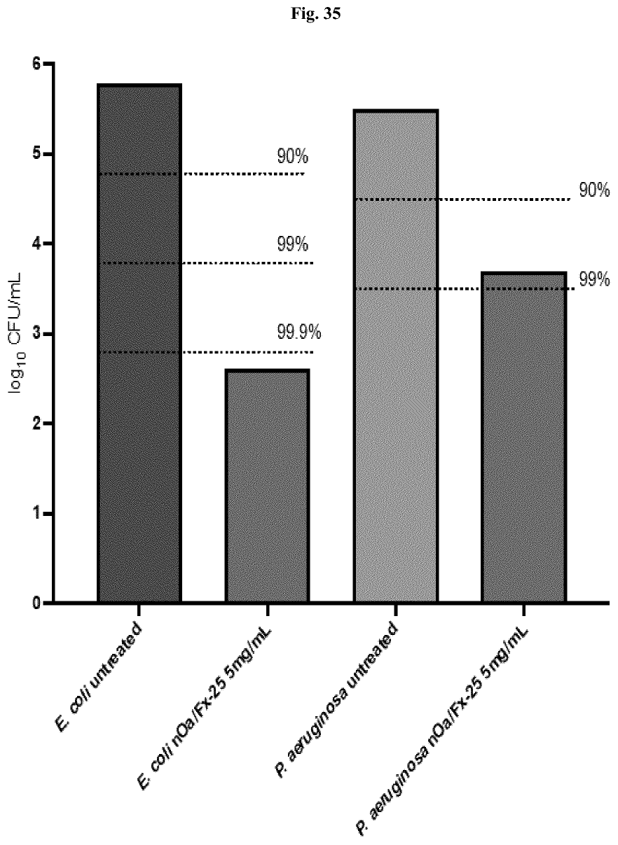

A bactericidal assay was conducted with 10\hat{ }6 E. coli or P. aeruginosa based on conditions used for the Preservative Efficacy Test. Untreated samples were diluted into PBS, while treated samples were diluted directly into the nEMO sanitizer formulation. After a period of 30 minutes, the samples were 10× serially diluted and plated at usual. Results are shown in FIG. 35.

A more rigorous time course will be conducted as a follow-up to this experiment, but it demonstrates that 2-3 log reduction of Gram-negative pathogens can be achieved with nEMOs when used at a high enough concentration.

### Example 47. Selective Bactericidal Activity Against C. acnes

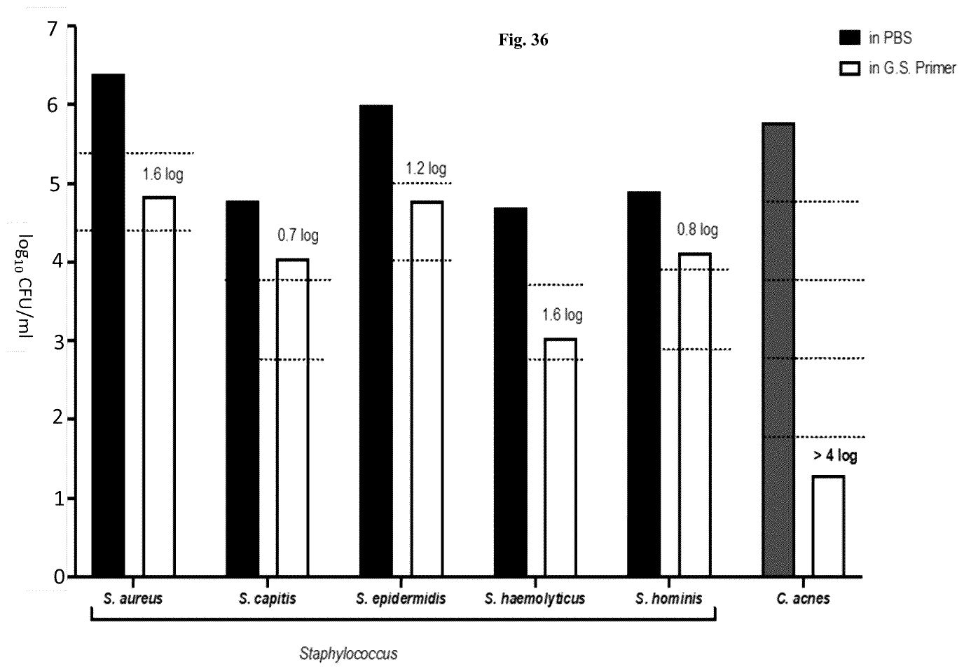

A restorative primer for facial skin that contains 0.5% hempseed EMO and 0.5% rosehip EMO was prepared and tested against bacterial strains as shown in FIG. 36 and Table 74.

TABLE 74

| | Log quantity after | Log Reduction after |
|---|---|---|
| Bacteria | 24 hr in PBS | 24 hr in G.S. Primer |
| S. aureus ATCC 29213 | 6.40 | 1.6 |
| S. capitis ATCC 35661 | 4.78 | 0.7 |
| S. epidermidis ATCC 12228 | 6.00 | 1.2 |
| S. haemolyticus ATCC | 4.70 | 1.6 |
| 29970 | | |
| S. hominis ATCC 27844 | 4.90 | 0.8 |
| C. acnes ATCC 6919 | 5.78 | 4.5 |

Due to the high concentration of EMO in the Primer formulation, some off-target killing of the Staphylococcal species was expected, but all fell within an acceptable range of 0.7 to 1.6 log reduction, which would quickly recover in a skin environment. The 4.5-log reduction in C. acnes that is shown in vitro is likely not reflective of what would occur in the pores of the skin where C. acnes resides, so it makes sense to develop a formula that has the maximum in vitro impact with the hopes of modulating some of the opportunistic pathogen capabilities of C. acnes in normal acne or cystic acne.

### Example 48. Analysis of Non-Oil Ingredients in Enzyme-Modified Oils

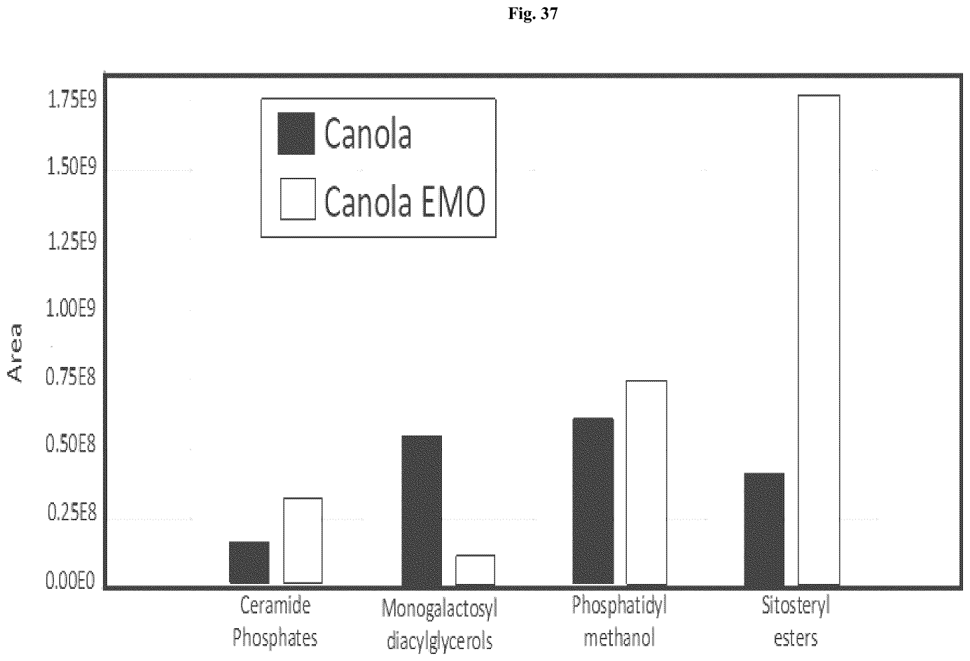

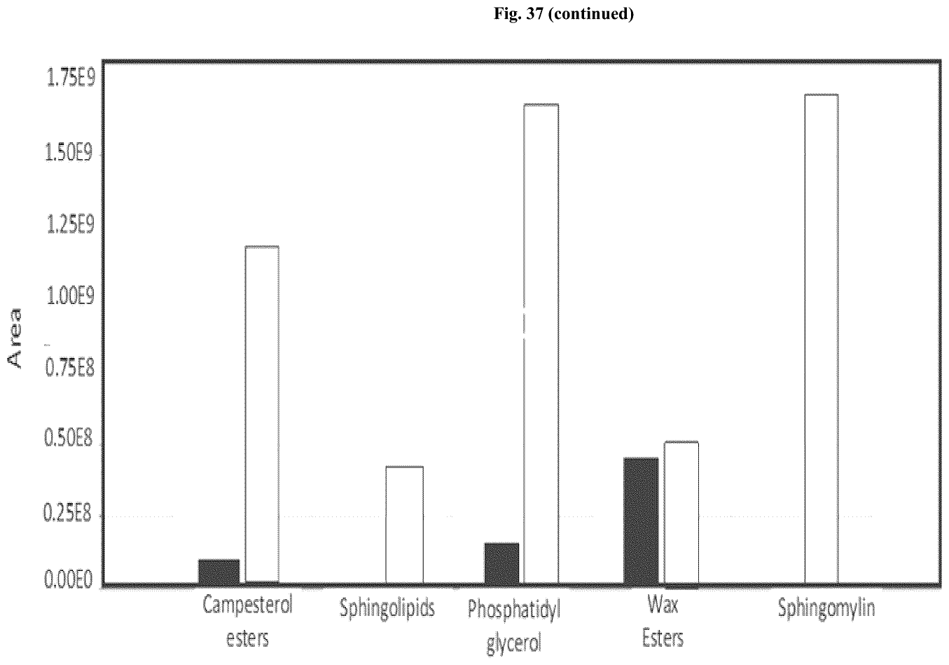

Samples of canola oil or canola EMO were derivatized with 3-picolylamide for LC/MS/MS analysis. The analysis was performed with in positive ionization mode for total fatty acid and lipodomic and negative ionization for lipodomics. Thermo Scientific Freestyle & LipidSearch was used for analysis and the Orbitrap fusion method included data dependent acquisition MS (total fatty acid) & MS. As shown in FIG. 37, non-oil ingredients were generally preserved or enhanced. Coenzyme was also found to be enhanced.

The foregoing description of specific embodiments of the present disclosure has been presented for purpose of illustration and description. The exemplary embodiments were chosen and described in order to best explain the principles of the disclosure and its practical application, to thereby enable others skilled in the art to best utilize the subject matter and various embodiments with various modifications are suited to the particular use contemplated. Different features and disclosures of the various embodiments within the present disclosure may be combined within the scope of the present disclosure.

What is claimed is:

1. A method of treating or preventing a microbial infection in a subject in need thereof, comprising:
administering to the subject an effective amount of an antimicrobial oil composition comprising at least 10% free fatty acids (FFAs) by weight out of the total weight of the antimicrobial oil composition, wherein the antimicrobial oil composition comprises less than 5% triacylglycerols (TAGs) by weight out of the total weight of the antimicrobial oil composition.

2. The method of claim 1, wherein the antimicrobial oil composition further comprises from about 5% to about 70% monoacylglycerols (MAGs) by weight out of the total weight of the antimicrobial oil composition.

3. The method of claim 2, wherein the MAGs are derived from a first oil source.

4. The method of claim 2, wherein the FFAs are derived from a first oil source and a second oil source.

5. The method of claim 4, wherein the first oil source and the second oil source a different oils. T

6. The method of claim 1, wherein the antimicrobial oil composition comprises from about 10% to about 100% FFAs by weight out of the total weight of the antimicrobial oil composition.

7. The method of claim 1, wherein the antimicrobial oil composition is administered in the form of a cream, a lotion, a body wash, or a lubricant, such as vaginal lubricant.

8. The method of claim 1, wherein the antimicrobial oil composition is a plant oil or a mix of two or more plant oils.

9. The method of claim 1, wherein the antimicrobial oil composition comprises a mixture of a first amount of an enzyme-modified oil (EMO) derived from a first oil source and a second amount of a FFA oil derived from a second oil source.

10. The method of claim 1, wherein the antimicrobial oil composition is part of a microemulsion.

11. The method of claim 1, wherein the antimicrobial oil composition is part of a nanoemulsion.

12. The method of claim 11, wherein the antimicrobial oil composition does not include any exogenous additive with surfactant and/or emulsifier properties that is not naturally present in the oil in an amount sufficient to enhance droplet stability.

13. The composition of claim 11, wherein the composition does not include any exogenous additive with thickening or crystallization inhibiting properties that is not naturally present in the oil in an amount sufficient to thicken the composition or inhibit crystallization formation, respectively

14. The method of claim 1, wherein the antimicrobial oil composition has a minimum inhibitory concentration (MIC) of 100 μg/mL or less against a bacteria selected from the group consisting of Bacillus cereus, Streptococcus mutans, Staphylococcus epidermidis, Enterococcus faecalis, Listeria monocytogenes, Streptococcus pyogenes, Streptococcus agalactiae, Cutibacterium acnes, Staphylococcus aureus, Helicobacter pylori, and Pseudomonas aeruginosa, or wherein the antimicrobial oil composition has a minimum bactericidal concentration (MBC) of 100 μg/mL or less against a bacteria selected from the group consisting of Bacillus cereus, Streptococcus mutans, Staphylococcus epidermidis, Enterococcus faecalis, Listeria monocytogenes, Streptococcus pyogenes, Streptococcus agalactiae, Cutibacterium acnes, Staphylococcus aureus, Helicobacter pylori, and Pseudomonas aeruginosa.

15. The method of claim 1, wherein the antimicrobial oil composition selectively inhibits or kills S. aureus more effectively than S. epidermidis, S. hominis, and S. capitis.

16. The method of claim 1, wherein the antimicrobial oil composition is administered topically.

17. An article comprising a condom and a lubricant composition on a surface of the condom, wherein the lubricant composition comprises an oil composition, wherein the oil composition comprises at least 10% free fatty acids (FFAs) by weight out of the total weight of the oil composition, wherein the oil composition comprises less than 5% triacylglycerols (TAGs) by weight out of the total weight of the oil composition.

18. The article of claim 17, wherein the oil composition is a plant-based oil.

19. A method for treating or preventing a skin condition in a subject in need thereof, comprising:
applying a cream composition to the skin of the subject, the cream composition further comprising an oil composition, wherein the oil composition comprises at least 10% free fatty acids (FFAs) by weight out of the total weight of the oil composition, wherein the oil composition comprises less than 5% triacylglycerols (TAGs) by weight out of the total weight of the oil composition.

20. The method of claim 19, wherein the skin condition is atopic dermatitis or eczema.
